(19) United States
(12) Patent Application Publication
(10) Pub. No.: US 2025/0026794 A1
(43) Pub. Date: Jan. 23, 2025

(54) METHODS OF PREVENTING, TREATING, OR REDUCING THE SEVERITY OF CORONAVIRUS DISEASE 2019 (COVID-19)

(71) Applicant: CITY OF HOPE, Duarte, CA (US)

(72) Inventors: Don J. DIAMOND, Duarte, CA (US); Flavia CHIUPPESI, Duarte, CA (US)

(73) Assignee: CITY OF HOPE, Duarte, CA (US)

(21) Appl. No.: 18/711,338

(22) PCT Filed: Nov. 17, 2022
(86) PCT No.: PCT/US2022/080068

§ 371 Date: May 17, 2024

Related U.S. Application Data
(60) Provisional application No. 63/280,533, filed on Nov. 17, 2021.
(60) Provisional application No. 63/280,546, filed on Nov. 17, 2021.

(51) Int. Cl. C07K 14/005; A61K 9/00; A61K 39/00; A61K 39/215; A61P 31/14; C12N 15/86

(52) U.S. Cl. CPC C07K 14/005; A61K 9/0019; A61K 39/215; A61P 31/14; C12N 15/86; A61K 2039/53; A61K 2039/545; A61K 2039/575; C12N 2710/24143; C12N 2770/20022; C12N 2770/20034

(57) ABSTRACT

Disclosed are methods of preventing or treating COVID-19 caused by a coronavirus infection or variants thereof by administration to a subject in need thereof a synthetic MVA-based vaccine. Also disclosed are methods of preventing or treating a coronavirus infection, or increasing an immune response in a subject who has previously received a SARS-CoV-2 vaccine by administration to the subject or a booster dose of a synthetic MVA-based vaccine.

### CROSS-REFERENCE TO RELATED APPLICATIONS

This application claims the benefit of U.S. Provisional Patent Application No. 63/280,533, filed on Nov. 17, 2021, and U.S. Provisional Patent Application No. 63/280,546, filed on Nov. 17, 2021, the entire contents of each of which are incorporated by reference.

### SEQUENCE LISTING

This application contains a Sequence Listing, which was submitted in XML format via Patent Center, and is hereby incorporated by reference in its entirety. The XML copy, created on Nov. 16, 2022, is named 0544358220WO00.xml and is 590 KB in size.

### BACKGROUND

On Feb. 4, 2020, the Secretary of Health and Human Services (HHS) determined that there was a public health emergency concerning the spread of a novel coronavirus, later named “severe acute respiratory syndrome coronavirus 2” (SARS-CoV-2), and the disease it causes was named “Coronavirus Disease 2019” (COVID-19). Since then, SARS-CoV-2 has caused a global pandemic with almost 250M cases and 5M fatalities (as of Nov. 1, 2021). Preventing the incidence of COVID-associated morbidity and mortality while allowing a return to normal activities may best be accomplished by prophylactic vaccination against SARS-CoV-2. Spike (S)-based vaccines appear to protect from hospitalization and severe disease, yet, as virus variants arise with mutations primarily within the virus S-protein, there is concern that vaccine-induced immunity might be insufficient to control disease. To hasten the end of the pandemic and protect against the spread of variants, a preventative SARS-CoV-2 vaccine, which targets both S and the less variant prone nucleocapsid (N) protein, was developed.

Following emergence of Alpha (B.1.1.7), Beta (B.1.351), Gamma (P.1), and Delta (B.1.617), Omicron subvariants have emerged as predominant SARS-CoV-2 VOC, which includes BA.1, BA.2, BA.2 sub-lineages such as BA.2.12.1, BA.4, BA.5, BA.2.75, and more recent subvariants such as BQ.1, BQ.1.1, and XBB. Omicron subvariants have exceptional capacity to evade neutralizing antibodies (NAb) due to numerous mutations in the S protein that dramatically exceed S mutations of other earlier occurring SARS-CoV-2 VOC, thereby posing a unique challenge for COVID-19 vaccination. Several studies reported reduced clinical effectiveness against Omicron variants by approved COVID-19 vaccines, which were designed to elicit protective immunity solely based on the S protein of the Wuhan-Hu-1 reference strain. While waning vaccine efficacy against VOC can be counteracted by repeated booster vaccination, COVID-19 booster vaccines with altered or variant-matched antigen design have been developed to specifically enhance the stimulation of cross-protective immunity against emerging SARS-CoV-2 VOC.

This disclosure provides vaccines or other immunogenic compositions using a synthetic MVA (sMVA) platform capable of expressing immunogenic viral proteins or antigens and administration doses and schedule thereof in preventing SARS-CoV-2 infection and/or COVID-19.

### SUMMARY

Disclosed are methods of preventing or treating COVID-19 caused by a coronavirus infection or variants thereof by administration to a subject in need a synthetic MVA-based vaccine. Also disclosed are methods of preventing or treating a coronavirus infection, or increasing an immune response in a subject who has previously received a SARS-CoV-2 vaccine by administration to the subject or a booster dose of a synthetic MVA-based vaccine.

In some aspects, provided are methods of vaccinating or protecting a subject against coronavirus disease 2019 (COVID-19) caused by a coronavirus infection, comprising administering a composition comprising a synthetic modified vaccinia Ankara (sMVA) vector or virus capable of expressing one or more DNA sequences encoding a spike (S) protein and a nucleocapsid (N) protein or variants or mutants thereof, wherein the method (i) prevents the coronavirus infection or (ii) prevents or reduces the severity of COVID-19 caused by the coronavirus infection. In some embodiments, the method (i) prevents the coronavirus infection. In some embodiments, the method (ii) prevents or reduces the severity of COVID-19 caused by the coronavirus infection.

In some aspects, provided are methods of preventing or reducing the severity of COVID-19 caused by a coronavirus infection in a subject, comprising administering a composition comprising a synthetic modified vaccinia Ankara (sMVA) vector or virus capable of expressing one or more DNA sequences encoding a spike (S) protein and a nucleocapsid (N) protein or variants or mutants thereof, wherein the method (i) prevents the coronavirus infection or (ii) prevents or reduces the severity of COVID-19 caused by the coronavirus infection. In some embodiments, the method (i) prevents the coronavirus infection. In some embodiments, the method (ii) prevents or reduces the severity of COVID-19 caused by the coronavirus infection.

In some aspects, provided are methods of treating COVID-19 in a subject, comprising administering a composition comprising a synthetic modified vaccinia Ankara (sMVA) vector or virus capable of expressing one or more DNA sequences encoding a spike (S) protein and a nucleocapsid (N) protein or variants or mutants thereof.

In some aspects, provided are methods of boosting an immune response to coronavirus infection in a subject, comprising administering a composition comprising a synthetic modified vaccinia Ankara (sMVA) vector or virus capable of expressing one or more DNA sequences encoding a spike (S) protein and a nucleocapsid (N) protein or variants or mutants thereof.

In some embodiments, the coronavirus infection is caused by the Wuhan-Hu-1 reference strain or a variant of concern (VOC) selected from the group consisting of B.1.1.7 (Alpha), B.1.351 (Beta), P.1 (Gamma), B.1.617.2 (Delta), B.1.621 (Mu), C.37 (Lambda), C.1.2, BA.1 (Omicron), BA.2 (Omicron), BA.2.12.1 (Omicron), BA.4 (Omicron), BA.5 (Omicron), BA.2.75 (Omicron), BQ.1 (Omicron), BQ.1.1 (Omicron), and XBB (Omicron).

In some embodiments, the composition is administered to the subject by intramuscular injection, intranasal instillation, intradermal injection, and/or scarification.

In some embodiments, the composition is administered to the subject in a single dose, two doses, three doses, four doses, or more than four doses. In some embodiments, the composition is administered to the subject in a single dose. In some embodiments, the composition is administered to the subject in two doses, wherein one of the doses is a booster dose. In some embodiments, the composition is administered to the subject in three doses, wherein at least one of the doses is a booster dose. In some embodiments, the composition is administered to the subject in four doses, wherein at least one of the doses is a booster dose. In some embodiments, the composition is administered to the subject in more than four doses, wherein at least one of the doses is a booster dose.

In some embodiments, the composition is administered in a prime dose and a first booster dose subsequent to the prime dose. In some embodiments, the interval between the prime dose and the first booster dose is about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, or more than 16 weeks.

In some embodiments, the composition is administered in a prime dose, a first booster dose subsequent to the prime dose, and two or more additional booster doses subsequent to the first booster dose. In some embodiments, the interval between each of the booster doses is about 8 weeks, about 9 weeks, about 10 weeks, about 11 weeks, about 12 weeks, about 13 weeks, about 14 weeks, about 15 weeks, about 16 weeks, about 6 months, about 1 year, or more than about 1 year.

In some embodiments, the prime dose is between about 1.0×10⁷ PFU/dose and 5.0×10⁸ PFU/dose, for example, about 1.0×10⁷ PFU/dose, about 1.5×10⁷ PFU/dose, about 2.0×10⁷ PFU/dose, about 2.5×10⁷ PFU/dose, about 3.0×10⁷ PFU/dose, about 3.5×10⁷ PFU/dose, about 4.0×10⁷ PFU/dose, about 4.5×10⁷ PFU/dose, about 5.0×10⁷ PFU/dose, about 5.5×10⁷ PFU/dose, about 6.0×10⁷ PFU/dose, about 6.5×10⁷ PFU/dose, about 7.0×10⁷ PFU/dose, about 7.5×10⁷ PFU/dose, about 8.0×10⁷ PFU/dose, about 8.5×10⁷ PFU/dose, about 9.0×10⁷ PFU/dose, about 9.5×10⁷ PFU/dose, about 1.0×10⁸ PFU/dose, about 1.5×10⁸ PFU/dose, about 2.0×10⁸ PFU/dose, about 2.5×10⁸ PFU/dose, about 3.0×10⁸ PFU/dose, about 3.5×10⁸ PFU/dose, about 4.0×10⁸ PFU/dose, about 4.5×10⁸ PFU/dose, or about 5.0×10⁸ PFU/dose.

In some embodiments, the first and/or additional booster doses are between 1.0×10⁷ PFU/dose and 5.0×10⁸ PFU/dose, about 1.0×10⁷ PFU/dose, about 1.5×10⁷ PFU/dose, about 2.0×10⁷ PFU/dose, about 2.5×10⁷ PFU/dose, about 3.0×10⁷ PFU/dose, about 3.5×10⁷ PFU/dose, about 4.0×10⁷ PFU/dose, about 4.5×10⁷ PFU/dose, about 5.0×10⁷ PFU/dose, about 5.5×10⁷ PFU/dose, about 6.0×10⁷ PFU/dose, about 6.5×10⁷ PFU/dose, about 7.0×10⁷ PFU/dose, about 7.5×10⁷ PFU/dose, about 8.0×10⁷ PFU/dose, about 8.5×10⁷ PFU/dose, about 9.0×10⁷ PFU/dose, about 9.5×10⁷ PFU/dose, about 1.0×10⁸ PFU/dose, about 1.5×10⁸ PFU/dose, about 2.0×10⁸ PFU/dose, about 2.5×10⁸ PFU/dose, about 3.0×10⁸ PFU/dose, about 3.5×10⁸ PFU/dose, about 4.0×10⁸ PFU/dose, about 4.5×10⁸ PFU/dose, or about 5.0×10⁸ PFU/dose.

In some embodiments, the first and/or additional booster doses are the same dosage as the prime dose or at a lower dosage than the prime dose.

In some embodiments, the subject has previously received a different SARS-CoV-2 vaccine. In some embodiments, the previously received SARS-CoV-2 vaccine is an mRNA-, adenovirus-, or protein-based vaccine. In some embodiments, the previously received SARS-CoV-2 vaccine is Pfizer-BioNTech COVID-19 vaccine (BNT162b2), Moderna COVID-19 vaccine (mRNA-1273), Janssen COVID-19 vaccine (Ad26.COV2.S), Novavax COVID-19 vaccine adjuvanted, or Oxford-AstraZeneca ChAdOx1 nCoV-19 vaccine (AZD1222). In some embodiments, the previously received SARS-CoV-2 vaccine comprises an S antigen or a coding sequence for an S antigen only.

In some embodiments, a Th1-biased immune response is elicited in the subject.

In some aspects, provided are methods of vaccinating or protecting against coronavirus disease 2019 (COVID-19) caused by a coronavirus infection in a subject who has previously received a SARS-CoV-2 vaccine, comprising administering a booster dose of a composition comprising a synthetic modified vaccinia Ankara (sMVA) vector or virus capable of expressing one or more DNA sequences encoding a spike (S) protein and a nucleocapsid (N) protein or variants or mutants thereof, wherein the method (i) prevents the coronavirus infection or (ii) prevents or reduces the severity of COVID-19 caused by the coronavirus infection. In some embodiments, the method (i) prevents the coronavirus infection. In some embodiments, the method (ii) prevents or reduces the severity of COVID-19 caused by the coronavirus infection.

In some aspects, provided are methods of preventing or reducing the severity of COVID-19 caused by a coronavirus infection in a subject who has previously received a SARS-CoV-2 vaccine, comprising administering a booster dose of a composition comprising a synthetic modified vaccinia Ankara (sMVA) vector or virus capable of expressing one or more DNA sequences encoding a spike (S) protein and a nucleocapsid (N) protein or variants or mutants thereof, wherein the method (i) prevents the coronavirus infection or (ii) prevents or reduces the severity of COVID-19 caused by the coronavirus infection. In some embodiments, the method (i) prevents the coronavirus infection. In some embodiments, the method (ii) prevents or reduces the severity of COVID-19 caused by the coronavirus infection.

In some aspects, provided are methods of treating COVID-19 in a subject who has previously received a SARS-CoV-2 vaccine, comprising administering a booster dose of a composition comprising a synthetic modified vaccinia Ankara (sMVA) vector or virus capable of expressing one or more DNA sequences encoding a spike (S) protein and a nucleocapsid (N) protein or variants or mutants thereof.

In some aspects, provided are methods of boosting an immune response to coronavirus infection in a subject who has previously received a SARS-CoV-2 vaccine, comprising administering a booster dose of a composition comprising a synthetic modified vaccinia Ankara (sMVA) vector or virus capable of expressing one or more DNA sequences encoding a spike (S) protein and a nucleocapsid (N) protein or variants or mutants thereof.

In some embodiments, the subject received the previous SARS-CoV-2 vaccine at least one month, at least two months, at least 3 months, at least 4 months, at least 5 months, at least 6 months, at least 7 months, at least 8 months, at least 9 months, at least 10 months, at least 11 months, or at least 12 months before receiving the booster dose.

In some embodiments, the composition is administered to the subject in a single booster dose, two booster doses, three booster doses, four booster doses, or more than four booster doses. In some embodiments, the composition is administered to the subject in a single booster dose. In some embodiments, the composition is administered to the subject in two booster doses. In some embodiments, the composition is administered to the subject in three booster doses. In some embodiments, the composition is administered to the subject in four booster doses. In some embodiments, the composition is administered to the subject in more than four booster doses.

In some embodiments, the interval between each of the doses is about 8 weeks, about 9 weeks, about 10 weeks, about 11 weeks, about 12 weeks, about 13 weeks, about 14 weeks, about 15 weeks, about 16 weeks, about 6 months, about 1 year, or more than about 1 year.

In some embodiments, the first booster dose is between about 1.0×10⁷ PFU/dose and 5.0×10⁸ PFU/dose, for example, about 1.0×10⁷ PFU/dose, about 1.5×10⁷ PFU/dose, about 2.0×10⁷ PFU/dose, about 2.5×10⁷ PFU/dose, about 3.0×10⁷ PFU/dose, about 3.5×10⁷ PFU/dose, about 4.0×10⁷ PFU/dose, about 4.5×10⁷ PFU/dose, about 5.0×10⁷ PFU/dose, about 5.5×10⁷ PFU/dose, about 6.0×10⁷ PFU/dose, about 6.5×10⁷ PFU/dose, about 7.0×10⁷ PFU/dose, about 7.5×10⁷ PFU/dose, about 8.0×10⁷ PFU/dose, about 8.5×10⁷ PFU/dose, about 9.0×10⁷ PFU/dose, about 9.5×10⁷ PFU/dose, about 1.0×10⁸ PFU/dose, about 1.5×10⁸ PFU/dose, about 2.0×10⁸ PFU/dose, about 2.5×10⁸ PFU/dose, about 3.0×10⁸ PFU/dose, about 3.5×10⁸ PFU/dose, about 4.0×10⁸ PFU/dose, about 4.5×10⁸ PFU/dose, or about 5.0×10⁸ PFU/dose.

In some embodiments, the second or additional booster doses are between 1.0×10⁷ PFU/dose and 5.0×10⁸ PFU/dose, about 1.0×10⁷ PFU/dose, about 1.5×10⁷ PFU/dose, about 2.0×10⁷ PFU/dose, about 2.5×10⁷ PFU/dose, about 3.0×10⁷ PFU/dose, about 3.5×10⁷ PFU/dose, about 4.0×10⁷ PFU/dose, about 4.5×10⁷ PFU/dose, about 5.0×10⁷ PFU/dose, about 5.5×10⁷ PFU/dose, about 6.0×10⁷ PFU/dose, about 6.5×10⁷ PFU/dose, about 7.0×10⁷ PFU/dose, about 7.5×10⁷ PFU/dose, about 8.0×10⁷ PFU/dose, about 8.5×10⁷ PFU/dose, about 9.0×10⁷ PFU/dose, about 9.5×10⁷ PFU/dose, about 1.0×10⁸ PFU/dose, about 1.5×10⁸ PFU/dose, about 2.0×10⁸ PFU/dose, about 2.5×10⁸ PFU/dose, about 3.0×10⁸ PFU/dose, about 3.5×10⁸ PFU/dose, about 4.0×10⁸ PFU/dose, about 4.5×10⁸ PFU/dose, or about 5.0×10⁸ PFU/dose.

In some embodiments, the second and/or additional booster doses are the same dosage as the first booster dose or at a lower dosage than the first booster dose.

In some aspects, provided are compositions for use in a method of vaccinating or protecting a subject against coronavirus disease 2019 (COVID-19) caused by a coronavirus infection, wherein the composition comprise a synthetic modified vaccinia Ankara (sMVA) vector or virus capable of expressing one or more DNA sequences encoding a spike (S) protein and a nucleocapsid (N) protein or variants or mutants thereof, and wherein the composition (i) prevents the coronavirus infection or (ii) prevents or reduces the severity of COVID-19 caused by the coronavirus infection. In some embodiments, the composition (i) prevents the coronavirus infection. In some embodiments, the composition (ii) prevents or reduces the severity of COVID-19 caused by the coronavirus infection.

In some aspects, provided are compositions for use in a method of preventing or reducing the severity of COVID-19 caused by a coronavirus infection in a subject, wherein the composition comprises a synthetic modified vaccinia Ankara (sMVA) vector or virus capable of expressing one or more DNA sequences encoding a spike (S) protein and a nucleocapsid (N) protein or variants or mutants thereof, and wherein the composition (i) prevents the coronavirus infection or (ii) prevents or reduces the severity of COVID-19 caused by the coronavirus infection. In some embodiments, the composition (i) prevents the coronavirus infection. In some embodiments, the composition (ii) prevents or reduces the severity of COVID-19 caused by the coronavirus infection.

In some aspects, provided are compositions for use in a method of treating COVID-19 in a subject, wherein the composition comprises a synthetic modified vaccinia Ankara (sMVA) vector or virus capable of expressing one or more DNA sequences encoding a spike (S) protein and a nucleocapsid (N) protein or variants or mutants thereof.

In some aspects, provided are compositions for use in a method of boosting an immune response to coronavirus infection in a subject, wherein the composition comprises a synthetic modified vaccinia Ankara (sMVA) vector or virus capable of expressing one or more DNA sequences encoding a spike (S) protein and a nucleocapsid (N) protein or variants or mutants thereof.

In some aspects, provided are booster doses of a composition for use in a method of vaccinating or protecting against coronavirus disease 2019 (COVID-19) caused by a coronavirus infection in a subject who has previously received a SARS-CoV-2 vaccine, wherein the composition comprises a synthetic modified vaccinia Ankara (sMVA) vector or virus capable of expressing one or more DNA sequences encoding a spike (S) protein and a nucleocapsid (N) protein or variants or mutants thereof, and wherein the composition (i) prevents the coronavirus infection or (ii) prevents or reduces the severity of COVID-19 caused by the coronavirus infection. In some embodiments, the booster dose of the composition (i) prevents the coronavirus infection. In some embodiments, the booster dose of the composition (ii) prevents or reduces the severity of COVID-19 caused by the coronavirus infection.

In some aspects, provided are booster doses of a composition for use in a method of preventing or reducing the severity of COVID-19 caused by a coronavirus infection in a subject who has previously received a SARS-CoV-2 vaccine, wherein the composition comprises a synthetic modified vaccinia Ankara (sMVA) vector or virus capable of expressing one or more DNA sequences encoding a spike (S) protein and a nucleocapsid (N) protein or variants or mutants thereof, and wherein the composition (i) prevents the coronavirus infection or (ii) prevents or reduces the severity of COVID-19 caused by the coronavirus infection. In some embodiments, the booster dose of the composition (i) prevents the coronavirus infection. In some embodiments, the booster dose of the composition (ii) prevents or reduces the severity of COVID-19 caused by the coronavirus infection.

In some aspects, provided are booster doses of a composition for use in a method of treating COVID-19 in a subject who has previously received a SARS-CoV-2 vaccine, wherein the composition comprises a synthetic modified vaccinia Ankara (sMVA) vector or virus capable of expressing one or more DNA sequences encoding a spike (S) protein and a nucleocapsid (N) protein or variants or mutants thereof.

In some aspects, provided are booster doses of a composition for use in a method of boosting an immune response to coronavirus infection in a subject who has previously received a SARS-CoV-2 vaccine, wherein the composition comprises a synthetic modified vaccinia Ankara (sMVA) vector or virus capable of expressing one or more DNA sequences encoding a spike (S) protein and a nucleocapsid (N) protein or variants or mutants thereof.

### BRIEF DESCRIPTION OF THE DRAWINGS

FIG. 1 illustrates trial profile (Consort diagram).

FIGS. 2A-2D show S, N, S or N, and receptor-binding domain (RBD) seroconversion rates in different treatment groups. The serum samples were evaluated for the presence of S-, RBD-, and N-specific IgG by ELISA and endpoint titers quantified. Seroconversion is shown as a four-fold increase in S (FIG. 2A), N (FIG. 2B), S or N (FIG. 2C), and RBD (FIG. 2D) IgG titers from day 0. Successful seroconversion was defined as a post-boost four-fold increase in S or N IgG titers from day 0. Post-prime is considered any time before booster vaccination, post-boost is considered the first month post-boost vaccination. DL, dose level; P, placebo.

FIGS. 3A-3D show sera SARS-CoV-2-specific binding and neutralizing antibody responses following COH04S1 vaccination with different DL and schedules. Spike (S, FIG. 3A), receptor binding domain (RBD, FIG. 3B) and nucleocapsid (N, FIG. 3C) IgG endpoint titers following COH04S1 vaccination were quantified by ELISA. SARS-CoV-2-specific neutralizing antibody titers (FIG. 3D) were evaluated using a SARS-CoV-2 pseudovirus based on the Wuhan S sequence with D614G substitution. Box plots extends from the 25th to the 75th percentiles, median values are shown as a line (key geometric means are discussed in the text), whiskers extend from minimum to maximum values. Individual values are superimposed. Reported are statistical testing results using Wilcoxon rank sum paired test and comparing each time point to baseline (day 0). Significance levels are indicated as follow: *=p<0.05, **=p<0.01, ***=p<0.001, ****=p<0.0001. Exact p values are shown in Tables 7-9. Dotted lines represent the lower limit of quantification. Arrowheads represent time of vaccination. DL, dose level; P, placebo.

FIGS. 4A-4D show the neutralizing responses to VOCs in sentinels. SARS-CoV-2-specific neutralizing antibody titers (NT50) to VOC were evaluated in samples from sentinel subjects using a SARS-CoV-2 PsV based on Wuhan S sequence with D614G substitution (FIG. 4A), Alpha (B.1.1.7) VOC originally isolated in the UK (FIG. 4B), Beta (B.1.351) VOC originally isolated in the RSA (FIG. 4C), and Gamma (P.1) VOC originally isolated in Brazil (FIG. 4D). Box plots extends from the 25^{th} to the 75^{th} percentiles, median values are shown as a line, whiskers extend from minimum to maximum values. Individual values are superimposed. Arrowheads represent time of vaccination. Dotted lines represent the lower limit of quantification. DL, dose level.

FIGS. 5A-5D show IFNγ and IL-4 T cell responses following COH04S1 vaccination with different DL and schedules. Spike(S)- (FIGS. 5A, 5C) and nucleocapsid (N) (FIGS. 5B, 5D)-specific T cells were quantified by IFNγ/IL-4 ELISPOT upon PBMCs stimulation with S and N peptide libraries. Shown are spot forming cells per 10⁶ PBMCs that were obtained after subtraction of spots in unstimulated controls from stimulated samples. Box plots extends from the 25^{th} to the 75^{th} percentiles, median values are shown as a line, whiskers extend from minimum to maximum values. Individual values are superimposed. Reported are statistical testing results using Wilcoxon rank sum paired test and comparing each time point to baseline (day 0). Significance levels are indicated as follow: *=p<0.05, **=p<0.01, ***=p<0.001, ****=p<0.0001. Exact p values are shown in Tables 11-14. Dotted lines represent the arbitrary threshold for positive response (50 spots/10⁶ PBMCs). Arrowheads represent time of vaccination. DL, dose level. P, placebo.

FIGS. 6A-6B show Spike and Nucleocapsid IFNγ/IL-4 T cell ratio. S- and N-specific IFNγ and IL-4 T cells were quantified by ELISPOT and ratios of S- (FIG. 6A) and N- (FIG. 6B) specific T cells secreting IFNγ and IL-4 calculated. Box plots extends from the 25^{th} to the 75^{th} percentiles, median values are shown as a line, whiskers extend from minimum to maximum values. Individual values are superimposed. Dotted lines indicate a ratio of 1. A Th1-biased response is >1, a Th2-biased response is <1. DL, dose level. P, placebo.

FIGS. 7A-7B show IFNγ/IL-4 T cell responses to SARS-CoV-2 membrane peptide library. Membrane-specific T cells were quantified by IFNγ (FIG. 7A) and IL-4 (FIG. 7B) ELISPOT upon PBMCs stimulation with a membrane peptide library. Shown are spot forming cells per 10⁶ PBMCs that were obtained after subtraction of spots in unstimulated controls from stimulated samples. Box plots extends from the 25^{th} to the 75^{th} percentiles, median values are shown as a line, whiskers extend from minimum to maximum values. Individual values are superimposed. Dotted lines represent the arbitrary threshold for positive response (50 spots/10⁶ PBMCs). Arrowheads represent time of vaccination. DL, dose level. P, placebo.

FIGS. 8A-8B show humoral and cellular responses in placebo recipients. IgG binding antibody titers specific for S, RBD, and N, and neutralizing antibody titers were evaluated in placebo recipients (N=5) up to day 56 post-prime immunization (FIG. 8A). Box plots extends from the 25^{th} to the 75^{th} percentiles, median values are shown as a line, whiskers extend from minimum to maximum values. Individual values are superimposed. Dotted line represents the lower limit of detection for the assays (ELISA=150, pvNT50=20). S- and N-specific T cells secreting IFNγ or IL-4 were quantified by ELISPOT (FIG. 8B). Dotted line represents the arbitrary threshold for positivity (50 spots/10⁶ cells). Arrowheads indicate time of placebo vaccinations.

FIGS. 9A-9D show Spike and Nucleocapsid CD137+ T cells in sentinels. Levels of S- and N-specific CD4+CD137+ and CD8+CD137+ T cells were measured in available samples from sentinel volunteers vaccinated with COH04S1 DL1 (4), DL2 (7), and DL3 (4). Shown are CD4+CD137+ and CD8+CD137+ T cell counts per μl of blood. Black lines indicate median values, lines indicate interquartile ranges. Two-sided, Wilcoxon rank sum paired test was performed and each time point was compared to baseline. P values are indicated above each time point. ns=not significant (p>0.05). Arrowheads indicate time of vaccinations.

FIGS. 10A-10C show humoral and cellular immune responses induced by COH04S1 in DL1-vaccinated volunteers with 28 or 56 days dose interval, and DL2/DL3 vaccinated individuals. FIG. 10A: Spike (S), receptor binding domain (RBD), and nucleocapsid (N)-specific binding antibody endpoint tiers. FIG. 10B: Neutralizing antibody titers (NT50) measured using a SARS-CoV-2 pseudovirus based on the original Wuhan Spike sequence with D614G substitution. FIG. 10C: Cellular immunity to Spike, Nucleocapsid and Membrane as measured using IFNγ ELISPOT.

FIGS. 11A-11C show comparative evaluation of post-boost humoral and cellular responses induced by COH04S1 at different doses and schedules in healthy volunteers. N=17 volunteers were immunized with DL1 two times in a 28-day interval. N=10 volunteers were immunized with two DL1 doses of which the second dose was delayed to day 56 (N=9) or day 90 (N=1). N=19 volunteers were immunized with two doses of DL2 or DL3 with a 28-day interval. Shown are binding antibodies (FIG. 11A), neutralizing antibodies (FIG. 11B), and cellular responses (FIG. 11C) measured at boost and up to 5 months post-boost. Pre-boost immune responses were omitted.

FIG. 12 shows binding antibody responses in COH04S1 DL1-immunized volunteers receiving one or more EUA vaccines at day 56 or beyond.

FIG. 13 shows neutralizing antibody responses in COH04S1 DL1-immunized volunteers receiving an EUA vaccine at day 56.

FIG. 14 shows IFNγ-specific cellular responses in COH04S1 DL1-immunized volunteers receiving one or more EUA vaccines at day 56 or beyond.

FIG. 15 is a study schema for COH04S1 investigational COVID-19 vaccine boost: AE—adverse events, EOT—end of treatment, PIA—primary immune assessment.

FIG. 16 shows the timeline following up with the participants who received the booster dose of COH04S1. AE: adverse events; EOT: end of treatment; PIA: primary immune assessment.

FIGS. 17A-17C show sMVA construction. FIG. 17A is a schematic of an MVA genome. The MVA genome is about 178 kbp in length and contains about 9.6 kbp inverted terminal repeat (ITR) sequences. FIG. 17B shows the three sMVA fragments, F1, F2, and F3. The three subgenomic sMVA fragments (F1-F3) comprise about 60 kbp of the left, central, and right parts of the MVA genome as indicated. sMVA F1/F2 and F2/F3 share about 3 kbp overlapping homologous sequences for recombination (dotted crossed lines). Approximate genome positions of commonly used MVA insertion (Del2, IGR69/70, Del3) are indicated. FIG. 17C shows terminal concatemer resolution-hairpin loop-concatemer resolution (CR/HL/CR) sequences. Each of the sMVA fragments contains at both ends a sequence composition comprising a duplex copy of the MVA terminal HL flanked by CR sequences. BAC=bacterial artificial chromosome vector.

FIG. 18 shows the DNA sequence of an sMVA backbone with possible locations of insertion sites Del2, IGR69/70, and Del3, according to some embodiments.

FIG. 19 shows the DNA sequence of an F1 nucleotide fragment that includes a portion of the full MVA backbone sequence flanked by an MVA CR/HL/CR sequence with insertion sites, according to some embodiments.

FIG. 20 shows the DNA sequence of an F2 nucleotide fragment that includes a portion of the full MVA backbone sequence flanked by an MVA CR/HL/CR sequence with insertion sites, according to some embodiments.

FIG. 21 shows the DNA sequence of an F3 nucleotide fragment that includes a portion of the full MVA backbone sequence flanked by an MVA CR/HL/CR sequence with insertion sites, according to some embodiments.

FIG. 22 shows Spike (S), receptor binding domain (RBD), and nucleocapsid (N) binding antibody titers (binding antibody units (BAU)/ml) in COH04S1 boosted volunteers at different time points post-vaccination. d=day. *=value above threshold, needs to be re-analyzed.

FIG. 23 shows a statistical evaluation of spike (S), receptor binding domain (RBD), and nucleocapsid (N) binding antibody titers (binding antibody units (BAU)/ml) in COH04S1 boosted volunteers at different time points post-vaccination. Wilcoxon matched pairs signed rank test was used. P values<0.05 are indicated above the bars. Dotted lines indicate the IgG titers measured in WHO 20/150 standard with high titers.

FIG. 24 shows fold-increase in Spike-specific titers compared to baseline as evaluated using COH IgG quantitative ELISA (left) and OrthoVitros quantitative ELISA (right). Dotted line marks a five-fold increase in titers.

FIG. 25 shows neutralizing antibodies against SARS-CoV-2 ancestral strain (D614G), Beta, Delta, and Omicron BA.1 variants in serum of individual volunteers at baseline and at different time points post-vaccination.

FIG. 26 shows a statistical evaluation of neutralizing antibodies against SARS-CoV-2 ancestral strain (D614G), Beta, Delta, and Omicron BA.1 variants in serum of COH04S1 vaccinated volunteers at baseline and at different time points post-vaccination.

FIG. 27 shows Spike (S)-, nucleocapsid (N)-, and membrane (M)-specific T cells evaluation using IFNγ ELISPOT on PBMC samples from individual COH04S1 boosted volunteers at different time points post-vaccination.

FIG. 28 shows a statistical evaluation of spike (S)-, nucleocapsid (N)-, and membrane (M)-specific T cells secreting IFNγ and IL-4 in PBMC samples of COH04S1 boosted volunteers at baseline and different time points post-vaccination.

FIG. 29 shows a statistical evaluation of spike (S)-, and nucleocapsid (N)-specific activation-induced marker (AIM)+ T cells in PBMC samples of COH04S1 boosted volunteers at baseline and at different time points post-vaccination.

### DETAILED DESCRIPTION

Disclosed herein are methods of vaccinating or protecting a subject against COVID-19 caused by a coronavirus, where the method results in preventing, treating, or reducing the severity of COVID-19 caused by a coronavirus infection or prevents infection by the coronavirus. The coronavirus infection that causes COVID-19 may be SARS-CoV-2 or any variants thereof according to the embodiments disclosed herein. In some embodiments, the coronavirus infection treated by the methods and compositions disclosed herein is caused by a variant of SARS-CoV-2. For example, a variant of SARS-CoV-2 may be a variant of concern including but not limited to B.1.1.7 (Alpha), B.1.351 (Beta), P.1 (Gamma), B.1.617.2 (Delta), B.1.621 (Mu), C.37 (Lambda), C.1.2, BA.1 (Omicron), BA.2 (Omicron), BA.2.12.1 (Omicron), BA.4 (Omicron), BA.5 (Omicron), BA.2.75 (Omicron), BQ.1 (Omicron), BQ.1.1 (Omicron), and XBB (Omicron).

The methods disclosed herein include a step of administering to the subject a composition that includes a recombinant synthetic modified vaccinia Ankara (sMVA) vector or reconstituted virus comprising, expressing, or is capable of expressing one or more heterologous DNA sequences encoding a SARS-CoV-2 spike (S) protein and a SARS-CoV-2 nucleocapsid (N) protein, or variants or mutants of the S protein and N protein. In some embodiments, the composition is a vaccine composition or any immunogenic composition capable of providing full or partial protection against infection by a coronavirus that causes COVID-19. In other embodiments, the composition is a vaccine composition or any immunogenic composition capable of providing full or partial protection from developing moderate or severe COVID-19 symptoms. In other embodiments, the composition is a vaccine composition or any immunogenic composition capable of providing protection from hospitalization or death as a result of developing COVID-19.

The composition may be administered to the subject in any suitable manner. In some embodiments, the composition is administered to the subject parenterally, e.g., by intramuscular injection. In some embodiments, the composition is administered to the subject by intranasal instillation. In some embodiments, the composition is administered to the subject by intradermal injection. In some embodiments, the composition is administered to the subject by scarification.

The compositions disclosed herein may be given to a subject as a single, stand-alone dose. Thus, in some embodiments, the composition is administered to the subject as a single dose. In other embodiments, the compositions may be given as a multiple-dose regimen. For example, in some embodiments, the composition is administered to the subject as a prime dose followed by a booster dose. In some embodiments, the composition is administered to the subject as a prime dose, followed by a first booster dose and a second booster dose. In some embodiments, one or more additional doses are administered to the subject after administration of the prime and booster doses.

According to the embodiments disclosed herein, the compositions for preventing, treating, or reducing the severity of COVID-19 caused by SARS-CoV-2 (or variants thereof) disclosed herein may be interchangeable with other commercially available COVID-19 vaccine compositions, such that the prime dose is different than the booster dose or doses, or such that the booster dose or doses are different from each other or the prime dose. In other words, each dose may be a different vaccine composition. In some embodiments, the subject has previously received a different SARS-CoV-2 vaccine before administration of the vaccine compositions disclosed herein. For example, the previously received SARS-CoV-2 vaccine is an mRNA vaccine or a vaccine composition comprising the S antigen only. In other embodiments, the subject receives a different SARS-CoV-2 vaccine after a prime dose of the compositions is given. The compositions disclosed herein may be given as any one or more of the doses administered to a subject.

In a multiple-dose regimen the first (or only) booster dose may be administered such that the interval between the prime dose and the booster is about 2 weeks, about 3 weeks, about 4 weeks, or about 30 days. Alternatively, the booster administration may be delayed, such that the interval between the prime dose and the booster is greater than 30 days, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, or longer than 16 weeks. In some embodiments, the interval between the prime dose and the booster is about 90 days or longer than 90 days. In certain embodiments, the interval between the prime dose and the booster is about 8 weeks.

In some embodiments, the multiple-dose regimen includes one or more additional booster doses (e.g., a second booster dose, a third booster dose, and so on). Said booster doses may be administered such that the interval between each booster dose is delayed as compared to the interval between the prime dose and the first booster. In certain embodiments, the interval between each booster dose is about 8 weeks, about 9 weeks, about 10 weeks, about 11 weeks, about 12 weeks, about 13 weeks, about 14 weeks, about 15 weeks, about 16 weeks, or longer than 16 weeks. In some embodiments, the interval between each booster dose is about 90 days or longer than 90 days. In some embodiments, the interval between each booster dose is about 90 days or longer than 90 days. In certain embodiments, the interval between each booster dose is between about 6 months to about 1 year. The interval between each booster may be on an annual or semi-annual schedule to account for additional variants that may arise each year.

In some embodiments, the prime dose is between 1.0×10⁷ PFU/dose and 5.0×10⁸ PFU/dose, for example, about 1.0×10⁷ PFU/dose, about 1.5×10⁷ PFU/dose, about 2.0×10⁷ PFU/dose, about 2.5×10⁷ PFU/dose, about 3.0×10⁷ PFU/dose, about 3.5×10⁷ PFU/dose, about 4.0×10⁷ PFU/dose, about 4.5×10⁷ PFU/dose, about 5.0×10⁷ PFU/dose, about 5.5×10⁷ PFU/dose, about 6.0×10⁷ PFU/dose, about 6.5×10⁷ PFU/dose, about 7.0×10⁷ PFU/dose, about 7.5×10⁷ PFU/dose, about 8.0×10⁷ PFU/dose, about 8.5×10⁷ PFU/dose, about 9.0×10⁷ PFU/dose, about 9.5×10⁷ PFU/dose, about 1.0×10⁸ PFU/dose, about 1.5×10⁸ PFU/dose, about 2.0×10⁸ PFU/dose, about 2.5×10⁸ PFU/dose, about 3.0×10⁸ PFU/dose, about 3.5×10⁸ PFU/dose, about 4.0×10⁸ PFU/dose, about 4.5×10⁸ PFU/dose, or about 5.0×10⁸ PFU/dose.

In some embodiments, the booster dose is in the same dosage as the prime dose. In some embodiments, the booster dose is a lower dosage than the prime dose. In some embodiments, the booster dose (e.g., the first booster dose or the second booster dose) is between 1.0×10⁷ PFU/dose and 5.0×10⁸ PFU/dose, for example, about 1.0×10⁷ PFU/dose, about 1.5×10⁷ PFU/dose, about 2.0×10⁷ PFU/dose, about 2.5×10⁷ PFU/dose, about 3.0×10⁷ PFU/dose, about 3.5×10⁷ PFU/dose, about 4.0×10⁷ PFU/dose, about 4.5×10⁷ PFU/dose, about 5.0×10⁷ PFU/dose, about 5.5×10⁷ PFU/dose, about 6.0×10⁷ PFU/dose, about 6.5×10⁷ PFU/dose, about 7.0×10⁷ PFU/dose, about 7.5×10⁷ PFU/dose, about 8.0×10⁷ PFU/dose, about 8.5×10⁷ PFU/dose, about 9.0×10⁷ PFU/dose, about 9.5×10⁷ PFU/dose, about 1.0×10⁸ PFU/dose, about 1.5×10⁸ PFU/dose, about 2.0×10⁸ PFU/dose, about 2.5×10⁸ PFU/dose, about 3.0×10⁸ PFU/dose, about 3.5×10⁸ PFU/dose, about 4.0×10⁸ PFU/dose, about 4.5×10⁸ PFU/dose, or about 5.0×10⁸ PFU/dose. The booster dose may be in a dosage the same as the prime dose or lower than the prime dose.

The recombinant sMVA vectors used in accordance with the embodiments disclosed herein include a synthetic MVA genome that serves as a viral backbone (referred to herein as the sMVA backbone or sMVA genome backbone) and one or more heterologous nucleotide sequences that encode (i) a SARS-CoV-2 S protein (or variant, mutant, and/or immunogenic fragment thereof) and (ii) a SARS-CoV-2 N protein (or variant, mutant, and/or immunogenic fragment thereof).

In some embodiments, the sMVA backbone includes an internal unique region (UR) of a parental MVA genome flanked on each side by at least a portion of an inverted terminal repeat (ITR) region of the parental MVA genome. The parental genome used to generate the sMVA backbone may be any known MVA strain or variant thereof including, but not limited to, MVA strain Acambis (Accession #AY603355) or MVA strain Antoine (NCBI Accession #U94848). In some embodiments, the sMVA backbone is identical to the corresponding full-length parental MVA genome sequence. In other embodiments, the sMVA backbone is identical or substantially identical to the corresponding full-length parental MVA genome sequence, but includes one or more unintentional mutations originating from chemical synthesis, cloning, propagation, or other errors resulting from the production of the synthetically made virus. For example, in some embodiments, the sMVA backbone has a single T to A nucleotide alteration located in the IGR at three base pairs downstream of open reading frame (ORF) 021 L. In some embodiments, the sMVA backbone has at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to the corresponding full-length parental MVA genome sequence.

Also disclosed herein is a heterologous booster regime using the sMVA SARS-CoV-2 vaccine platform to provide additional recognition elements (both S antigen and N antigen) over a homologous boost from the EUA Moderna vaccine or the FDA-approved Pfizer vaccine, which is directed only toward SARS-CoV-2 Spike protein. The vaccine's MVA backbone may be more effective at inducing COVID-19 immunity since MVA strongly induces T cell responses even in a background of immunosuppression. In addition, vaccine targeting of both Spike and Nucleocapsid antigens, may offer greater protection against the significant sequence variation observed with the Spike antigen. The sMVA-based SARS-CoV-2 vaccine at a dose of 1.0×10⁷ PFU or 1.0×10⁸ PFU is used as a booster vaccination for a subject who has been previously vaccinated with the same or a different SARS-CoV-2 vaccine.

Also disclosed herein a method of preventing, treating, or reducing the severity of COVID-19 caused by a coronavirus infection; preventing infection by the coronavirus, or increasing an immune response in a subject who has previously received one or more doses of a SARS-CoV-2 vaccine. The method comprises administering to the subject a booster dose of a composition comprising a recombinant sMVA vector comprising, expressing, or is capable of expressing one or more heterologous DNA sequences encoding a SARS-CoV-2 Spike (S) protein and a SARS-CoV-2 Nucleocapsid (N) protein or variants or mutants of the S protein and N protein.

The coronavirus infection that causes COVID-19 may be SARS-CoV-2 or any variants thereof according to the embodiments disclosed herein. In some embodiments, the coronavirus infection treated by the methods and compositions disclosed herein is caused by a variant of SARS-CoV-2. For example, a variant of SARS-CoV-2 may be a variant of concern including but not limited to B.1.1.7 (Alpha), B.1.351 (Beta), P.1 (Gamma), B.1.617.2 (Delta), B.1.621 (Mu), C.37 (Lambda), C.1.2, BA.1 (Omicron), BA.2 (Omicron), BA.2.12.1 (Omicron), BA.4 (Omicron), BA.5 (Omicron), BA.2.75 (Omicron), BQ.1 (Omicron), BQ.1.1 (Omicron), and XBB (Omicron).

In some embodiments, the composition is a vaccine composition or any immunogenic composition capable of providing full or partial protection against infection by a coronavirus that causes COVID-19. In other embodiments, the composition is a vaccine composition or any immunogenic composition capable of providing full or partial protection from developing moderate or severe COVID-19 symptoms. In other embodiments, the composition is a vaccine composition or any immunogenic composition capable of providing protection from hospitalization or death as a result of developing COVID-19.

In some embodiments, the previously received SARS-CoV-2 vaccine is an mRNA-based SARS-CoV-2 vaccine composition. In some embodiments, the previously received SARS-CoV-2 vaccine is a SARS-CoV-2 vaccine composition targeting the S antigen only. In some embodiments, the subject received the previous SARS-CoV-2 vaccine at least one month, at least two months, at least 3 months, at least 4 months, at least 5 months, at least 6 months, at least 7 months, at least 8 months, at least 9 months, at least 10 months, at least 11 months, or at least 12 months before receiving the booster dose of the sMVA vaccine composition disclosed herein.

In some embodiments, the composition is administered to the subject as a single dose. In some embodiments, the single dose is between 1.0×10⁷ PFU/dose and 1.0×10⁸ PFU/dose, for example, about 1.0×10⁷ PFU/dose, about 1.5×10⁷ PFU/dose, about 2.0×10⁷ PFU/dose, about 2.5×10⁷ PFU/dose, about 3.0×10⁷ PFU/dose, about 3.5×10⁷ PFU/dose, about 4.0×10⁷ PFU/dose, about 4.5×10⁷ PFU/dose, about 5.0×10⁷ PFU/dose, about 5.5×10⁷ PFU/dose, about 6.0×10⁷ PFU/dose, about 6.5×10⁷ PFU/dose, about 7.0×10⁷ PFU/dose, about 7.5×10⁷ PFU/dose, about 8.0×10⁷ PFU/dose, about 8.5×10⁷ PFU/dose, about 9.0×10⁷ PFU/dose, about 9.5×10⁷ PFU/dose, or about 1.0×10⁸ PFU/dose.

The recombinant sMVA vectors used in accordance with the embodiments disclosed herein include a synthetic MVA genome that serves as a viral backbone (referred to herein as the sMVA backbone or sMVA genome backbone) and one or more heterologous nucleotide sequences that encode (i) a SARS-CoV-2 S protein (or variant, mutant, and/or immunogenic fragment thereof) and (ii) a SARS-CoV-2 N protein (or variant, mutant, and/or immunogenic fragment thereof).

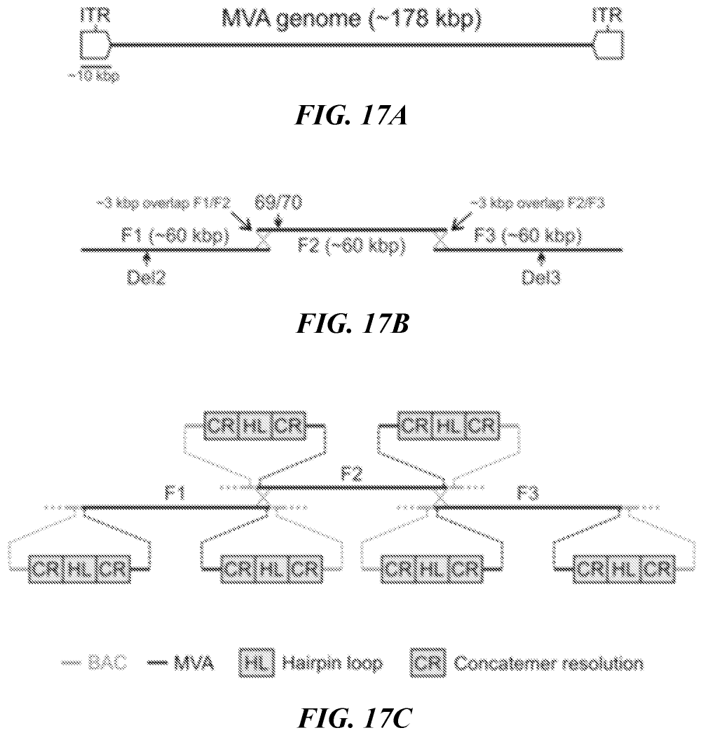

In some embodiments, the sMVA backbone includes an internal unique region (UR) of a parental MVA genome flanked on each side by at least a portion of an inverted terminal repeat (ITR) region of the parental MVA genome (FIG. 17A). The parental genome used to generate the sMVA backbone may be any known MVA strain or variant thereof including, but not limited to, MVA strain Acambis (Accession #AY603355) or MVA strain Antoine (NCBI Accession #U94848). In some embodiments, the sMVA backbone is identical to the corresponding full-length parental MVA genome sequence. In other embodiments, the sMVA backbone is identical or substantially identical to the corresponding full-length parental MVA genome sequence, but includes one or more unintentional mutations originating from chemical synthesis, cloning, propagation, or other errors resulting from the production of the synthetically made virus. For example, in some embodiments, the sMVA backbone has a single T to A nucleotide alteration located in the IGR at three base pairs downstream of open reading frame (ORF) 021 L. In some embodiments, the sMVA backbone has at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to the corresponding full-length parental MVA genome sequence.

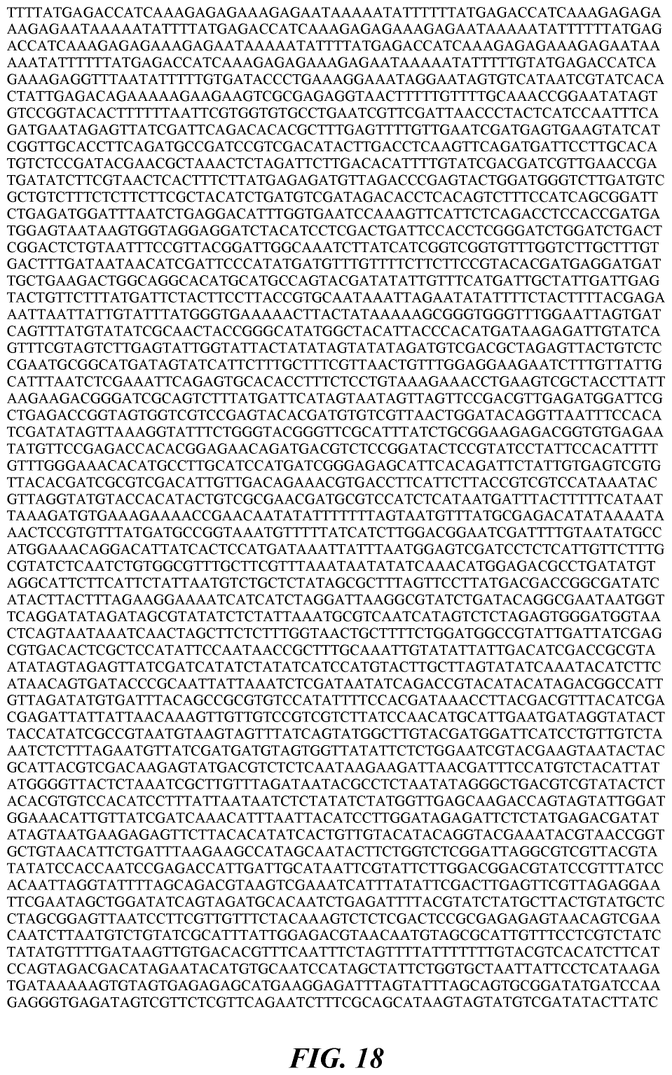

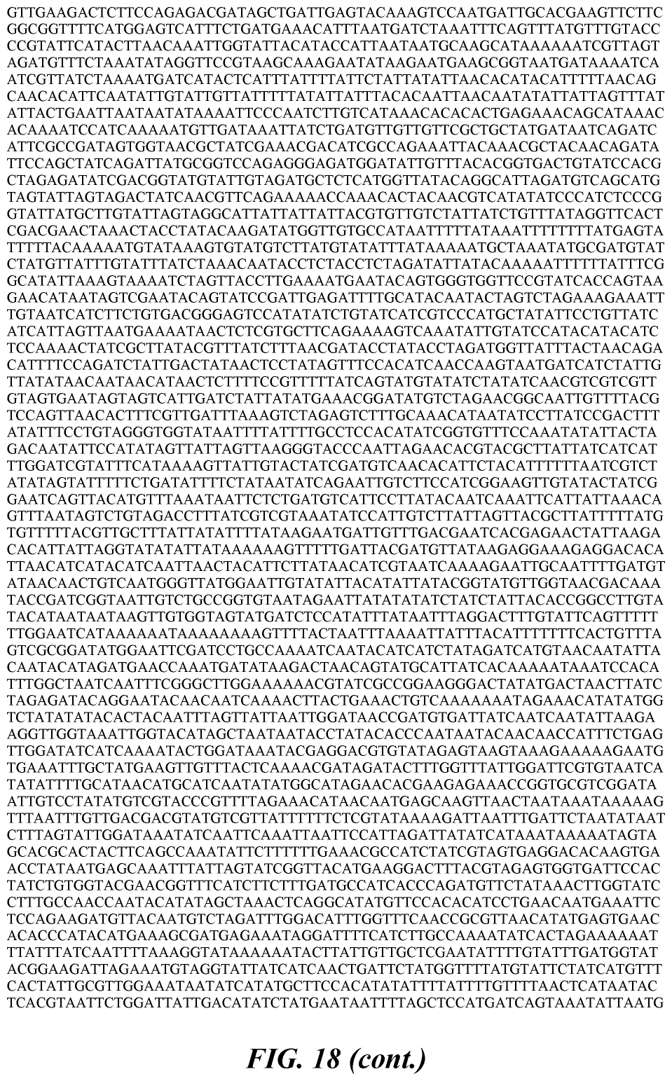

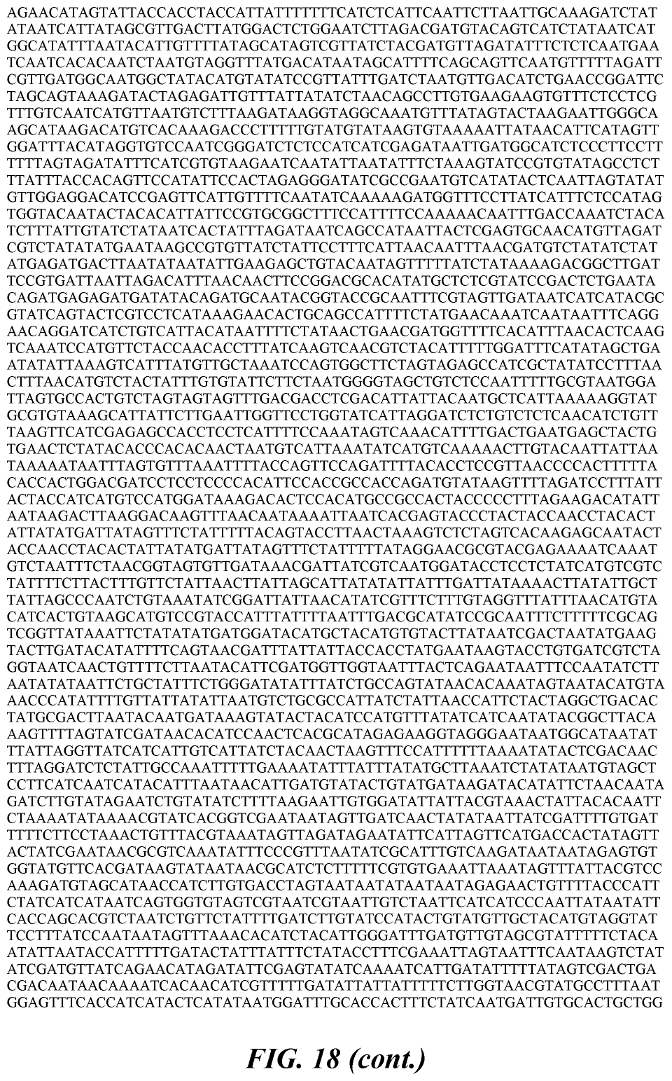

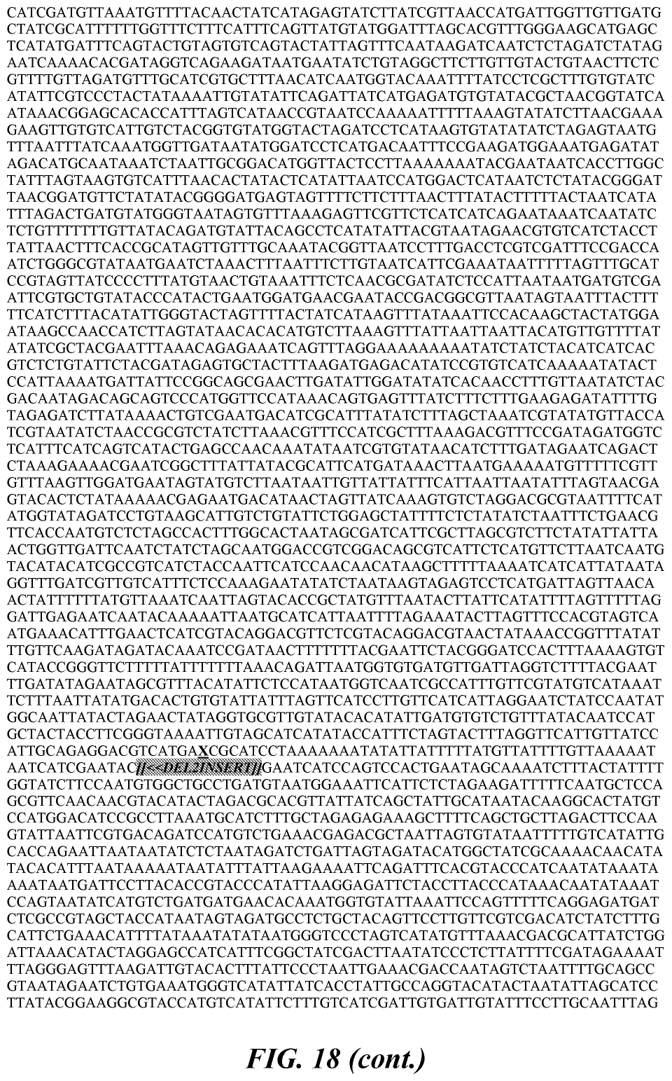

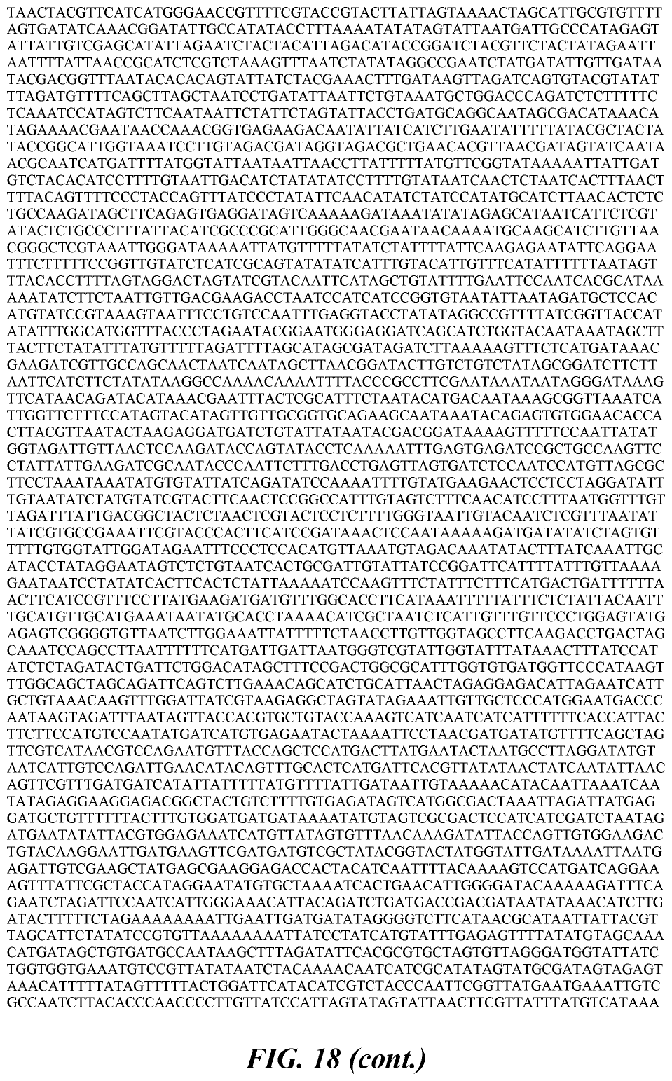

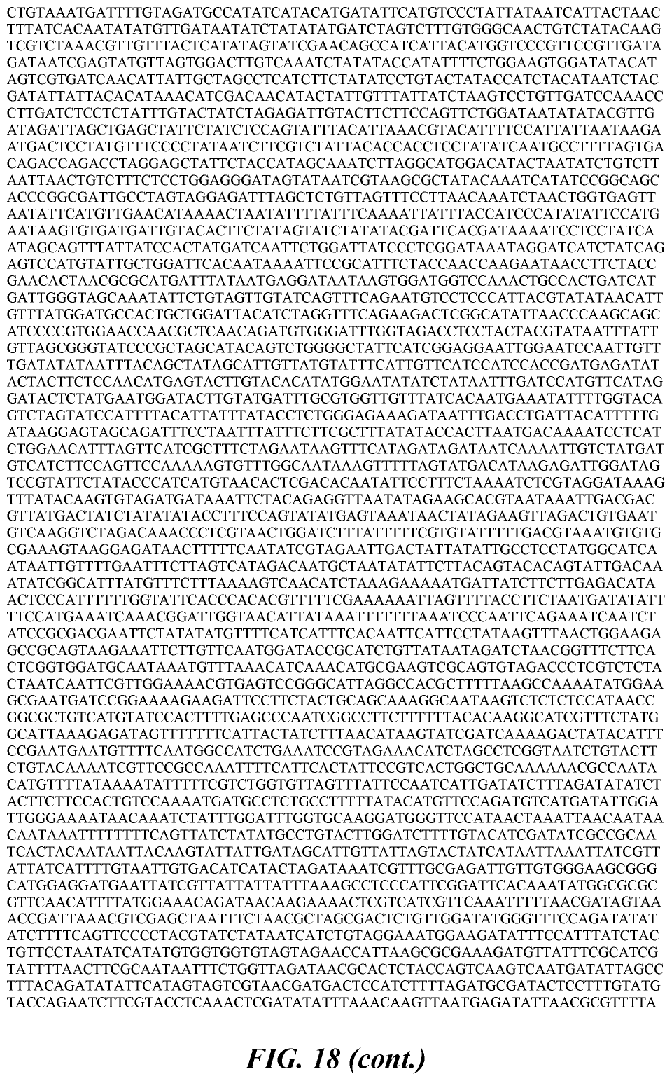

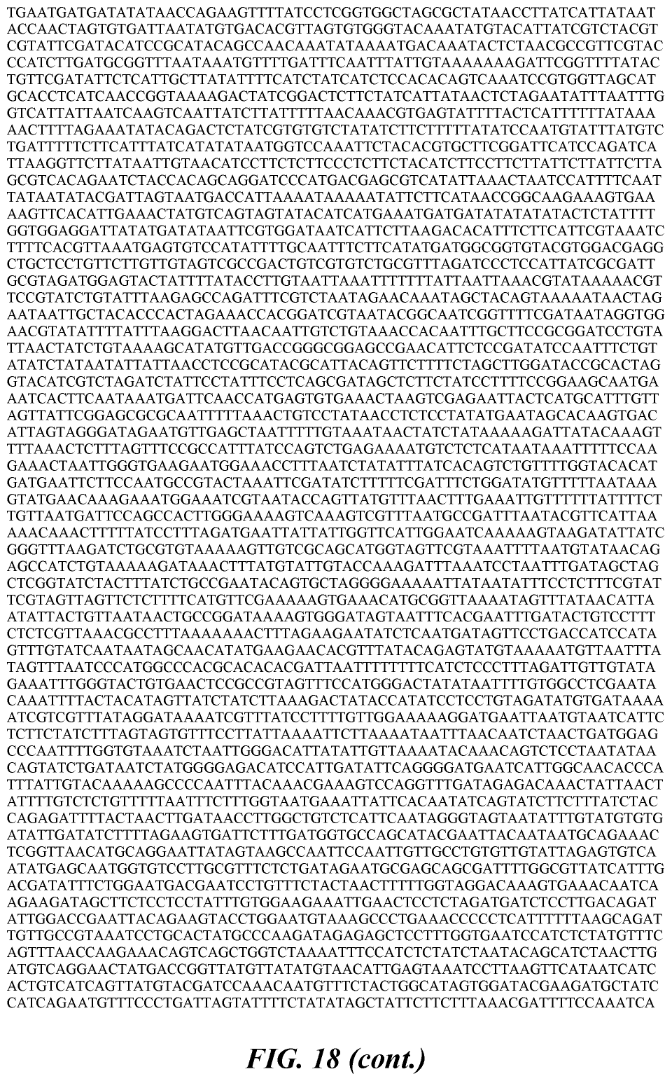

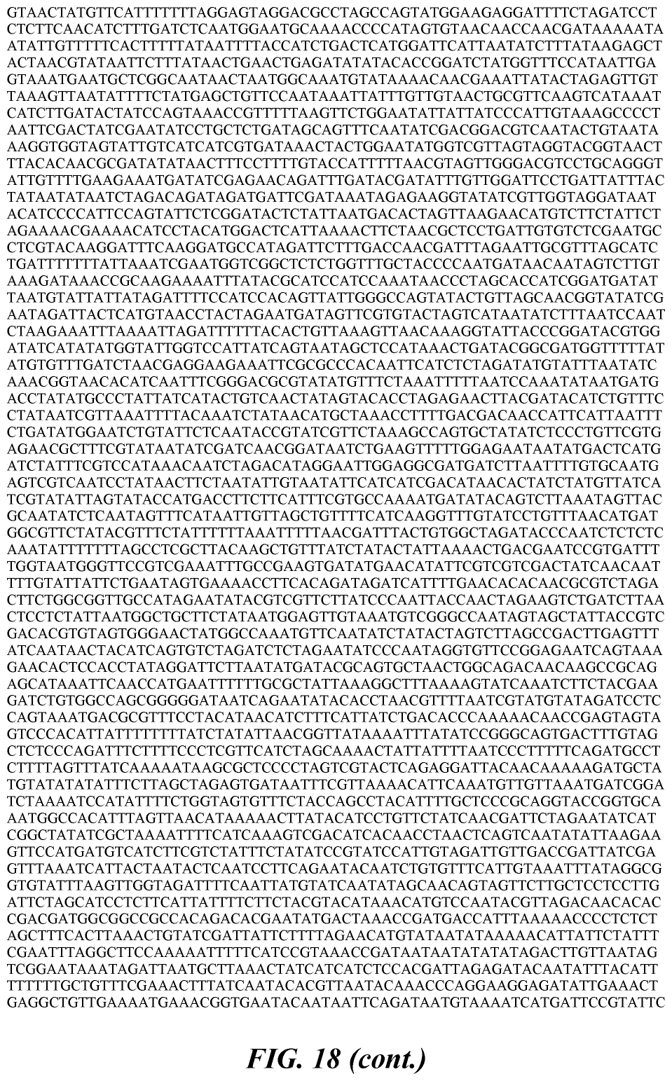

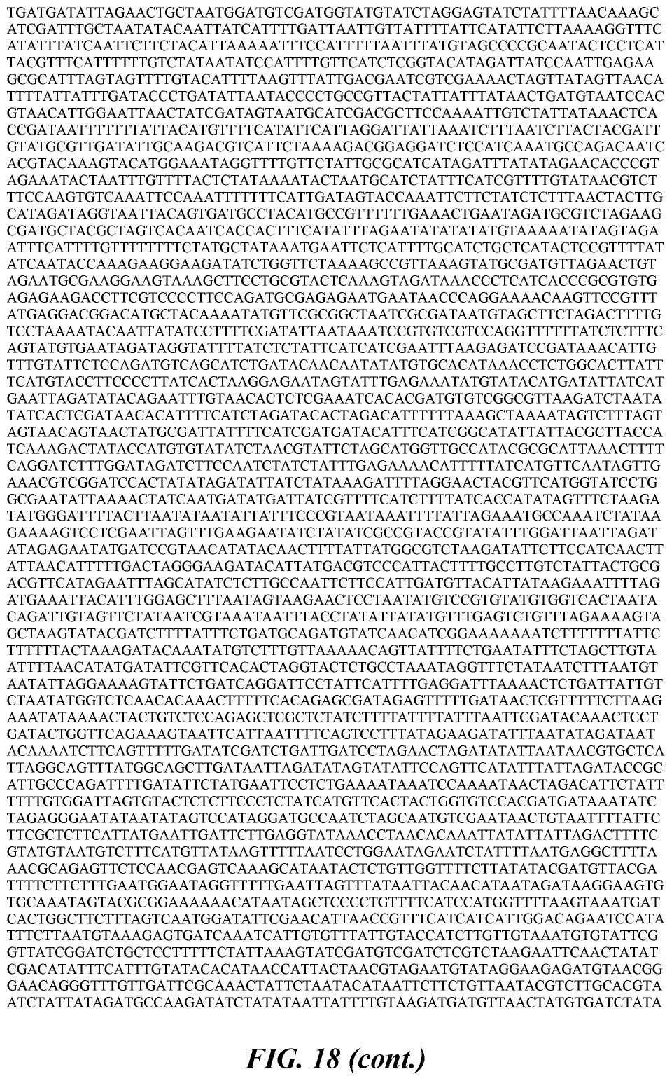

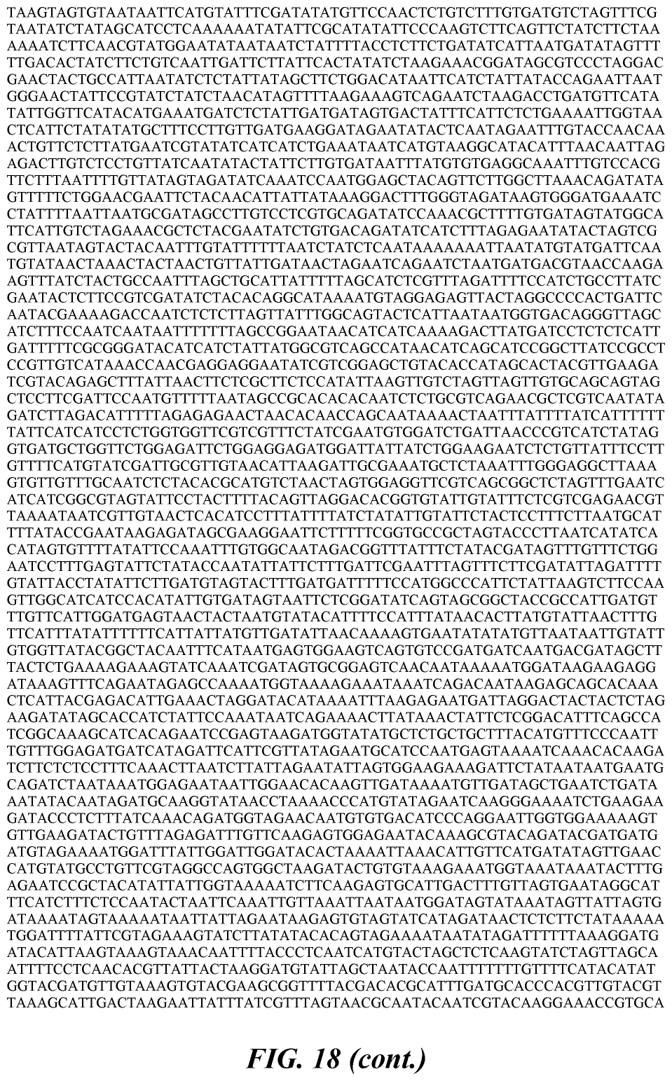

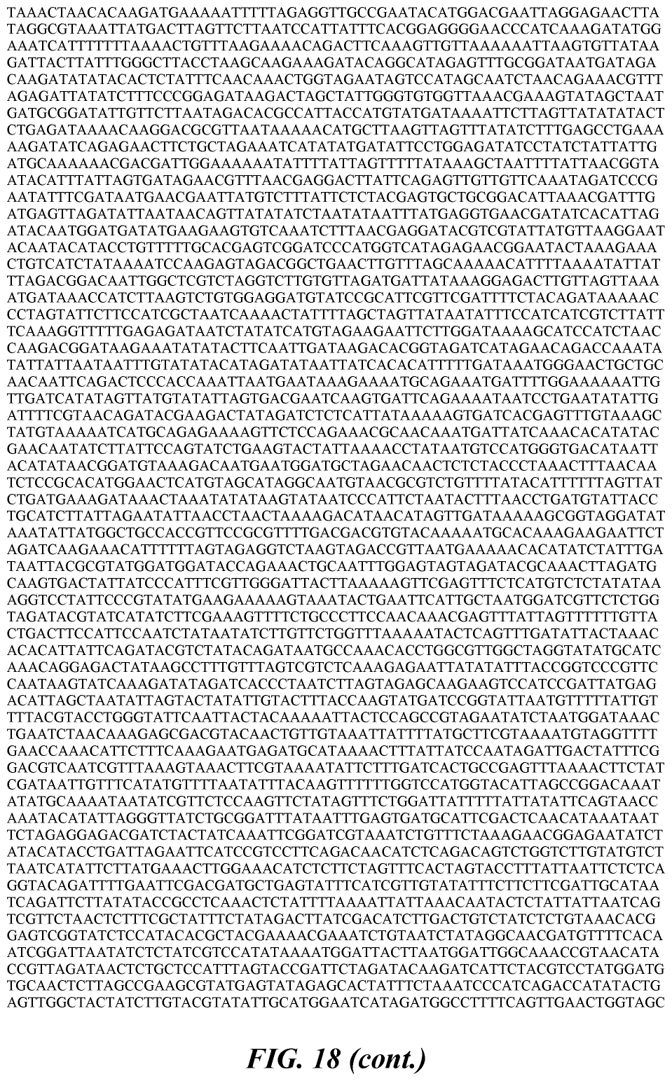

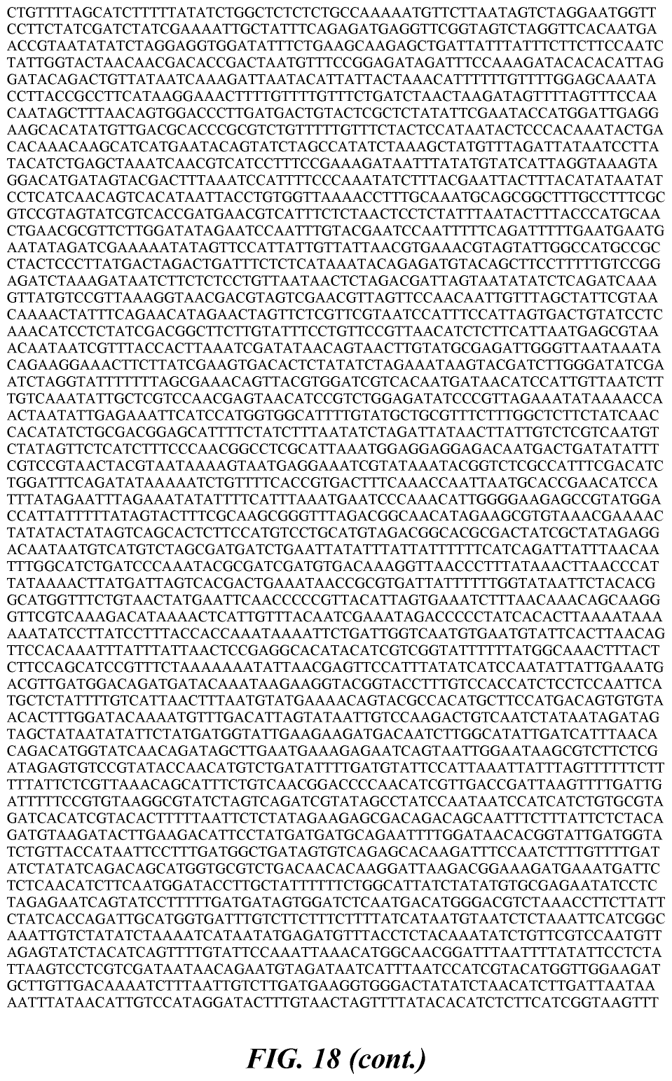

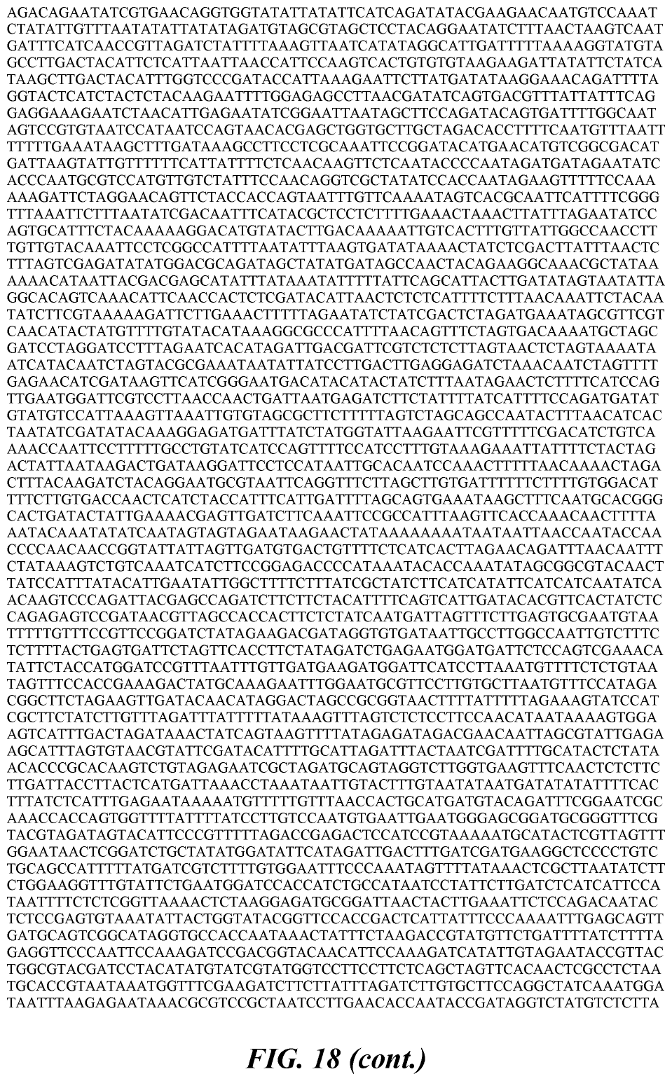

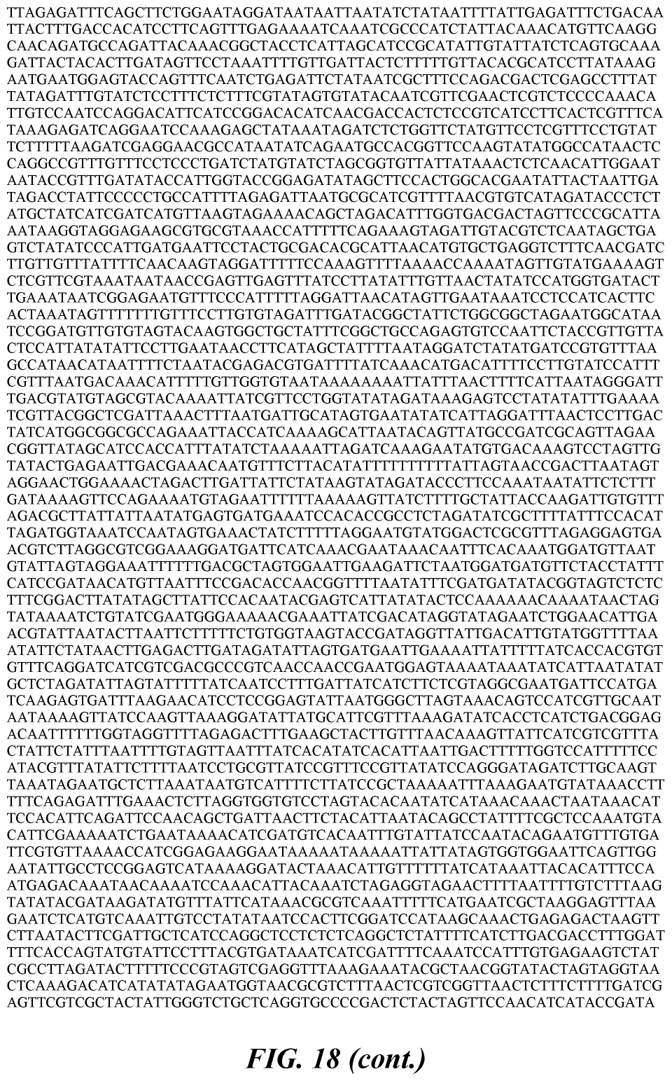

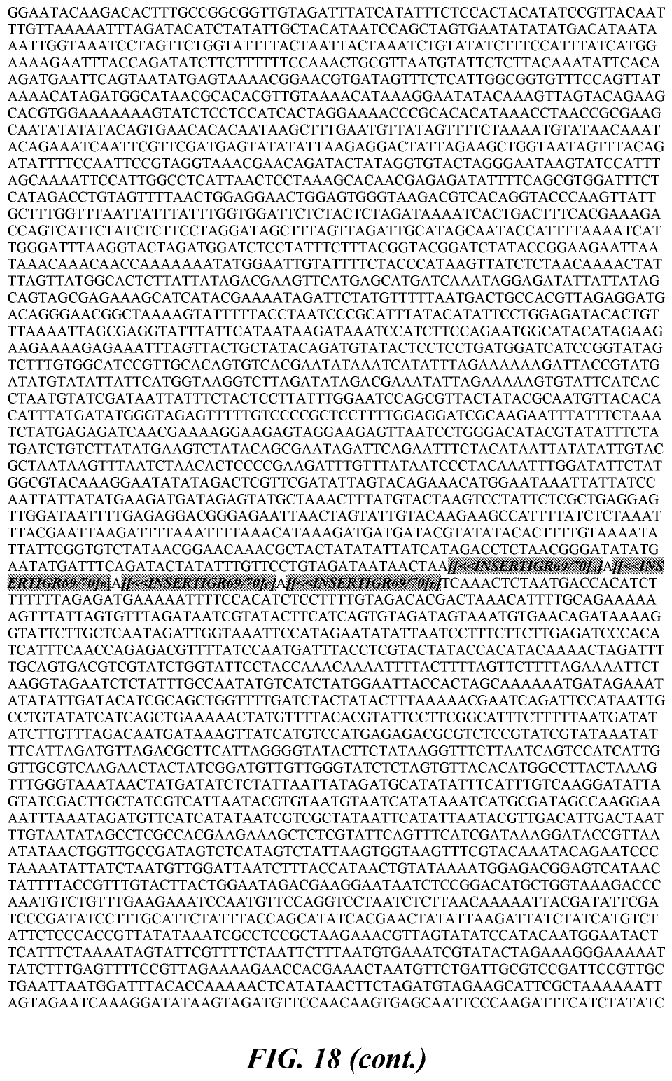

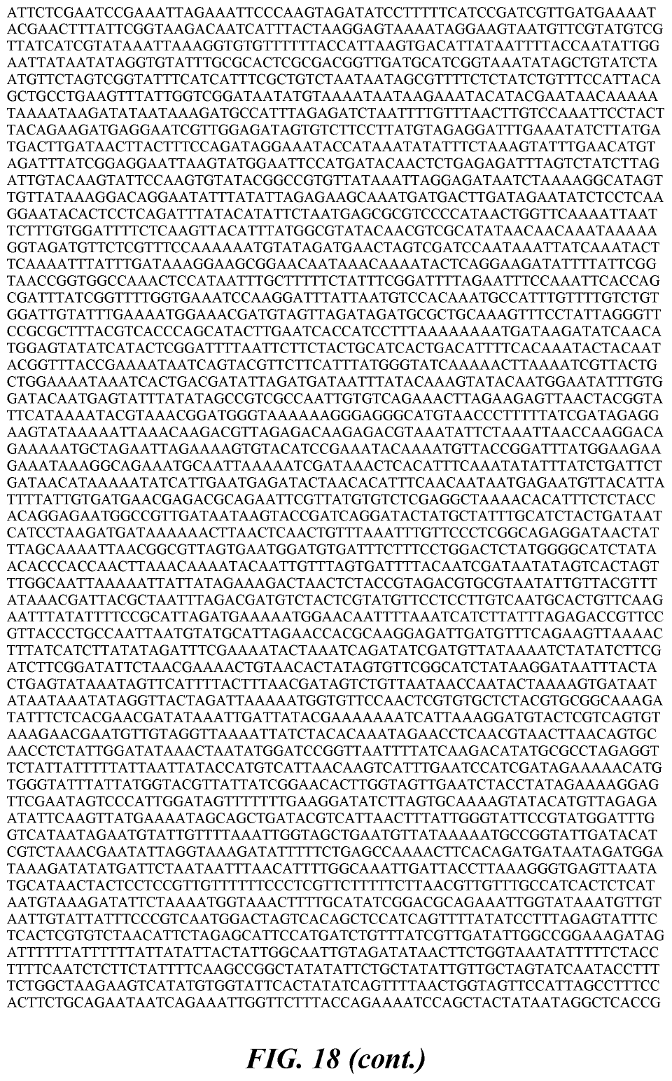

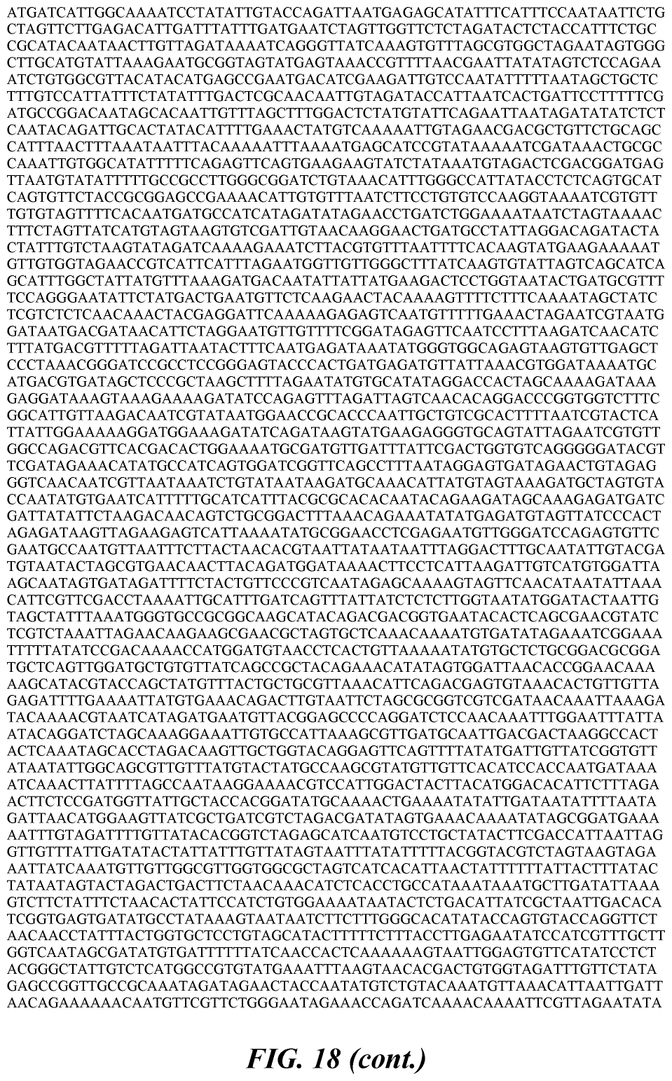

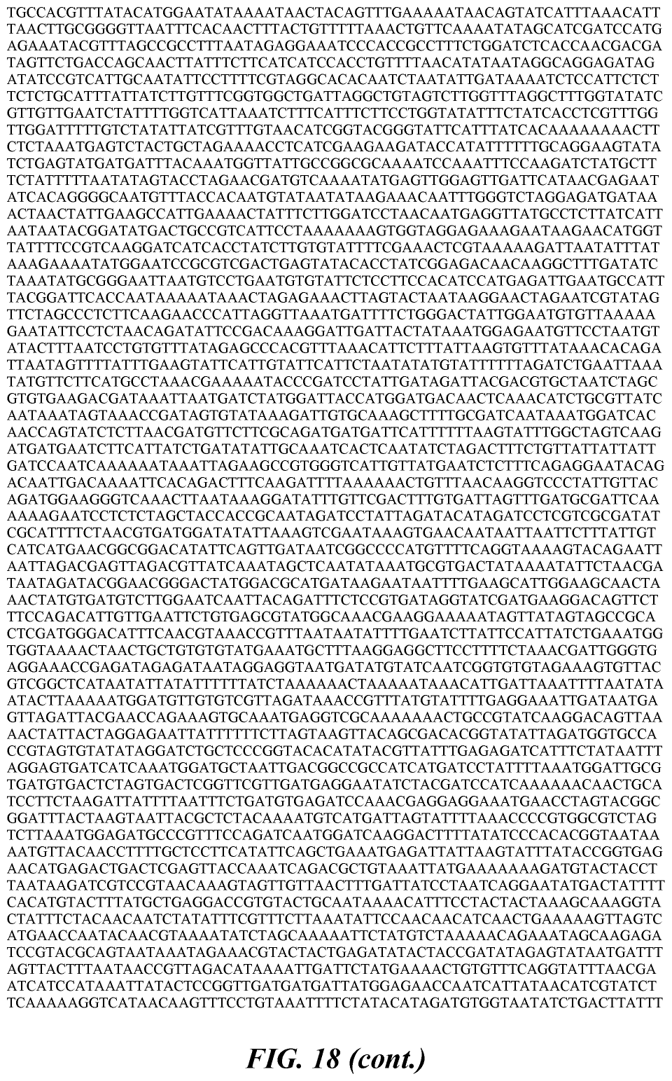

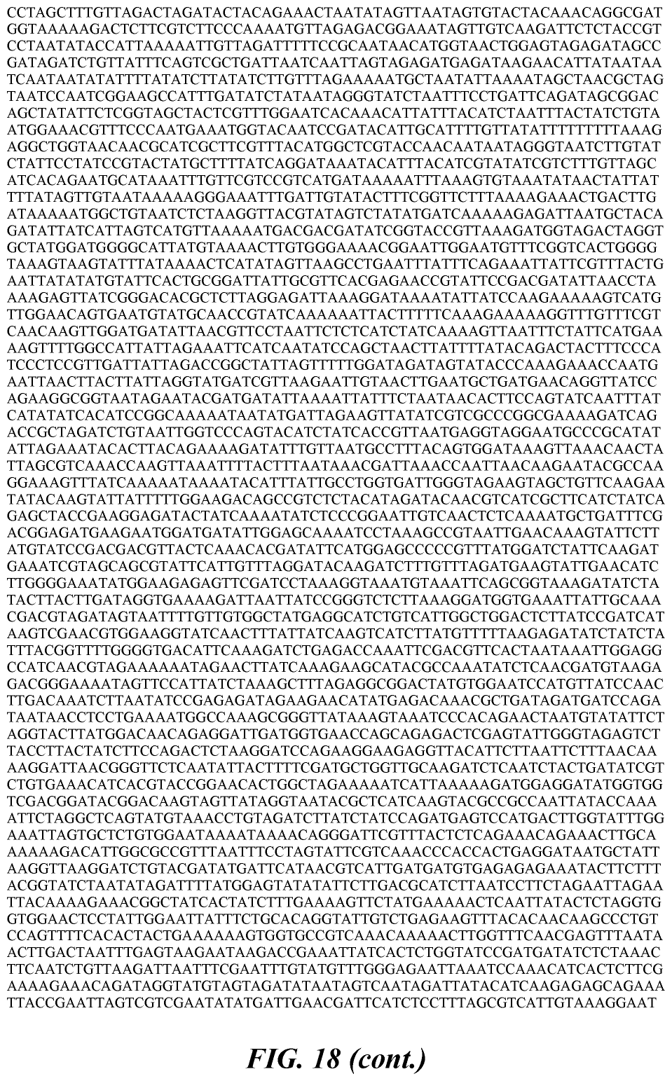

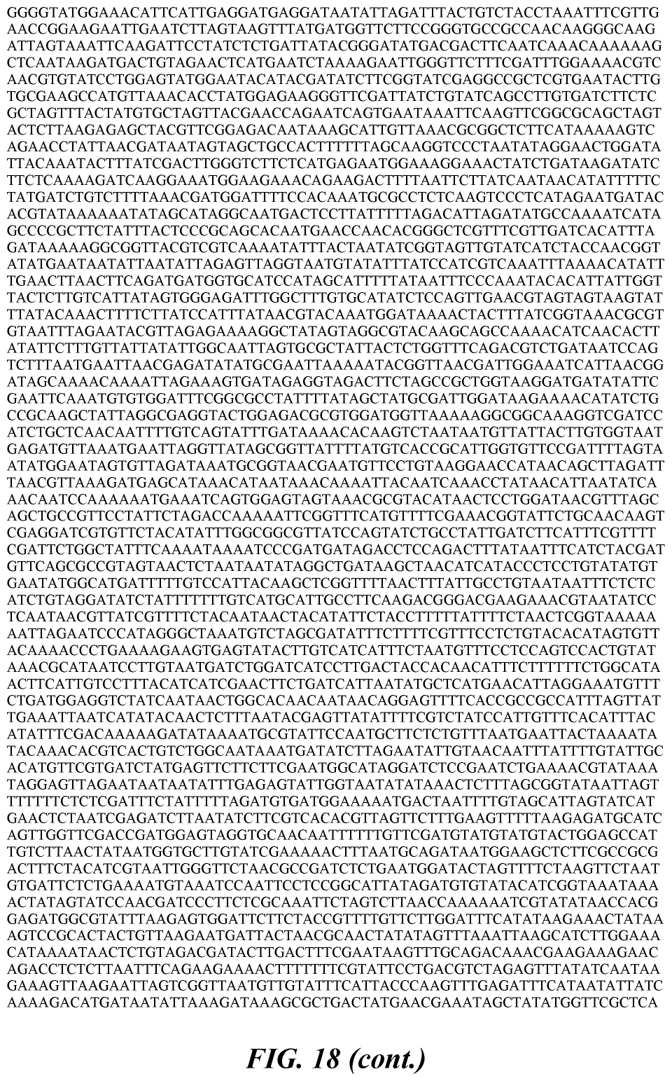

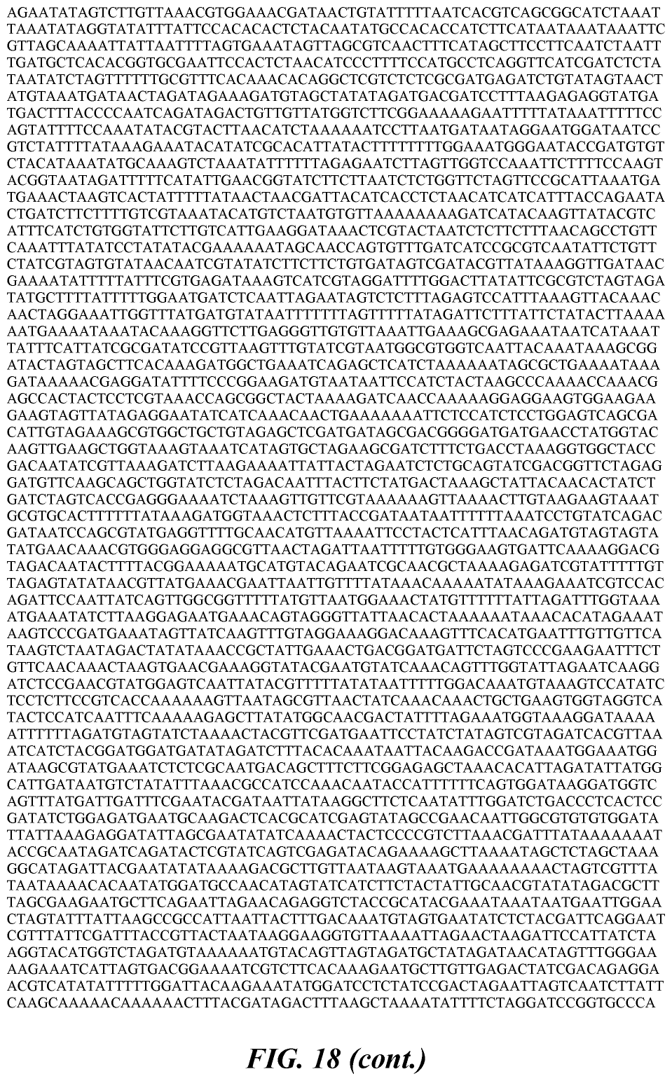

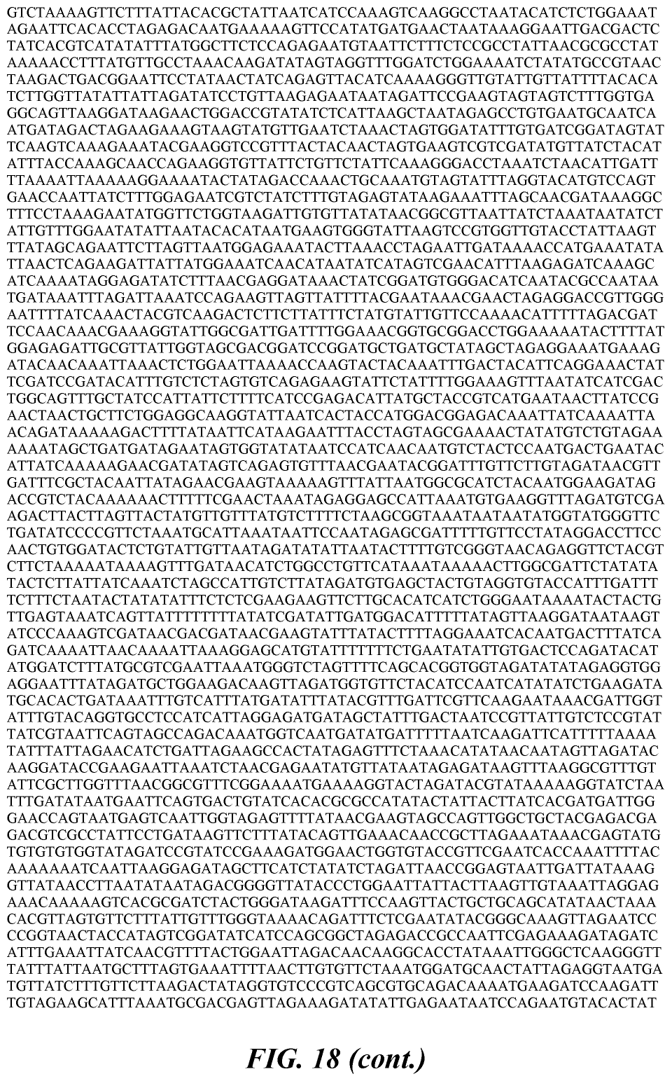

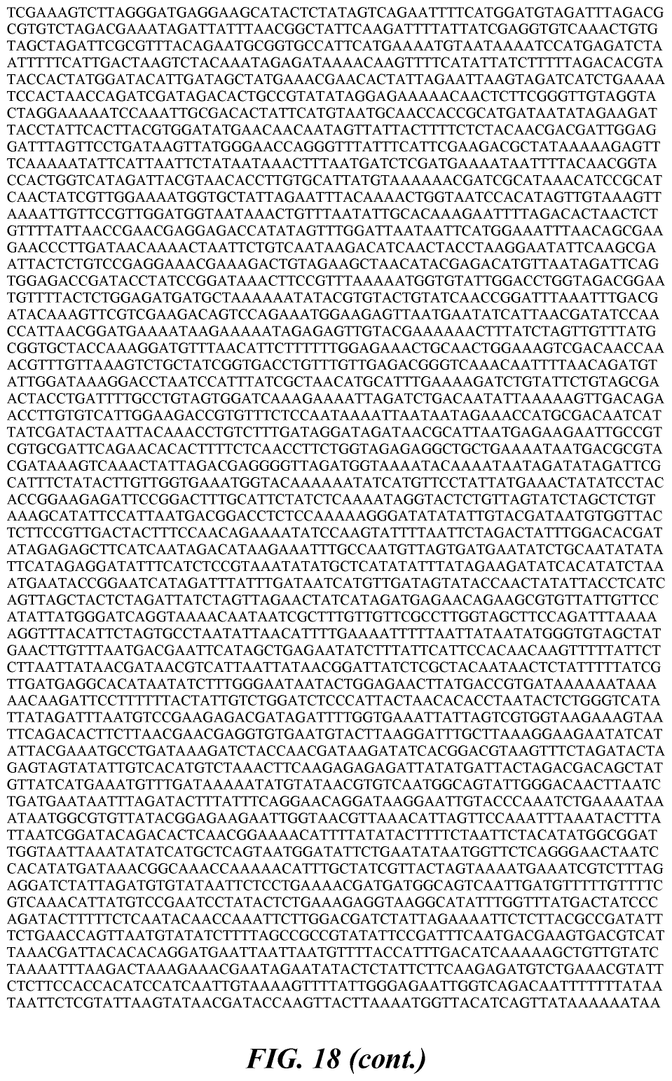

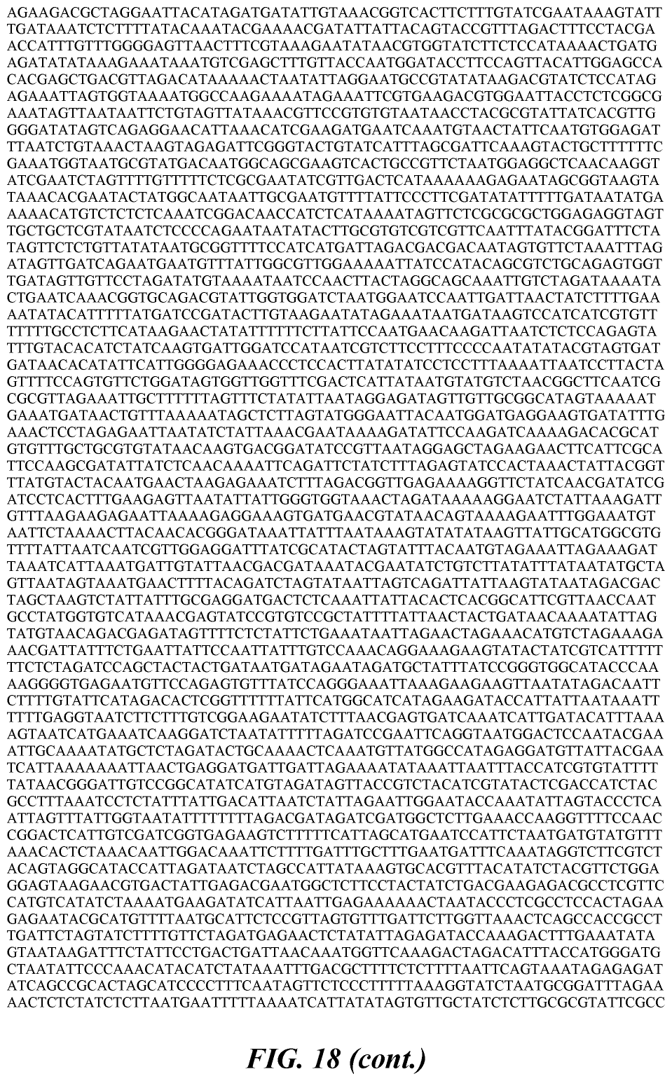

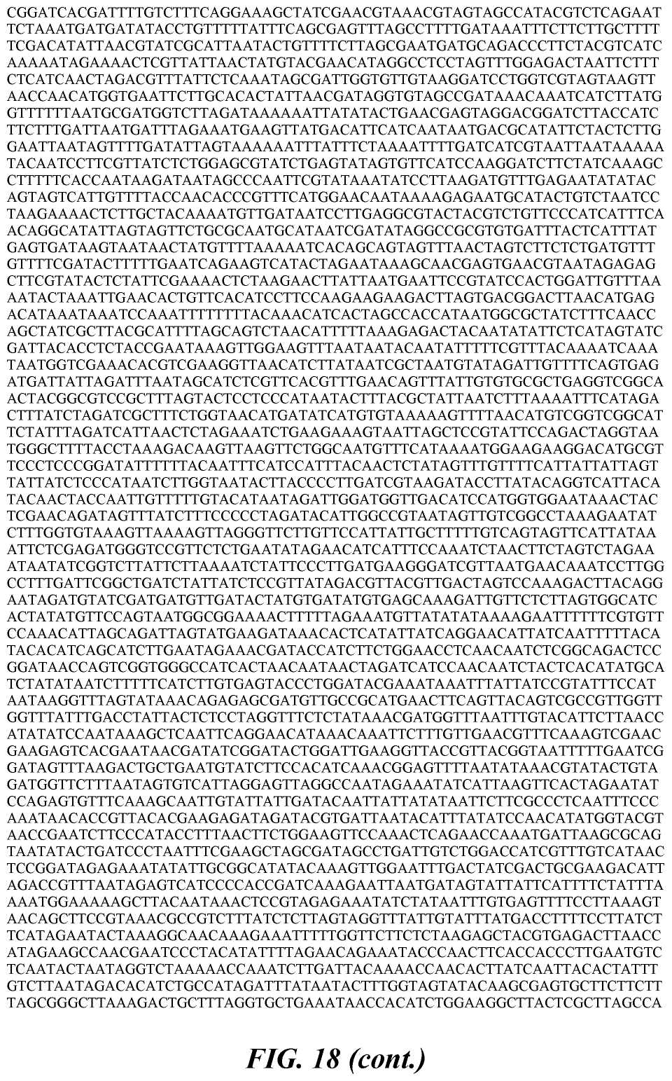

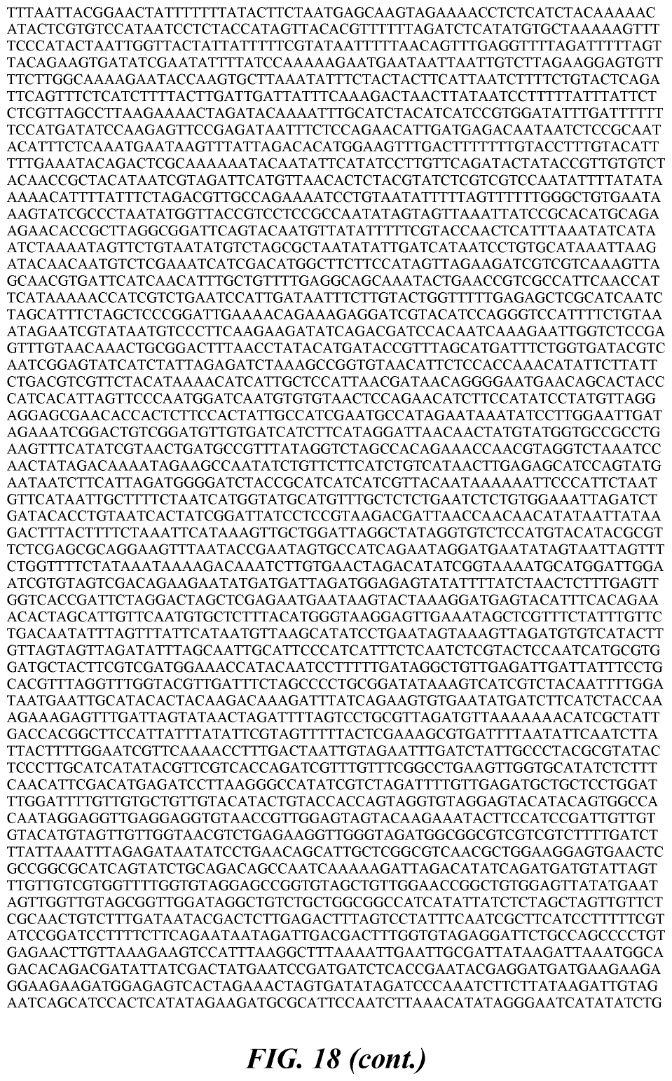

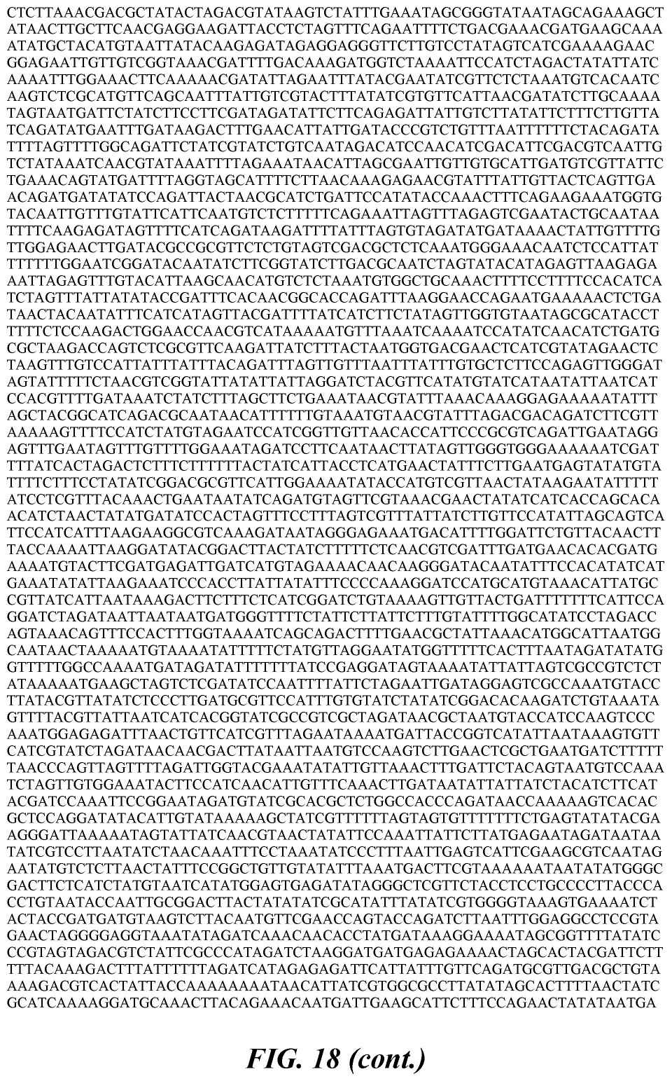

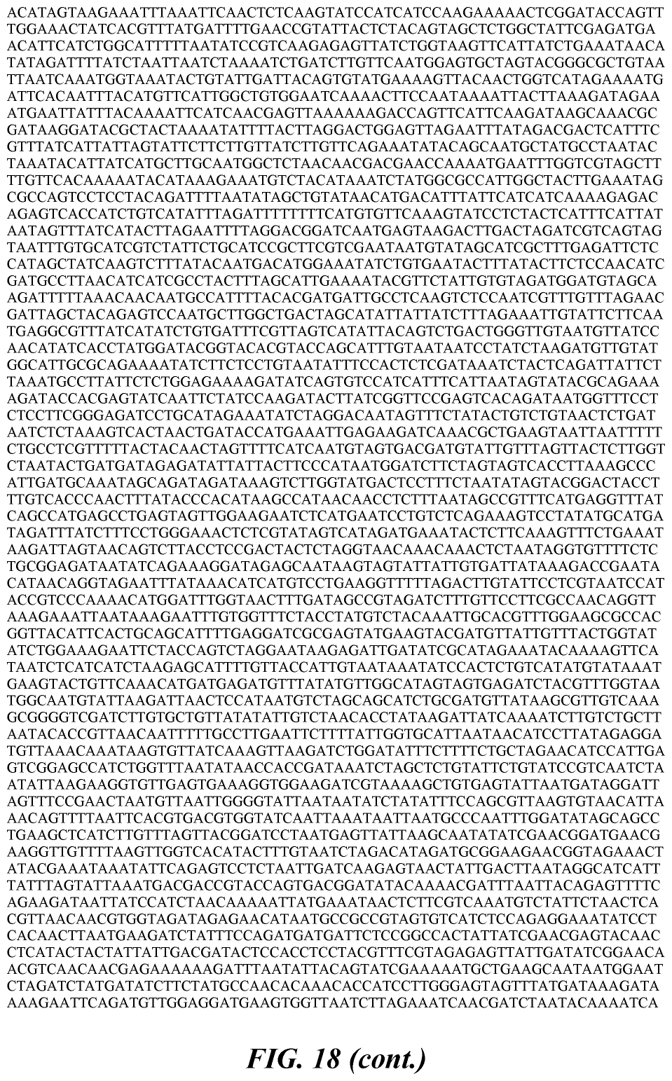

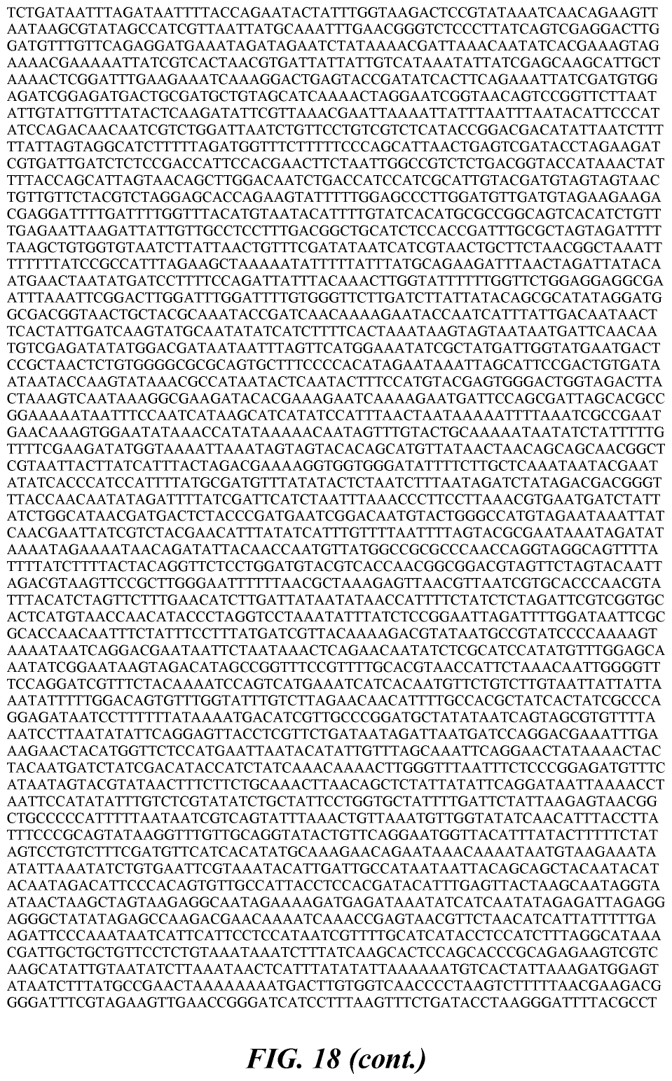

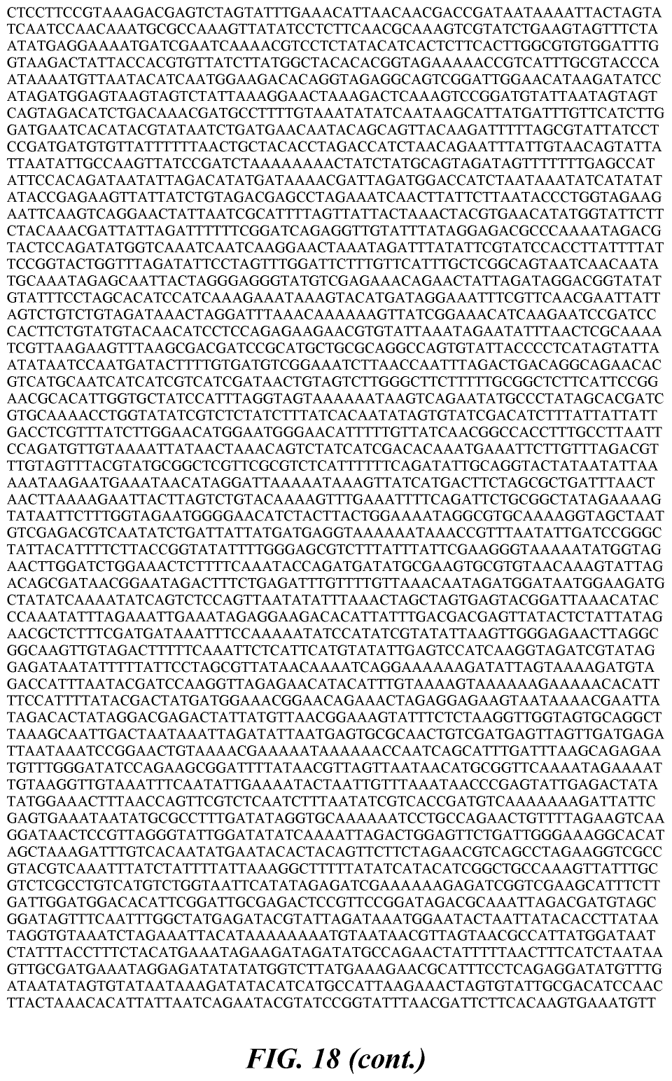

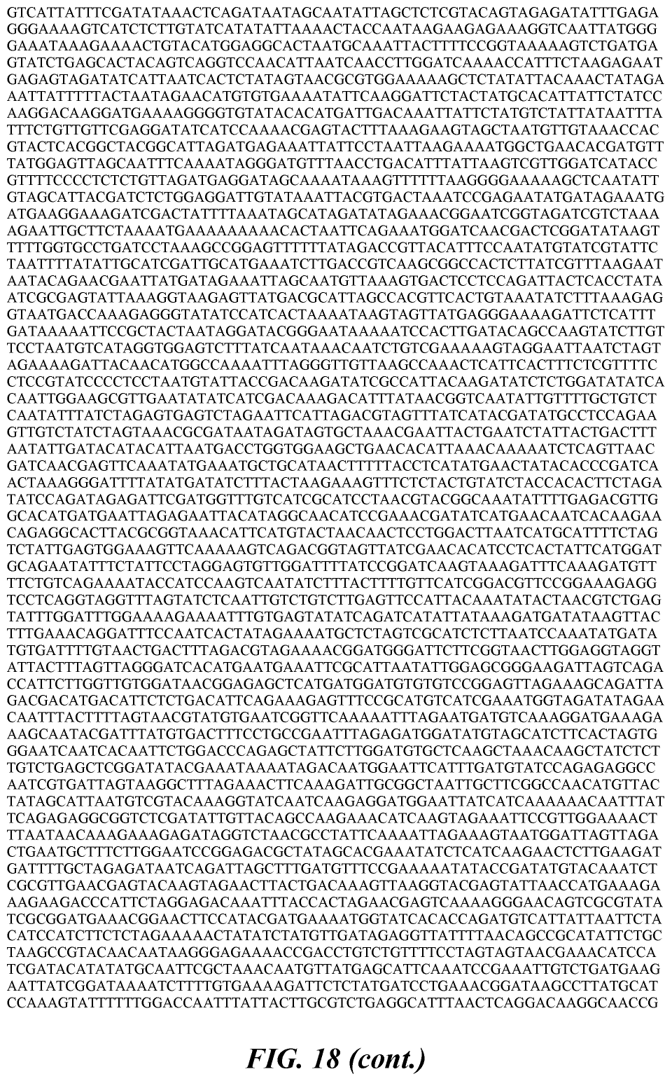

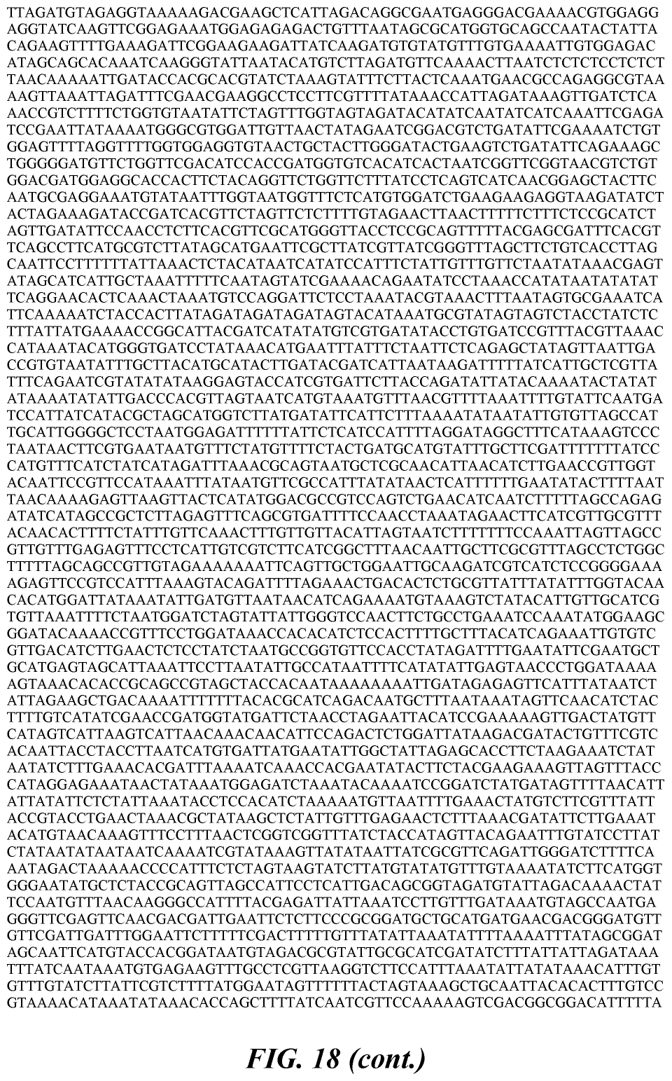

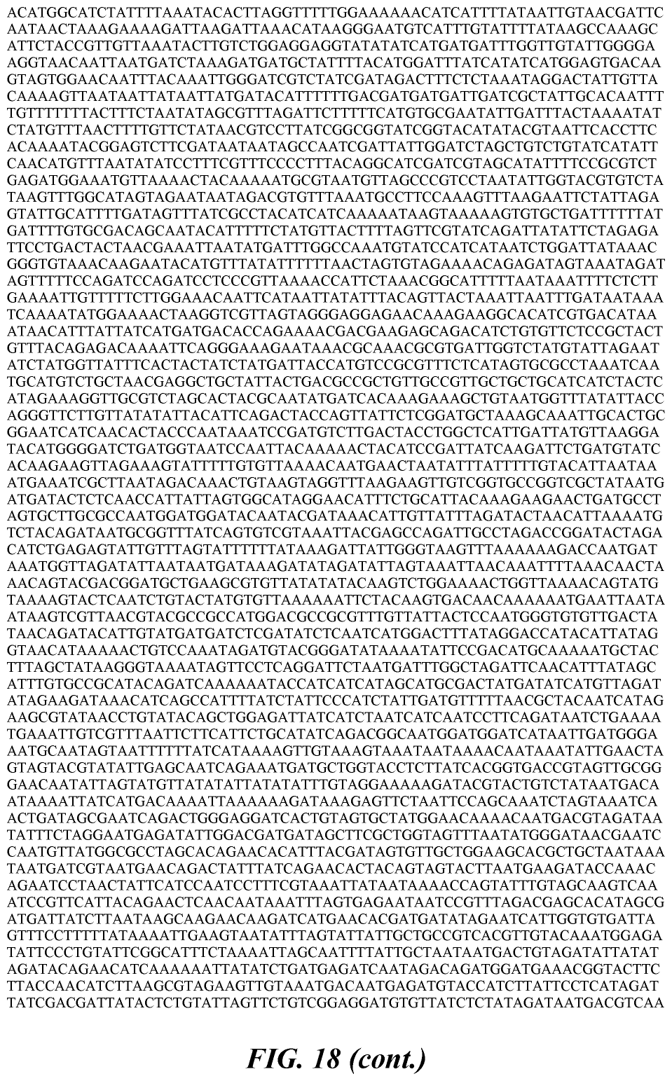

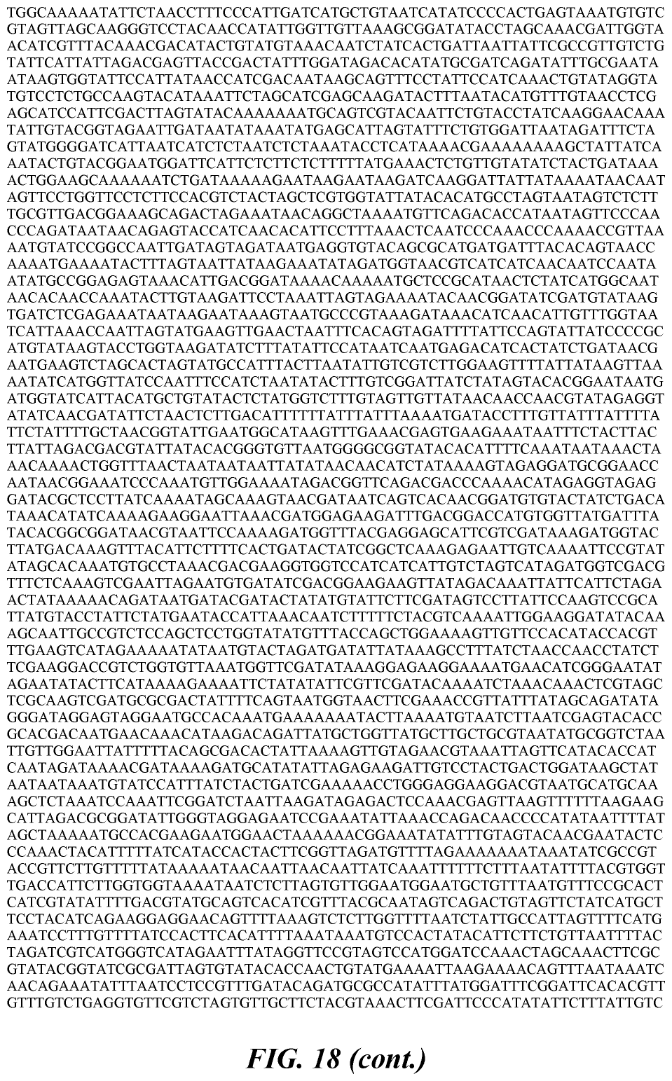

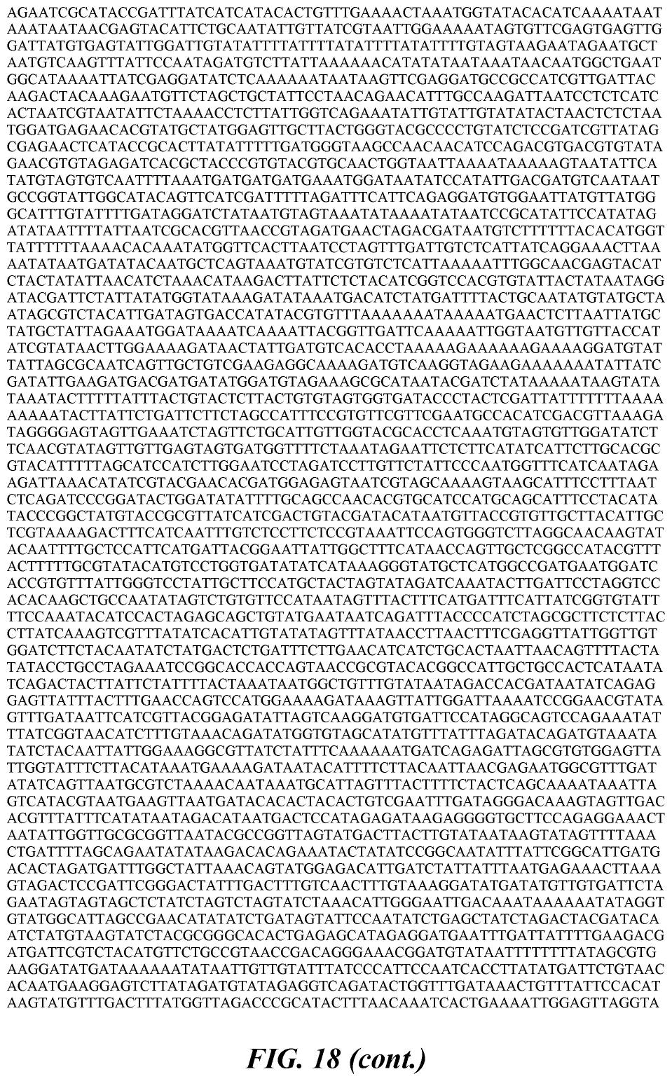

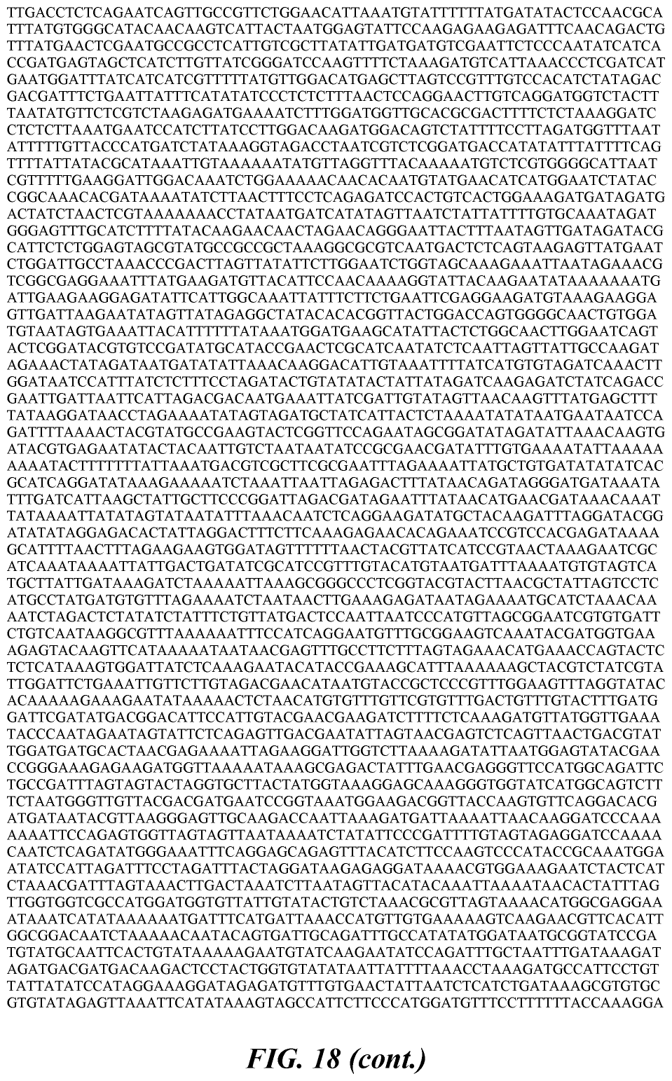

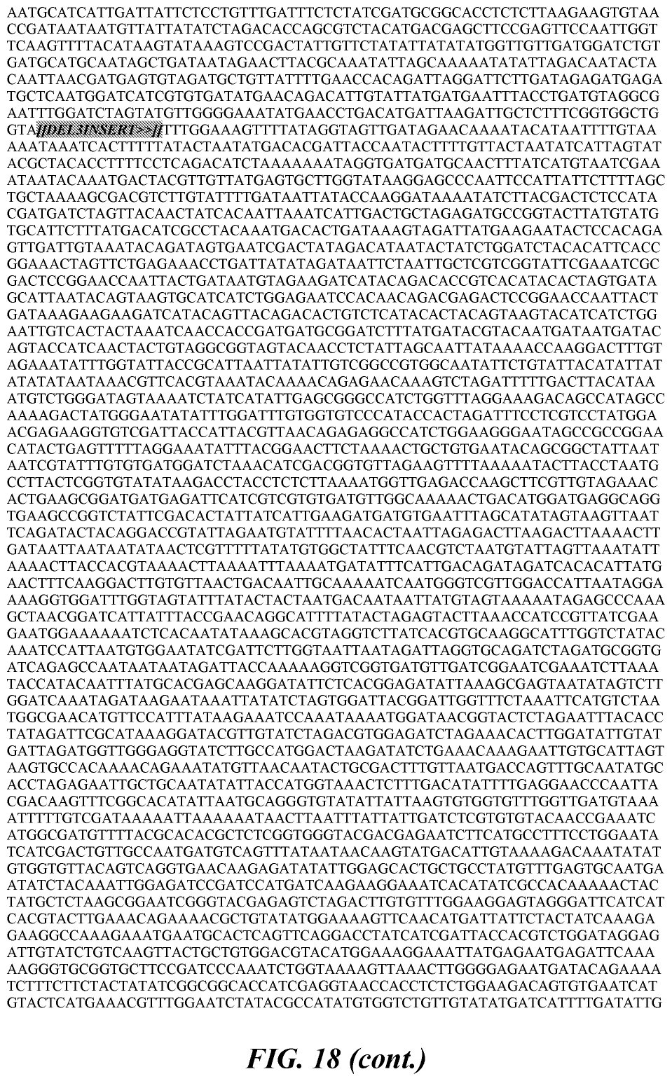

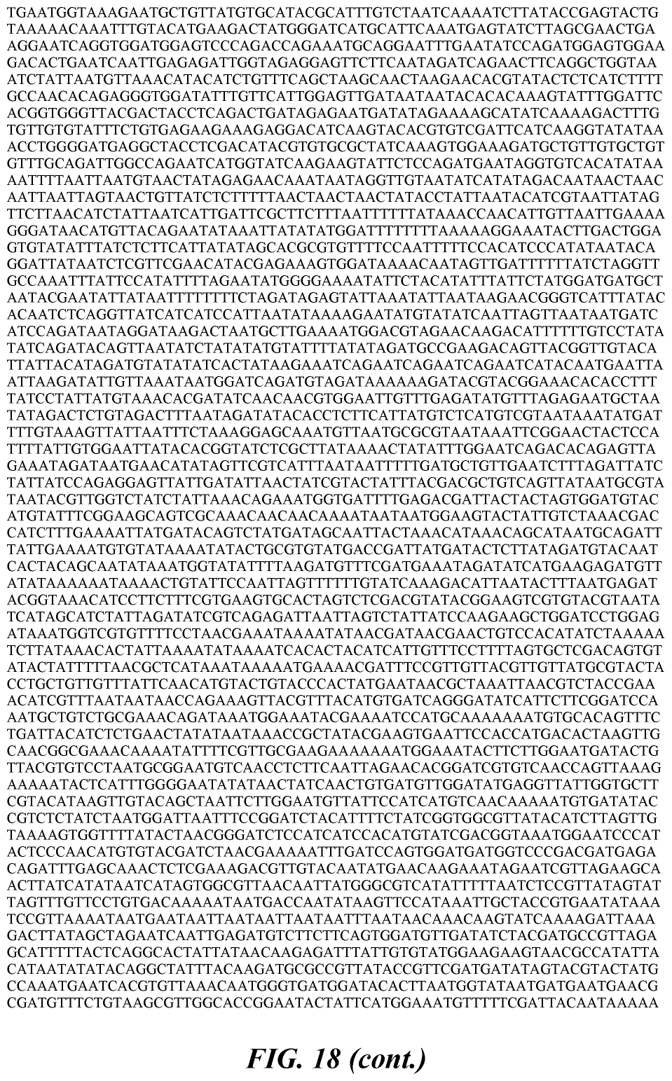

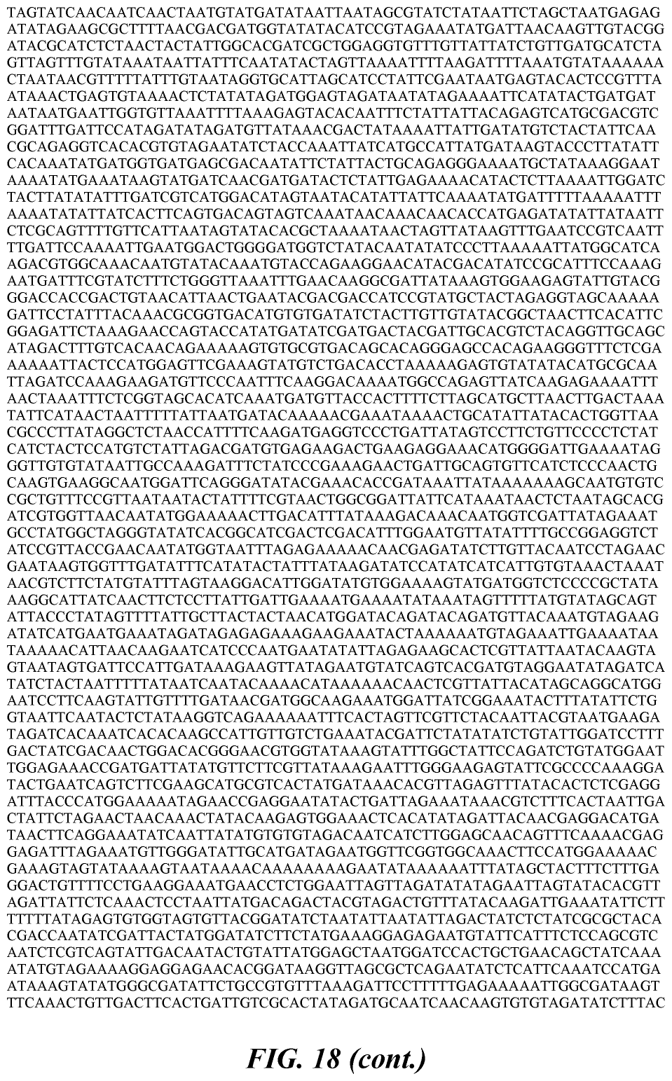

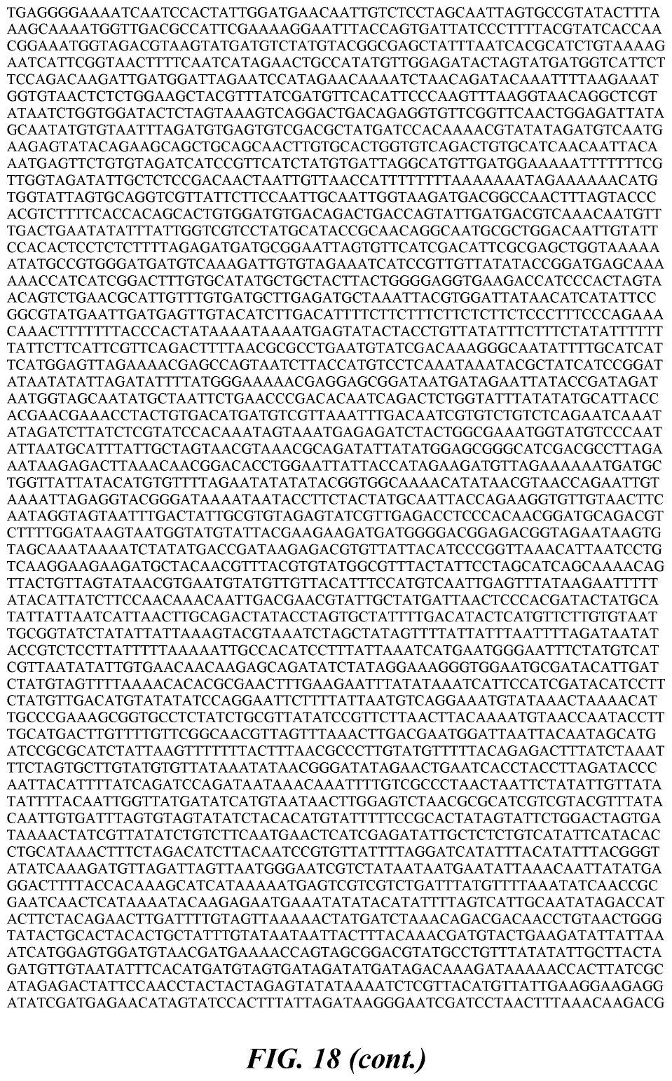

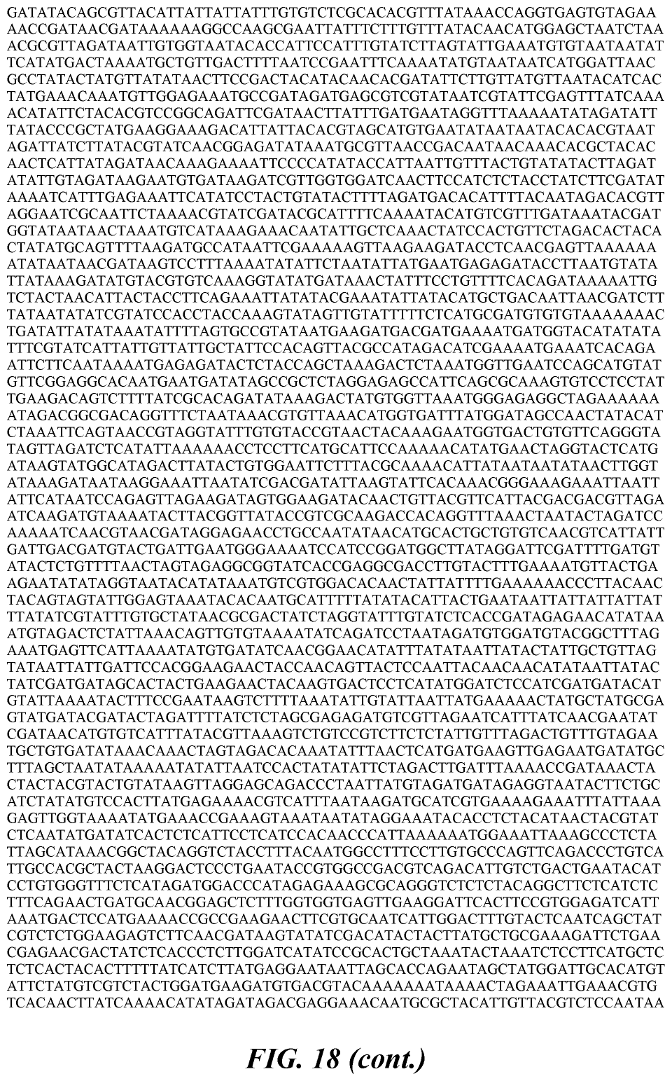

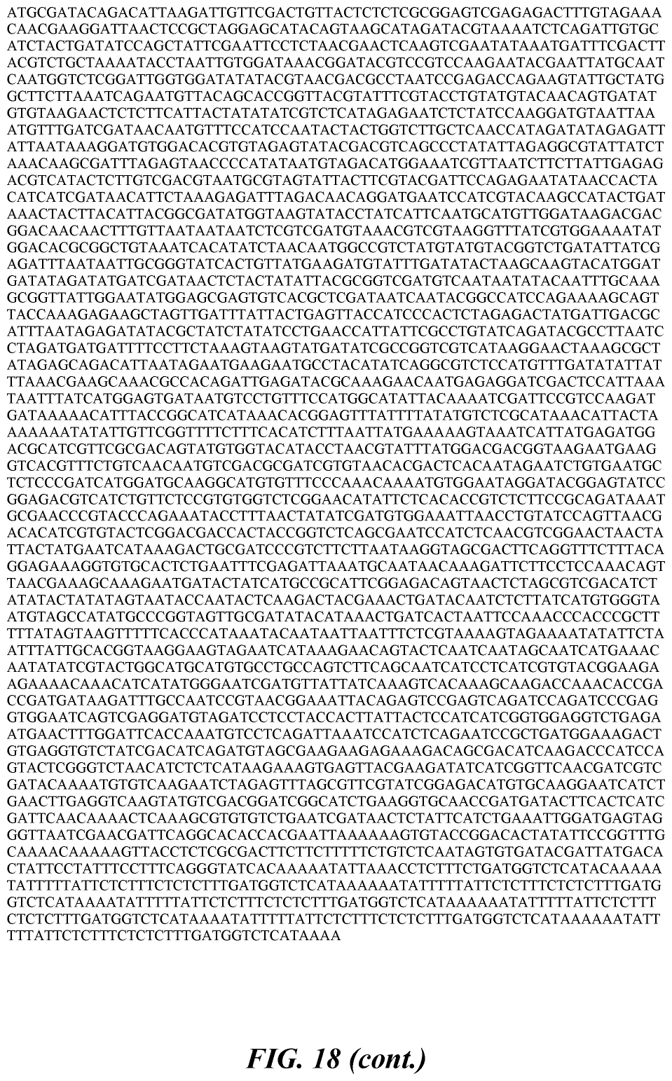

In some embodiments heterologous nucleotide sequences that encode one or more SARS-CoV-2 proteins (or variants, mutants, and/or immunogenic fragments thereof) are inserted into one or more MVA insertion sites. Non-limiting examples of insertion sites that may be used to insert the heterologous nucleotide sequences include, but are not limited to, Del2, IGR69-70, and Del3. FIG. 18 shows the sequence of an sMVA backbone (SEQ ID NO: 1) and the possible locations of insertion sites Del2 ([[<<DEL2INSERT]], where “<<” indicates the heterologous nucleotide insert at Del2 is the reverse complement of the SARS-CoV-2 protein sequence (or variants, mutants, and/or immunogenic fragments thereof) sequence), IGR69-70 ([[<<INSERTIGR69/70]_{A}], [[<<INSERTIGR69/70]_{B}], [[<<INSERTIGR69/70]_{C}], or [[<<INSERTIGR69/70]D], where “<<” indicates the heterologous nucleotide insert at Del2 is the reverse complement of the SARS-CoV-2 protein sequence (or variants, mutants, and/or immunogenic fragments thereof), and each insertion site is representative of four alternative insertion sites for IGR69/70: A, B, C, or D), and Del3 ([[DEL3INSERT>>]], where “>>” indicates the heterologous nucleotide insert at Del3 is the forward SARS-CoV-2 protein sequence (or variant, mutant, and/or immunogenic fragment thereof) according to some embodiments. FIG. 18 also includes an “X” (in bold underline) that may be a T or an A according to some embodiments.

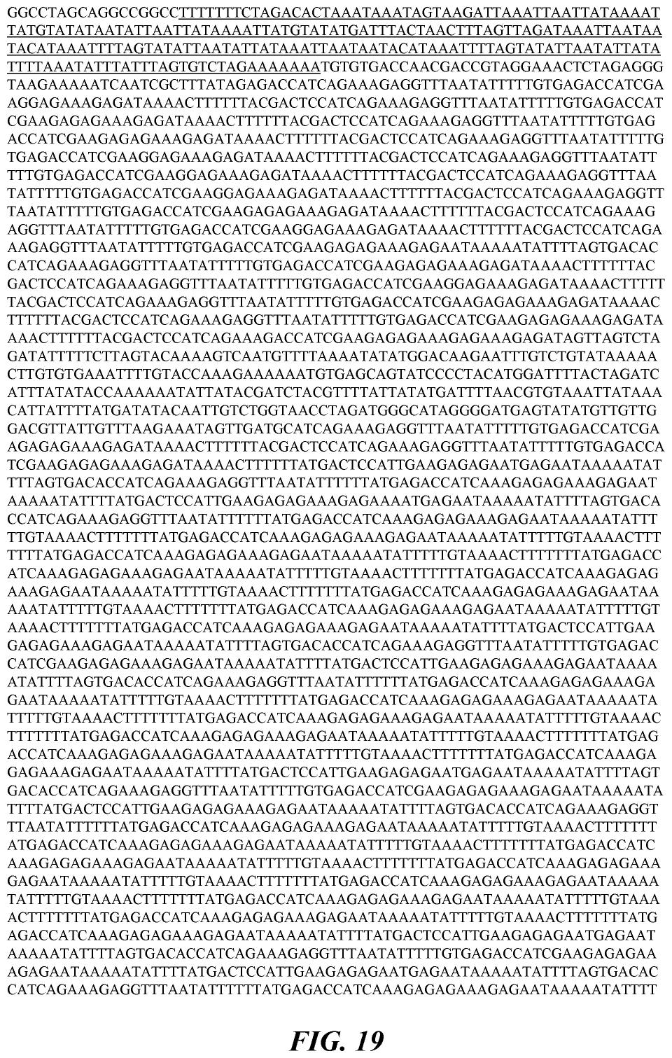

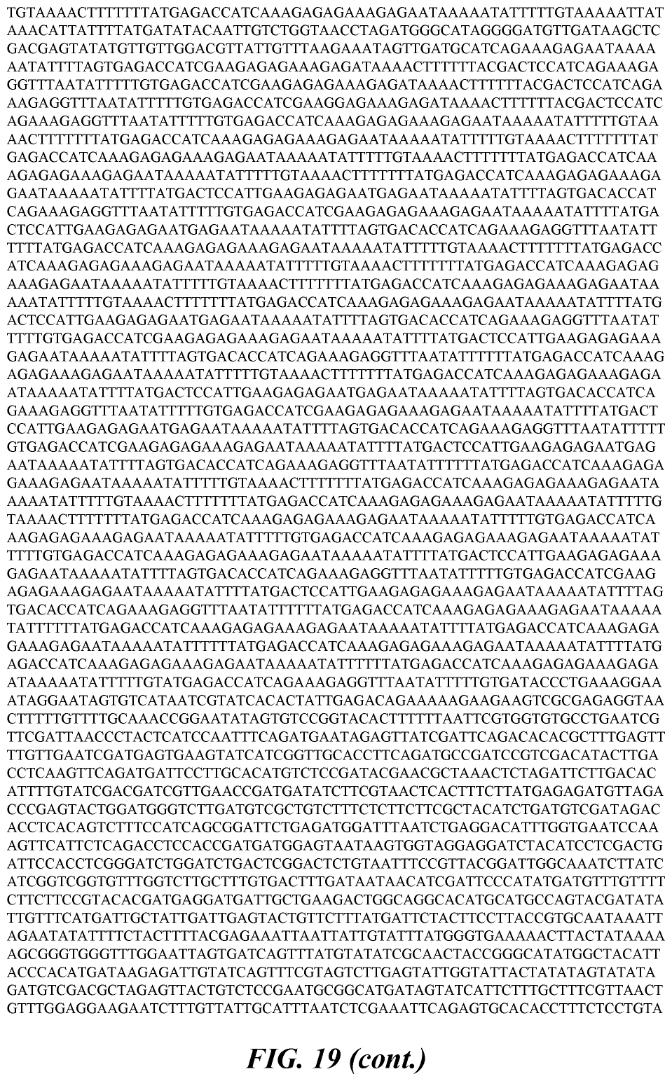

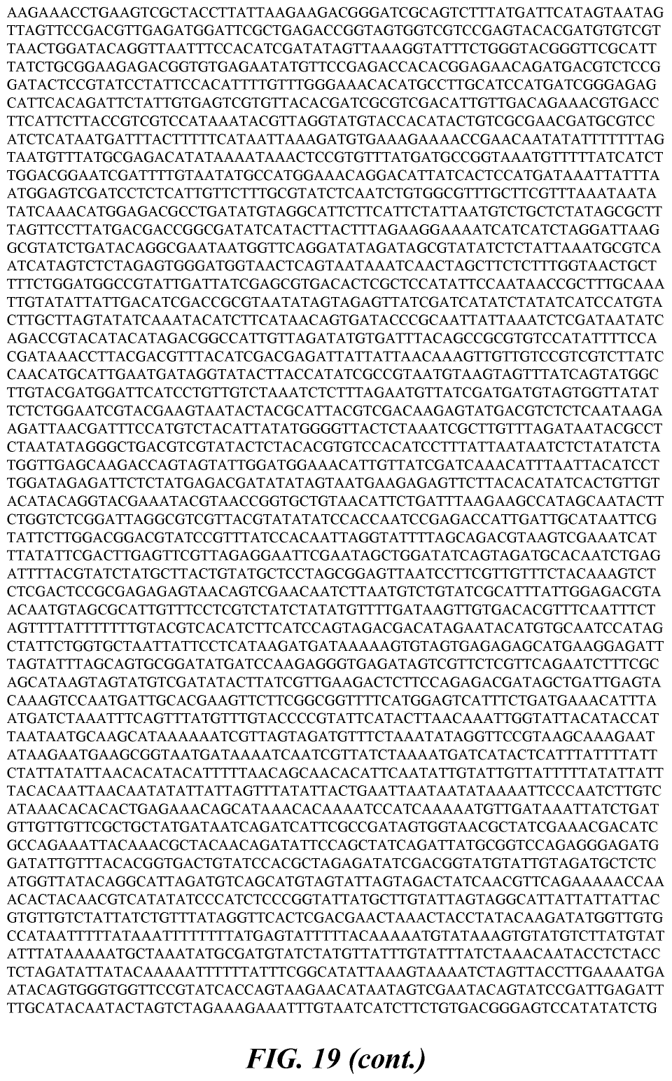

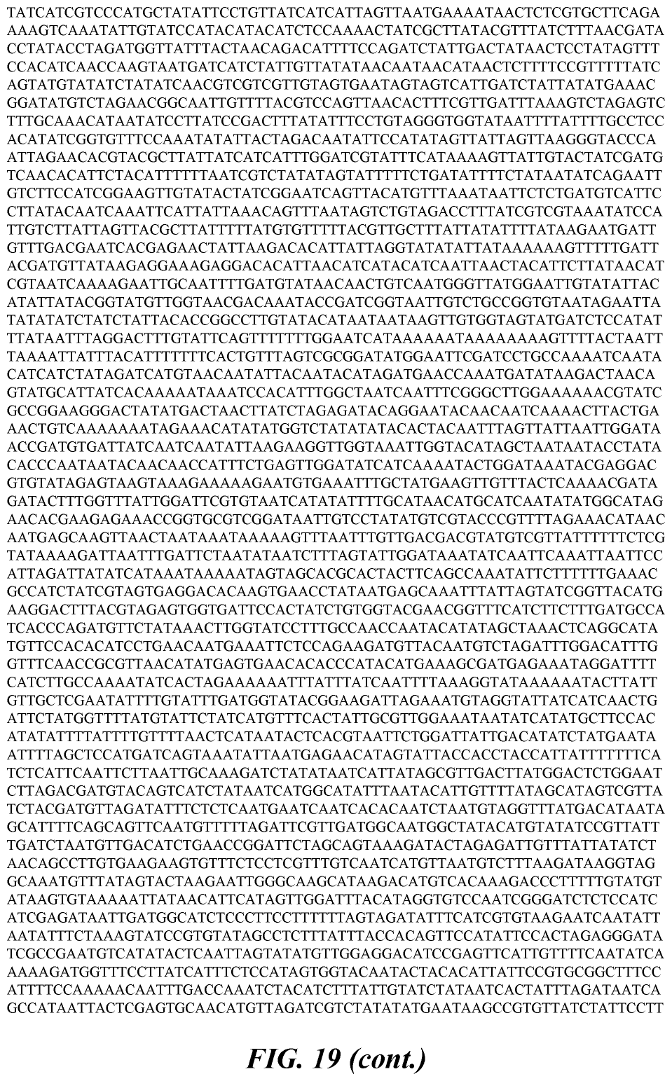

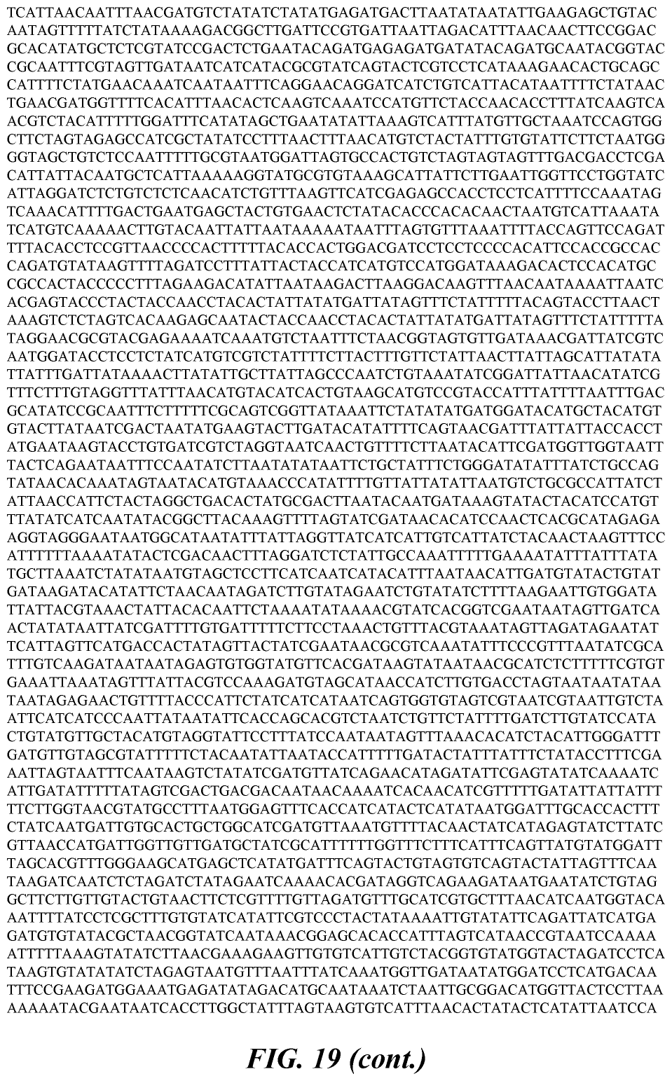

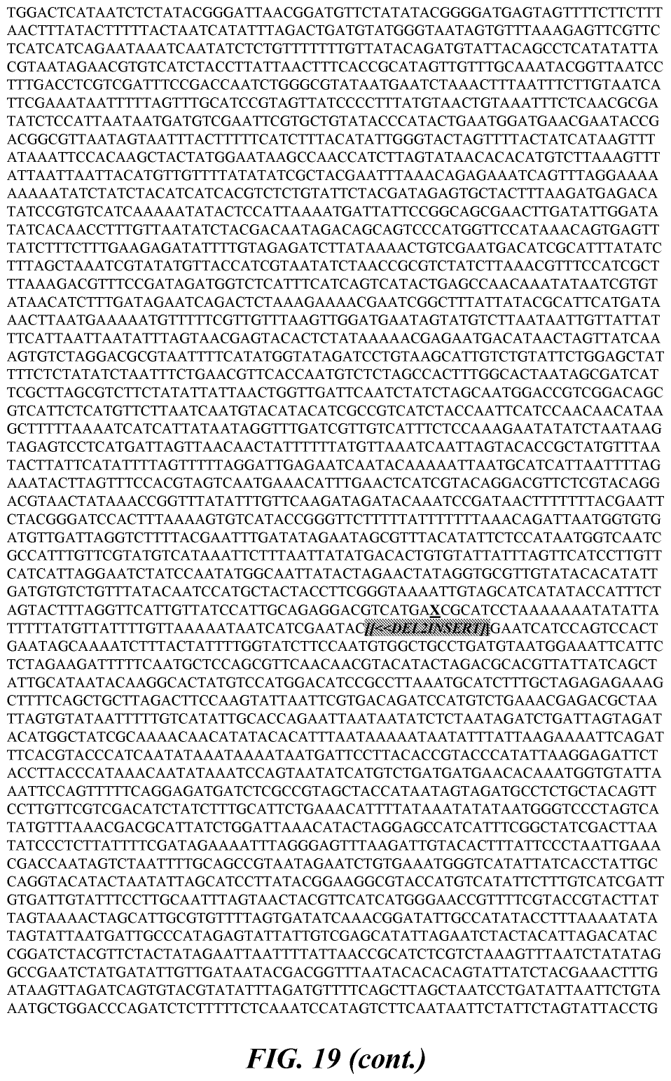

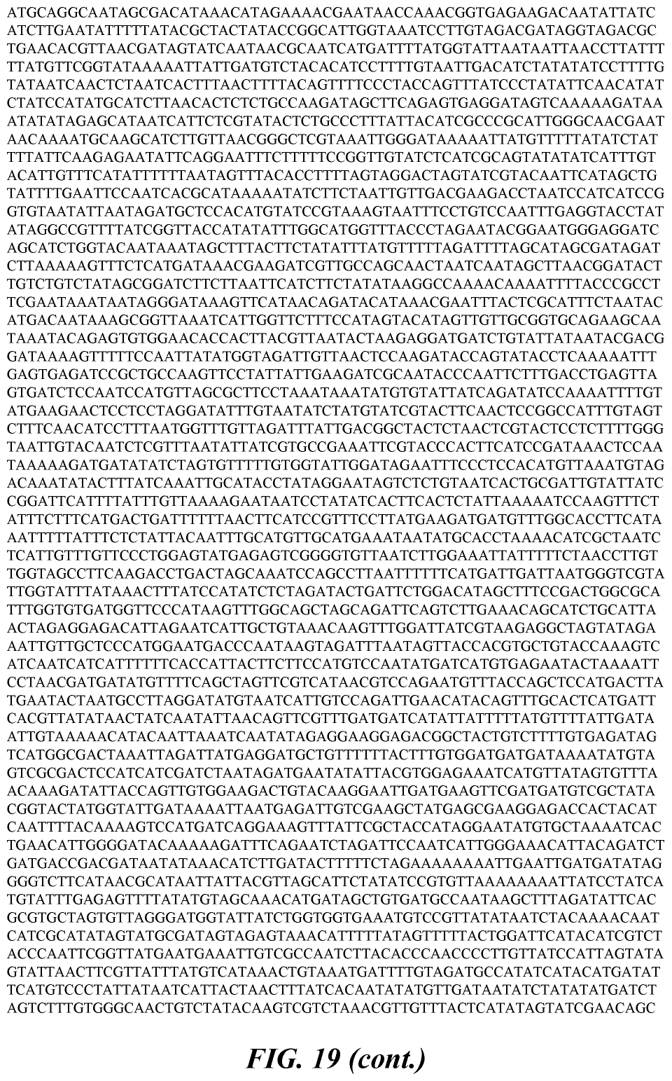

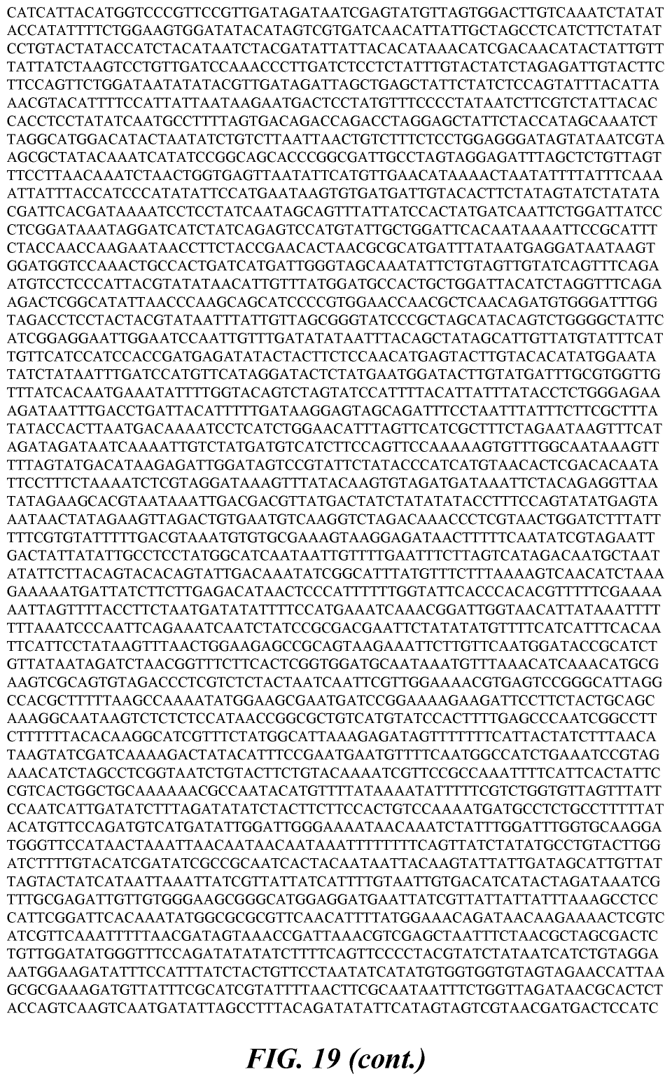

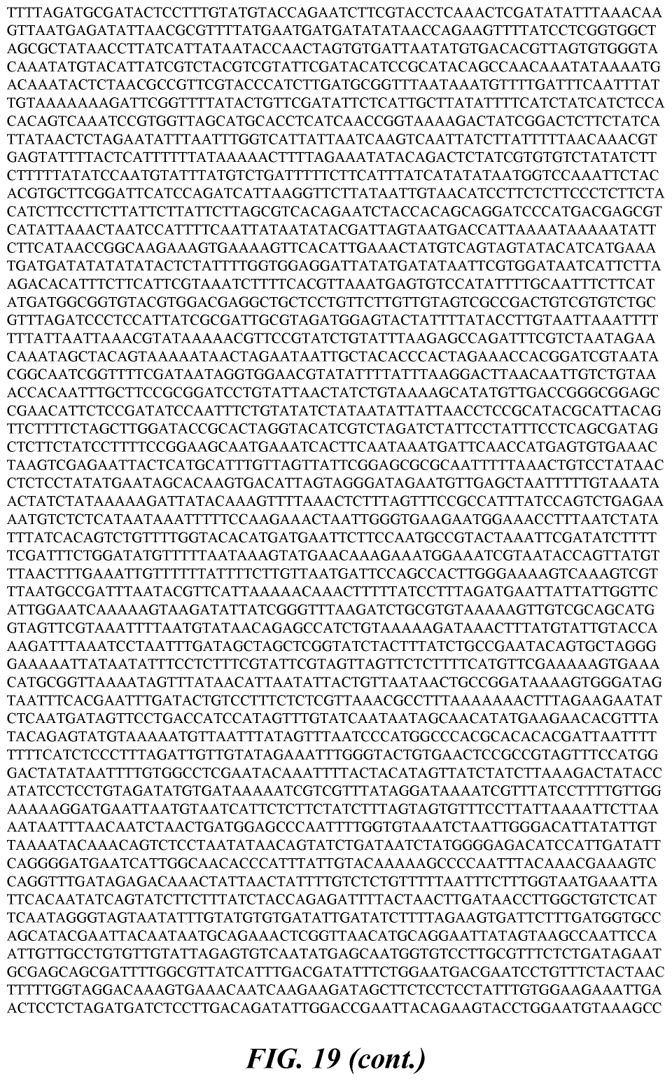

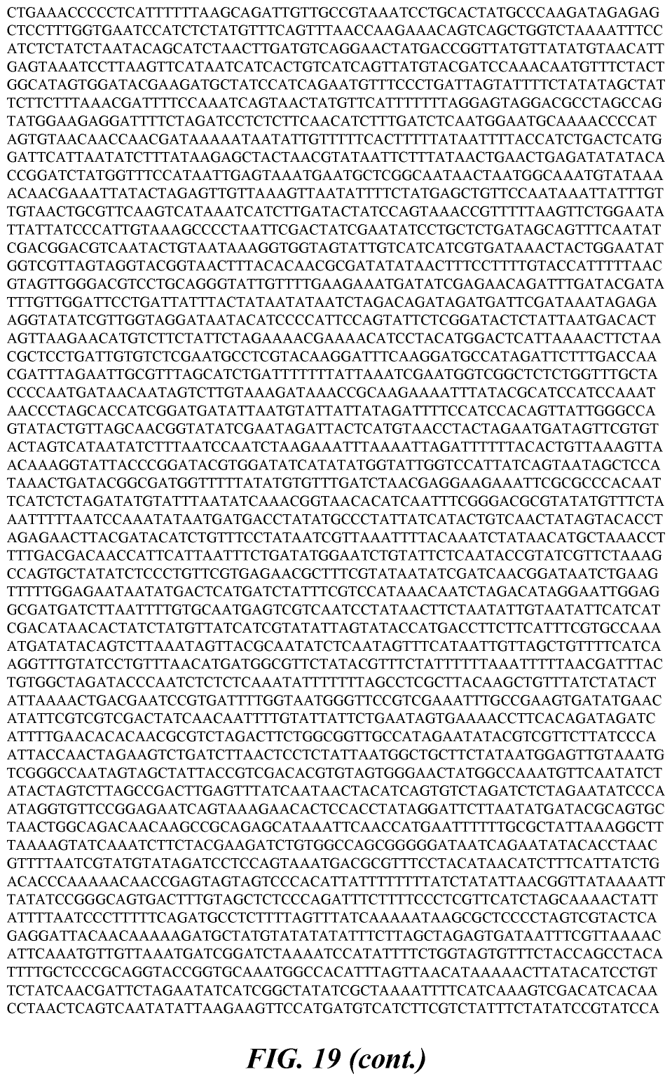

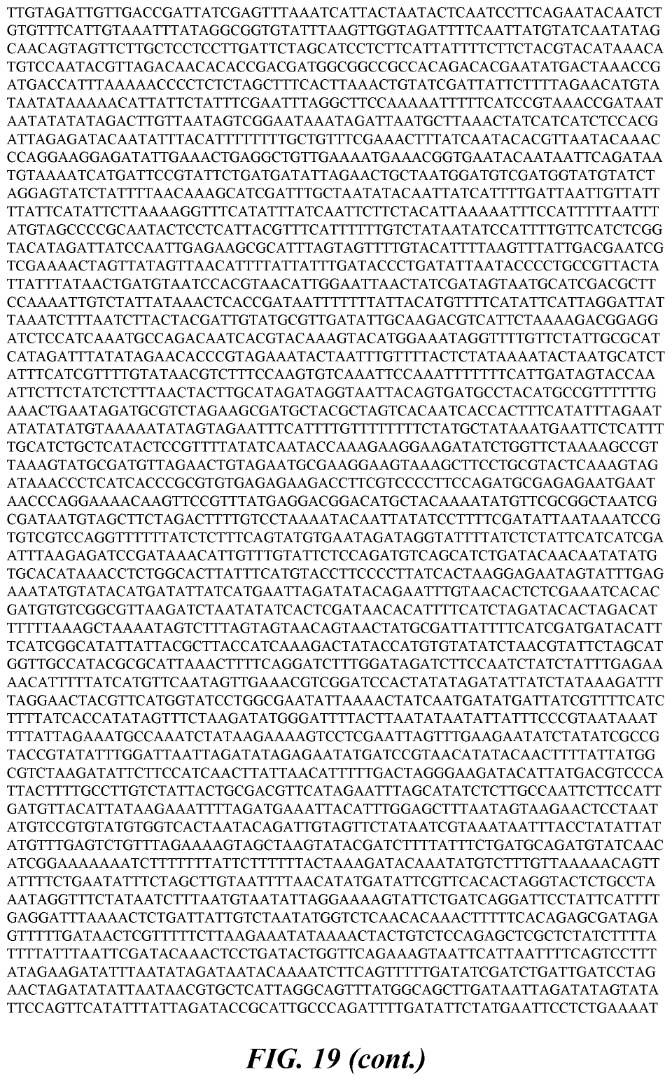

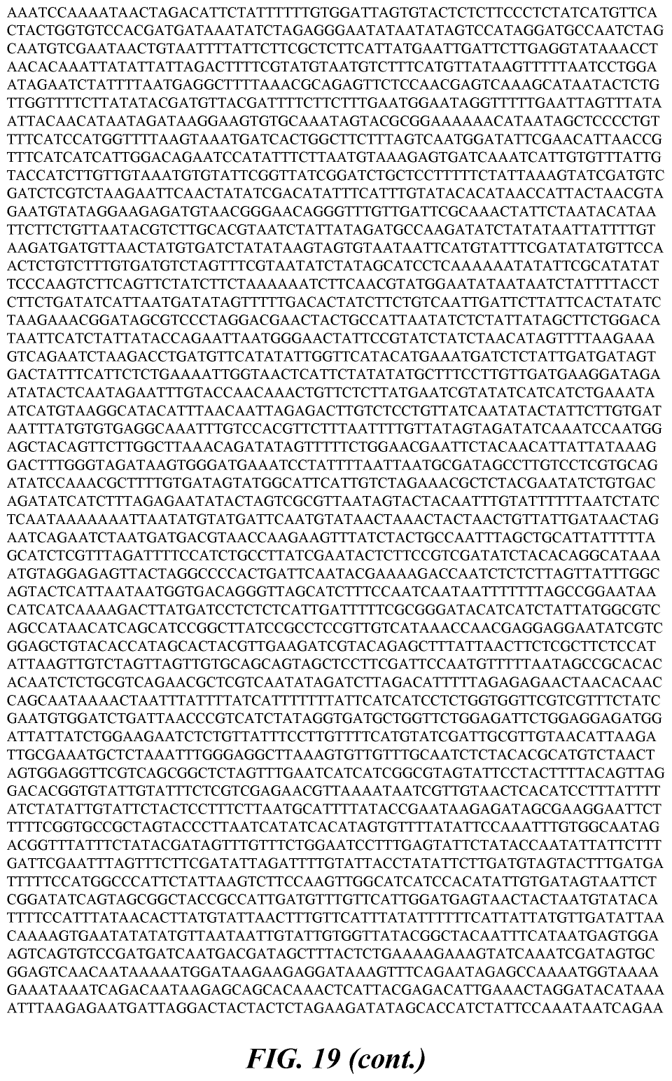

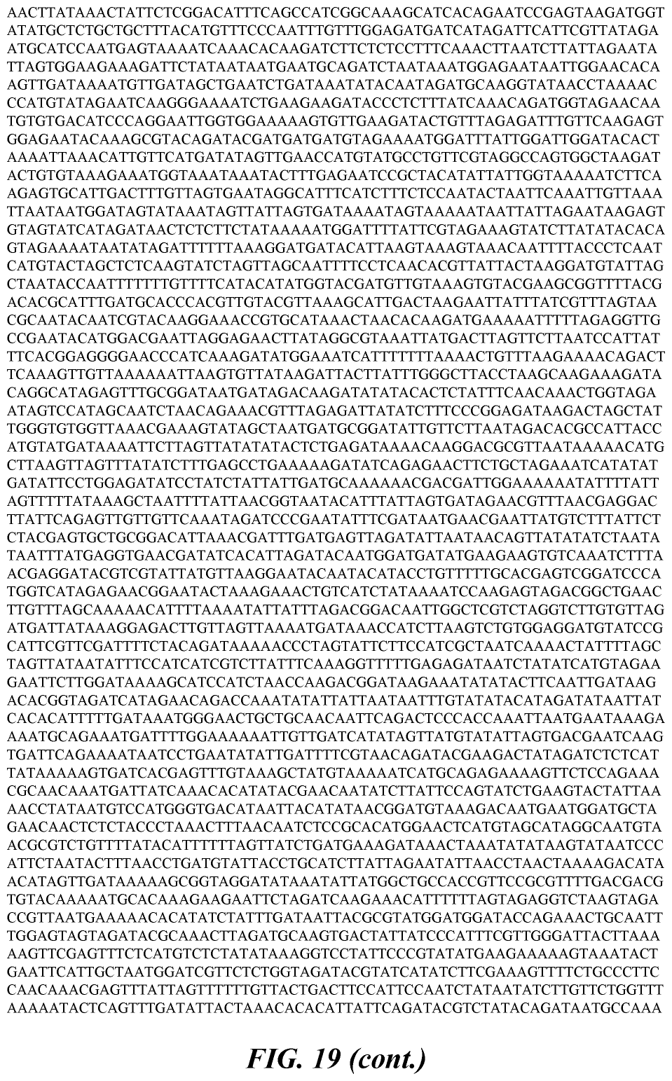

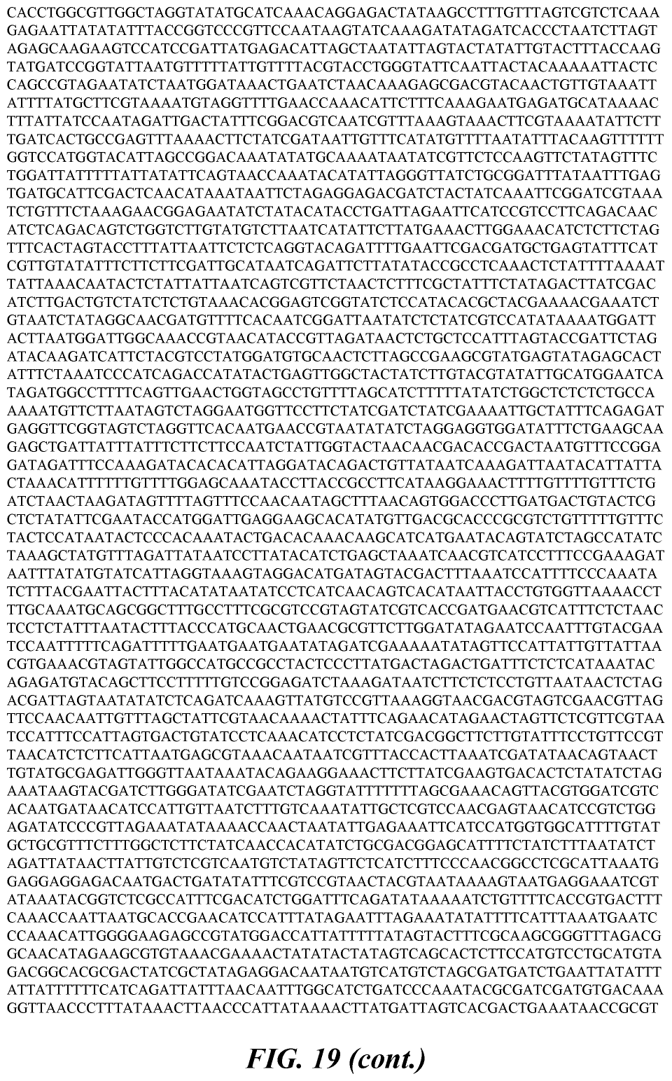

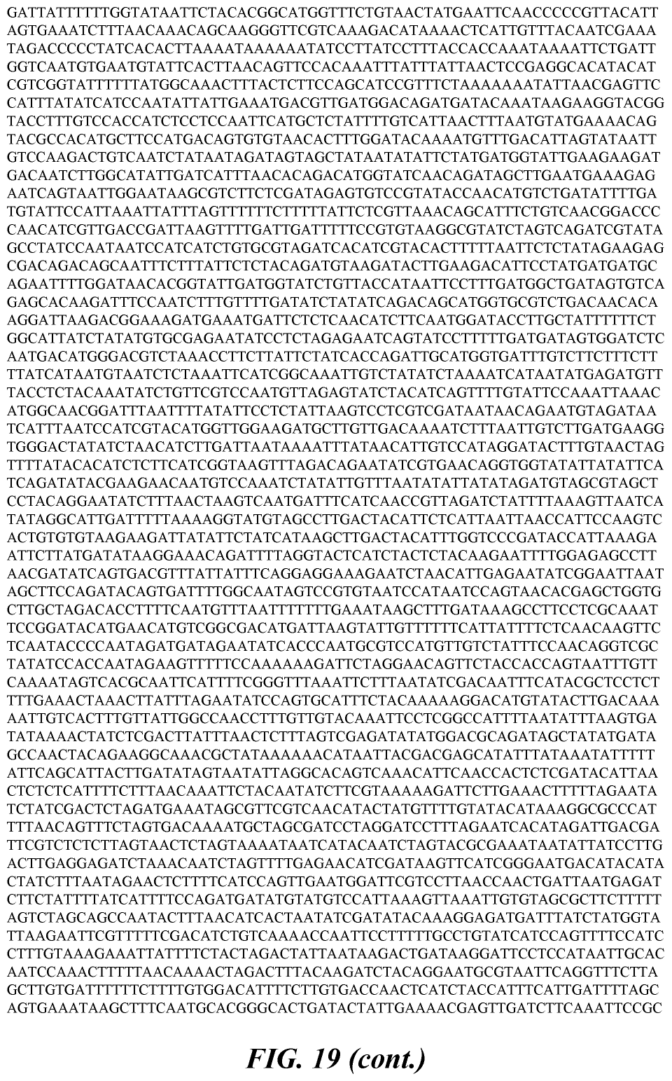

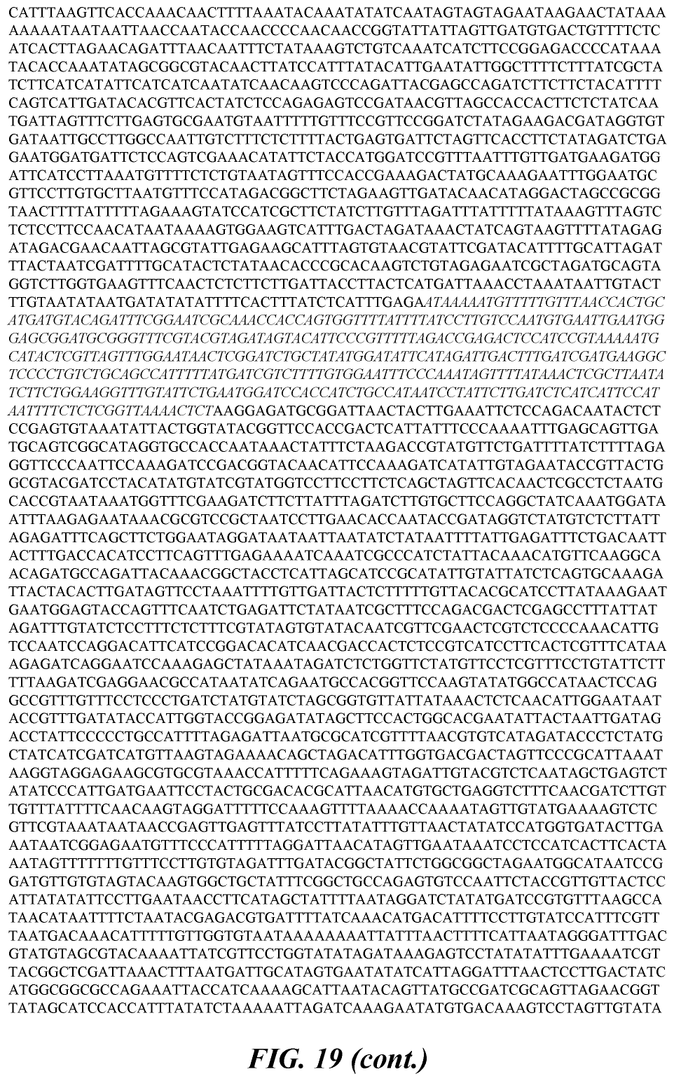

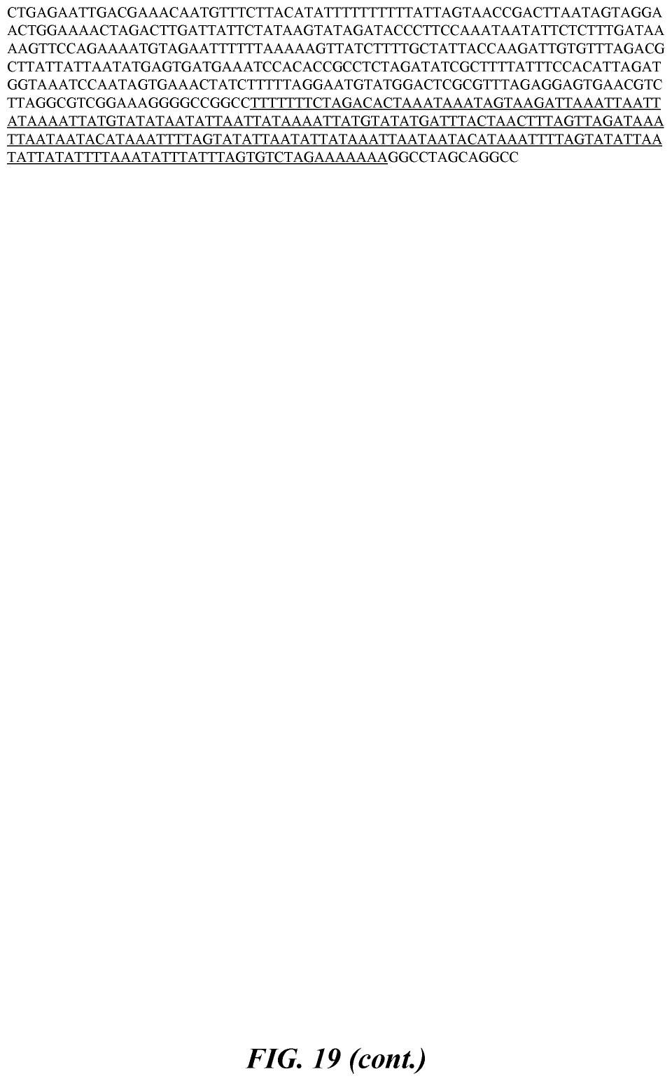

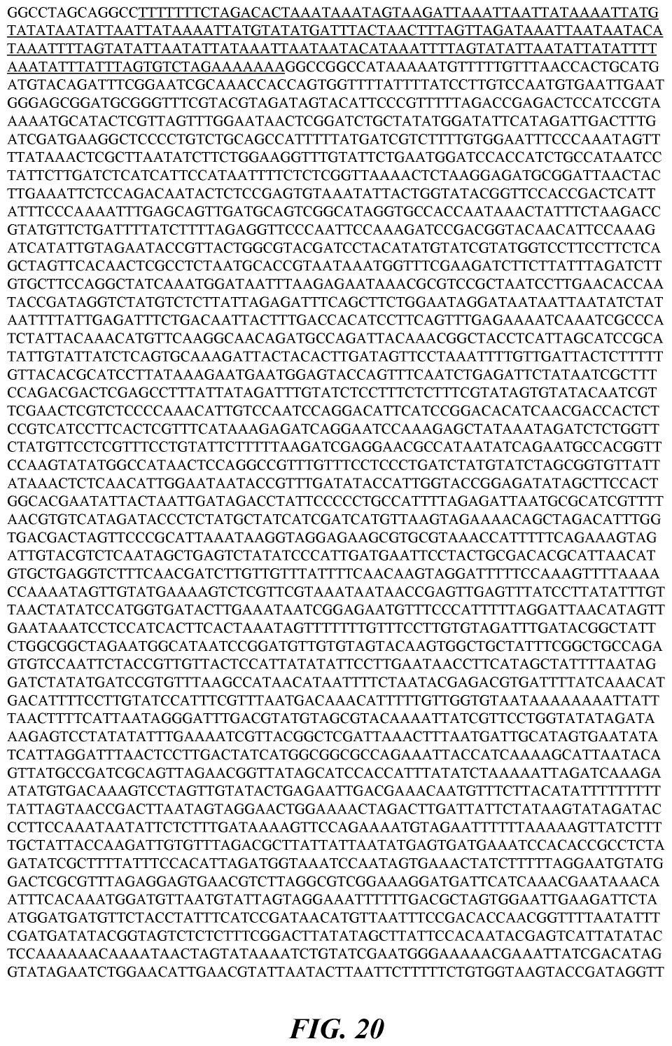

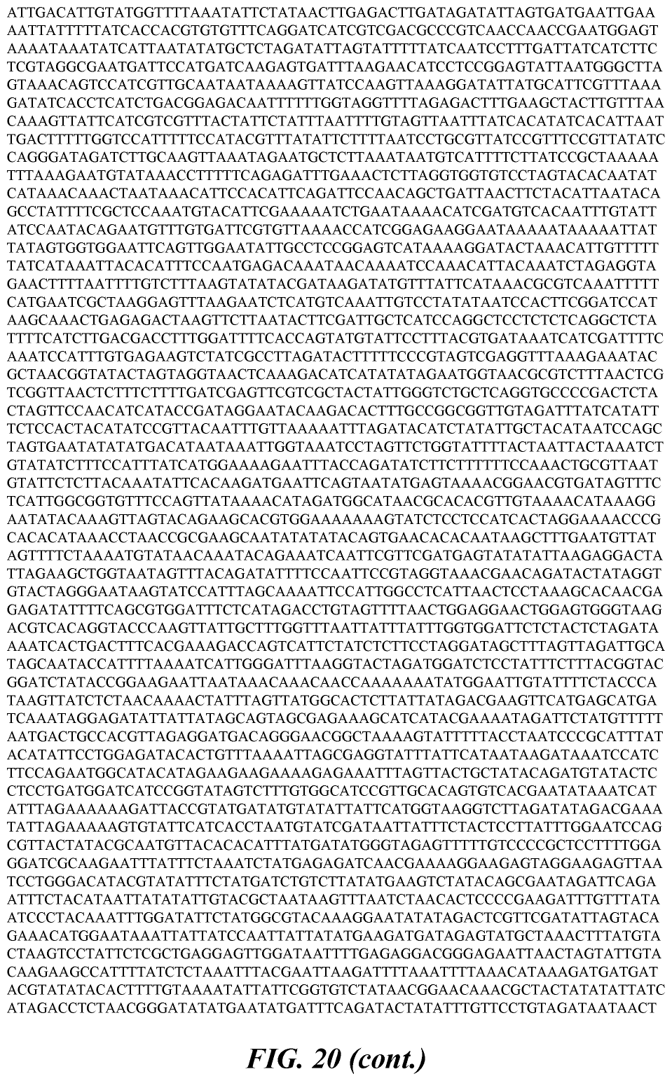

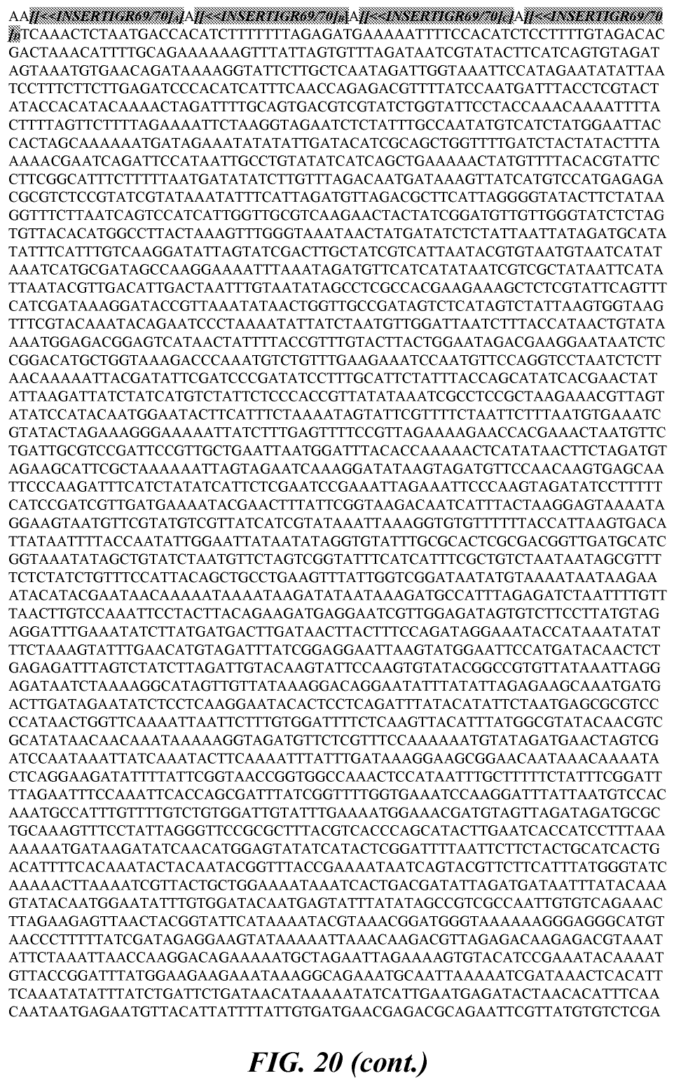

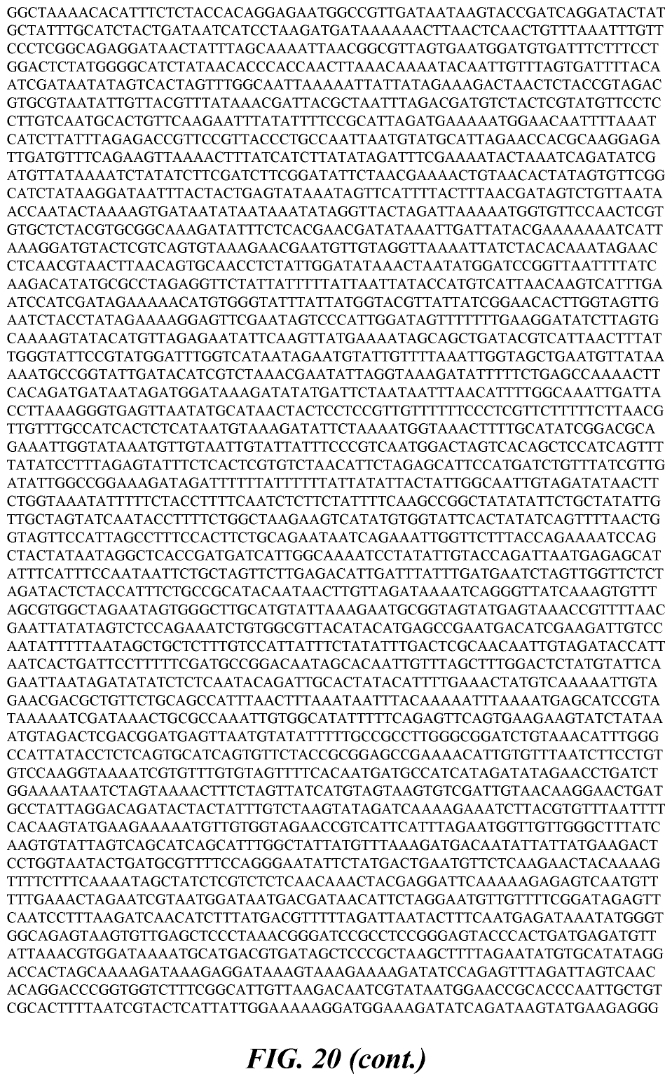

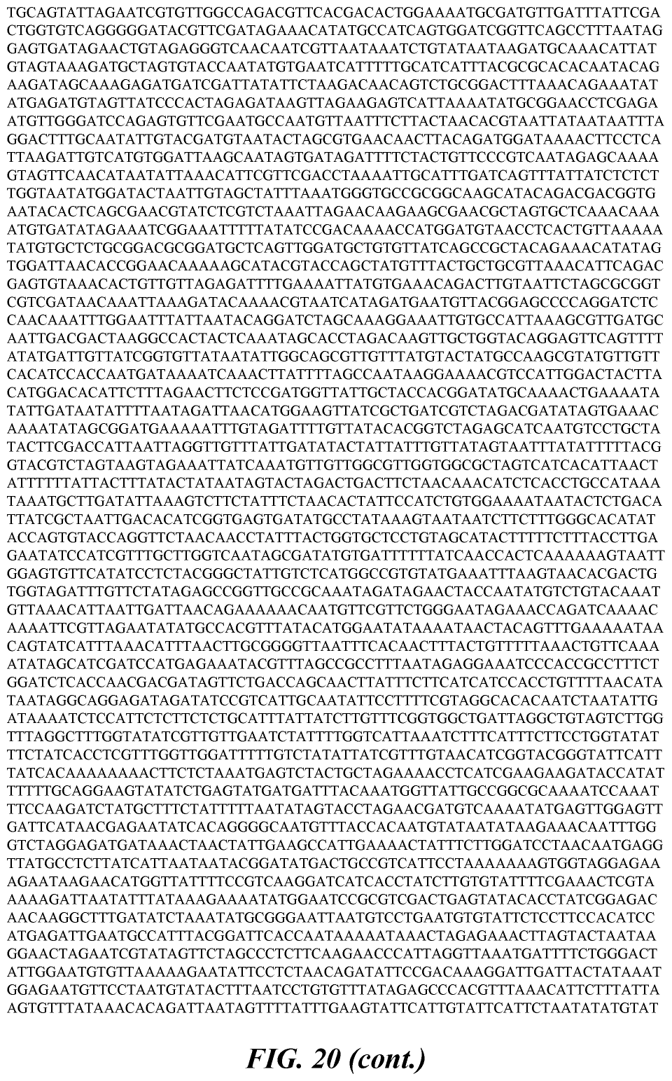

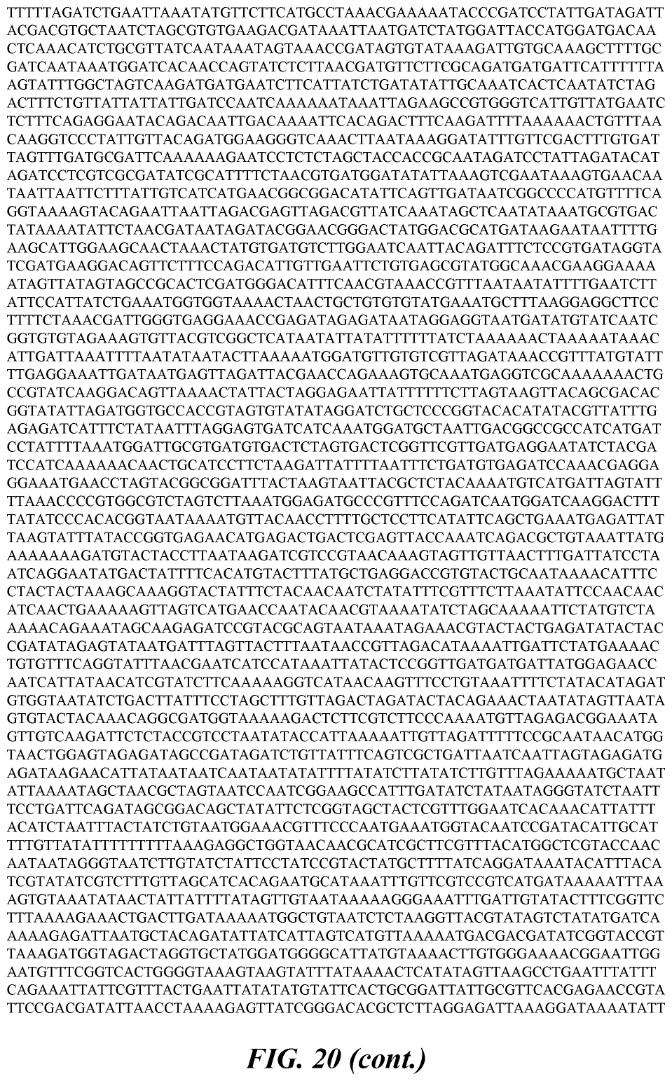

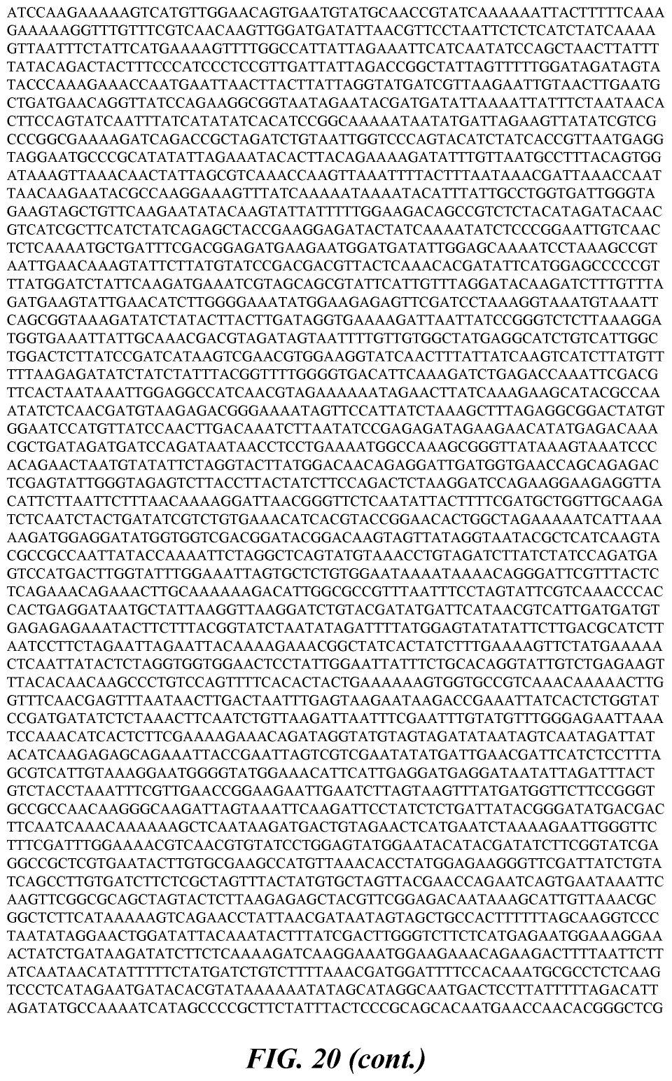

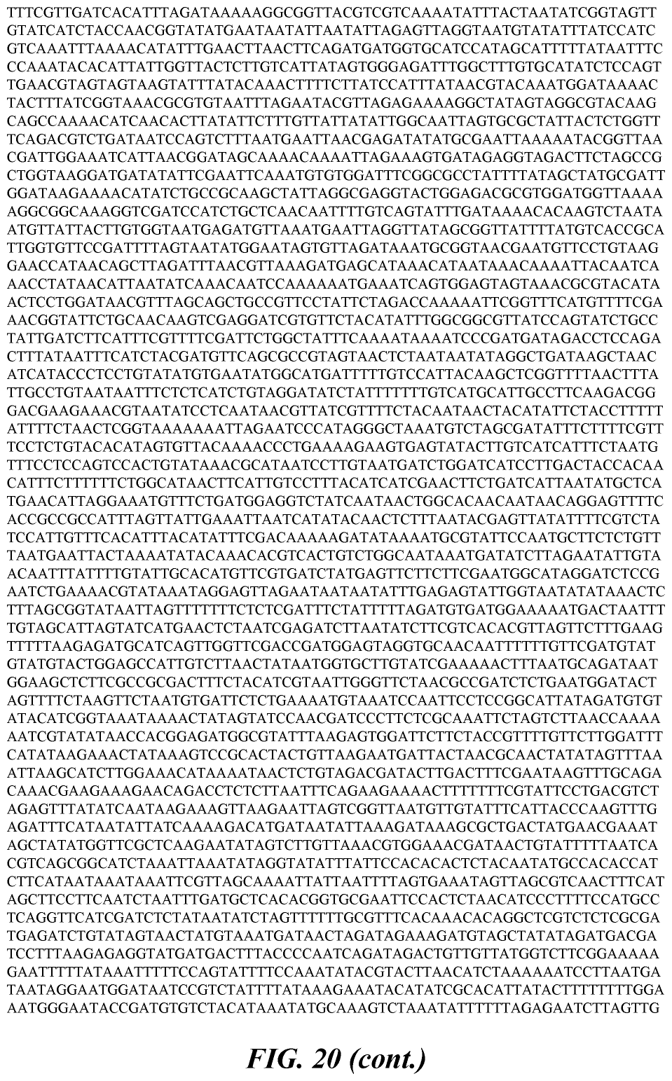

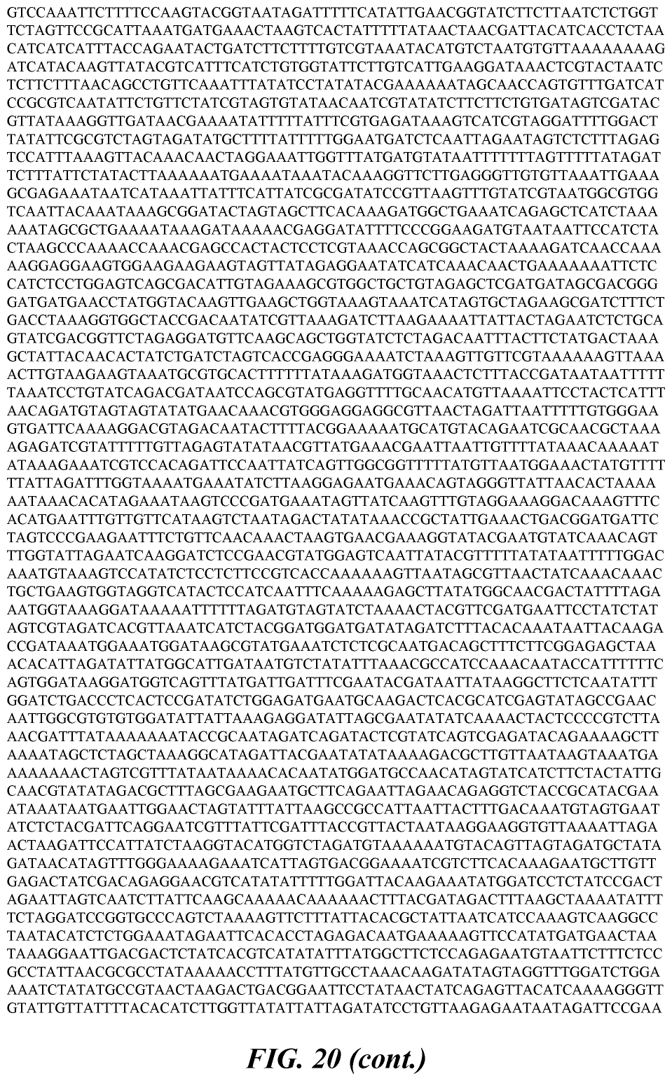

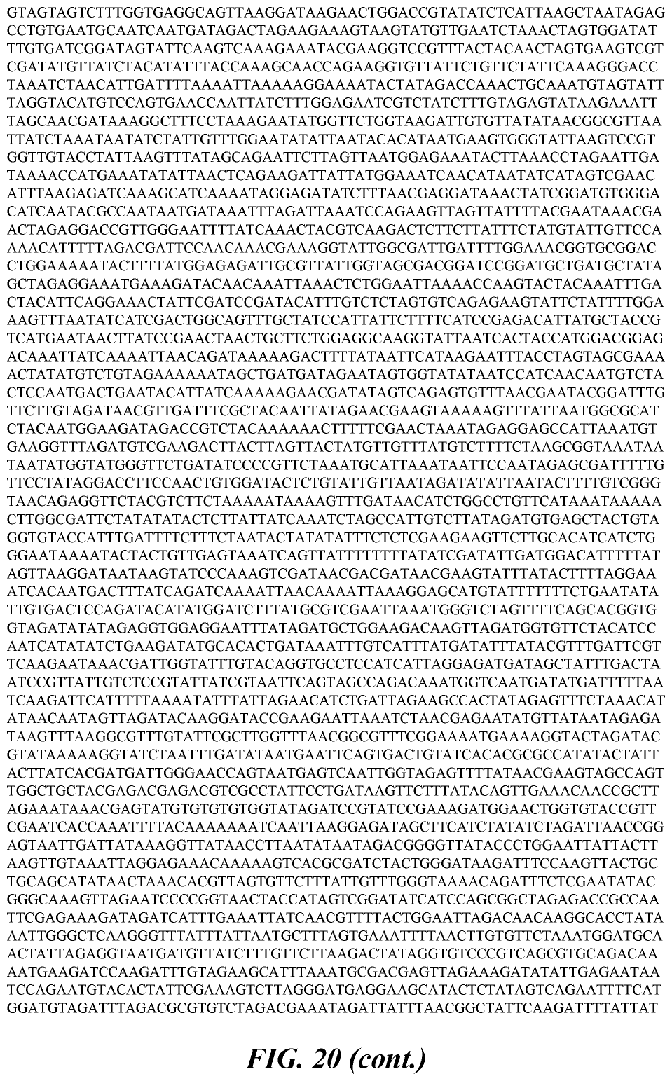

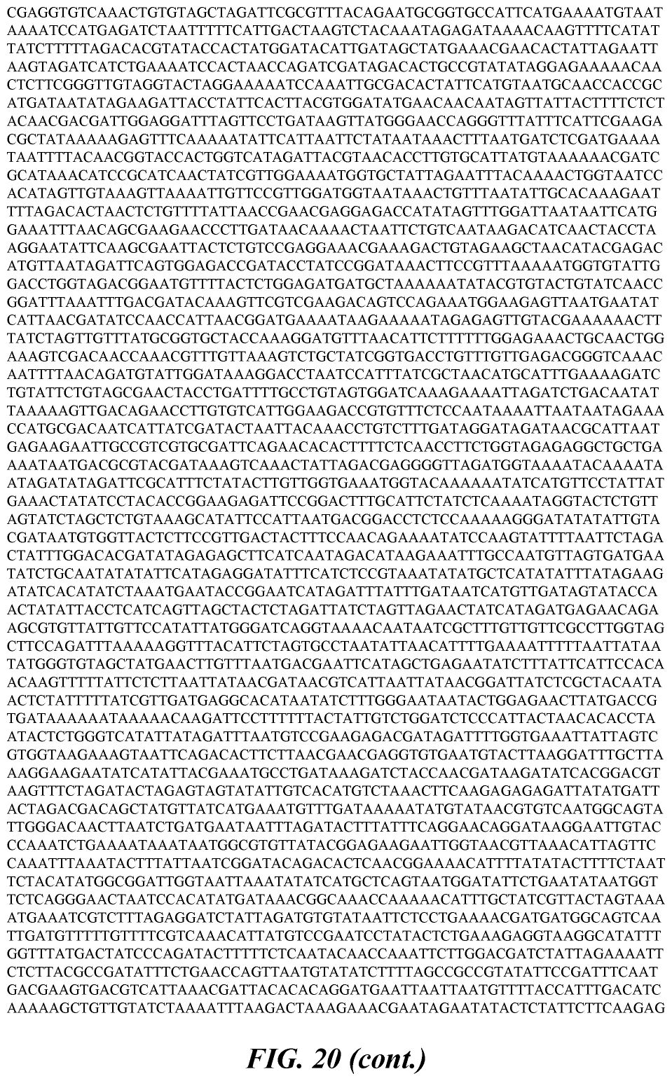

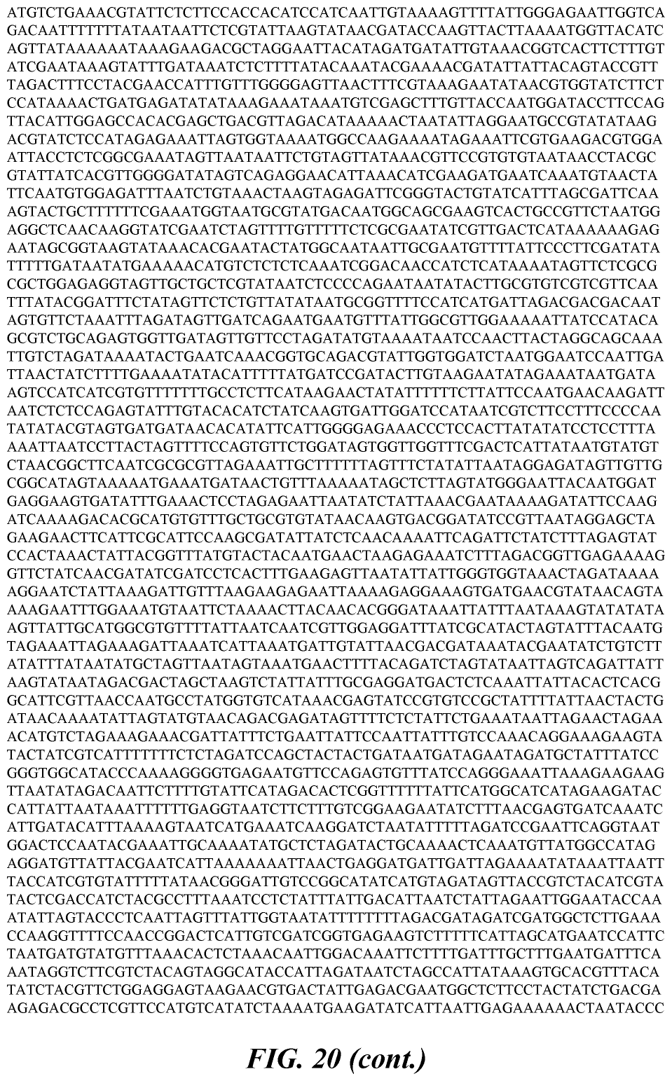

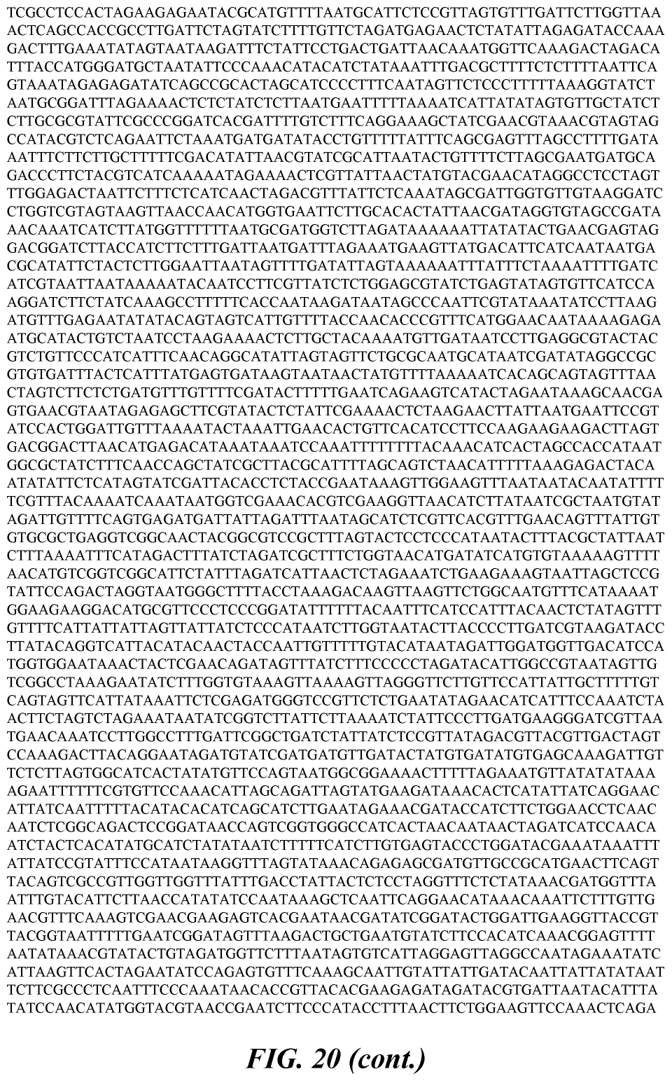

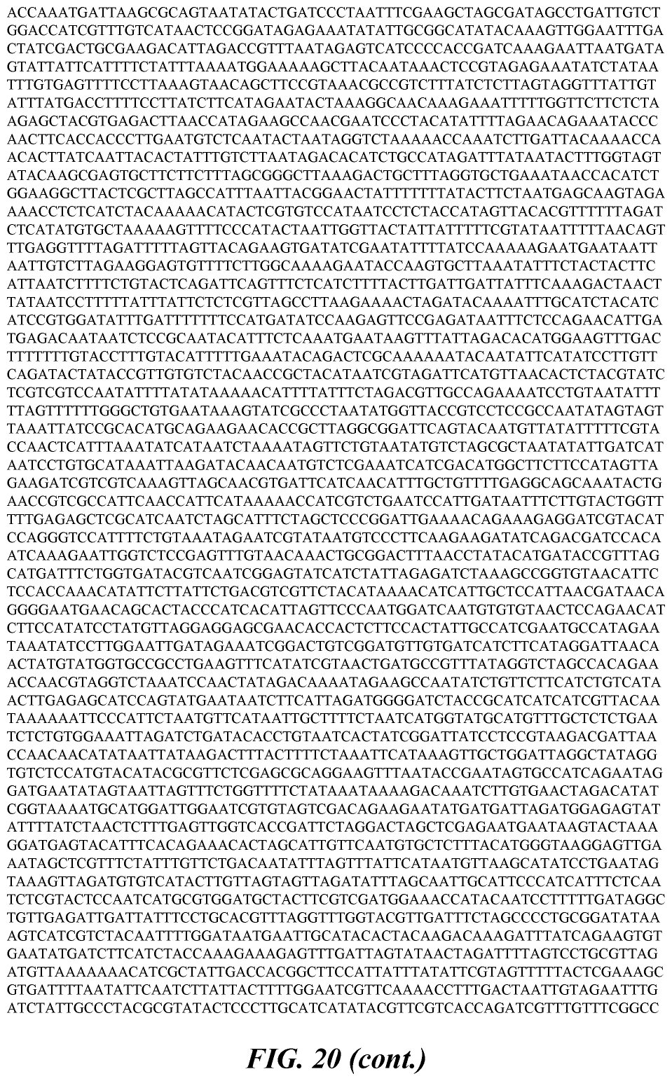

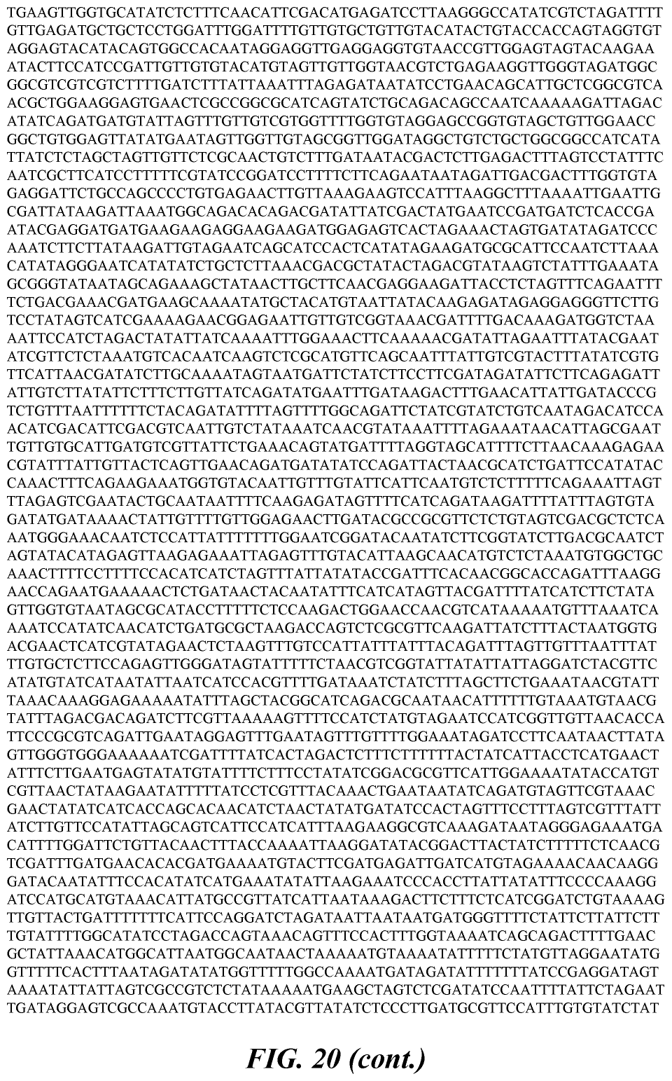

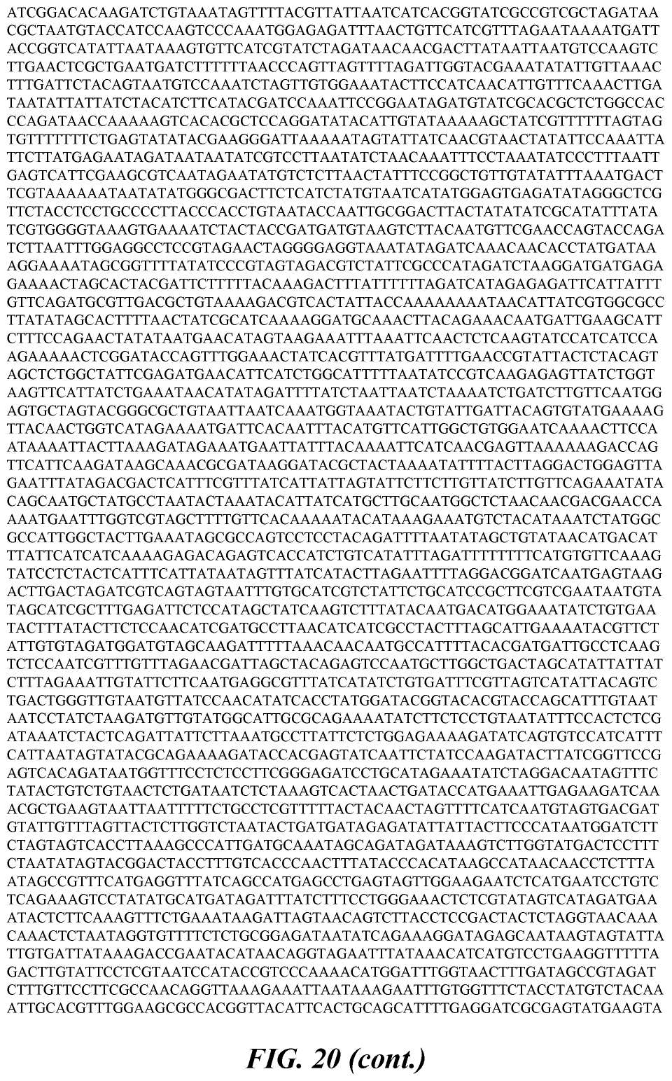

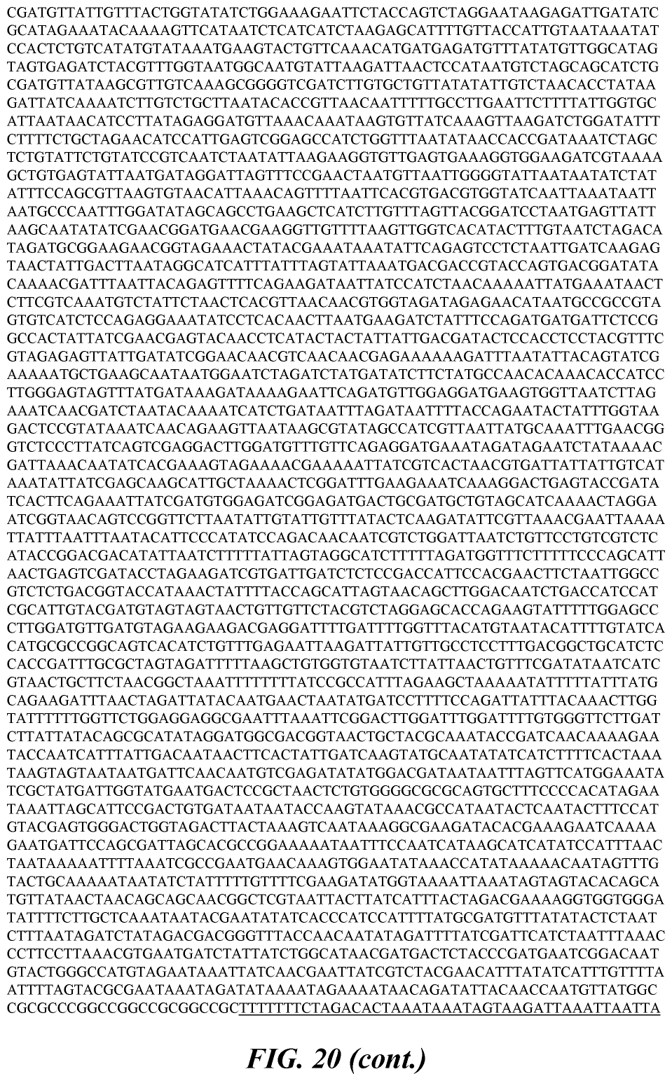

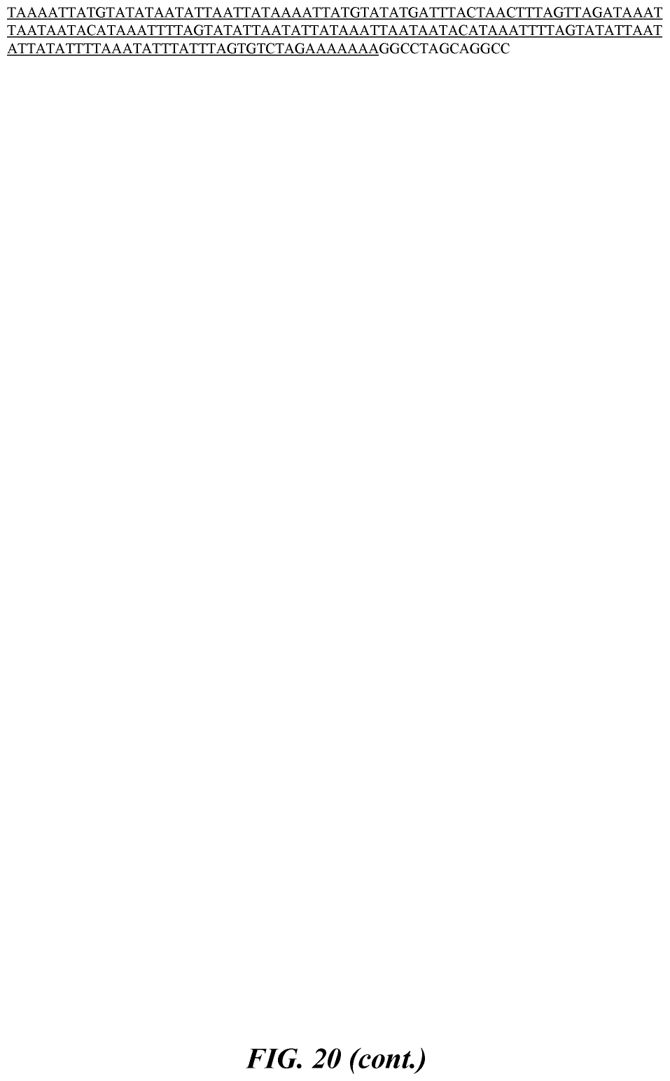

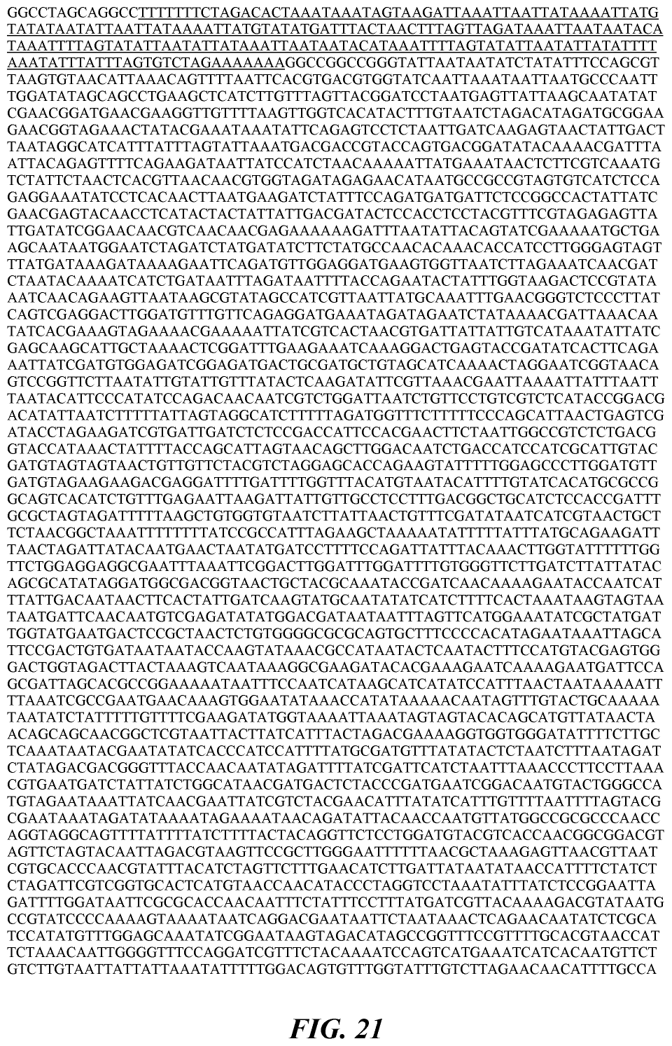

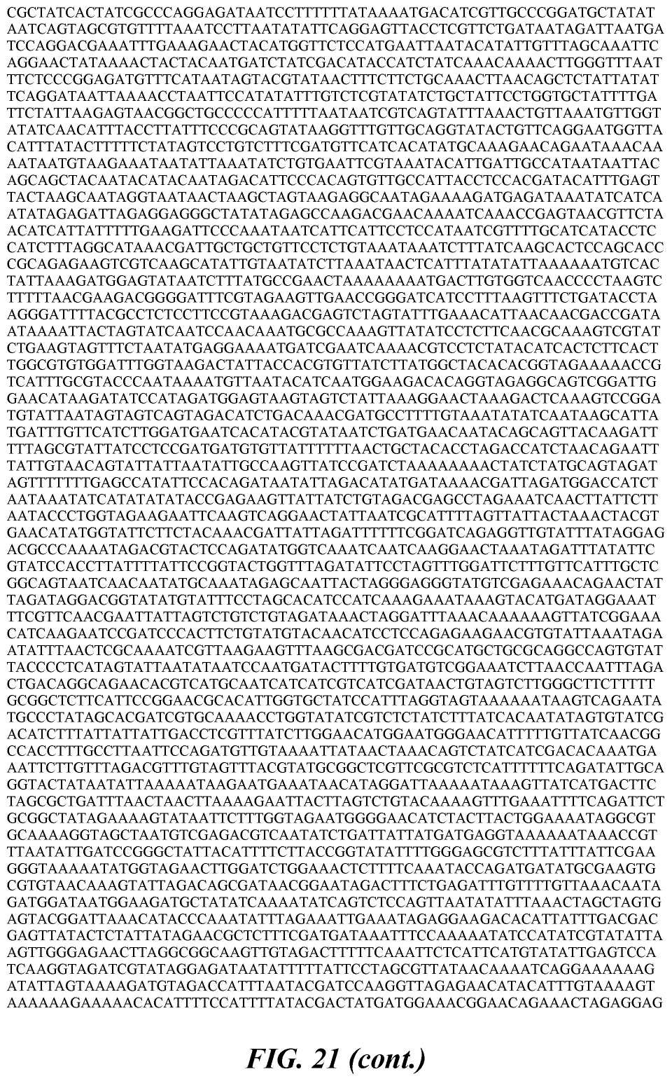

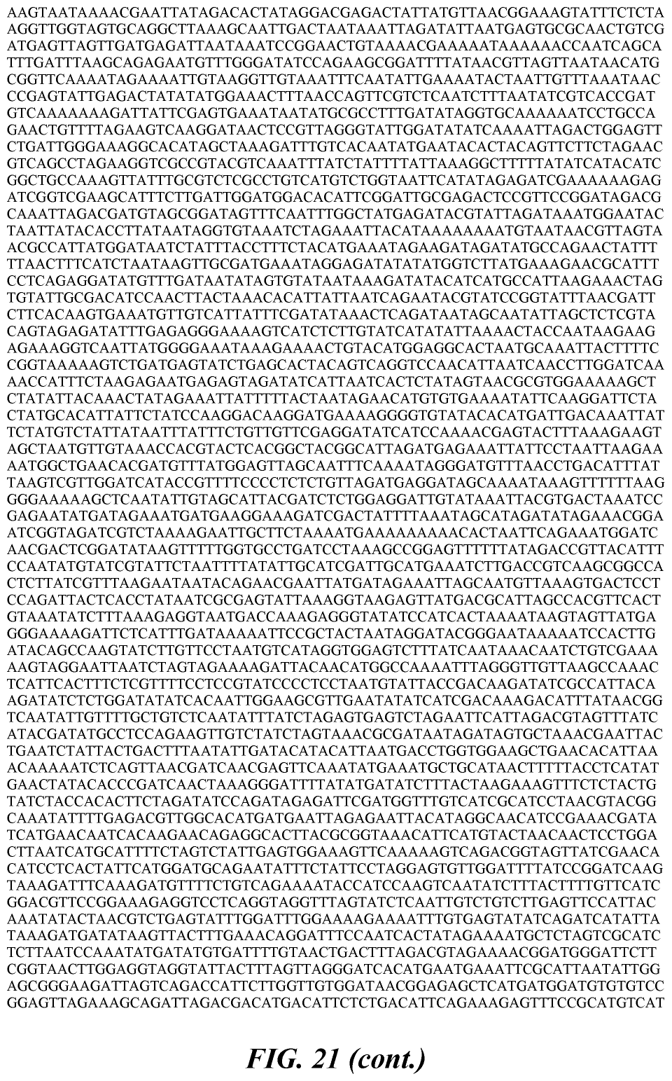

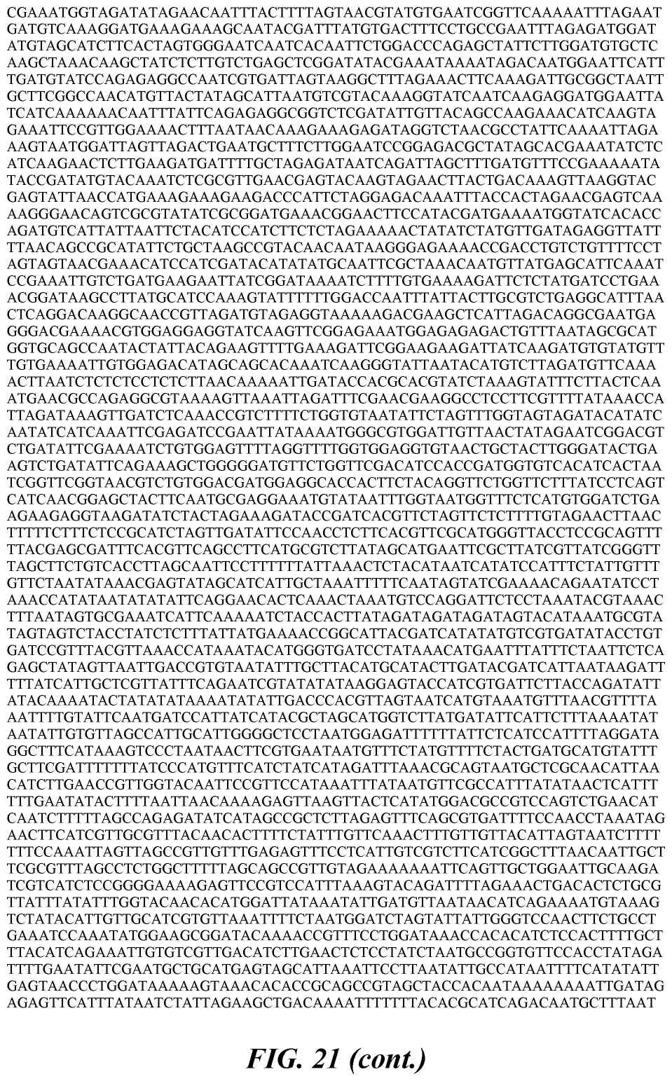

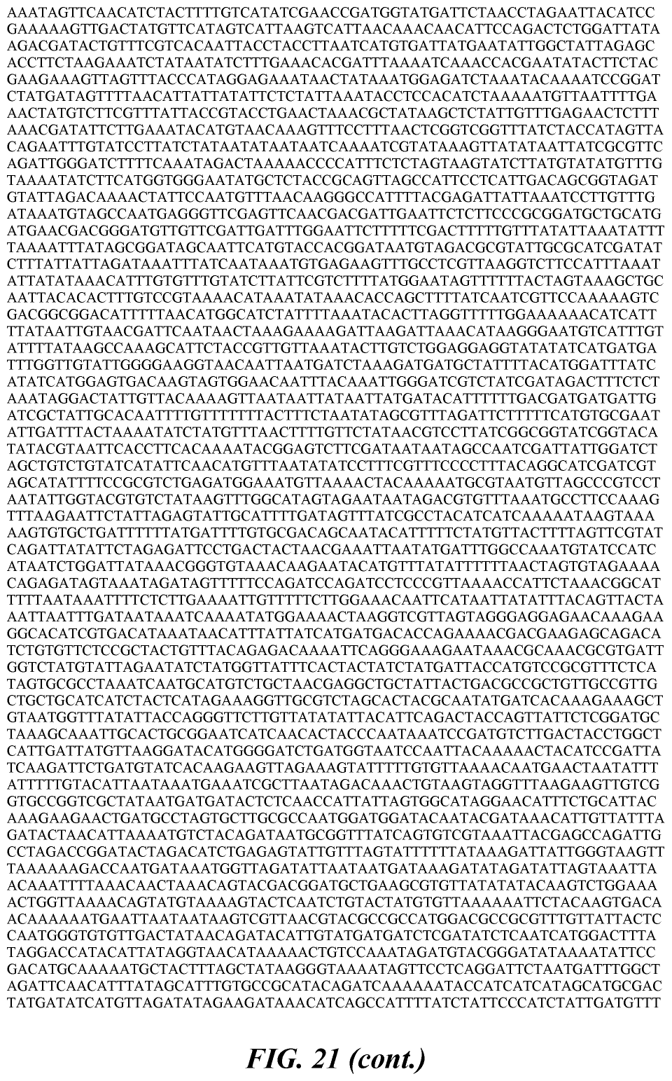

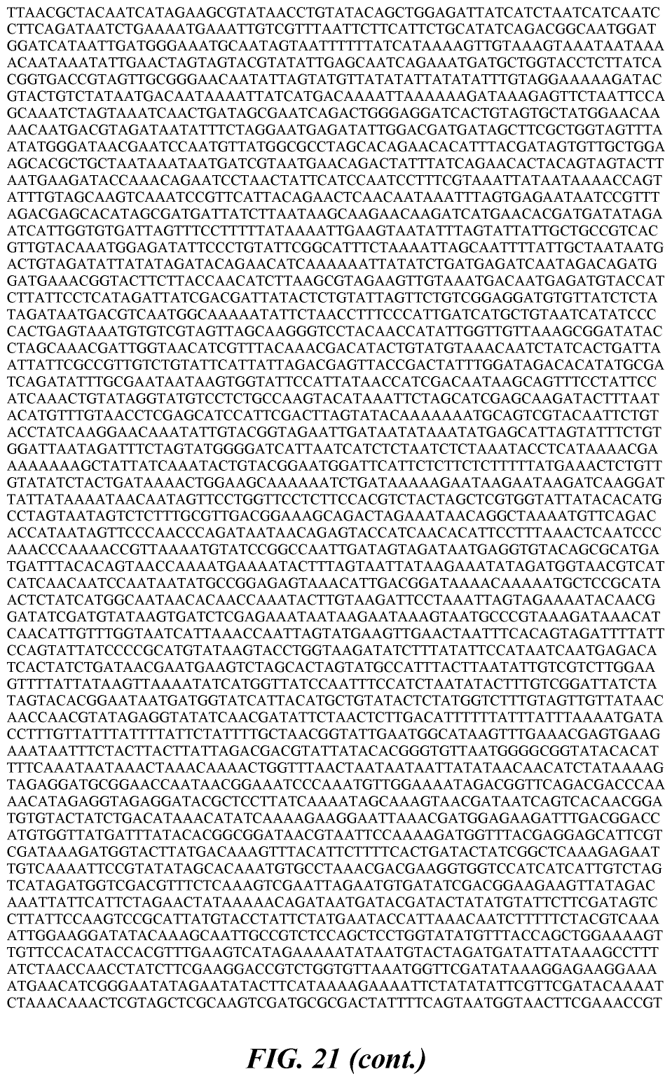

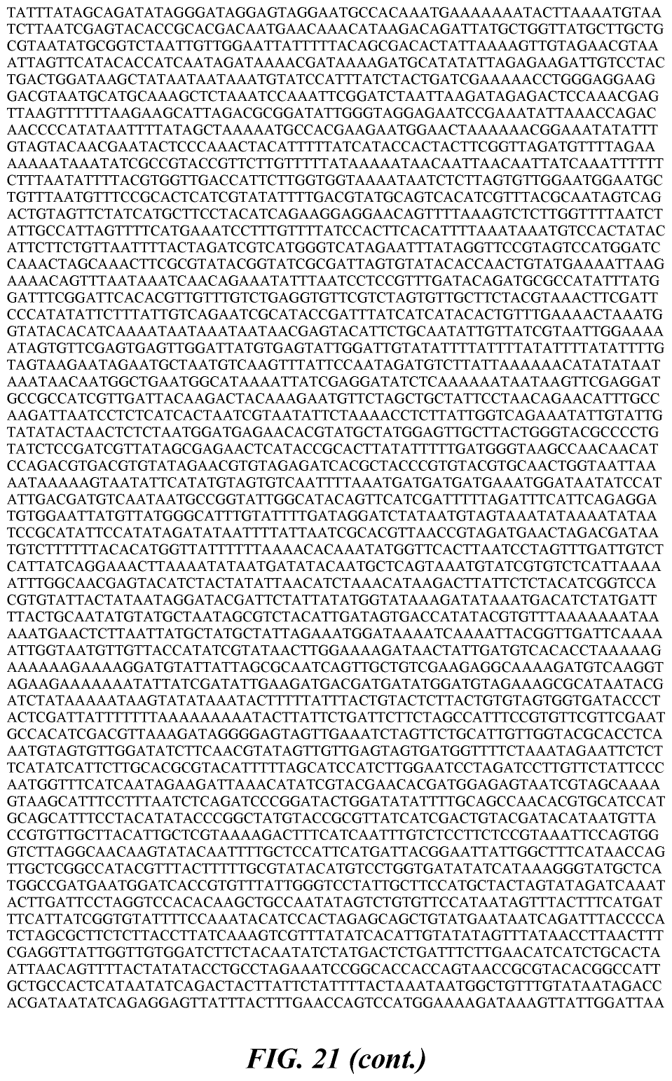

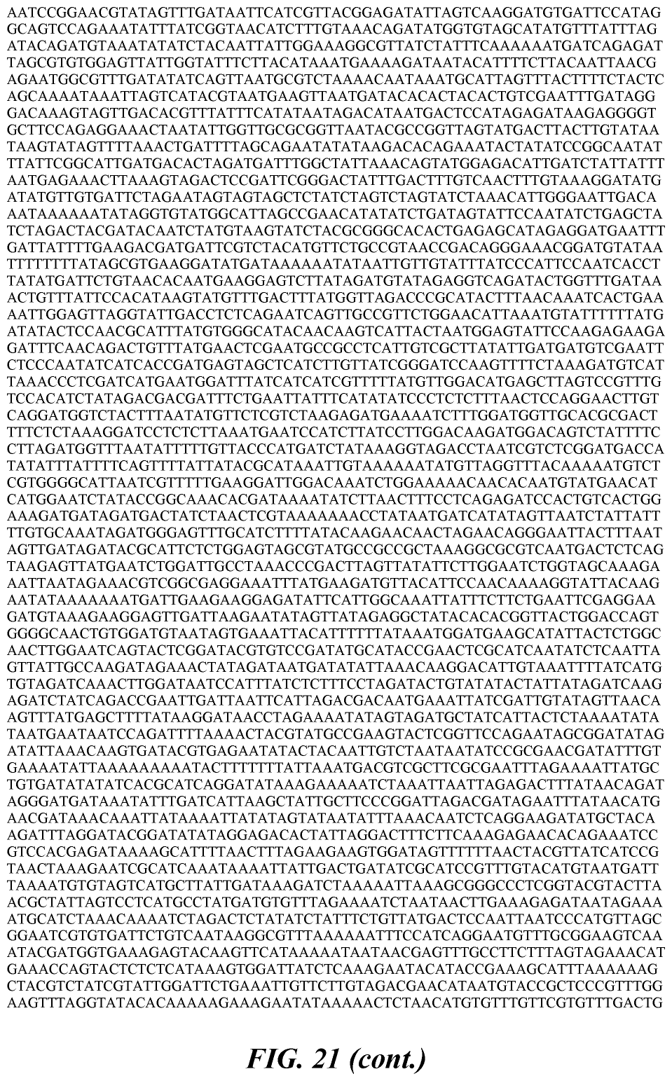

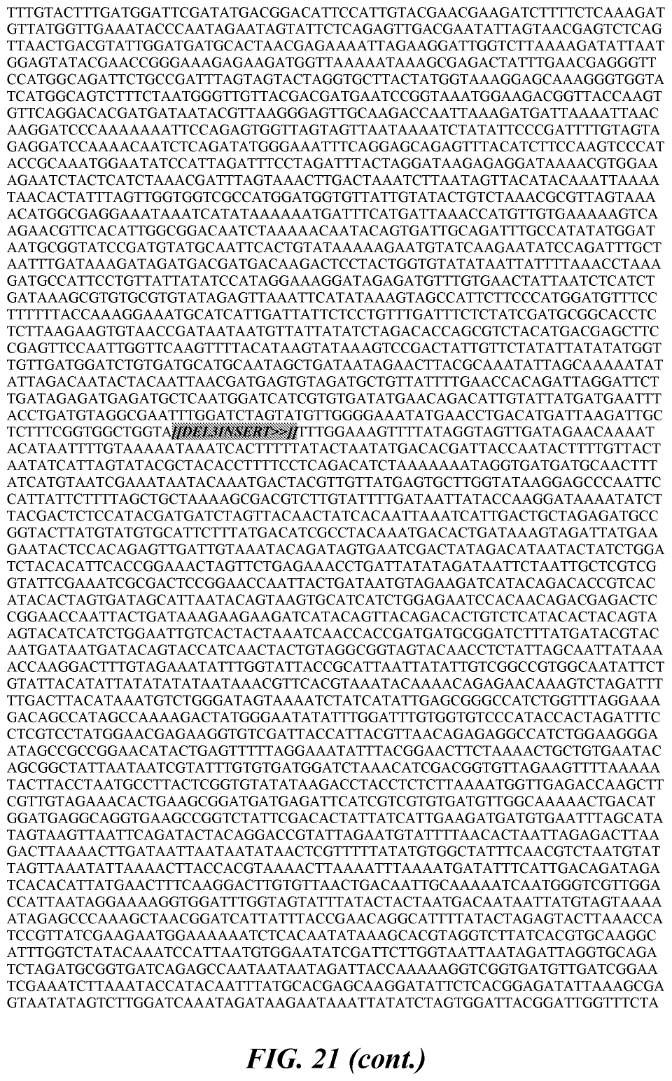

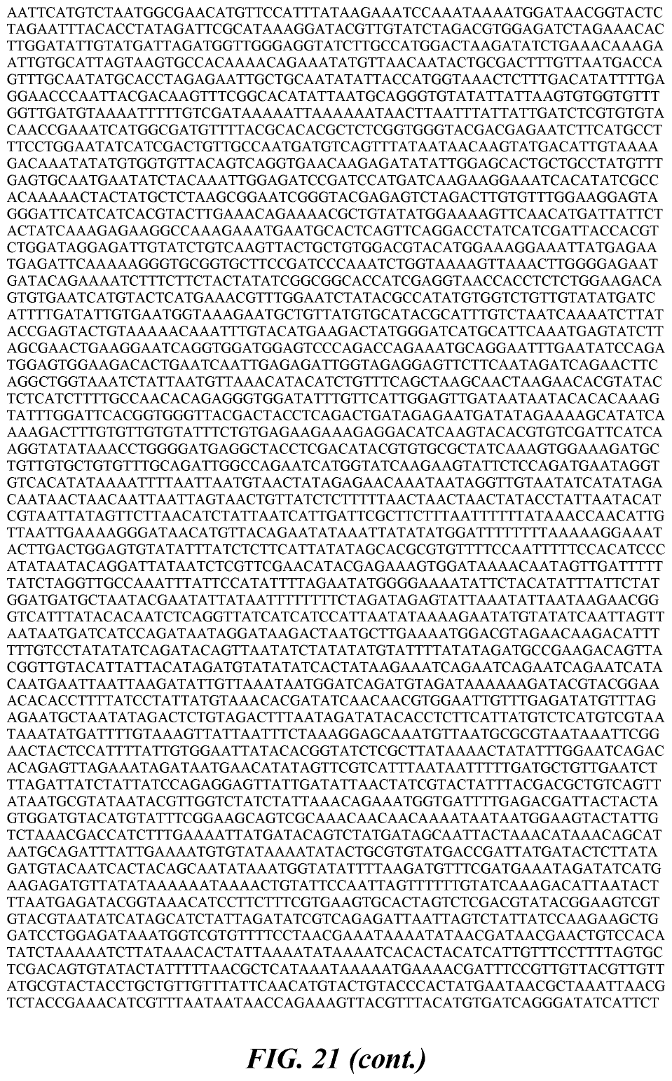

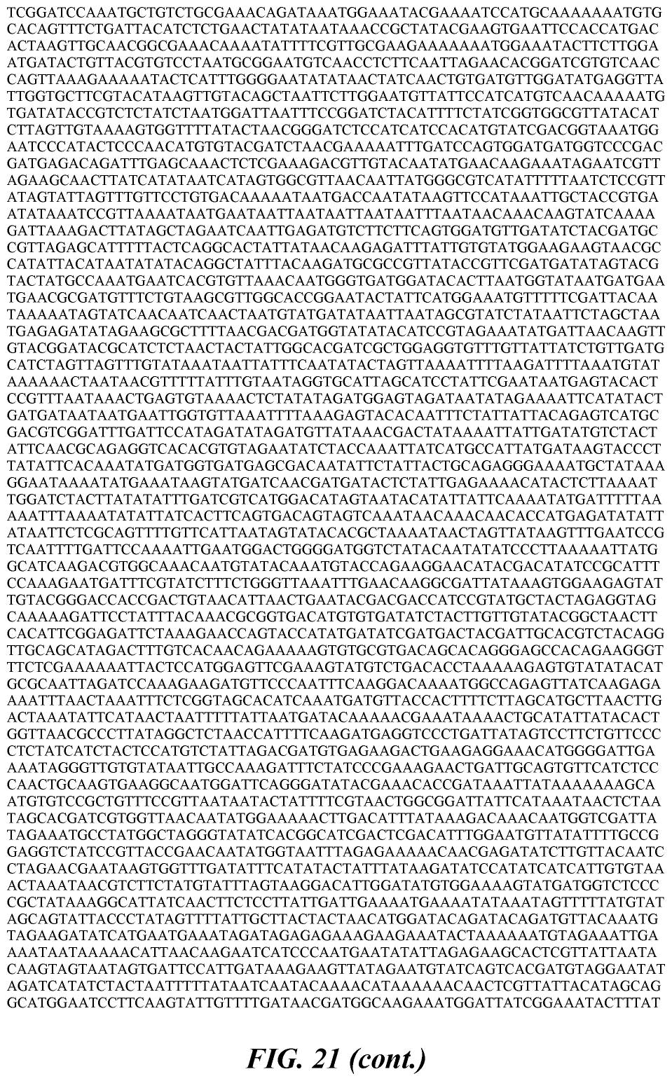

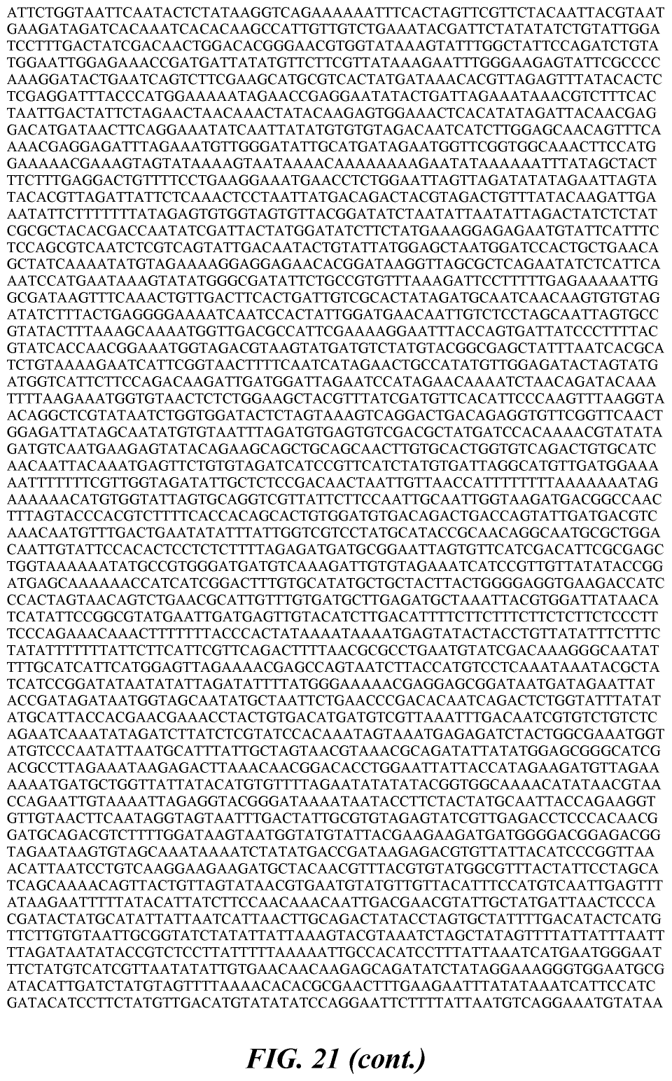

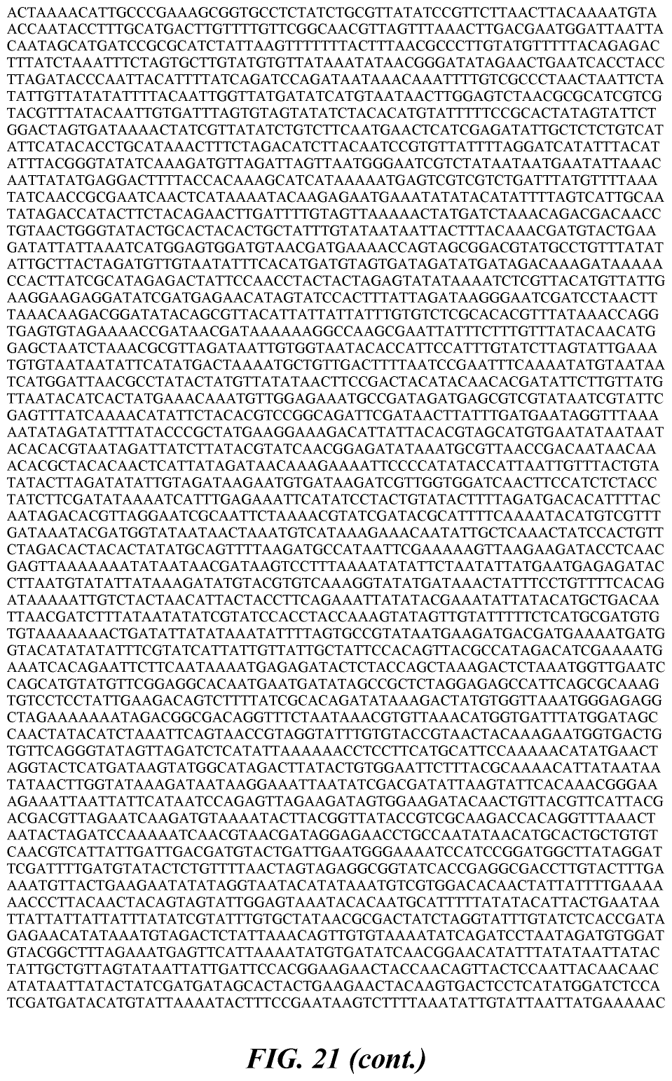

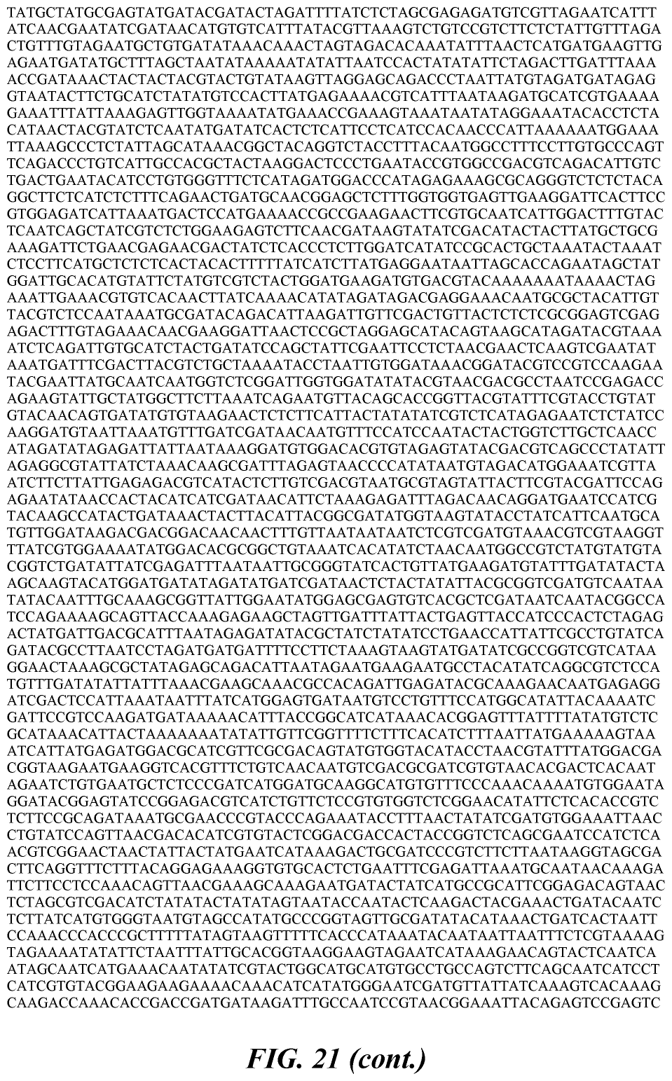

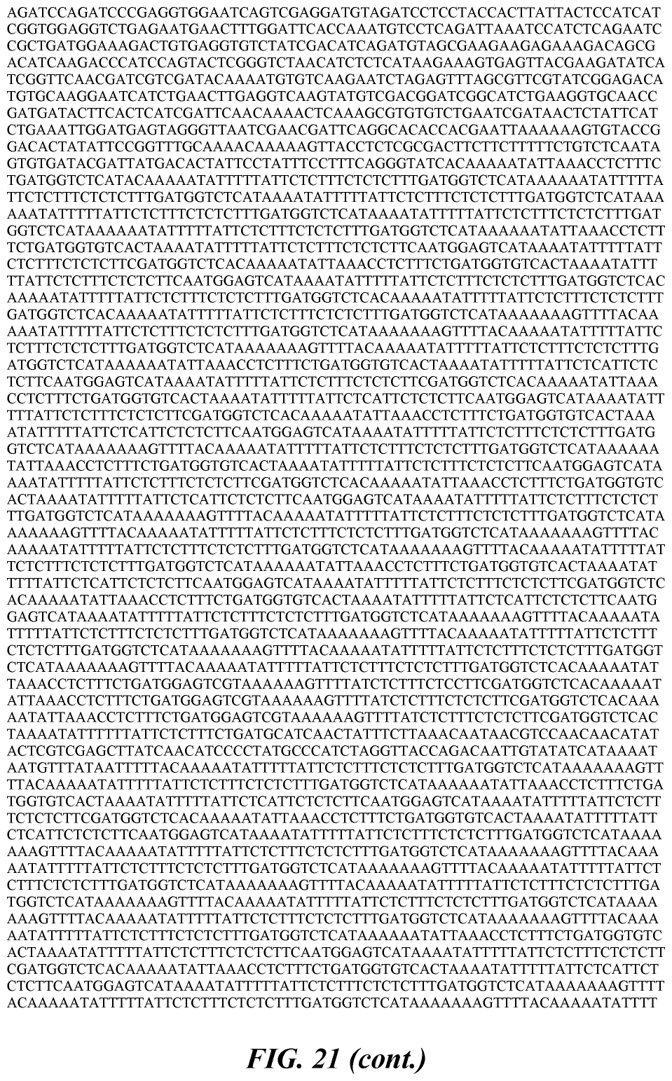

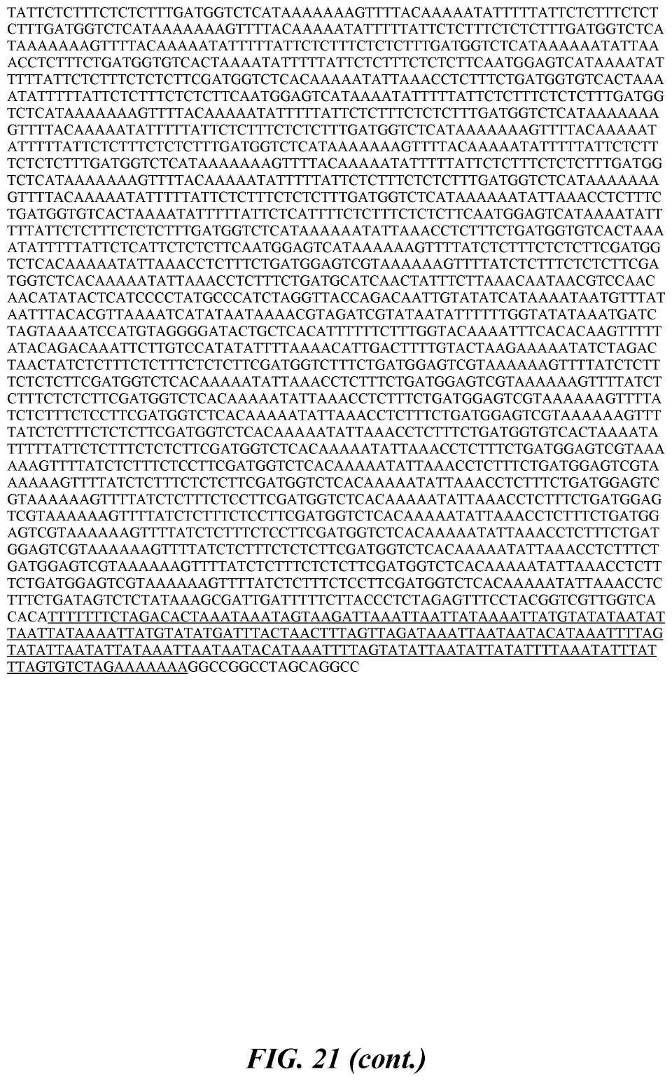

According to some embodiments, the recombinant sMVA viral vector that is used in the compositions disclosed herein is generated according to the methods disclosed in International Application Publication No. WO 2021/236550, which is hereby incorporated by reference as if fully disclosed herein and as indicated in the working examples below. In certain embodiments, three nucleotide fragments, F1, F2, and F3 are synthesized, each fragment including a portion of the full MVA backbone sequence flanked by an MVA concatemer resolution-hairpin loop-concatemer resolution (CR/HL/CR) sequence (FIGS. 17B-17C). F1 encompasses the left ITR and ˜50 kbp of the left end of the internal UR of the MVA genome; F2 contains ˜60 kbp of the middle part of the internal UR of the MVA genome; and F3 encompasses ˜50 kbp of the right end of the internal UR and the right ITR of the MVA genome. sMVA F1 and F2 as well as sMVA F2 and F3 are designed to share ˜3 kbp overlapping sequences to allow the reconstitution of the complete MVA genome by homologous recombination (FIG. 17B). A duplex copy of the 165-nucleotide-long MVA terminal hairpin loop (HL) flanked by MVA CR sequences is added to both ends of each of the three fragments to promote MVA genome resolution and packaging (FIG. 17C). The three sMVA fragments are cloned and maintained in E. coli (DH10B, EPI300, GS1783) by a yeast-bacterial shuttle vector, termed pCCI-Brick (GeneScript), which contains a bacterial mini-F replicon element that can be used as a bacterial artificial chromosome (BAC) vector to stably propagate the three fragments at a low copy number in bacteria. Next-generation sequencing analysis confirmed the integrity of the MVA genomic sequences of the fragments, with the notable exception of an unknown single-point mutation within sMVA fragment F1 located in a non-coding determining region at 3 bp downstream of 021 L. FIGS. 19-21 (SEQ ID NOs: 2-4) show sequences of F1, F2, F3, respectively, according to some embodiments. The CR/HL/CR sequences are underlined. The Del2, IGR69/70, and Del3 insertion sites are also indicated in FIGS. 19-21. As such, in some embodiments, each fragment may serve to carry a separate SARS-CoV-2 protein sequence (or variant, mutant, and/or immunogenic fragment thereof). F1, F2, and F3 are then co-transfected into host cells with a helper virus (e.g., fowl pox virus) wherein the full recombinant sMVA virus is reconstituted and capable of expressing the SARS-CoV-2 protein sequence (or variant, mutant, and/or immunogenic fragment thereof) or sequences inserted therein. Each fragment, F1, F2, and F3, includes an overlapping sequence with the adjacent sequence such that when reconstituted, the MVA genome is sequentially reconstituted in the order F1→F2→F3 via homologous recombination between fragments according to some embodiments.

In some embodiments, a nucleotide sequence that encodes a SARS-CoV-2 S protein (or variant, mutant, and/or immunogenic fragment thereof) is inserted into the Del2 site within F1, the IGR69/70 site within F2, or the Del3 site within F3.

In some embodiments, a nucleotide sequence that encodes a SARS-CoV-2 N protein (or variant, mutant, and/or immunogenic fragment thereof) is inserted into the Del2 site within F1 and a nucleotide sequence that encodes a SARS-CoV-2 S protein (or variant, mutant, and/or immunogenic fragment thereof) is inserted into the Del3 site within F3.

In some embodiments, a nucleotide sequence that encodes a SARS-CoV-2 S protein (or variant, mutant, and/or immunogenic fragment thereof) is inserted into the Del2 site within F1 and a nucleotide sequence that encodes a SARS-CoV-2 N protein (or variant, mutant, and/or immunogenic fragment thereof) is inserted into the Del3 site within F3.

In some embodiments, a nucleotide sequence that encodes a SARS-CoV-2 S protein (or variant, mutant, and/or immunogenic fragment thereof) is inserted into the IGR69/70 site within F2 and a nucleotide sequence that encodes a SARS-CoV-2 N protein (or variant, mutant, and/or immunogenic fragment thereof) is inserted into the Del3 site within F3.

In some embodiments, a nucleotide sequence that encodes a SARS-CoV-2 N protein (or variant, mutant, and/or immunogenic fragment thereof) is inserted into the IGR69/70 site within F2 and a nucleotide sequence that encodes a SARS-CoV-2 S protein (or variant, mutant, and/or immunogenic fragment thereof) is inserted into the Del3 site within F3.

In some embodiments, a nucleotide sequence that encodes a SARS-CoV-2 S protein (or variant, mutant, and/or immunogenic fragment thereof) is inserted into the Del2 site within F1 and a nucleotide sequence that encodes a SARS-CoV-2 N protein (or variant, mutant, and/or immunogenic fragment thereof) is inserted into the IGR69/70 site within F2.

In some embodiments, a nucleotide sequence that encodes a SARS-CoV-2 N protein (or variant, mutant, and/or immunogenic fragment thereof) is inserted into the Del2 site within F1 and a nucleotide sequence that encodes a SARS-CoV-2 S protein (or variant, mutant, and/or immunogenic fragment thereof) is inserted into the IGR69/70 site within F2.

The SARS-CoV-2 S protein (or variant, mutant, and/or immunogenic fragment thereof) and/or the SARS-CoV-2 N protein (or variant, mutant, and/or immunogenic fragment thereof) that are inserted into the F1, F2, and/or F3 fragments as discussed herein are encoded by any SARS-CoV2 S or N protein, including a reference sequence or any variant or mutants thereof. Exemplary SARS-CoV2 S or N proteins that are encoded by the heterologous sequences that can be inserted into the F1, F2, and/or F3 fragments as discussed herein (and thus incorporated into the sMVA vector and reconstituted sMVA virus) are found in additional sequences and mutations discussed below.

In some embodiments, the recombinant sMVA vector comprises one or more heterologous DNA sequences encoding the S protein and/or N protein of the SARS-CoV-2 Wuhan-Hu-1 reference stain. In some embodiments, the recombinant sMVA vector comprises one or more heterologous DNA sequences encoding a mutant S protein and/or N protein such as mutant S protein and/or N protein based on a variant of concern, including B.1.1.7 (Alpha), B.1.351 (Beta), P.1 (Gamma), B.1.617.2 (Delta), B.1.621 (Mu), C.37 (Lambda), C.1.2, BA.1 (Omicron), BA.2 (Omicron), BA.2.12.1 (Omicron), BA.4 (Omicron), BA.5 (Omicron), BA.2.75 (Omicron), BQ.1 (Omicron), BQ.1.1 (Omicron), and XBB (Omicron). Exemplary sequences of the variants of the S proteins and N proteins are illustrated in Table 1 below. According to some embodiments, the recombinant sMVA vectors are reconstituted recombinant sMVA viruses that express or are capable of expressing the one or more S proteins and/or N proteins (or mutants thereof) disclosed herein.

TABLE 1
Exemplary sequences of SARS-CoV-2 S and N proteins

| SEQ | | |
|---|---|---|
| ID NO: | Sequences | Descriptions |
| 5 | atgtttgtctttcttgtcttattgccactagtctctagtcagtgtgttaatcttacaaccaga | Nucleotide |
| | actcaattaccacctgcatacactaattctttcacacgtggtgtttattaccctgacaa | sequence/ORF |
| | agtattcagatcctcagtattacattcaactcaggacttgttcttacctttcttctccaat | (5′ to 3′ end) of |
| | gttacttggttccatgctatacatgtctctgggaccaatggtactaagaggtttgataa | the SARS-CoV- |
| | ccctgtcctaccatttaatgatggtgtttatttcgcttccactgagaagtctaacataat | 2 S antigen |
| | aagaggctggatatttggtactactttagattcgaagacccagtccctacttattgtta | sequence based |
| | ataacgctactaatgttgttattaaagtctgtgaatttcaattctgtaatgatccattcttg | on the Wuhan- |
| | ggtgtttattaccacaagaacaacaagagttggatggaaagtgagttcagagttta | Hu-1 reference |
| | ttctagtgcgaataattgcactttcgaatatgtctctcagcctttccttatggaccttgaa | strain |
| | ggtaaacagggtaatttcaagaatcttagggaatttgtgtttaagaatattgatggtta | (#NC_045512) |
| | tttcaagatatattctaagcacacgcctattaatttagtgcgtgatctccctcagggttt | and optimized |
| | ctcggctttagaaccattggtagatttgccaataggtattaacatcactaggtttcaa | for stability in |
| | actttacttgctttacatagaagttatttgactcctggtgattcttcttcaggttggacagc | vaccinia by |
| | tggtgctgcagcttattatgtgggttatcttcaacctaggactttcctattgaaatataat | silent codon |
| | gagaatggaaccattacagatgctgtagactgtgcacttgaccctctctcagaaac | alteration to |
| | aaagtgtacgttgaaatccttcactgtagagaaaggaatctatcaaacttctaacttt | avoid 4 or more |
| | agagtccaaccaacagaatctattgttagatttcctaatattacaaacttgtgcccttt | of the same |
| | cggtgaagtatttaacgccaccagatttgcatctgtttatgcttggaacaggaagag | nucleotides in |
| | aatcagcaactgtgttgctgattattctgtcctatataattccgcatcattctccacattt | consecutive |
| | aagtgttatggagtgtctcctactaaattaaatgatctctgctttactaatgtctatgca | order |
| | gattcatttgtaattagaggtgatgaagtcagacaaatcgctccagggcaaactgg | |
| | aaagattgctgattataattataaattaccagatgactttacaggctgcgttatagctt | |
| | ggaattctaacaatcttgattctaaggttggtggtaattataattacctgtatagattgtt | |
| | taggaagtctaatctcaaacctttcgagagagatatttcaactgaaatctatcaggc | |
| | cggtagcacaccttgtaatggtgttgaaggatttaattgttactttcctttacaatcatat | |
| | ggtttccaacccactaatggtgttggttaccaaccatacagagtagtagtactttcatt | |
| | tgaacttctacatgcaccagcaactgtttgtggacctaagaagtctactaatttggtt | |
| | aagaacaaatgtgtcaatttcaacttcaatggtttaacaggcacaggtgttcttactg | |
| | agtctaacaagaagtttctgcctttccaacaatttggcagagacattgctgacacta | |
| | ctgatgctgtccgtgatccacagacacttgagattcttgacattacaccatgttcattt | |
| | ggtggtgtcagtgttataacaccaggaacaaatacttctaaccaggttgctgttcttt | |
| | atcaggatgttaactgcacagaagtccctgttgctattcatgcagatcaacttactcc | |
| | tacttggcgtgtttattctacaggttctaatgtatttcaaacacgtgcaggctgtttaata | |
| | ggagctgaacatgtcaacaactcatatgagtgtgacatacccattggtgcaggtat | |
| | atgcgctagttatcagactcagactaattctcctcggcgggcacgtagtgtagctag | |
| | tcaatccatcattgcctacactatgtcacttggtgcagagaattcagttgcttactcta | |
| | ataactctattgccatacccacaaactttactattagtgttaccacagaaattctacc | |
| | agtgtctatgaccaagacatcagtagattgtacaatgtacatttgtggtgattcaact | |
| | gaatgcagcaatctattgttgcaatatggcagtttctgtacacaattaaaccgtgcttt | |
| | aactggaatagctgttgaacaagacaagaacacccaagaagtatttgcacaagt | |
| | caaacaaatttacaagacaccaccaattaaagatttcggtggatttaatttctcaca | |
| | aatattaccagatccatctaaaccaagcaagaggtcatttattgaagatctactatt | |
| | caacaaagtgacacttgcagatgctggcttcatcaaacaatatggtgattgccttg | |
| | gtgatattgctgctagagacctcatttgtgcacagaagtttaacggccttactgtattg | |
| | ccacctttgctcacagatgaaatgattgctcaatacacttctgcactgttagcgggta | |
| | caatcacttctggttggacctttggtgcaggtgctgcattacaaataccatttgctatg | |
| | caaatggcttataggtttaatggtattggagttacacagaatgttctctatgagaacc | |
| | agaaattgattgccaaccaatttaatagtgctattggcaagattcaagactcactttc | |
| | ttccacagcaagtgcacttggtaaacttcaagatgtggtcaaccagaatgcacaa | |
| | gctttaaacacgcttgttaaacaacttagctccaactttggtgcaatttcaagtgtatt | |
| | aaatgatatcctttcacgtcttgacaaagttgaggctgaagtgcaaattgataggttg | |
| | atcacaggcagacttcaaagtttgcagacatatgtgactcaacaattaattagagc | |
| | ggacaatctaagagagttgatttctgtggaaagggctatcatcttatgtccttccctc | |
| | agtcagcacctcatggtgtagtcttcttgcatgtgacttatgtccctgcacaagagaa | |
| | gaacttcacaactgctcctgccatttgtcatgatggtaaagcacactttcctcgtgaa | |
| | ggtgtctttgtttcaaatggcacacactggtttgtaacacaaaggaatttctatgaac | |
| | cacaaatcattactacagacaacacatttgtgtctggtaactgtgatgttgtaatagg | |
| | aattgtcaacaacacagtttatgatcctttgcaacctgaattagactcattcaagga | |
| | ggagttagataaatatttcaagaatcatacatcaccagatgttgatttaggtgacatc | |
| | tctggcattaatgcttcagttgtaaacattcagaaagaaattgaccgcctcaatgag | |
| | gttgccaagaatttaaatgaatctctcatcgatctccaagaacttggaaagtatgag | |
| | cagtatatcaaatggccatggtacatttggctaggtttcatagctggcttgattgccat | |
| | agtaatggtgacaattatgctttgctgtatgaccagttgctgtagttgtctcaagggct | |
| | gttgttcttgtggatcctgctgcaaatttgatgaagacgactctgagccagtgctcaa | |
| | aggagtcaaattacattacacataa | |
| 6 | MFVFLVLLPLVSSQCVNLTTRTQLPPAYTNSFTRGVYYPD | Amino acid |
| | KVFRSSVLHSTQDLFLPFFSNVTWFHAIHVSGTNGTKRFD | sequence (N- to |
| | NPVLPFNDGVYFASTEKSNIIRGWIFGTTLDSKTQSLLIVNN | C-terminus) of |
| | ATNVVIKVCEFQFCNDPFLGVYYHKNNKSWMESEFRVYS | the SARS-CoV- |
| | SANNCTFEYVSQPFLMDLEGKQGNFKNLREFVFKNIDGYF | 2 S antigen |
| | KIYSKHTPINLVRDLPQGFSALEPLVDLPIGINITRFQTLLAL | sequence based |
| | HRSYLTPGDSSSGWTAGAAAYYVGYLQPRTFLLKYNENG | on the Wuhan- |
| | TITDAVDCALDPLSETKCTLKSFTVEKGIYQTSNFRVQPTE | Hu-1 reference |
| | SIVRFPNITNLCPFGEVFNATRFASVYAWNRKRISNCVADY | strain |
| | SVLYNSASFSTFKCYGVSPTKLNDLCFTNVYADSFVIRGD | (#NC_045512) |
| | EVRQIAPGQTGKIADYNYKLPDDFTGCVIAWNSNNLDSKV | |
| | GGNYNYLYRLFRKSNLKPFERDISTEIYQAGSTPCNGVEG | |
| | FNCYFPLQSYGFQPTNGVGYQPYRVVVLSFELLHAPATV | |
| | CGPKKSTNLVKNKCVNFNFNGLTGTGVLTESNKKFLPFQ | |
| | QFGRDIADTTDAVRDPQTLEILDITPCSFGGVSVITPGTNT | |
| | SNQVAVLYQDVNCTEVPVAIHADQLTPTWRVYSTGSNVF | |
| | QTRAGCLIGAEHVNNSYECDIPIGAGICASYQTQTNSPRR | |
| | ARSVASQSIIAYTMSLGAENSVAYSNNSIAIPTNFTISVTTEI | |
| | LPVSMTKTSVDCTMYICGDSTECSNLLLQYGSFCTQLNRA | |
| | LTGIAVEQDKNTQEVFAQVKQIYKTPPIKDFGGFNFSQILP | |
| | DPSKPSKRSFIEDLLFNKVTLADAGFIKQYGDCLGDIAARD | |
| | LICAQKFNGLTVLPPLLTDEMIAQYTSALLAGTITSGWTFG | |
| | AGAALQIPFAMQMAYRFNGIGVTQNVLYENQKLIANQFNS | |
| | AIGKIQDSLSSTASALGKLQDVVNQNAQALNTLVKQLSSNF | |
| | GAISSVLNDILSRLDKVEAEVQIDRLITGRLQSLQTYVTQQL | |
| | IRAAEIRASANLAATKMSECVLGQSKRVDFCGKGYHLMSF | |
| | PQSAPHGVVFLHVTYVPAQEKNFTTAPAICHDGKAHFPRE | |
| | GVFVSNGTHWFVTQRNFYEPQIITTDNTFVSGNCDVVIGIV | |
| | NNTVYDPLQPELDSFKEELDKYFKNHTSPDVDLGDISGINA | |
| | SVVNIQKEIDRLNEVAKNLNESLIDLQELGKYEQYIKWPWY | |
| | IWLGFIAGLIAIVMVTIMLCCMTSCCSCLKGCCSCGSCCKF | |
| | DEDDSEPVLKGVKLHYT | |
| 7 | atgtctgataacggaccacagaatcagcgaaacgcaccacgcattacgtttggtg | Nucleotide |
| | gaccctcagattcaactggcagtaaccagaatggagaacgcagtggagcgcga | sequence/ORF |
| | tctaaacaacgtcggcctcaaggtttacccaataatactgcgtcttggttcaccgctc | (5′ to 3′ end) of |
| | tcactcaacatggcaaggaagaccttaaattccctcgaggacaaggcgttccaat | the SARS-CoV- |
| | taacaccaatagcagtccagatgaccaaattggctactaccgaagagctaccag | 2 N antigen |
| | acgaattcgtggtggtgacggtaagatgaaagatctcagtccaagatggtatttcta | sequence based |
| | ctacctaggaactgggccagaagctggacttccctatggtgctaacaaagacgg | on the Wuhan- |
| | catcatatgggttgcaactgagggagccttgaatacacctaaagatcacattggc | Hu-1 reference |
| | acccgcaatcctgctaacaatgctgcaatcgtgctacaacttcctcaaggaacaa | strain |
| | cattgcctaaaggcttctacgcagaagggagcagaggcggcagtcaagcctctt | (#NC_045512) |
| | ctcgttcctcatcacgtagtcgcaacagttcaagaaattcaactccaggcagcagt | and optimized |
| | agaggaacttctcctgctagaatggctggcaatggcggtgatgctgctcttgctttgc | for stability in |
| | tgctgcttgacagattgaaccagcttgagagcaagatgtctggtaaaggccaaca | vaccinia by |
| | acaacaaggccaaactgtcactaagaaatctgctgctgaggcttctaagaagcct | silent codon |
| | cggcagaaacgtactgccactaaagcatacaatgtaacacaagctttcggcaga | alteration to |
| | cgtggtccagaacaaacccaaggaaactttggagaccaggaactaatcagaca | avoid 4 or more |
| | aggaactgattacaaacattggccgcaaattgcacaatttgctcccagcgcttcag | of the same |
| | cgttcttcggaatgtcgcgcattggcatggaagtcacaccttcgggaacgtggttga | nucleotides in |
| | cctacacaggtgccatcaaattggatgacaaagatccaaatttcaaagatcaagt | consecutive |
| | catattgctgaataagcatattgacgcatacaagacattcccaccaacagagcct | order |
| | aagaaggacaagaagaagaaggctgatgaaactcaagccttaccgcagaga | |
| | cagaagaaacagcaaactgtgactcttcttcctgctgcagatttggatgatttctcca | |
| | aacaattgcaacaatccatgagcagtgctgactcaactcaggcctaa | |
| 8 | MSDNGPQNQRNAPRITFGGPSDSTGSNQNGERSGARSK | Amino acid |
| | QRRPQGLPNNTASWFTALTQHGKEDLKFPRGQGVPINTN | sequence (N- to |
| | SSPDDQIGYYRRATRRIRGGDGKMKDLSPRWYFYYLGTG | C-terminus) of |
| | PEAGLPYGANKDGIIWVATEGALNTPKDHIGTRNPANNAAI | the SARS-CoV- |
| | VLQLPQGTTLPKGFYAEGSRGGSQASSRSSSRSRNSSRN | 2 N antigen |
| | STPGSSRGTSPARMAGNGGDAALALLLLDRLNQLESKMS | sequence based |
| | GKGQQQQGQTVTKKSAAEASKKPRQKRTATKAYNVTQA | on the Wuhan- |
| | FGRRGPEQTQGNFGDQELIRQGTDYKHWPQIAQFAPSAS | Hu-1 reference |
| | AFFGMSRIGMEVTPSGTWLTYTGAIKLDDKDPNFKDQVILL | strain |
| | NKHIDAYKTFPPTEPKKDKKKKADETQALPQRQKKQQTVT | (#NC_045512) |
| | LLPAADLDDFSKQLQQSMSSADSTQA | |
| 9 | atgtttgtctttcttgtcttattgccactagtctctagtcagtgtgttaatcttagaaccaga | Nucleotide |
| | actcaattaccacctgcatacactaattctttcacacgtggtgtttattaccctgacaa | sequence/ORF |
| | agtattcagatcctcagtattacattcaactcaggacttgttcttacctttcttctccaat | (5′ to 3′ end) of |
| | gttacttggttccatgctatacatgtctctgggaccaatggtactaagaggtttgataa | a codon- |
| | ccctgtcctaccatttaatgatggtgtttatttcgcttccactgagaagtctaacataat | optimized |
| | aagaggctggatatttggtactactttagattcgaagacccagtccctacttattgtta | SARS-CoV-2 S |
| | ataacgctactaatgttgttattaaagtctgtgaatttcaattctgtaatgatccattcttg | antigen |
| | gatgtttattaccacaagaacaacaagagttggatggaaagtggagtttattctagt | sequence based |
| | gcgaataattgcactttcgaatatgtctctcagcctttccttatggaccttgaaggtaa | on the Wuhan- |
| | acagggtaatttcaagaatcttagggaatttgtgtttaagaatattgatggttatttcaa | Hu-1 reference |
| | gatatattctaagcacacgcctattaatttagtgcgtgatctccctcagggtttctcgg | strain |
| | ctttagaaccattggtagatttgccaataggtattaacatcactaggtttcaaacttta | (#NC_045512) |
| | cttgctttacatagaagttatttgactcctggtgattcttcttcaggttggacagctggtg | and optimized |
| | ctgcagcttattatgtgggttatcttcaacctaggactttcctattgaaatataatgaga | for vaccinia, |
| | atggaaccattacagatgctgtagactgtgcacttgaccctctctcagaaacaaag | which is further |
| | tgtacgttgaaatccttcactgtagagaaaggaatctatcaaacttctaactttagag | modified to |
| | tccaaccaacagaatctattgttagatttcctaatattacaaacttgtgccctttcggtg | encode for an S |
| | aagtatttaacgccaccagatttgcatctgtttatgcttggaacaggaagagaatca | antigen that |
| | gcaactgtgttgctgattattctgtcctatataattccgcatcattctccacatttaagtgt | includes |
| | tatggagtgtctcctactaaattaaatgatctctgctttactaatgtctatgcagattcat | mutations of the |
| | ttgtaattagaggtgatgaagtcagacaaatcgctccagggcaaactggaaagat | B.1.617.2 |
| | tgctgattataattataaattaccagatgactttacaggctgcgttatagcttggaattc | variant lineage |
| | taacaatcttgattctaaggttggtggtaattataattaccggtatagattgtttaggaa | identified in |
| | gtctaatctcaaacctttcgagagagatatttcaactgaaatctatcaggccggtag | India, including |
| | caaaccttgtaatggtgttgaaggatttaattgttactttcctttacaatcatatggtttcc | T19R, G142D, |
| | aacccactaatggtgttggttaccaaccatacagagtagtagtactttcatttgaact | Del156/157, |
| | tctacatgcaccagcaactgtttgtggacctaagaagtctactaatttggttaagaa | R158G, L452R, |
| | caaatgtgtcaatttcaacttcaatggtttaacaggcacaggtgttcttactgagtcta | T478K, D614G, |
| | acaagaagtttctgcctttccaacaatttggcagagacattgctgacactactgatg | P681R, and |
| | ctgtccgtgatccacagacacttgagattcttgacattacaccatgttcatttggtggt | D950N |
| | gtcagtgttataacaccaggaacaaatacttctaaccaggttgctgttctttatcagg | |
| | gtgttaactgcacagaagtccctgttgctattcatgcagatcaacttactcctacttgg | |
| | cgtgtttattctacaggttctaatgtatttcaaacacgtgcaggctgtttaataggagct | |
| | gaacatgtcaacaactcatatgagtgtgacatacccattggtgcaggtatatgcgc | |
| | tagttatcagactcagactaattctcgtcggcgggcacgtagtgtagctagtcaatc | |
| | catcattgcctacactatgtcacttggtgcagagaattcagttgcttactctaataact | |
| | ctattgccatacccacaaactttactattagtgttaccacagaaattctaccagtgtct | |
| | atgaccaagacatcagtagattgtacaatgtacatttgtggtgattcaactgaatgc | |
| | agcaatctattgttgcaatatggcagtttctgtacacaattaaaccgtgctttaactgg | |
| | aatagctgttgaacaagacaagaacacccaagaagtatttgcacaagtcaaac | |
| | aaatttacaagacaccaccaattaaagatttcggtggatttaatttctcacaaatatt | |
| | accagatccatctaaaccaagcaagaggtcatttattgaagatctactattcaaca | |
| | aagtgacacttgcagatgctggcttcatcaaacaatatggtgattgccttggtgatat | |
| | tgctgctagagacctcatttgtgcacagaagtttaacggccttactgtattgccacctt | |
| | tgctcacagatgaaatgattgctcaatacacttctgcactgttagcgggtacaatca | |
| | cttctggttggacctttggtgcaggtgctgcattacaaataccatttgctatgcaaatg | |
| | gcttataggtttaatggtattggagttacacagaatgttctctatgagaaccagaaat | |
| | tgattgccaaccaatttaatagtgctattggcaagattcaagactcactttcttccaca | |
| | gcaagtgcacttggtaaacttcagaatgtggtcaaccagaatgcacaagctttaa | |
| | acacgcttgttaaacaacttagctccaactttggtgcaatttcaagtgtattaaatgat | |
| | atcctttcacgtcttgacaaagttgaggctgaagtgcaaattgataggttgatcaca | |
| | ggcagacttcaaagtttgcagacatatgtgactcaacaattaattagagctgcaga | |
| | aatcagagcttctgctaatcttgctgctactaagatgtcagagtgtgtacttggacaa | |
| | tctaagagagttgatttctgtggaaagggctatcatcttatgtccttccctcagtcagc | |
| | acctcatggtgtagtcttcttgcatgtgacttatgtccctgcacaagagaagaacttc | |
| | acaactgctcctgccatttgtcatgatggtaaagcacactttcctcgtgaaggtgtctt | |
| | tgtttcaaatggcacacactggtttgtaacacaaaggaatttctatgaaccacaaat | |
| | cattactacagacaacacatttgtgtctggtaactgtgatgttgtaataggaattgtca | |
| | acaacacagtttatgatcctttgcaacctgaattagactcattcaaggaggagttag | |
| | ataaatatttcaagaatcatacatcaccagatgttgatttaggtgacatctctggcatt | |
| | aatgcttcagttgtaaacattcagaaagaaattgaccgcctcaatgaggttgccaa | |
| | gaatttaaatgaatctctcatcgatctccaagaacttggaaagtatgagcagtatat | |
| | caaatggccatggtacatttggctaggtttcatagctggcttgattgccatagtaatg | |
| | gtgacaattatgctttgctgtatgaccagttgctgtagttgtctcaagggctgttgttctt | |
| | gtggatcctgctgcaaatttgatgaagacgactctgagccagtgctcaaaggagt | |
| | caaattacattacacataa | |
| 10 | MFVFLVLLPLVSSQCVNLRTRTQLPPAYTNSFTRGVYYPD | Amino acid |
| | KVFRSSVLHSTQDLFLPFFSNVTWFHAIHVSGTNGTKRFD | sequence (N- to |
| | NPVLPFNDGVYFASTEKSNIIRGWIFGTTLDSKTQSLLIVNN | C-terminus) of a |
| | ATNVVIKVCEFQFCNDPFLDVYYHKNNKSWMESGVYSSA | codon-optimized |
| | NNCTFEYVSQPFLMDLEGKQGNFKNLREFVFKNIDGYFKI | SARS-CoV-2 S |
| | YSKHTPINLVRDLPQGFSALEPLVDLPIGINITRFQTLLALH | antigen |
| | RSYLTPGDSSSGWTAGAAAYYVGYLQPRTFLLKYNENGTI | sequence based |
| | TDAVDCALDPLSETKCTLKSFTVEKGIYQTSNFRVQPTESI | on the Wuhan- |
| | VRFPNITNLCPFGEVFNATRFASVYAWNRKRISNCVADYS | Hu-1 reference |
| | VLYNSASFSTFKCYGVSPTKLNDLCFTNVYADSFVIRGDE | strain |
| | VRQIAPGQTGKIADYNYKLPDDFTGCVIAWNSNNLDSKVG | (#NC_045512) |
| | GNYNYRYRLFRKSNLKPFERDISTEIYQAGSKPCNGVEGF | and optimized |
| | NCYFPLQSYGFQPTNGVGYQPYRVVVLSFELLHAPATVC | for vaccinia, |
| | GPKKSTNLVKNKCVNFNFNGLTGTGVLTESNKKFLPFQQF | which is further |
| | GRDIADTTDAVRDPQTLEILDITPCSFGGVSVITPGTNTSN | modified to |
| | QVAVLYQGVNCTEVPVAIHADQLTPTWRVYSTGSNVFQT | encode for an S |
| | RAGCLIGAEHVNNSYECDIPIGAGICASYQTQTNSRRRAR | antigen that |
| | SVASQSIIAYTMSLGAENSVAYSNNSIAIPTNFTISVTTEILP | includes |
| | VSMTKTSVDCTMYICGDSTECSNLLLQYGSFCTQLNRALT | mutations of the |
| | GIAVEQDKNTQEVFAQVKQIYKTPPIKDFGGFNFSQILPDP | B.1.617.2 |
| | SKPSKRSFIEDLLFNKVTLADAGFIKQYGDCLGDIAARDLIC | variant lineage |
| | AQKFNGLTVLPPLLTDEMIAQYTSALLAGTITSGWTFGAGA | identified in |
| | ALQIPFAMQMAYRFNGIGVTQNVLYENQKLIANQFNSAIGK | India, including |
| | IQDSLSSTASALGKLQNVVNQNAQALNTLVKQLSSNFGAIS | T19R, G142D, |
| | SVLNDILSRLDKVEAEVQIDRLITGRLQSLQTYVTQQLIRAA | Del156/157, |
| | EIRASANLAATKMSECVLGQSKRVDFCGKGYHLMSFPQS | R158G, L452R, |
| | APHGVVFLHVTYVPAQEKNFTTAPAICHDGKAHFPREGVF | T478K, D614G, |
| | VSNGTHWFVTQRNFYEPQIITTDNTFVSGNCDVVIGIVNNT | P681R, and |
| | VYDPLQPELDSFKEELDKYFKNHTSPDVDLGDISGINASVV | D950N |
| | NIQKEIDRLNEVAKNLNESLIDLQELGKYEQYIKWPWYIWL | |
| | GFIAGLIAIVMVTIMLCCMTSCCSCLKGCCSCGSCCKFDED | |
| | DSEPVLKGVKLHYT | |
| 11 | atgtttgtctttcttgtcttattgccactagtctctagtcagtgtgttaatcttagaaccaga | Nucleotide |
| | actcaattaccacctgcatacactaattctttcacacgtggtgtttattaccctgacaa | sequence/ORF |
| | agtattcagatcctcagtattacattcaactcaggacttgttcttacctttcttctccaat | (5′ to 3′ end) of |
| | gttacttggttccatgctatacatgtctctgggaccaatggtactaagaggtttgataa | a codon- |
| | ccctgtcctaccatttaatgatggtgtttatttcgcttccactgagaagtctaacataat | optimized |
| | aagaggctggatatttggtactactttagattcgaagacccagtccctacttattgtta | SARS-CoV-2 S |
| | ataacgctactaatgttgttattaaagtctgtgaatttcaattctgtaatgatccattcttg | antigen |
| | ggtgtttattaccacaagaacaacaagagttggatggaaagtggtgtttattctagt | sequence based |
| | gcgaataattgcactttcgaatatgtctctcagcctttccttatggaccttgaaggtaa | on the Wuhan- |
| | acagggtaatttcaagaatcttagggaatttgtgtttaagaatattgatggttatttcaa | Hu-1 reference |
| | gatatattctaagcacacgcctattaatttagtgcgtgatctccctcagggtttctcgg | strain |
| | ctttagaaccattggtagatttgccaataggtattaacatcactaggtttcaaacttta | (#NC_045512) |
| | cttgctttacatagaagttatttgactcctggtgattctttctcaggttggacagctggtg | and optimized |
| | ctgcagcttattatgtgggttatcttcaacctaggactttcctattgaaatataatgaga | for vaccinia, |
| | atggaaccattacagatgctgtagactgtgcacttgaccctctctcagaaacaaag | which is further |
| | tgtacgttgaaatccttcactgtagagaaaggaatctatcaaacttctaactttagag | modified to |
| | tccaaccaacagaatctattgttagatttcctaatattacaaacttgtgccctttcggtg | encode for an S |
| | aagtatttaacgccaccagatttgcatctgtttatgcttggaacaggaagagaatca | antigen that |
| | gcaactgtgttgctgattattctgtcctatataattccgcatcattctccacatttaagtgt | includes |
| | tatggagtgtctcctactaaattaaatgatctctgctttactaatgtctatgcagattcat | mutations of the |
| | ttgtaattagaggtgatgaagtcagacaaatcgctccagggcaaactggaaagat | B.1.617.2 |
| | tgctgattataattataaattaccagatgactttacaggctgcgttatagcttggaattc | variant lineage |
| | taacaatcttgattctaaggttggtggtaattataattaccggtatagattgtttaggaa | identified in |
| | gtctaatctcaaacctttcgagagagatatttcaactgaaatctatcaggccggtag | India, including |
| | caaaccttgtaatggtgttgaaggatttaattgttactttcctttacaatcatatggtttcc | T19R, E156G, |
| | aacccactaatggtgttggttaccaaccatacagagtagtagtactttcatttgaact | Del157/158, |
| | tctacatgcaccagcaactgtttgtggacctaagaagtctactaatttggttaagaa | S255F, L452R, |
| | caaatgtgtcaatttcaacttcaatggtttaacaggcacaggtgttcttactgagtcta | T478K, D614G, |
| | acaagaagtttctgcctttccaacaatttggcagagacattgctgacactactgatg | P681R, and |
| | ctgtccgtgatccacagacacttgagattcttgacattacaccatgttcatttggtggt | D950N |
| | gtcagtgttataacaccaggaacaaatacttctaaccaggttgctgttctttatcagg | |
| | gtgttaactgcacagaagtccctgttgctattcatgcagatcaacttactcctacttgg | |
| | cgtgtttattctacaggttctaatgtatttcaaacacgtgcaggctgtttaataggagct | |
| | gaacatgtcaacaactcatatgagtgtgacatacccattggtgcaggtatatgcgc | |
| | tagttatcagactcagactaattctcgtcggcgggcacgtagtgtagctagtcaatc | |
| | catcattgcctacactatgtcacttggtgcagagaattcagttgcttactctaataact | |
| | ctattgccatacccacaaactttactattagtgttaccacagaaattctaccagtgtct | |
| | atgaccaagacatcagtagattgtacaatgtacatttgtggtgattcaactgaatgc | |
| | agcaatctattgttgcaatatggcagtttctgtacacaattaaaccgtgctttaactgg | |
| | aatagctgttgaacaagacaagaacacccaagaagtatttgcacaagtcaaac | |
| | aaatttacaagacaccaccaattaaagatttcggtggatttaatttctcacaaatatt | |
| | accagatccatctaaaccaagcaagaggtcatttattgaagatctactattcaaca | |
| | aagtgacacttgcagatgctggcttcatcaaacaatatggtgattgccttggtgatat | |
| | tgctgctagagacctcatttgtgcacagaagtttaacggccttactgtattgccacctt | |
| | tgctcacagatgaaatgattgctcaatacacttctgcactgttagcgggtacaatca | |
| | cttctggttggacctttggtgcaggtgctgcattacaaataccatttgctatgcaaatg | |
| | gcttataggtttaatggtattggagttacacagaatgttctctatgagaaccagaaat | |
| | tgattgccaaccaatttaatagtgctattggcaagattcaagactcactttcttccaca | |
| | gcaagtgcacttggtaaacttcagaatgtggtcaaccagaatgcacaagctttaa | |
| | acacgcttgttaaacaacttagctccaactttggtgcaatttcaagtgtattaaatgat | |
| | atcctttcacgtcttgacaaagttgaggctgaagtgcaaattgataggttgatcaca | |
| | ggcagacttcaaagtttgcagacatatgtgactcaacaattaattagagctgcaga | |
| | aatcagagcttctgctaatcttgctgctactaagatgtcagagtgtgtacttggacaa | |
| | tctaagagagttgatttctgtggaaagggctatcatcttatgtccttccctcagtcagc | |
| | acctcatggtgtagtcttcttgcatgtgacttatgtccctgcacaagagaagaacttc | |
| | acaactgctcctgccatttgtcatgatggtaaagcacactttcctcgtgaaggtgtctt | |
| | tgtttcaaatggcacacactggtttgtaacacaaaggaatttctatgaaccacaaat | |
| | cattactacagacaacacatttgtgtctggtaactgtgatgttgtaataggaattgtca | |
| | acaacacagtttatgatcctttgcaacctgaattagactcattcaaggaggagttag | |
| | ataaatatttcaagaatcatacatcaccagatgttgatttaggtgacatctctggcatt | |
| | aatgcttcagttgtaaacattcagaaagaaattgaccgcctcaatgaggttgccaa | |
| | gaatttaaatgaatctctcatcgatctccaagaacttggaaagtatgagcagtatat | |
| | caaatggccatggtacatttggctaggtttcatagctggcttgattgccatagtaatg | |
| | gtgacaattatgctttgctgtatgaccagttgctgtagttgtctcaagggctgttgttctt | |
| | gtggatcctgctgcaaatttgatgaagacgactctgagccagtgctcaaaggagt | |
| | caaattacattacacataa | |
| 12 | MFVFLVLLPLVSSQCVNLRTRTQLPPAYTNSFTRGVYYPD | Amino acid |
| | KVFRSSVLHSTQDLFLPFFSNVTWFHAIHVSGTNGTKRFD | sequence (N- to |
| | NPVLPFNDGVYFASTEKSNIIRGWIFGTTLDSKTQSLLIVNN | C-terminus) of a |
| | ATNVVIKVCEFQFCNDPFLGVYYHKNNKSWMESGVYSSA | codon-optimized |
| | NNCTFEYVSQPFLMDLEGKQGNFKNLREFVFKNIDGYFKI | SARS-CoV-2 S |
| | YSKHTPINLVRDLPQGFSALEPLVDLPIGINITRFQTLLALH | antigen |
| | RSYLTPGDSFSGWTAGAAAYYVGYLQPRTFLLKYNENGTI | sequence based |
| | TDAVDCALDPLSETKCTLKSFTVEKGIYQTSNFRVQPTESI | on the Wuhan- |
| | VRFPNITNLCPFGEVFNATRFASVYAWNRKRISNCVADYS | Hu-1 reference |
| | VLYNSASFSTFKCYGVSPTKLNDLCFTNVYADSFVIRGDE | strain |
| | VRQIAPGQTGKIADYNYKLPDDFTGCVIAWNSNNLDSKVG | (#NC_045512) |
| | GNYNYRYRLFRKSNLKPFERDISTEIYQAGSKPCNGVEGF | and optimized |
| | NCYFPLQSYGFQPTNGVGYQPYRVVVLSFELLHAPATVC | for vaccinia, |
| | GPKKSTNLVKNKCVNFNFNGLTGTGVLTESNKKFLPFQQF | which is further |
| | GRDIADTTDAVRDPQTLEILDITPCSFGGVSVITPGTNTSN | modified to |
| | QVAVLYQGVNCTEVPVAIHADQLTPTWRVYSTGSNVFQT | encode for an S |
| | RAGCLIGAEHVNNSYECDIPIGAGICASYQTQTNSRRRAR | antigen that |
| | SVASQSIIAYTMSLGAENSVAYSNNSIAIPTNFTISVTTEILP | includes |
| | VSMTKTSVDCTMYICGDSTECSNLLLQYGSFCTQLNRALT | mutations of the |
| | GIAVEQDKNTQEVFAQVKQIYKTPPIKDFGGFNFSQILPDP | B.1.617.2 |
| | SKPSKRSFIEDLLFNKVTLADAGFIKQYGDCLGDIAARDLIC | variant lineage |
| | AQKFNGLTVLPPLLTDEMIAQYTSALLAGTITSGWTFGAGA | identified in |
| | ALQIPFAMQMAYRFNGIGVTQNVLYENQKLIANQFNSAIGK | India, including |
| | IQDSLSSTASALGKLQNVVNQNAQALNTLVKQLSSNFGAIS | T19R, E156G, |
| | SVLNDILSRLDKVEAEVQIDRLITGRLQSLQTYVTQQLIRAA | Del157/158, |
| | EIRASANLAATKMSECVLGQSKRVDFCGKGYHLMSFPQS | S255F, L452R, |
| | APHGVVFLHVTYVPAQEKNFTTAPAICHDGKAHFPREGVF | T478K, D614G, |
| | VSNGTHWFVTQRNFYEPQIITTDNTFVSGNCDVVIGIVNNT | P681R, and |
| | VYDPLQPELDSFKEELDKYFKNHTSPDVDLGDISGINASVV | D950N |
| | NIQKEIDRLNEVAKNLNESLIDLQELGKYEQYIKWPWYIWL | |
| | GFIAGLIAIVMVTIMLCCMTSCCSCLKGCCSCGSCCKFDED | |
| | DSEPVLKGVKLHYT | |
| 13 | atgtttgtctttcttgtcttattgccactagtctctagtcagtgtgttaatcttacaaccaga | Nucleotide |
| | actcaattaccacctgcatacactaattctttcacacgtggtgtttattaccctgacaa | sequence/ORF |
| | agtattcagatcctcagtattacattcaactcaggacttgttcttacctttcttctccaat | (5′ to 3′ end) of |
| | gttacttggttccatgctatacatgtctctgggaccaatggtactaagaggtttgataa | a codon- |
| | ccctgtcctaccatttaatgatggtgtttatttcgcttccattgagaagtctaacataata | optimized |
| | agaggctggatatttggtactactttagattcgaagacccagtccctacttattgttaa | SARS-CoV-2 S |
| | taacgctactaatgttgttattaaagtctgtgaatttcaattctgtaatgatccattcttgg | antigen |
| | gtgtttctaaccacaagaacaacaagagttggatggaaagtgagttcagagtttat | sequence based |
| | tctagtgcgaataattgcactttcgaatatgtctctcagcctttccttatggaccttgaa | on the Wuhan- |
| | ggtaaacagggtaatttcaagaatcttagggaatttgtgtttaagaatattgatggtta | Hu-1 reference |
| | tttcaagatatattctaagcacacgcctattaatttagtgcgtgatctccctcagggttt | strain |
| | ctcggctttagaaccattggtagatttgccaataggtattaacatcactaggtttcaa | (#NC_045512) |
| | actttacttgctttacatagaagttatttgactcctggtgattcttcttcaggttggacagc | and optimized |
| | tggtgctgcagcttattatgtgggttatcttcaacctaggactttcctattgaaatataat | for vaccinia, |
| | gagaatggaaccattacagatgctgtagactgtgcacttgaccctctctcagaaac | which is further |
| | aaagtgtacgttgaaatccttcactgtagagaaaggaatctatcaaacttctaacttt | modified to |
| | agagtccaaccaacagaatctattgttagatttcctaatattacaaacttgtgcccttt | encode for an S |
| | cggtgaagtatttaacgccaccaaatttgcatctgtttatgcttggaacaggaagag | antigen that |
| | aatcagcaactgtgttgctgattattctgtcctatataattccgcatcattctccacattt | includes |
| | aagtgttatggagtgtctcctactaaattaaatgatctctgctttactaatgtctatgca | mutations of the |
| | gattcatttgtaattagaggtgatgaagtcagacaaatcgctccagggcaaactgg | B.1.621 variant |
| | aaagattgctgattataattataaattaccagatgactttacaggctgcgttatagctt | lineage, |
| | ggaattctaacaatcttgattctaaggttggtggtaattataattacctgtatagattgtt | including T95N, |
| | taggaagtctaatctcaaacctttcgagagagatatttcaactgaaatctatcaggc | Y144S, Y145N, |
| | cggtagcacaccttgtaatggtgttaaaggatttaattgttactttcctttacaatcatat | R346K, E484K, |
| | ggtttccaacccacttatggtgttggttaccaaccatacagagtagtagtactttcattt | N501Y, D614G, |
| | gaacttctacatgcaccagcaactgtttgtggacctaagaagtctactaatttggtta | P681H, and |
| | agaacaaatgtgtcaatttcaacttcaatggtttaacaggcacaggtgttcttactga | D950N |
| | gtctaacaagaagtttctgcctttccaacaatttggcagagacattgctgacactact | |
| | gatgctgtccgtgatccacagacacttgagattcttgacattacaccatgttcatttgg | |
| | tggtgtcagtgttataacaccaggaacaaatacttctaaccaggttgctgttctttatc | |
| | agggtgttaactgcacagaagtccctgttgctattcatgcagatcaacttactcctac | |
| | ttggcgtgtttattctacaggttctaatgtatttcaaacacgtgcaggctgtttaatagg | |
| | agctgaacatgtcaacaactcatatgagtgtgacatacccattggtgcaggtatat | |
| | gcgctagttatcagactcagactaattctcatcggcgggcacgtagtgtagctagtc | |
| | aatccatcattgcctacactatgtcacttggtgcagagaattcagttgcttactctaat | |
| | aactctattgccatacccacaaactttactattagtgttaccacagaaattctaccag | |
| | tgtctatgaccaagacatcagtagattgtacaatgtacatttgtggtgattcaactga | |
| | atgcagcaatctattgttgcaatatggcagtttctgtacacaattaaaccgtgctttaa | |
| | ctggaatagctgttgaacaagacaagaacacccaagaagtatttgcacaagtca | |
| | aacaaatttacaagacaccaccaattaaagatttcggtggatttaatttctcacaaa | |
| | tattaccagatccatctaaaccaagcaagaggtcatttattgaagatctactattca | |
| | acaaagtgacacttgcagatgctggcttcatcaaacaatatggtgattgccttggtg | |
| | atattgctgctagagacctcatttgtgcacagaagtttaacggccttactgtattgcca | |
| | cctttgctcacagatgaaatgattgctcaatacacttctgcactgttagcgggtacaa | |
| | tcacttctggttggacctttggtgcaggtgctgcattacaaataccatttgctatgcaa | |
| | atggcttataggtttaatggtattggagttacacagaatgttctctatgagaaccaga | |
| | aattgattgccaaccaatttaatagtgctattggcaagattcaagactcactttcttcc | |
| | acagcaagtgcacttggtaaacttcagaatgtggtcaaccagaatgcacaagctt | |
| | taaacacgcttgttaaacaacttagctccaactttggtgcaatttcaagtgtattaaat | |
| | gatatcctttcacgtcttgacaaagttgaggctgaagtgcaaattgataggttgatca | |
| | caggcagacttcaaagtttgcagacatatgtgactcaacaattaattagagctgca | |
| | gaaatcagagcttctgctaatcttgctgctactaagatgtcagagtgtgtacttggac | |
| | aatctaagagagttgatttctgtggaaagggctatcatcttatgtccttccctcagtca | |
| | gcacctcatggtgtagtcttcttgcatgtgacttatgtccctgcacaagagaagaact | |
| | tcacaactgctcctgccatttgtcatgatggtaaagcacactttcctcgtgaaggtgt | |
| | ctttgtttcaaatggcacacactggtttgtaacacaaaggaatttctatgaaccaca | |
| | aatcattactacagacaacacatttgtgtctggtaactgtgatgttgtaataggaattg | |
| | tcaacaacacagtttatgatcctttgcaacctgaattagactcattcaaggaggagt | |
| | tagataaatatttcaagaatcatacatcaccagatgttgatttaggtgacatctctgg | |
| | cattaatgcttcagttgtaaacattcagaaagaaattgaccgcctcaatgaggttgc | |
| | caagaatttaaatgaatctctcatcgatctccaagaacttggaaagtatgagcagt | |
| | atatcaaatggccatggtacatttggctaggtttcatagctggcttgattgccatagta | |
| | atggtgacaattatgctttgctgtatgaccagttgctgtagttgtctcaagggctgttgtt | |
| | cttgtggatcctgctgcaaatttgatgaagacgactctgagccagtgctcaaagga | |
| | gtcaaattacattacacataa | |
| 14 | MFVFLVLLPLVSSQCVNLTTRTQLPPAYTNSFTRGVYYPD | Amino acid |
| | KVFRSSVLHSTQDLFLPFFSNVTWFHAIHVSGTNGTKRFD | sequence (N- to |
| | NPVLPFNDGVYFASIEKSNIIRGWIFGTTLDSKTQSLLIVNN | C-terminus) of a |
| | ATNVVIKVCEFQFCNDPFLGVSNHKNNKSWMESEFRVYS | codon-optimized |
| | SANNCTFEYVSQPFLMDLEGKQGNFKNLREFVFKNIDGYF | SARS-CoV-2 S |
| | KIYSKHTPINLVRDLPQGFSALEPLVDLPIGINITRFQTLLAL | antigen |
| | HRSYLTPGDSSSGWTAGAAAYYVGYLQPRTFLLKYNENG | sequence based |
| | TITDAVDCALDPLSETKCTLKSFTVEKGIYQTSNFRVQPTE | on the Wuhan- |
| | SIVRFPNITNLCPFGEVFNATKFASVYAWNRKRISNCVADY | Hu-1 reference |
| | SVLYNSASFSTFKCYGVSPTKLNDLCFTNVYADSFVIRGD | strain |
| | EVRQIAPGQTGKIADYNYKLPDDFTGCVIAWNSNNLDSKV | (#NC_045512) |
| | GGNYNYLYRLFRKSNLKPFERDISTEIYQAGSTPCNGVKG | and optimized |
| | FNCYFPLQSYGFQPTYGVGYQPYRVVVLSFELLHAPATVC | for vaccinia, |
| | GPKKSTNLVKNKCVNFNFNGLTGTGVLTESNKKFLPFQQF | which is further |
| | GRDIADTTDAVRDPQTLEILDITPCSFGGVSVITPGTNTSN | modified to |
| | QVAVLYQGVNCTEVPVAIHADQLTPTWRVYSTGSNVFQT | encode for an S |
| | RAGCLIGAEHVNNSYECDIPIGAGICASYQTQTNSHRRAR | antigen that |
| | SVASQSIIAYTMSLGAENSVAYSNNSIAIPTNFTISVTTEILP | includes |
| | VSMTKTSVDCTMYICGDSTECSNLLLQYGSFCTQLNRALT | mutations of the |
| | GIAVEQDKNTQEVFAQVKQIYKTPPIKDFGGFNFSQILPDP | B.1.621 variant |
| | SKPSKRSFIEDLLFNKVTLADAGFIKQYGDCLGDIAARDLIC | lineage, |
| | AQKFNGLTVLPPLLTDEMIAQYTSALLAGTITSGWTFGAGA | including T95N, |
| | ALQIPFAMQMAYRFNGIGVTQNVLYENQKLIANQFNSAIGK | Y144S, Y145N, |
| | IQDSLSSTASALGKLQNVVNQNAQALNTLVKQLSSNFGAIS | R346K, E484K, |
| | SVLNDILSRLDKVEAEVQIDRLITGRLQSLQTYVTQQLIRAA | N501Y, D614G, |
| | EIRASANLAATKMSECVLGQSKRVDFCGKGYHLMSFPQS | P681H, and |
| | APHGVVFLHVTYVPAQEKNFTTAPAICHDGKAHFPREGVF | D950N |
| | VSNGTHWFVTQRNFYEPQIITTDNTFVSGNCDVVIGIVNNT | |
| | VYDPLQPELDSFKEELDKYFKNHTSPDVDLGDISGINASVV | |
| | NIQKEIDRLNEVAKNLNESLIDLQELGKYEQYIKWPWYIWL | |
| | GFIAGLIAIVMVTIMLCCMTSCCSCLKGCCSCGSCCKFDED | |
| | DSEPVLKGVKLHYT | |
| 15 | atgtttgtctttcttgtcttattgctactagtctctagtcagtgtgttaatcttacaaccaga | Nucleotide |
| | actcaattaccacctgcatacactaattctttcacacgtggtgtttattaccctgacaa | sequence/ORF |
| | agtattcagatcctcagtattacattcaactcaggacttgttcttacctttcttctccaat | (5′ to 3′ end) of |
| | gttacttggttccatgctatacatgtctctgggaccaatggtactaagaggtttgataa | a codon- |
| | ccctgtcctaccatttaatgatggtgtttatttcgcttccactgagaagtctaacataat | optimized |
| | aagaggctggatatttggtactactttagattcgaagacccagtccctacttattgtta | SARS-CoV-2 S |
| | ataacgctactaatgttgttattaaagtctgtgaatttcaattctttaatgatccattcttg | antigen |
| | ggtgtttaccacaagaacaacaagagttggatggaaagtgagttcagagtttattc | sequence based |
| | tagtgcgaataattgcactttcgaatatgtctctcagcctttccttatggaccttgaagg | on the Wuhan- |
| | taaacagggtaatttcaagaatcttagcgaatttgtgtttaagaatattgatggttattt | Hu-1 reference |
| | caagatatattctaagcacacgcctattaatttagtgcgtggtctccctcagggtttct | strain |
| | cggctttagaaccattggtagatttgccaataggtattaacatcactaggtttcaaac | (#NC_045512) |
| | tttattacatagaagttatttgactcctggtgattcttcttcaggttggacagctggtgct | and optimized |
| | gcagcttattatgtgggttatcttcaacctaggactttcctattgaaatataatgagaat | for vaccinia, |
| | ggaaccattacagatgctgtagactgtgcacttgaccctctctcagaaacaaagtg | which is further |
| | tacgttgaaatccttcactgtagagaaaggaatctatcaaacttctaactttagagtc | modified to |
| | caaccaacagaatctattgttagatttcctaatattacaaacttgtgccctttcggtga | encode for an S |
| | agtatttaacgccaccagatttgcatctgtttatgcttggaacaggaagagaatcag | antigen that |
| | caactgtgttgctgattattctgtcctatataattccgcatcattctccacatttaagtgtt | includes |
| | atggagtgtctcctactaaattaaatgatctctgctttactaatgtctatgcagattcatt | mutations of the |
| | tgtaattagaggtgatgaagtcagacaaatcgctccagggcaaactggaaagatt | C.1.2 variant |
| | gctgattataattataaattaccagatgactttacaggctgcgttatagcttggaattct | lineage, |
| | aacaatcttgattctaaggttggtggtaatcataattacctgtatagattgtttaggaa | including P9L, |
| | gtctaatctcaaacctttcgagagagatatttcaactgaaatctatcaggccggtag | C136F, R190S, |
| | cacaccttgtaatggtgttaaaggatttaattgttactttcctttacaatcatatggtttcc | D215G, |
| | aacccacttatggtgttggttaccaaccatacagagtagtagtactttcatttgaactt | Del242/243, |
| | ctacatgcaccagcaactgtttgtggacctaagaagtctactaatttggttaagaac | Y449Y, E484K, |
| | aaatgtgtcaatttcaacttcaatggtttaacaggcacaggtgttcttactgagtctaa | N501Y, D614G, |
| | caagaagtttctgcctttccaacaatttggcagagacattgctgacactactgatgct | H655Y, N679K, |
| | gtccgtgatccacagacacttgagattcttgacattacaccatgttcatttggtggtgt | T716I, and |
| | cagtgttataacaccaggaacaaatacttctaaccaggttgctgttctttatcagggt | T859N |
| | gttaactgcacagaagtccctgttgctattcatgcagatcaacttactcctacttggc | |
| | gtgtttattctacaggttctaatgtatttcaaacacgtgcaggctgtttaataggagctg | |
| | aatatgtcaacaactcatatgagtgtgacatacccattggtgcaggtatatgcgcta | |
| | gttatcagactcagactaagtctcctcggcgggcacgtagtgtagctagtcaatcc | |
| | atcattgcctacactatgtcacttggtgcagagaattcagttgcttactctaataactct | |
| | attgccatacccataaactttactattagtgttaccacagaaattctaccagtgtctat | |
| | gaccaagacatcagtagattgtacaatgtacatttgtggtgattcaactgaatgcag | |
| | caatctattgttgcaatatggcagtttctgtacacaattaaaccgtgctttaactggaa | |
| | tagctgttgaacaagacaagaacacccaagaagtatttgcacaagtcaaacaa | |
| | atttacaagacaccaccaattaaagatttcggtggatttaatttctcacaaatattac | |
| | cagatccatctaaaccaagcaagaggtcatttattgaagatctactattcaacaaa | |
| | gtgacacttgcagatgctggcttcatcaaacaatatggtgattgccttggtgatattg | |
| | ctgctagagacctcatttgtgcacagaagtttaacggccttaatgtattgccacctttg | |
| | ctcacagatgaaatgattgctcaatacacttctgcactgttagcgggtacaatcactt | |
| | ctggttggacctttggtgcaggtgctgcattacaaataccatttgctatgcaaatggc | |
| | ttataggtttaatggtattggagttacacagaatgttctctatgagaaccagaaattg | |
| | attgccaaccaatttaatagtgctattggcaagattcaagactcactttcttccacag | |
| | caagtgcacttggtaaacttcaagatgtggtcaaccagaatgcacaagctttaaa | |
| | cacgcttgttaaacaacttagctccaactttggtgcaatttcaagtgtattaaatgata | |
| | tcctttcacgtcttgacaaagttgaggctgaagtgcaaattgataggttgatcacag | |
| | gcagacttcaaagtttgcagacatatgtgactcaacaattaattagagctgcagaa | |
| | atcagagcttctgctaatcttgctgctactaagatgtcagagtgtgtacttggacaat | |
| | ctaagagagttgatttctgtggaaagggctatcatcttatgtccttccctcagtcagca | |
| | cctcatggtgtagtcttcttgcatgtgacttatgtccctgcacaagagaagaacttca | |
| | caactgctcctgccatttgtcatgatggtaaagcacactttcctcgtgaaggtgtcttt | |
| | gtttcaaatggcacacactggtttgtaacacaaaggaatttctatgaaccacaaat | |
| | cattactacagacaacacatttgtgtctggtaactgtgatgttgtaataggaattgtca | |
| | acaacacagtttatgatcctttgcaacctgaattagactcattcaaggaggagttag | |
| | ataaatatttcaagaatcatacatcaccagatgttgatttaggtgacatctctggcatt | |
| | aatgcttcagttgtaaacattcagaaagaaattgaccgcctcaatgaggttgccaa | |
| | gaatttaaatgaatctctcatcgatctccaagaacttggaaagtatgagcagtatat | |
| | caaatggccatggtacatttggctaggtttcatagctggcttgattgccatagtaatg | |
| | gtgacaattatgctttgctgtatgaccagttgctgtagttgtctcaagggctgttgttctt | |
| | gtggatcctgctgcaaatttgatgaagacgactctgagccagtgctcaaaggagt | |
| | caaattacattacacataa | |
| 16 | MFVFLVLLLLVSSQCVNLTTRTQLPPAYTNSFTRGVYYPD | Amino acid |
| | KVFRSSVLHSTQDLFLPFFSNVTWFHAIHVSGTNGTKRFD | sequence (N- to |
| | NPVLPFNDGVYFASTEKSNIIRGWIFGTTLDSKTQSLLIVNN | C-terminus) of a |
| | ATNVVIKVCEFQFFNDPFLGVYHKNNKSWMESEFRVYSS | codon-optimized |
| | ANNCTFEYVSQPFLMDLEGKQGNFKNLSEFVFKNIDGYFK | SARS-CoV-2 S |
| | IYSKHTPINLVRGLPQGFSALEPLVDLPIGINITRFQTLLHRS | antigen |
| | YLTPGDSSSGWTAGAAAYYVGYLQPRTFLLKYNENGTITD | sequence based |
| | AVDCALDPLSETKCTLKSFTVEKGIYQTSNFRVQPTESIVR | on the Wuhan- |
| | FPNITNLCPFGEVFNATRFASVYAWNRKRISNCVADYSVL | Hu-1 reference |
| | YNSASFSTFKCYGVSPTKLNDLCFTNVYADSFVIRGDEVR | strain |
| | QIAPGQTGKIADYNYKLPDDFTGCVIAWNSNNLDSKVGGN | (#NC_045512) |
| | HNYLYRLFRKSNLKPFERDISTEIYQAGSTPCNGVKGFNC | and optimized |
| | YFPLQSYGFQPTYGVGYQPYRVVVLSFELLHAPATVCGP | for vaccinia, |
| | KKSTNLVKNKCVNFNFNGLTGTGVLTESNKKFLPFQQFGR | which is further |
| | DIADTTDAVRDPQTLEILDITPCSFGGVSVITPGTNTSNQVA | modified to |
| | VLYQGVNCTEVPVAIHADQLTPTWRVYSTGSNVFQTRAG | encode for an S |
| | CLIGAEYVNNSYECDIPIGAGICASYQTQTKSPRRARSVAS | antigen that |
| | QSIIAYTMSLGAENSVAYSNNSIAIPINFTISVTTEILPVSMTK | includes |
| | TSVDCTMYICGDSTECSNLLLQYGSFCTQLNRALTGIAVE | mutations of the |
| | QDKNTQEVFAQVKQIYKTPPIKDFGGFNFSQILPDPSKPSK | C.1.2 variant |
| | RSFIEDLLFNKVTLADAGFIKQYGDCLGDIAARDLICAQKFN | lineage, |
| | GLNVLPPLLTDEMIAQYTSALLAGTITSGWTFGAGAALQIP | including P9L, |
| | FAMQMAYRFNGIGVTQNVLYENQKLIANQFNSAIGKIQDSL | C136F, R190S, |
| | SSTASALGKLQDVVNQNAQALNTLVKQLSSNFGAISSVLN | D215G, |
| | DILSRLDKVEAEVQIDRLITGRLQSLQTYVTQQLIRAAEIRA | Del242/243, |
| | SANLAATKMSECVLGQSKRVDFCGKGYHLMSFPQSAPH | Y449Y, E484K, |
| | GVVFLHVTYVPAQEKNFTTAPAICHDGKAHFPREGVFVSN | N501Y, D614G, |
| | GTHWFVTQRNFYEPQIITTDNTFVSGNCDVVIGIVNNTVYD | H655Y, N679K, |
| | PLQPELDSFKEELDKYFKNHTSPDVDLGDISGINASVVNIQ | T716I, and |
| | KEIDRLNEVAKNLNESLIDLQELGKYEQYIKWPWYIWLGFI | T859N |
| | AGLIAIVMVTIMLCCMTSCCSCLKGCCSCGSCCKFDEDDS | |
| | EPVLKGVKLHYT | |
| 17 | atgtctgataacggaccacagaatcagcgaaacgcaccacgcattacgtttggtg | Nucleotide |
| | gaccctcagattcaactggcagtaaccagaatggagaacgcagtggagcgcga | sequence/ORF |
| | tctaaacaacgtcggcctcaaggtttacccaataatactgcgtcttggttcaccgctc | (5′ to 3′ end) of |
| | tcactcaacatggcaaggaaggccttaaattccctcgaggacaaggcgttccaat | a codon- |
| | taacaccaatagcagtccagatgaccaaattggctactaccgaagagctaccag | optimized |
| | acgaattcgtggtggtgacggtaagatgaaagatctcagtccaagatggtatttcta | SARS-CoV-2 N |
| | ctacctaggaactgggccagaagctggacttccctatggtgctaacaaagacgg | antigen |
| | catcatatgggttgcaactgagggagccttgaatacacctaaagatcacattggc | sequence based |
| | acccgcaatcctgctaacaatgctgcaatcgtgctacaacttcctcaaggaacaa | on the Wuhan- |
| | cattgcctaaaggcttctacgcagaagggagcagaggcggcagtcaagcctctt | Hu-1 reference |
| | ctcgttcctcatcacgtagtcgcaacagttcaagaaattcaactccaggcagcagt | strain |
| | atgggaacttctcctgctagaatggctggcaatggctgtgatgctgctcttgctttgct | (#NC_045512) |
| | gctgcttgacagattgaaccagcttgagagcaagatgtctggtaaaggccaaca | and optimized |
| | acaacaaggccaaactgtcactaagaaatctgctgctgaggcttctaagaagcct | for vaccinia, |
| | cggcagaaacgtactgccactaaagcatacaatgtaacacaagctttcggcaga | which is further |
| | cgtggtccagaacaaacccaaggaaactttggagaccaggaactaatcagaca | modified to |
| | aggaactgattacaaacattggccgcaaattgcacaatttgctcccagcgcttcag | encode for an N |
| | cgttcttcggaatgtcgcgcattggcatggaagtcacaccttcgggaacgtggttga | antigen that |
| | cctacacaggtgccatcaaattggatgacaaagatccaaatttcaaagatcaagt | includes |
| | catattgctgaataagcatattgacgcatacaagacattcccaccaacagagcct | mutations of the |
| | aagaaggacaagaagaagaaggcttatgaaactcaagccttaccgcagagac | B.1.617.2 |
| | agaagaaacagcaaactgtgactcttcttcctgctgcagatttggatgatttctccaa | variant lineage |
| | acaattgcaacaatccatgagcagtgctgactcaactcaggcctaa | identified in |
| | | India, including |
| | | D63G, R203M, |
| | | G215C, and |
| | | D377Y |
| 18 | MSDNGPQNQRNAPRITFGGPSDSTGSNQNGERSGARSK | Amino acid |
| | QRRPQGLPNNTASWFTALTQHGKEGLKFPRGQGVPINTN | sequence (N- to |
| | SSPDDQIGYYRRATRRIRGGDGKMKDLSPRWYFYYLGTG | C-terminus) of a |
| | PEAGLPYGANKDGIIWVATEGALNTPKDHIGTRNPANNAAI | codon-optimized |
| | VLQLPQGTTLPKGFYAEGSRGGSQASSRSSSRSRNSSRN | SARS-CoV-2 N |
| | STPGSSMGTSPARMAGNGCDAALALLLLDRLNQLESKMS | antigen |
| | GKGQQQQGQTVTKKSAAEASKKPRQKRTATKAYNVTQA | sequence based |
| | FGRRGPEQTQGNFGDQELIRQGTDYKHWPQIAQFAPSAS | on the Wuhan- |
| | AFFGMSRIGMEVTPSGTWLTYTGAIKLDDKDPNFKDQVILL | Hu-1 reference |
| | NKHIDAYKTFPPTEPKKDKKKKAYETQALPQRQKKQQTVT | strain |
| | LLPAADLDDFSKQLQQSMSSADSTQA | (#NC_045512) |
| | | and optimized |
| | | for vaccinia, |
| | | which is further |
| | | modified to |
| | | encode for an N |
| | | antigen that |
| | | includes |
| | | mutations of the |
| | | B.1.617.2 |
| | | variant lineage |
| | | identified in |
| | | India, including |
| | | D63G, R203M, |
| | | G215C, and |
| | | D377Y |
| 19 | atgtctgataacggaccacagaatcagcgaaacgcactacgcattacgtttggtg | Nucleotide |
| | gaccctcagattcaactggcagtaaccagaatggagaacgcagtggagcgcga | sequence/ORF |
| | tctaaacaacgtcggcctcaaggtttacccaataatactgcgtcttggttcaccgctc | (5′ to 3′ end) of |
| | tcactcaacatggcaaggaagaccttaaattccctcgaggacaaggcgttccaat | a codon- |
| | taacaccaatagcagtccagatgaccaaattggctactaccgaagagctaccag | optimized |
| | acgaattcgtggtggtgacggtaagatgaaagatctcagtccaagatggtatttcta | SARS-CoV-2 N |
| | ctacctaggaactgggccagaagctggacttccctatggtgctaacaaagacgg | antigen |
| | catcatatgggttgcaactgagggagccttgaatacacctaaagatcacattggc | sequence based |
| | acccgcaatcctgctaacaatgctgcaatcgtgctacaacttcctcaaggaacaa | on the Wuhan- |
| | cattgcctaaaggcttctacgcagaagggagcagaggcggcagtcaagcctctt | Hu-1 reference |
| | ctcgttcctcatcacgtagtcgcaacagttcaagaaattcaactccaggcagcagt | strain |
| | aaacgaacttctcctgctagaatggctggcaatggcggtgatgctgctcttgctttgc | (#NC_045512) |
| | tgctgcttgacagattgaaccagcttgagagcaagatgtctggtaaaggccaaca | and optimized |
| | acaacaaggccaaactgtcactaagaaatctgctgctgaggcttctaagaagcct | for vaccinia, |
| | cggcagaaacgtactgccactaaagcatacaatgtaacacaagctttcggcaga | which is further |
| | cgtggtccagaacaaacccaaggaaactttggagaccaggaactaatcagaca | modified to |
| | aggaactgattacaaacattggccgcaaattgcacaatttgctcccagcgcttcag | encode for an N |
| | cgttcttcggaatgtcgcgcattggcatggaagtcacaccttcgggaacgtggttga | antigen that |
| | cctacacaggtgccatcaaattggatgacaaagatccaaatttcaaagatcaagt | includes |
| | catattgctgaataagcatattgacgcatacaagacattcccaccaacagagcct | mutations of the |
| | aagaaggacaagaagaagaaggctgatgaaactcaagccttaccgcacaga | C.1.2 variant |
| | cagaagaaacagcaaactgtgactcttcttcctgctgcagatttggatgatttctcca | lineage, |
| | aacaattgcaacaatccatgagcagtgctgactcaactcaggcctaa | including P13L, |
| | | R203K, G204R, |
| | | and Q384H |
| 20 | MSDNGPQNQRNALRITFGGPSDSTGSNQNGERSGARSK | Amino acid |
| | QRRPQGLPNNTASWFTALTQHGKEDLKFPRGQGVPINTN | sequence (N- to |
| | SSPDDQIGYYRRATRRIRGGDGKMKDLSPRWYFYYLGTG | C-terminus) of a |
| | PEAGLPYGANKDGIIWVATEGALNTPKDHIGTRNPANNAAI | codon-optimized |
| | VLQLPQGTTLPKGFYAEGSRGGSQASSRSSSRSRNSSRN | SARS-CoV-2 N |
| | STPGSSKRTSPARMAGNGGDAALALLLLDRLNQLESKMS | antigen |
| | GKGQQQQGQTVTKKSAAEASKKPRQKRTATKAYNVTQA | sequence based |
| | FGRRGPEQTQGNFGDQELIRQGTDYKHWPQIAQFAPSAS | on the Wuhan- |
| | AFFGMSRIGMEVTPSGTWLTYTGAIKLDDKDPNFKDQVILL | Hu-1 reference |
| | NKHIDAYKTFPPTEPKKDKKKKADETQALPHRQKKQQTVT | strain |
| | LLPAADLDDFSKQLQQSMSSADSTQA | (#NC_045512) |
| | | and optimized |
| | | for vaccinia, |
| | | which is further |
| | | modified to |
| | | encode for an N |
| | | antigen that |
| | | includes |
| | | mutations of the |
| | | C.1.2 variant |
| | | lineage, |
| | | including P13L, |
| | | R203K, G204R, |
| | | and Q384H |
| 21 | atgtttgtctttcttgtcttattgccactagtctctagtcagtgtgttaactttacaaccaga | Nucleotide |
| | actcaattaccacctgcatacactaattctttcacacgtggtgtttattaccctgacaa | sequence/ORF |
| | agtattcagatcctcagtattacattcaactcaggacttgttcttacctttcttctccaat | (5′ to 3′ end) of |
| | gttacttggttccatgctatacatgtctctgggaccaatggtactaagaggtttgctaa | a codon- |
| | ccctgtcctaccatttaatgatggtgtttatttcgcttccactgagaagtctaacataat | optimized |
| | aagaggctggatatttggtactactttagattcgaagacccagtccctacttattgtta | SARS-CoV-2 S |
| | ataacgctactaatgttgttattaaagtctgtgaatttcaattctgtaatgatccattcttg | antigen |
| | ggtgtttattaccacaagaacaacaagagttggatggaaagtgagttcagagttta | sequence based |
| | ttctagtgcgaataattgcactttcgaatatgtctctcagcctttccttatggaccttgaa | on the Wuhan- |
| | ggtaaacagggtaatttcaagaatcttagggaatttgtgtttaagaatattgatggtta | Hu-1 reference |
| | tttcaagatatattctaagcacacgcctattaatttagtgcgtggtctccctcagggttt | strain |
| | ctcggctttagaaccattggtagatttgccaataggtattaacatcactaggtttcaa | (#NC_045512) |
| | actttacatataagttatttgactcctggtgattcttcttcaggttggacagctggtgctg | and optimized |
| | cagcttattatgtgggttatcttcaacctaggactttcctattgaaatataatgagaatg | for vaccinia, |
| | gaaccattacagatgctgtagactgtgcacttgaccctctctcagaaacaaagtgt | which is further |
| | acgttgaaatccttcactgtagagaaaggaatctatcaaacttctaactttagagtc | modified to |
| | caaccaacagaatctattgttagatttcctaatattacaaacttgtgccctttcggtga | encode for an S |
| | agtatttaacgccaccagatttgcatctgtttatgcttggaacaggaagagaatcag | antigen that |
| | caactgtgttgctgattattctgtcctatataattccgcatcattctccacatttaagtgtt | includes |
| | atggagtgtctcctactaaattaaatgatctctgctttactaatgtctatgcagattcatt | mutations of the |
| | tgtaattagaggtgatgaagtcagacaaatcgctccagggcaaactggaaatatt | B.1.351 variant |
| | gctgattataattataaattaccagatgactttacaggctgcgttatagcttggaattct | lineage, |
| | aacaatcttgattctaaggttggtggtaattataattacctgtatagattgtttaggaag | including L18F, |
| | tctaatctcaaacctttcgagagagatatttcaactgaaatctatcaggccggtagc | D80A, D215G, |
| | acaccttgtaatggtgttaaaggatttaattgttactttcctttacaatcatatggtttcca | Del242-244, |
| | acccacttatggtgttggttaccaaccatacagagtagtagtactttcatttgaacttct | R246I, N501Y, |
| | acatgcaccagcaactgtttgtggacctaagaagtctactaatttggttaagaaca | E484K, K417N, |
| | aatgtgtcaatttcaacttcaatggtttaacaggcacaggtgttcttactgagtctaac | D614G, and |
| | aagaagtttctgcctttccaacaatttggcagagacattgctgacactactgatgctg | A701V |
| | tccgtgatccacagacacttgagattcttgacattacaccatgttcatttggtggtgtc | |
| | agtgttataacaccaggaacaaatacttctaaccaggttgctgttctttatcagggtg | |
| | ttaactgcacagaagtccctgttgctattcatgcagatcaacttactcctacttggcgt | |
| | gtttattctacaggttctaatgtatttcaaacacgtgcaggctgtttaataggagctga | |
| | acatgtcaacaactcatatgagtgtgacatacccattggtgcaggtatatgcgcta | |
| | gttatcagactcagactaattctcctcggcgggcacgtagtgtagctagtcaatcca | |
| | tcattgcctacactatgtcacttggtgtagagaattcagttgcttactctaataactctat | |
| | tgccatacccacaaactttactattagtgttaccacagaaattctaccagtgtctatg | |
| | accaagacatcagtagattgtacaatgtacatttgtggtgattcaactgaatgcagc | |
| | aatctattgttgcaatatggcagtttctgtacacaattaaaccgtgctttaactggaat | |
| | agctgttgaacaagacaagaacacccaagaagtatttgcacaagtcaaacaaa | |
| | tttacaagacaccaccaattaaagatttcggtggatttaatttctcacaaatattacc | |
| | agatccatctaaaccaagcaagaggtcatttattgaagatctactattcaacaaag | |
| | tgacacttgcagatgctggcttcatcaaacaatatggtgattgccttggtgatattgct | |
| | gctagagacctcatttgtgcacagaagtttaacggccttactgtattgccacctttgct | |
| | cacagatgaaatgattgctcaatacacttctgcactgttagcgggtacaatcacttct | |
| | ggttggacctttggtgcaggtgctgcattacaaataccatttgctatgcaaatggctt | |
| | ataggtttaatggtattggagttacacagaatgttctctatgagaaccagaaattgat | |
| | tgccaaccaatttaatagtgctattggcaagattcaagactcactttcttccacagca | |
| | agtgcacttggtaaacttcaagatgtggtcaaccagaatgcacaagctttaaaca | |
| | cgcttgttaaacaacttagctccaactttggtgcaatttcaagtgtattaaatgatatc | |
| | ctttcacgtcttgacaaagttgaggctgaagtgcaaattgataggttgatcacaggc | |
| | agacttcaaagtttgcagacatatgtgactcaacaattaattagagctgcagaaat | |
| | cagagcttctgctaatcttgctgctactaagatgtcagagtgtgtacttggacaatct | |
| | aagagagttgatttctgtggaaagggctatcatcttatgtccttccctcagtcagcac | |
| | ctcatggtgtagtcttcttgcatgtgacttatgtccctgcacaagagaagaacttcac | |
| | aactgctcctgccatttgtcatgatggtaaagcacactttcctcgtgaaggtgtctttgt | |
| | ttcaaatggcacacactggtttgtaacacaaaggaatttctatgaaccacaaatca | |
| | ttactacagacaacacatttgtgtctggtaactgtgatgttgtaataggaattgtcaac | |
| | aacacagtttatgatcctttgcaacctgaattagactcattcaaggaggagttagat | |
| | aaatatttcaagaatcatacatcaccagatgttgatttaggtgacatctctggcatta | |
| | atgcttcagttgtaaacattcagaaagaaattgaccgcctcaatgaggttgccaag | |
| | aatttaaatgaatctctcatcgatctccaagaacttggaaagtatgagcagtatatc | |
| | aaatggccatggtacatttggctaggtttcatagctggcttgattgccatagtaatggt | |
| | gacaattatgctttgctgtatgaccagttgctgtagttgtctcaagggctgttgttcttgt | |
| | ggatcctgctgcaaatttgatgaagacgactctgagccagtgctcaaaggagtca | |
| | aattacattacacataa | |
| 22 | MFVFLVLLPLVSSQCVNFTTRTQLPPAYTNSFTRGVYYPD | Amino acid |
| | KVFRSSVLHSTQDLFLPFFSNVTWFHAIHVSGTNGTKRFA | sequence (N- to |
| | NPVLPFNDGVYFASTEKSNIIRGWIFGTTLDSKTQSLLIVNN | C-terminus) of a |
| | ATNVVIKVCEFQFCNDPFLGVYYHKNNKSWMESEFRVYS | codon-optimized |
| | SANNCTFEYVSQPFLMDLEGKQGNFKNLREFVFKNIDGYF | SARS-CoV-2 S |
| | KIYSKHTPINLVRGLPQGFSALEPLVDLPIGINITRFQTLHIS | antigen |
| | YLTPGDSSSGWTAGAAAYYVGYLQPRTFLLKYNENGTITD | sequence based |
| | AVDCALDPLSETKCTLKSFTVEKGIYQTSNFRVQPTESIVR | on the Wuhan- |
| | FPNITNLCPFGEVFNATRFASVYAWNRKRISNCVADYSVL | Hu-1 reference |
| | YNSASFSTFKCYGVSPTKLNDLCFTNVYADSFVIRGDEVR | strain |
| | QIAPGQTGNIADYNYKLPDDFTGCVIAWNSNNLDSKVGGN | (#NC_045512) |
| | YNYLYRLFRKSNLKPFERDISTEIYQAGSTPCNGVKGFNC | and optimized |
| | YFPLQSYGFQPTYGVGYQPYRVVVLSFELLHAPATVCGP | for vaccinia, |
| | KKSTNLVKNKCVNFNFNGLTGTGVLTESNKKFLPFQQFGR | which is further |
| | DIADTTDAVRDPQTLEILDITPCSFGGVSVITPGTNTSNQVA | modified to |
| | VLYQGVNCTEVPVAIHADQLTPTWRVYSTGSNVFQTRAG | encode for an S |
| | CLIGAEHVNNSYECDIPIGAGICASYQTQTNSPRRARSVAS | antigen that |
| | QSIIAYTMSLGVENSVAYSNNSIAIPTNFTISVTTEILPVSMT | includes |
| | KTSVDCTMYICGDSTECSNLLLQYGSFCTQLNRALTGIAV | mutations of the |
| | EQDKNTQEVFAQVKQIYKTPPIKDFGGFNFSQILPDPSKPS | B.1.351 variant |
| | KRSFIEDLLFNKVTLADAGFIKQYGDCLGDIAARDLICAQKF | lineage, |
| | NGLTVLPPLLTDEMIAQYTSALLAGTITSGWTFGAGAALQI | including L18F, |
| | PFAMQMAYRFNGIGVTQNVLYENQKLIANQFNSAIGKIQD | D80A, D215G, |
| | SLSSTASALGKLQDVVNQNAQALNTLVKQLSSNFGAISSV | Del242-244, |
| | LNDILSRLDKVEAEVQIDRLITGRLQSLQTYVTQQLIRAAEI | R246I, N501Y, |
| | RASANLAATKMSECVLGQSKRVDFCGKGYHLMSFPQSAP | E484K, K417N, |
| | HGVVFLHVTYVPAQEKNFTTAPAICHDGKAHFPREGVFVS | D614G, and |
| | NGTHWFVTQRNFYEPQIITTDNTFVSGNCDVVIGIVNNTVY | A701V |
| | DPLQPELDSFKEELDKYFKNHTSPDVDLGDISGINASVVNI | |
| | QKEIDRLNEVAKNLNESLIDLQELGKYEQYIKWPWYIWLGF | |
| | IAGLIAIVMVTIMLCCMTSCCSCLKGCCSCGSCCKFDEDDS | |
| | EPVLKGVKLHYT | |
| 23 | atgtctgataacggaccacagaatcagcgaaacgcaccacgcattacgtttggtg | Nucleotide |
| | gaccctcagattcaactggcagtaaccagaatggagaacgcagtggagcgcga | sequence/ORF |
| | tctaaacaacgtcggcctcaaggtttacccaataatactgcgtcttggttcaccgctc | (5′ to 3′ end) of |
| | tcactcaacatggcaaggaagaccttaaattccctcgaggacaaggcgttccaat | a codon- |
| | taacaccaatagcagtccagatgaccaaattggctactaccgaagagctaccag | optimized |
| | acgaattcgtggtggtgacggtaagatgaaagatctcagtccaagatggtatttcta | SARS-CoV-2 N |
| | ctacctaggaactgggccagaagctggacttccctatggtgctaacaaagacgg | antigen |
| | catcatatgggttgcaactgagggagccttgaatacacctaaagatcacattggc | sequence based |
| | acccgcaatcctgctaacaatgctgcaatcgtgctacaacttcctcaaggaacaa | on the Wuhan- |
| | cattgcctaaaggcttctacgcagaagggagcagaggcggcagtcaagcctctt | Hu-1 reference |
| | ctcgttcctcatcacgtagtcgcaacagttcaagaaattcaactccaggcagcagt | strain |
| | agaggaatctctcctgctagaatggctggcaatggcggtgatgctgctcttgctttgc | (#NC_045512) |
| | tgctgcttgacagattgaaccagcttgagagcaagatgtctggtaaaggccaaca | and optimized |
| | acaacaaggccaaactgtcactaagaaatctgctgctgaggcttctaagaagcct | for vaccinia, |
| | cggcagaaacgtactgccactaaagcatacaatgtaacacaagctttcggcaga | which is further |
| | cgtggtccagaacaaacccaaggaaactttggagaccaggaactaatcagaca | modified to |
| | aggaactgattacaaacattggccgcaaattgcacaatttgctcccagcgcttcag | encode for an N |
| | cgttcttcggaatgtcgcgcattggcatggaagtcacaccttcgggaacgtggttga | antigen that |
| | cctacacaggtgccatcaaattggatgacaaagatccaaatttcaaagatcaagt | includes |
| | catattgctgaataagcatattgacgcatacaagacattcccaccaacagagcct | mutations of the |
| | aagaaggacaagaagaagaaggctgatgaaactcaagccttaccgcagaga | B.1.351 variant |
| | cagaagaaacagcaaactgtgactcttcttcctgctgcagatttggatgatttctcca | lineage, |
| | aacaattgcaacaatccatgagcagtgctgactcaactcaggcctaa | including T205I |
| 24 | MSDNGPQNQRNAPRITFGGPSDSTGSNQNGERSGARSK | Amino acid |
| | QRRPQGLPNNTASWFTALTQHGKEDLKFPRGQGVPINTN | sequence (N- to |
| | SSPDDQIGYYRRATRRIRGGDGKMKDLSPRWYFYYLGTG | C-terminus) of a |
| | PEAGLPYGANKDGIIWVATEGALNTPKDHIGTRNPANNAAI | codon-optimized |
| | VLQLPQGTTLPKGFYAEGSRGGSQASSRSSSRSRNSSRN | SARS-CoV-2 N |
| | STPGSSRGISPARMAGNGGDAALALLLLDRLNQLESKMS | antigen |
| | GKGQQQQGQTVTKKSAAEASKKPRQKRTATKAYNVTQA | sequence based |
| | FGRRGPEQTQGNFGDQELIRQGTDYKHWPQIAQFAPSAS | on the Wuhan- |
| | AFFGMSRIGMEVTPSGTWLTYTGAIKLDDKDPNFKDQVILL | Hu-1 reference |
| | NKHIDAYKTFPPTEPKKDKKKKADETQALPQRQKKQQTVT | strain |
| | LLPAADLDDFSKQLQQSMSSADSTQA | (#NC_045512) |
| | | and optimized |
| | | for vaccinia, |
| | | which is further |
| | | modified to |
| | | encode for an N |
| | | antigen that |
| | | includes |
| | | mutations of the |
| | | B.1.351 variant |
| | | lineage, |
| | | including T205I |
| 25 | atgtttgtctttcttgtcttattgccactagtctctagtcagtgtgttaatcttacaaccaga | Nucleotide |
| | actcaattaccacctgcatacactaattctttcacacgtggtgtttattaccctgacaa | sequence/ORF |
| | agtattcagatcctcagtattacattcaactcaggacttgttcttacctttcttctccaat | (5′ to 3′ end) of |
| | gttacttggttccatgttatatctgggaccaatggtactaagaggtttgataaccctgt | a codon- |
| | cctaccatttaatgatggtgtttatttcgcttccattgagaagtctaacataataagag | optimized |
| | gctggatatttggtactactttagattcgaagacccagtccctacttattgttaataac | SARS-CoV-2 S |
| | gctactaatgttgttattaaagtctgtgaatttcaattctgtaatgatccattcttggatca | antigen |
| | caagaacaacaagagttggatggaaagtgagttcagagtttattctagtgcgaat | sequence based |
| | aattgcactttcgaatatgtctctcagcctttccttatggaccttgaaggtaaacaggg | on the Wuhan- |
| | taatttcaagaatcttagggaatttgtgtttaagaatattgatggttatttcaagatatatt | Hu-1 reference |
| | ctaagcacacgcctattatagtgcgtgaacctgaagatctccctcagggtttctcgg | strain |
| | ctttagaaccattggtagatttgccaataggtattaacatcactaggtttcaaacttta | (#NC_045512) |
| | cttgctttacatagaagttatttgactcctggtgattcttcttcaggttggacagctggtg | and optimized |
| | ctgcagcttattatgtgggttatcttcaacctaggactttcctattgaaatataatgaga | for vaccinia, |
| | atggaaccattacagatgctgtagactgtgcacttgaccctctctcagaaacaaag | which is further |
| | tgtacgttgaaatccttcactgtagagaaaggaatctatcaaacttctaactttagag | modified to |
| | tccaaccaacagaatctattgttagatttcctaatattacaaacttgtgccctttcgac | encode for an S |
| | gaagtatttaacgccaccagatttgcatctgtttatgcttggaacaggaagagaatc | antigen that |
| | agcaactgtgttgctgattattctgtcctatataatctcgcaccattcttcacatttaagt | includes |
| | gttatggagtgtctcctactaaattaaatgatctctgctttactaatgtctatgcagattc | mutations of the |
| | atttgtaattagaggtgatgaagtcagacaaatcgctccagggcaaactggaaat | B.1.1.529/BA.1 |
| | attgctgattataattataaattaccagatgactttacaggctgcgttatagcttggaat | variant lineage, |
| | tctaacaaacttgattctaaggttagtggtaattataattacctgtatagattgtttagg | including A67V, |
| | aagtctaatctcaaacctttcgagagagatatttcaactgaaatctatcaggccggt | Del69-70 (HV), |
| | aacaaaccttgtaatggtgttgcaggatttaattgttactttcctttacgatcatatagttt | T95I, G142D, |
| | ccgacccacttatggtgttggtcaccaaccatacagagtagtagtactttcatttgaa | Del143-145 |
| | cttctacatgcaccagcaactgtttgtggacctaagaagtctactaatttggttaaga | (VYY), Del211 |
| | acaaatgtgtcaatttcaacttcaatggtttgaaaggcacaggtgttcttactgagtct | (N), L212I, |
| | aacaagaagtttctgcctttccaacaatttggcagagacattgctgacactactgat | Ins214 (EPE), |
| | gctgtccgtgatccacagacacttgagattcttgacattacaccatgttcatttggtgg | G339D, S371L, |
| | tgtcagtgttataacaccaggaacaaatacttctaaccaggttgctgttctttatcag | S373P, S375F, |
| | ggtgttaactgcacagaagtccctgttgctattcatgcagatcaacttactcctacttg | K417N, N440K, |
| | gcgtgtttattctacaggttctaatgtatttcaaacacgtgcaggctgtttaataggag | G446S, S477N, |
| | ctgaatatgtcaacaactcatatgagtgtgacatacccattggtgcaggtatatgcg | T478K, E484A, |
| | ctagttatcagactcagactaagtctcatcggcgggcacgtagtgtagctagtcaat | Q493R, G496S, |
| | ccatcattgcctacactatgtcacttggtgcagagaattcagttgcttactctaataac | Q498R, N501Y, |
| | tctattgccatacccacaaactttactattagtgttaccacagaaattctaccagtgtc | Y505H, T547K, |
| | tatgaccaagacatcagtagattgtacaatgtacatttgtggtgattcaactgaatgc | D614G, H655Y, |
| | agcaatctattgttgcaatatggcagtttctgtacacaattaaagcgtgctttaactgg | N679K, P681H, |
| | aatagctgttgaacaagacaagaacacccaagaagtatttgcacaagtcaaac | N764K, D796Y, |
| | aaatttacaagacaccaccaattaagtatttcggtggatttaatttctcacaaatatta | N856K, Q954H, |
| | ccagatccatctaaaccaagcaagaggtcatttattgaagatctactattcaacaa | N969K, and |
| | agtgacacttgcagatgctggcttcatcaaacaatatggtgattgccttggtgatatt | L981F |
| | gctgctagagacctcatttgtgcacagaagtttaagggccttactgtattgccaccttt | |
| | gctcacagatgaaatgattgctcaatacacttctgcactgttaggggtacaatcac | |
| | ttctggttggacctttggtgcaggtgctgcattacaaataccatttgctatgcaaatgg | |
| | cttataggtttaatggtattggagttacacagaatgttctctatgagaaccagaaatt | |
| | gattgccaaccaatttaatagtgctattggcaagattcaagactcactttcttccaca | |
| | gcaagtgcacttggtaaacttcaagatgtggtcaaccacaatgcacaagctttaa | |
| | acacgcttgttaaacaacttagctccaagtttggtgcaatttcaagtgtattaaatgat | |
| | atcttctcacgtcttgacaaagttgaggctgaagtgcaaattgataggttgatcaca | |
| | ggcagacttcaaagtttgcagacatatgtgactcaacaattaattagagctgcaga | |
| | aatcagagcttctgctaatcttgctgctactaagatgtcagagtgtgtacttggacaa | |
| | tctaagagagttgatttctgtggaaagggctatcatcttatgtccttccctcagtcagc | |
| | acctcatggtgtagtcttcttgcatgtgacttatgtccctgcacaagagaagaacttc | |
| | acaactgctcctgccatttgtcatgatggtaaagcacactttcctcgtgaaggtgtctt | |
| | tgtttcaaatggcacacactggtttgtaacacaaaggaatttctatgaaccacaaat | |
| | cattactacagacaacacatttgtgtctggtaactgtgatgttgtaataggaattgtca | |
| | acaacacagtttatgatcctttgcaacctgaattagactcattcaaggaggagttag | |
| | ataaatatttcaagaatcatacatcaccagatgttgatttaggtgacatctctggcatt | |
| | aatgcttcagttgtaaacattcagaaagaaattgaccgcctcaatgaggttgccaa | |
| | gaatttaaatgaatctctcatcgatctccaagaacttggaaagtatgagcagtatat | |
| | caaatggccatggtacatttggctaggtttcatagctggcttgattgccatagtaatg | |
| | gtgacaattatgctttgctgtatgaccagttgctgtagttgtctcaagggctgttgttctt | |
| | gtggatcctgctgcaaatttgatgaagacgactctgagccagtgctcaaaggagt | |
| | caaattacattacacataa | |
| 26 | MFVFLVLLPLVSSQCVNLTTRTQLPPAYTNSFTRGVYYPD | Amino acid |
| | KVFRSSVLHSTQDLFLPFFSNVTWFHVISGTNGTKRFDNP | sequence (N- to |
| | VLPFNDGVYFASIEKSNIIRGWIFGTTLDSKTQSLLIVNNAT | C-terminus) of a |
| | NVVIKVCEFQFONDPFLDHKNNKSWMESEFRVYSSANNC | codon-optimized |
| | TFEYVSQPFLMDLEGKQGNFKNLREFVFKNIDGYFKIYSK | SARS-CoV-2 S |
| | HTPIIVREPEDLPQGFSALEPLVDLPIGINITRFQTLLALHRS | antigen |
| | YLTPGDSSSGWTAGAAAYYVGYLQPRTFLLKYNENGTITD | sequence based |
| | AVDCALDPLSETKCTLKSFTVEKGIYQTSNFRVQPTESIVR | on the Wuhan- |
| | FPNITNLCPFDEVFNATRFASVYAWNRKRISNCVADYSVL | Hu-1 reference |
| | YNLAPFFTFKCYGVSPTKLNDLCFTNVYADSFVIRGDEVR | strain |
| | QIAPGQTGNIADYNYKLPDDFTGCVIAWNSNKLDSKVSGN | (#NC_045512) |
| | YNYLYRLFRKSNLKPFERDISTEIYQAGNKPCNGVAGFNC | and optimized |
| | YFPLRSYSFRPTYGVGHQPYRVVVLSFELLHAPATVCGPK | for vaccinia, |
| | KSTNLVKNKCVNFNFNGLKGTGVLTESNKKFLPFQQFGR | which is further |
| | DIADTTDAVRDPQTLEILDITPCSFGGVSVITPGTNTSNQVA | modified to |
| | VLYQGVNCTEVPVAIHADQLTPTWRVYSTGSNVFQTRAG | encode for an S |
| | CLIGAEYVNNSYECDIPIGAGICASYQTQTKSHRRARSVAS | antigen that |
| | QSIIAYTMSLGAENSVAYSNNSIAIPTNFTISVTTEILPVSMT | includes |
| | KTSVDCTMYICGDSTECSNLLLQYGSFCTQLKRALTGIAVE | mutations of the |
| | QDKNTQEVFAQVKQIYKTPPIKYFGGFNFSQILPDPSKPSK | B.1.1.529/BA.1 |
| | RSFIEDLLFNKVTLADAGFIKQYGDCLGDIAARDLICAQKFK | variant lineage, |
| | GLTVLPPLLTDEMIAQYTSALLAGTITSGWTFGAGAALQIP | including A67V, |
| | FAMQMAYRFNGIGVTQNVLYENQKLIANQFNSAIGKIQDSL | Del69-70 (HV), |
| | SSTASALGKLQDVVNHNAQALNTLVKQLSSKFGAISSVLN | T95I, G142D, |
| | DIFSRLDKVEAEVQIDRLITGRLQSLQTYVTQQLIRAAEIRA | Del143-145 |
| | SANLAATKMSECVLGQSKRVDFCGKGYHLMSFPQSAPH | (VYY), Del211 |
| | GVVFLHVTYVPAQEKNFTTAPAICHDGKAHFPREGVFVSN | (N), L212I, |
| | GTHWFVTQRNFYEPQIITTDNTFVSGNCDVVIGIVNNTVYD | Ins214 (EPE), |
| | PLQPELDSFKEELDKYFKNHTSPDVDLGDISGINASVVNIQ | G339D, S371L, |
| | KEIDRLNEVAKNLNESLIDLQELGKYEQYIKWPWYIWLGFI | S373P, S375F, |
| | AGLIAIVMVTIMLCCMTSCCSCLKGCCSCGSCCKFDEDDS | K417N, N440K, |
| | EPVLKGVKLHYT | G446S, S477N, |
| | | T478K, E484A, |
| | | Q493R, G496S, |
| | | Q498R, N501Y, |
| | | Y505H, T547K, |
| | | D614G, H655Y, |
| | | N679K, P681H, |
| | | N764K, D796Y, |
| | | N856K, Q954H, |
| | | N969K, and |
| | | L981F |
| 27 | atgtctgataacggaccacagaatcagcgaaacgcactacgcattacgtttggtg | Nucleotide |
| | gaccctcagattcaactggcagtaaccagaatggaggagcgcgatctaaacaa | sequence/ORF |
| | cgtcggcctcaaggtttacccaataatactgcgtcttggttcaccgctctcactcaac | (5′ to 3′ end) of |
| | atggcaaggaagaccttaaattccctcgaggacaaggcgttccaattaacacca | a codon- |
| | atagcagtccagatgaccaaattggctactaccgaagagctaccagacgaattc | optimized |
| | gtggtggtgacggtaagatgaaagatctcagtccaagatggtatttctactacctag | SARS-CoV-2 N |
| | gaactgggccagaagctggacttccctatggtgctaacaaagacggcatcatatg | antigen |
| | ggttgcaactgagggagccttgaatacacctaaagatcacattggcacccgcaat | sequence based |
| | cctgctaacaatgctgcaatcgtgctacaacttcctcaaggaacaacattgcctaa | on the Wuhan- |
| | aggcttctacgcagaagggagcagaggcggcagtcaagcctcttctcgttcctca | Hu-1 reference |
| | tcacgtagtcgcaacagttcaagaaattcaactccaggcagcagtaaacgaactt | strain |
| | ctcctgctagaatggctggcaatggcggtgatgctgctcttgctttgctgctgcttgac | (#NC_045512) |
| | agattgaaccagcttgagagcaagatgtctggtaaaggccaacaacaacaagg | and optimized |
| | ccaaactgtcactaagaaatctgctgctgaggcttctaagaagcctcggcagaaa | for vaccinia, |
| | cgtactgccactaaagcatacaatgtaacacaagctttcggcagacgtggtccag | which is further |
| | aacaaacccaaggaaactttggagaccaggaactaatcagacaaggaactga | modified to |
| | ttacaaacattggccgcaaattgcacaatttgctcccagcgcttcagcgttcttcgga | encode for an N |
| | atgtcgcgcattggcatggaagtcacaccttcgggaacgtggttgacctacacag | antigen that |
| | gtgccatcaaattggatgacaaagatccaaatttcaaagatcaagtcatattgctg | includes |
| | aataagcatattgacgcatacaagacattcccaccaacagagcctaagaagga | mutations of the |
| | caagaagaagaaggctgatgaaactcaagccttaccgcagagacagaagaa | B.1.1.529/BA.1 |
| | acagcaaactgtgactcttcttcctgctgcagatttggatgatttctccaaacaattgc | variant lineage, |
| | aacaatccatgagcagtgctgactcaactcaggcctaa | including P13L, |
| | | Del31-33 (ERS), |
| | | R203K, G204R |
| 28 | MSDNGPQNQRNALRITFGGPSDSTGSNQNGGARSKQRR | Amino acid |
| | PQGLPNNTASWFTALTQHGKEDLKFPRGQGVPINTNSSP | sequence (N- to |
| | DDQIGYYRRATRRIRGGDGKMKDLSPRWYFYYLGTGPEA | C-terminus) of a |
| | GLPYGANKDGIIWVATEGALNTPKDHIGTRNPANNAAIVLQ | codon-optimized |
| | LPQGTTLPKGFYAEGSRGGSQASSRSSSRSRNSSRNSTP | SARS-CoV-2 N |
| | GSSKRTSPARMAGNGGDAALALLLLDRLNQLESKMSGKG | antigen |
| | QQQQGQTVTKKSAAEASKKPRQKRTATKAYNVTQAFGR | sequence based |
| | RGPEQTQGNFGDQELIRQGTDYKHWPQIAQFAPSASAFF | on the Wuhan- |
| | GMSRIGMEVTPSGTWLTYTGAIKLDDKDPNFKDQVILLNK | Hu-1 reference |
| | HIDAYKTFPPTEPKKDKKKKADETQALPQRQKKQQTVTLL | strain |
| | PAADLDDFSKQLQQSMSSADSTQA | (#NC_045512) |
| | | and optimized |
| | | for vaccinia, |
| | | which is further |
| | | modified to |
| | | encode for an N |
| | | antigen that |
| | | includes |
| | | mutations of the |
| | | B.1.1.529/BA.1 |
| | | variant lineage, |
| | | including P13L, |
| | | Del31-33 (ERS), |
| | | R203K, G204R |
| 29 | atgtttgtctttcttgtcttattgccactagtctctagtcagtgtgttaactttacaaacaga | Nucleotide |
| | actcaattaccatctgcatacactaattctttcacacgtggtgtttattaccctgacaa | sequence/ORF |
| | agtattcagatcctcagtattacattcaactcaggacttgttcttacctttcttctccaat | (5′ to 3′ end) of |
| | gttacttggttccatgctatacatgtctctgggaccaatggtactaagaggtttgataa | a codon- |
| | ccctgtcctaccatttaatgatggtgtttatttcgcttccactgagaagtctaacataat | optimized |
| | aagaggctggatatttggtactactttagattcgaagacccagtccctacttattgtta | SARS-CoV-2 S |
| | ataacgctactaatgttgttattaaagtctgtgaatttcaattctgtaattatccattcttg | antigen |
| | ggtgtttattaccacaagaacaacaagagttggatggaaagtgagttcagagttta | sequence based |
| | ttctagtgcgaataattgcactttcgaatatgtctctcagcctttccttatggaccttgaa | on the Wuhan- |
| | ggtaaacagggtaatttcaagaatcttagcgaatttgtgtttaagaatattgatggtta | Hu-1 reference |
| | tttcaagatatattctaagcacacgcctattaatttagtgcgtgatctccctcagggttt | strain |
| | ctcggctttagaaccattggtagatttgccaataggtattaacatcactaggtttcaa | (#NC_045512) |
| | actttacttgctttacatagaagttatttgactcctggtgattcttcttcaggttggacagc | and optimized |
| | tggtgctgcagcttattatgtgggttatcttcaacctaggactttcctattgaaatataat | for vaccinia, |
| | gagaatggaaccattacagatgctgtagactgtgcacttgaccctctctcagaaac | which is further |
| | aaagtgtacgttgaaatccttcactgtagagaaaggaatctatcaaacttctaacttt | modified to |
| | agagtccaaccaacagaatctattgttagatttcctaatattacaaacttgtgcccttt | encode for an S |
| | cggtgaagtatttaacgccaccagatttgcatctgtttatgcttggaacaggaagag | antigen that |
| | aatcagcaactgtgttgctgattattctgtcctatataattccgcatcattctccacattt | includes |
| | aagtgttatggagtgtctcctactaaattaaatgatctctgctttactaatgtctatgca | mutations of the |
| | gattcatttgtaattagaggtgatgaagtcagacaaatcgctccagggcaaactgg | P.1 variant |
| | aacgattgctgattataattataaattaccagatgactttacaggctgcgttatagctt | lineage, |
| | ggaattctaacaatcttgattctaaggttggtggtaattataattacctgtatagattgtt | including L18F, |
| | taggaagtctaatctcaaacctttcgagagagatatttcaactgaaatctatcaggc | T20N, P26S, |
| | cggtagcacaccttgtaatggtgttaaaggatttaattgttactttcctttacaatcatat | D138Y, R190S, |
| | ggtttccaacccacttatggtgttggttaccaaccatacagagtagtagtactttcattt | K417T, E484K, |
| | gaacttctacatgcaccagcaactgtttgtggacctaagaagtctactaatttggtta | N501Y, D614G, |
| | agaacaaatgtgtcaatttcaacttcaatggtttaacaggcacaggtgttcttactga | H655Y, T1027I, |
| | gtctaacaagaagtttctgcctttccaacaatttggcagagacattgctgacactact | and V1167F |
| | gatgctgtccgtgatccacagacacttgagattcttgacattacaccatgttcatttgg | |
| | tggtgtcagtgttataacaccaggaacaaatacttctaaccaggttgctgttctttatc | |
| | agggtgttaactgcacagaagtccctgttgctattcatgcagatcaacttactcctac | |
| | ttggcgtgtttattctacaggttctaatgtatttcaaacacgtgcaggctgtttaatagg | |
| | agctgaatatgtcaacaactcatatgagtgtgacatacccattggtgcaggtatatg | |
| | cgctagttatcagactcagactaattctcctcggcgggcacgtagtgtagctagtca | |
| | atccatcattgcctacactatgtcacttggtgcagagaattcagttgcttactctaata | |
| | actctattgccatacccacaaactttactattagtgttaccacagaaattctaccagt | |
| | gtctatgaccaagacatcagtagattgtacaatgtacatttgtggtgattcaactgaa | |
| | tgcagcaatctattgttgcaatatggcagtttctgtacacaattaaaccgtgctttaac | |
| | tggaatagctgttgaacaagacaagaacacccaagaagtatttgcacaagtcaa | |
| | acaaatttacaagacaccaccaattaaagatttcggtggatttaatttctcacaaat | |
| | attaccagatccatctaaaccaagcaagaggtcatttattgaagatctactattcaa | |
| | caaagtgacacttgcagatgctggcttcatcaaacaatatggtgattgccttggtga | |
| | tattgctgctagagacctcatttgtgcacagaagtttaacggccttactgtattgccac | |
| | ctttgctcacagatgaaatgattgctcaatacacttctgcactgttagcgggtacaat | |
| | cacttctggttggacctttggtgcaggtgctgcattacaaataccatttgctatgcaaa | |
| | tggcttataggtttaatggtattggagttacacagaatgttctctatgagaaccagaa | |
| | attgattgccaaccaatttaatagtgctattggcaagattcaagactcactttcttcca | |
| | cagcaagtgcacttggtaaacttcaagatgtggtcaaccagaatgcacaagcttt | |
| | aaacacgcttgttaaacaacttagctccaactttggtgcaatttcaagtgtattaaat | |
| | gatatcctttcacgtcttgacaaagttgaggctgaagtgcaaattgataggttgatca | |
| | caggcagacttcaaagtttgcagacatatgtgactcaacaattaattagagctgca | |
| | gaaatcagagcttctgctaatcttgctgctattaagatgtcagagtgtgtacttggac | |
| | aatctaagagagttgatttctgtggaaagggctatcatcttatgtccttccctcagtca | |
| | gcacctcatggtgtagtcttcttgcatgtgacttatgtccctgcacaagagaagaact | |
| | tcacaactgctcctgccatttgtcatgatggtaaagcacactttcctcgtgaaggtgt | |
| | ctttgtttcaaatggcacacactggtttgtaacacaaaggaatttctatgaaccaca | |
| | aatcattactacagacaacacatttgtgtctggtaactgtgatgttgtaataggaattg | |
| | tcaacaacacagtttatgatcctttgcaacctgaattagactcattcaaggaggagt | |
| | tagataaatatttcaagaatcatacatcaccagatgttgatttaggtgacatctctgg | |
| | cattaatgcttcatttgtaaacattcagaaagaaattgaccgcctcaatgaggttgc | |
| | caagaatttaaatgaatctctcatcgatctccaagaacttggaaagtatgagcagt | |
| | atatcaaatggccatggtacatttggctaggtttcatagctggcttgattgccatagta | |
| | atggtgacaattatgctttgctgtatgaccagttgctgtagttgtctcaagggctgttgtt | |
| | cttgtggatcctgctgcaaatttgatgaagacgactctgagccagtgctcaaagga | |
| | gtcaaattacattacacataa | |
| 30 | MFVFLVLLPLVSSQCVNFTNRTQLPSAYTNSFTRGVYYPD | Amino acid |
| | KVFRSSVLHSTQDLFLPFFSNVTWFHAIHVSGTNGTKRFD | sequence (N- to |
| | NPVLPFNDGVYFASTEKSNIIRGWIFGTTLDSKTQSLLIVNN | C-terminus) of a |
| | ATNVVIKVCEFQFCNYPFLGVYYHKNNKSWMESEFRVYS | codon-optimized |
| | SANNCTFEYVSQPFLMDLEGKQGNFKNLSEFVFKNIDGYF | SARS-CoV-2 S |
| | KIYSKHTPINLVRDLPQGFSALEPLVDLPIGINITRFQTLLAL | antigen |
| | HRSYLTPGDSSSGWTAGAAAYYVGYLQPRTFLLKYNENG | sequence based |
| | TITDAVDCALDPLSETKCTLKSFTVEKGIYQTSNFRVQPTE | on the Wuhan- |
| | SIVRFPNITNLCPFGEVFNATRFASVYAWNRKRISNCVADY | Hu-1 reference |
| | SVLYNSASFSTFKCYGVSPTKLNDLCFTNVYADSFVIRGD | strain |
| | EVRQIAPGQTGTIADYNYKLPDDFTGCVIAWNSNNLDSKV | (#NC_045512) |
| | GGNYNYLYRLFRKSNLKPFERDISTEIYQAGSTPCNGVKG | and optimized |
| | FNCYFPLQSYGFQPTYGVGYQPYRVVVLSFELLHAPATVC | for vaccinia, |
| | GPKKSTNLVKNKCVNFNFNGLTGTGVLTESNKKFLPFQQF | which is further |
| | GRDIADTTDAVRDPQTLEILDITPCSFGGVSVITPGTNTSN | modified to |
| | QVAVLYQGVNCTEVPVAIHADQLTPTWRVYSTGSNVFQT | encode for an S |
| | RAGCLIGAEYVNNSYECDIPIGAGICASYQTQTNSPRRARS | antigen that |
| | VASQSIIAYTMSLGAENSVAYSNNSIAIPTNFTISVTTEILPV | includes |
| | SMTKTSVDCTMYICGDSTECSNLLLQYGSFCTQLNRALTG | mutations of the |
| | IAVEQDKNTQEVFAQVKQIYKTPPIKDFGGFNFSQILPDPS | P.1 variant |
| | KPSKRSFIEDLLFNKVTLADAGFIKQYGDCLGDIAARDLICA | lineage, |
| | QKFNGLTVLPPLLTDEMIAQYTSALLAGTITSGWTFGAGAA | including L18F, |
| | LQIPFAMQMAYRFNGIGVTQNVLYENQKLIANQFNSAIGKI | T20N, P26S, |
| | QDSLSSTASALGKLQDVVNQNAQALNTLVKQLSSNFGAIS | D138Y, R190S, |
| | SVLNDILSRLDKVEAEVQIDRLITGRLQSLQTYVTQQLIRAA | K417T, E484K, |
| | EIRASANLAAIKMSECVLGQSKRVDFCGKGYHLMSFPQSA | N501Y, D614G, |
| | PHGVVFLHVTYVPAQEKNFTTAPAICHDGKAHFPREGVFV | H655Y, T1027I, |
| | SNGTHWFVTQRNFYEPQIITTDNTFVSGNCDVVIGIVNNTV | and V1167F |
| | YDPLQPELDSFKEELDKYFKNHTSPDVDLGDISGINASFVN | |
| | IQKEIDRLNEVAKNLNESLIDLQELGKYEQYIKWPWYIWLG | |
| | FIAGLIAIVMVTIMLCCMTSCCSCLKGCCSCGSCCKFDEDD | |
| | SEPVLKGVKLHYT | |
| 31 | atgtctgataacggaccacagaatcagcgaaacgcaccacgcattacgtttggtg | Nucleotide |
| | gaccctcagattcaactggcagtaaccagaatggagaacgcagtggagcgcga | sequence/ORF |
| | tctaaacaacgtcggcctcaaggtttacccaataatactgcgtcttggttcaccgctc | (5′ to 3′ end) of |
| | tcactcaacatggcaaggaagaccttaaattccctcgaggacaaggcgttccaat | a codon- |
| | taacaccaatagcagtcgagatgaccaaattggctactaccgaagagctaccag | optimized |
| | acgaattcgtggtggtgacggtaagatgaaagatctcagtccaagatggtatttcta | SARS-CoV-2 N |
| | ctacctaggaactgggccagaagctggacttccctatggtgctaacaaagacgg | antigen |
| | catcatatgggttgcaactgagggagccttgaatacacctaaagatcacattggc | sequence based |
| | acccgcaatcctgctaacaatgctgcaatcgtgctacaacttcctcaaggaacaa | on the Wuhan- |
| | cattgcctaaaggcttctacgcagaagggagcagaggcggcagtcaagcctctt | Hu-1 reference |
| | ctcgttcctcatcacgtagtcgcaacagttcaagaaattcaactccaggcagcagt | strain |
| | aaacgaacttctcctgctagaatggctggcaatggcggtgatgctgctcttgctttgc | (#NC_045512) |
| | tgctgcttgacagattgaaccagcttgagagcaagatgtctggtaaaggccaaca | and optimized |
| | acaacaaggccaaactgtcactaagaaatctgctgctgaggcttctaagaagcct | for vaccinia, |
| | cggcagaaacgtactgccactaaagcatacaatgtaacacaagctttcggcaga | which is further |
| | cgtggtccagaacaaacccaaggaaactttggagaccaggaactaatcagaca | modified to |
| | aggaactgattacaaacattggccgcaaattgcacaatttgctcccagcgcttcag | encode for an N |
| | cgttcttcggaatgtcgcgcattggcatggaagtcacaccttcgggaacgtggttga | antigen that |
| | cctacacaggtgccatcaaattggatgacaaagatccaaatttcaaagatcaagt | includes |
| | catattgctgaataagcatattgacgcatacaagacattcccaccaacagagcct | mutations of the |
| | aagaaggacaagaagaagaaggctgatgaaactcaagccttaccgcagaga | P.1 variant |
| | cagaagaaacagcaaactgtgactcttcttcctgctgcagatttggatgatttctcca | lineage, |
| | aacaattgcaacaatccatgagcagtgctgactcaactcaggcctaa | including P80R, |
| | | R203K, and |
| | | G204K |
| 32 | MSDNGPQNQRNAPRITFGGPSDSTGSNQNGERSGARSK | Amino acid |
| | QRRPQGLPNNTASWFTALTQHGKEDLKFPRGQGVPINTN | sequence (N- to |
| | SSRDDQIGYYRRATRRIRGGDGKMKDLSPRWYFYYLGTG | C-terminus) of a |
| | PEAGLPYGANKDGIIWVATEGALNTPKDHIGTRNPANNAAI | codon-optimized |
| | VLQLPQGTTLPKGFYAEGSRGGSQASSRSSSRSRNSSRN | SARS-CoV-2 N |
| | STPGSSKRTSPARMAGNGGDAALALLLLDRLNQLESKMS | antigen |
| | GKGQQQQGQTVTKKSAAEASKKPRQKRTATKAYNVTQA | sequence based |
| | FGRRGPEQTQGNFGDQELIRQGTDYKHWPQIAQFAPSAS | on the Wuhan- |
| | AFFGMSRIGMEVTPSGTWLTYTGAIKLDDKDPNFKDQVILL | Hu-1 reference |
| | NKHIDAYKTFPPTEPKKDKKKKADETQALPQRQKKQQTVT | strain |
| | LLPAADLDDFSKQLQQSMSSADSTQA | (#NC_045512) |
| | | and optimized |
| | | for vaccinia, |
| | | which is further |
| | | modified to |
| | | encode for an N |
| | | antigen that |
| | | includes |
| | | mutations of the |
| | | P.1 variant |
| | | lineage, |
| | | including P80R, |
| | | R203K, and |
| | | G204K |

In some embodiments, the DNA sequence encoding the S protein is based on the Wuhan-Hu-1 reference strain or is derived from a variant of concern (VOC). In some embodiments, the DNA sequence encoding the N protein is based on the Wuhan-Hu-1 reference strain or is derived from a VOC. In some embodiments, the VOC is selected from the group consisting of B.1.1.7 (Alpha), B.1.351 (Beta), P.1 (Gamma), B.1.617.2 (Delta), B.1.621 (Mu), 0.37 (Lambda), C.1.2, BA.1 (Omicron), BA.2 (Omicron), BA.2.12.1 (Omicron), BA.4 (Omicron), BA.5 (Omicron), BA.2.75 (Omicron), BQ.1 (Omicron), BQ.1.1 (Omicron), and XBB (Omicron).

In some embodiments, the DNA sequence encoding the S protein comprises a nucleotide sequence that is at least 80% identical to any one of SEQ ID NOs: 5, 9, 11, 13, 15, 21, 25, and 29. In some embodiments, the DNA sequence encoding the N protein comprises a nucleotide sequence that is at least 80% identical to any one of SEQ ID NOs: 7, 17, 19, 23, 27, and 31.

In some embodiments, the DNA sequences encode an S protein and an N protein based on the ancestral Wuhan-Hu-1 reference strain. In certain of these embodiments, the DNA sequences comprise or consist of nucleotide sequences set forth in SEQ ID NOs: 5 and 7, respectively, or nucleotide sequences that are at least about 80% identical (e.g., at least about 80%, at least about 85%, at least about 90%, at least about 95%, at least about 96%, at least about 97%, at least about 98%, at least about 99%, or about 100% identical) to the nucleotide sequences set forth in SEQ ID NOs: 5 and 7, respectively. The corresponding S protein and N protein encoded by the]DNA sequences comprise or consist of amino acid sequences set forth in SEQ ID NOs: 6 and 8, respectively, or amino acid sequences that are at least about 80% identical (e.g., at least about 80%, at least about 85%, at least about 90%, at least about 95%, at least about 96%, at least about 97%, at least about 98%, at least about 99%, or about 100% identical) to the amino acid sequences set forth in SEQ ID NOs: 6 and 8, respectively.

In some embodiments, the DNA sequences encode an S protein and an N protein based on the B.1.351 (Beta) strain. In certain of these embodiments, the DNA sequences comprise or consist of nucleotide sequences set forth in SEQ ID NOs: 21 and 23, respectively, or nucleotide sequences that are at least about 80% identical (e.g., at least about 80%, at least about 85%, at least about 90%, at least about 95%, at least about 96%, at least about 97%, at least about 98%, at least about 99%, or about 100% identical) to the nucleotide sequences set forth in SEQ ID NOs: 21 and 23, respectively. The corresponding S protein and N protein encoded by the DNA sequences comprise or consist of amino acid sequences set forth in SEQ ID NOs: 22 and 24, respectively, or amino acid sequences that are at least about 80% identical (e.g., at least about 80%, at least about 85%, at least about 90%, at least about 95%, at least about 96%, at least about 97%, at least about 98%, at least about 99%, or about 100% identical) to the amino acid sequences set forth in SEQ ID NOs: 22 and 24, respectively.

In some embodiments, the DNA sequences encode an S protein and an N protein based on the P.1 (Gamma) strain. In certain of these embodiments, the DNA sequences comprise or consist of nucleotide sequences set forth in SEQ ID NOs: 29 and 3′, respectively, or nucleotide sequences that are at least about 80% identical (e.g., at least about 80%, at least about 85%, at least about 90%, at least about 95%, at least about 96%, at least about 97%, at least about 98%, at least about 99%, or about 100% identical) to the nucleotide sequences set forth in SEQ ID NOs: 29 and 31, respectively. The corresponding S protein and N protein encoded by the DNA sequences comprise or consist of amino acid sequences set forth in SEQ ID NOs: 30 and 32, respectively, or amino acid sequences that are at least about 80% identical (e.g., at least about 80%, at least about 85%, at least about 90%, at least about 95%, at least about 96%, at least about 97%, at least about 98%, at least about 99%, or about 100% identical) to the amino acid sequences set forth in SEQ ID NOs: 30 and 32, respectively.

In some embodiments, the DNA sequences encode an S protein and an N protein based on the B.1.617.2 (Delta) strain. In certain of these embodiments, the DNA sequences comprise or consist of nucleotide sequences set forth in SEQ ID NOs: 9 and 17, respectively, or nucleotide sequences that are at least about 80% identical (e.g., at least about 80%, at least about 85%, at least about 90%, at least about 95%, at least about 96%, at least about 97%, at least about 98%, at least about 99%, or about 100% identical) to the nucleotide sequences set forth in SEQ ID NOs: 9 and 17, respectively. The corresponding S protein and N protein encoded by the DNA sequences comprise or consist of amino acid sequences set forth in SEQ ID NOs: 10 and 18, respectively, or amino acid sequences that are at least about 80% identical (e.g., at least about 80%, at least about 85%, at least about 90%, at least about 95%, at least about 96%, at least about 97%, at least about 98%, at least about 99%, or about 100% identical) to the amino acid sequences set forth in SEQ ID NOs: 10 and 18, respectively.

In some embodiments, the DNA sequences encode an S protein and an N protein based on the B.1.617.2 (Delta) strain. In certain of these embodiments, the DNA sequences comprise or consist of nucleotide sequences set forth in SEQ ID NOs: 11 and 17, respectively, or nucleotide sequences that are at least about 80% identical (e.g., at least about 80%, at least about 85%, at least about 90%, at least about 95%, at least about 96%, at least about 97%, at least about 98%, at least about 99%, or about 100% identical) to the nucleotide sequences set forth in SEQ ID NOs: 11 and 17, respectively. The corresponding S protein and N protein encoded by the hDNA sequences comprise or consist of amino acid sequences set forth in SEQ ID NOs: 12 and 18, respectively, or amino acid sequences that are at least about 80% identical (e.g., at least about 80%, at least about 85%, at least about 90%, at least about 95%, at least about 96%, at least about 97%, at least about 98%, at least about 99%, or about 100% identical) to the amino acid sequences set forth in SEQ ID NOs: 12 and 18, respectively.

In some embodiments, the DNA sequences encode an S protein and an N protein based on the C.1.2 strain. In certain of these embodiments, the DNA sequences comprise or consist of nucleotide sequences set forth in SEQ ID NOs: 15 and 19, respectively, or nucleotide sequences that are at least about 80% identical (e.g., at least about 80%, at least about 85%, at least about 90%, at least about 95%, at least about 96%, at least about 97%, at least about 98%, at least about 99%, or about 100% identical) to the nucleotide sequences set forth in SEQ ID NOs: 15 and 19, respectively. The corresponding S protein and N protein encoded by the DNA sequences comprise or consist of amino acid sequences set forth in SEQ ID NOs: 16 and 20, respectively, or amino acid sequences that are at least about 80% identical (e.g., at least about 80%, at least about 85%, at least about 90%, at least about 95%, at least about 96%, at least about 97%, at least about 98%, at least about 99%, or about 100% identical) to the amino acid sequences set forth in SEQ ID NOs: 16 and 20, respectively.

In some embodiments, the DNA sequences encode an S protein and an N protein based on the B.1.1.529/BA.1 (Omicron) strain. In certain of these embodiments, the DNA sequences comprise or consist of nucleotide sequences set forth in SEQ ID NOs: 25 and 27, respectively, or nucleotide sequences that are at least about 80% identical (e.g., at least about 80%, at least about 85%, at least about 90%, at least about 95%, at least about 96%, at least about 97%, at least about 98%, at least about 99%, or about 100% identical) to the nucleotide sequences set forth in SEQ ID NOs: 25 and 27, respectively. The corresponding S protein and N protein encoded by the DNA sequences comprise or consist of amino acid sequences set forth in SEQ ID NOs: 26 and 28, respectively, or amino acid sequences that are at least about 80% identical (e.g., at least about 80%, at least about 85%, at least about 90%, at least about 95%, at least about 96%, at least about 97%, at least about 98%, at least about 99%, or about 100% identical) to the amino acid sequences set forth in SEQ ID NOs: 26 and 28, respectively.

In some embodiments, the SARS-CoV-2 comprises the Wuhan-Hu-1 reference strain or a VOC. In some embodiments, the VOC is selected from the group consisting of B.1.1.7 (Alpha), B.1.351 (Beta), P.1 (Gamma), B.1.617.2 (Delta), B.1.621 (Mu), C.37 (Lambda), C.1.2, BA.1 (Omicron), BA.2 (Omicron), BA.2.12.1 (Omicron), BA.4 (Omicron), BA.5 (Omicron), BA.2.75 (Omicron), BQ.1 (Omicron), BQ.1.1 (Omicron), and XBB (Omicron).

In some embodiments, the vaccines and/or immunogenic compositions comprise one or more sMVA vectors, wherein the one or more sMVA vectors comprise one or more heterologous DNA sequences encoding the S protein and/or N protein of the SARS-CoV-2 Wuhan-Hu-1 reference stain. In some embodiments, the one or more sMVA vectors comprise one or more heterologous DNA sequences encoding a mutant S protein and/or N protein such as mutant S protein and/or N protein based on a variant of concern (VOC), including B.1.1.7 (Alpha), B.1.351 (Beta), P.1 (Gamma), B.1.617.2 (Delta), B.1.621 (Mu), C.37 (Lambda), C.1.2, BA.1 (Omicron), BA.2 (Omicron), BA.2.12.1 (Omicron), BA.4 (Omicron), BA.5 (Omicron), BA.2.75 (Omicron), BQ.1 (Omicron), BQ.1.1 (Omicron), and XBB (Omicron).

In some embodiments, the vaccines and/or immunogenic compositions are used for preventing or treating a coronavirus infection, for example, SARS-CoV-2 infection, caused by the ancestral Wuhan-Hu-1 reference strain or a VOC, including, but not limited to, B.1.1.7 (Alpha), B.1.351 (Beta), P.1 (Gamma), B.1.617.2 (Delta), B.1.621 (Mu), C.37 (Lambda), C.1.2, BA.1 (Omicron), BA.2 (Omicron), BA.2.12.1 (Omicron), BA.4 (Omicron), BA.5 (Omicron), BA.2.75 (Omicron), BQ.1 (Omicron), BQ.1.1 (Omicron), and XBB (Omicron).

In some embodiments, the encoded mutant S protein based on the B.1.617.2 lineage comprises one or more of the following mutations: T19R, E156G, Del157/158, S255F, L452R, T478K, D614G, P681R, D950N, G142D, Del156/157, R158G, A222, L5F, R21T, T51I, H66Y, K77T, D80Y, T95I, G181V, R214H, P251L, D253A, V289I, V308L, A411S, G446V, T547I, A570S, T572I, Q613H, S640F, E661D, Q675H, T719I, P809S, A845S, I850L, A879S, D979E, A1078S, H1101Y, D1127G, L1141W, G1167V, K1191N, G1291V, and V1264L. Other mutations such as K417T may also be included.

In some embodiments, the encoded mutant S protein based on VOC lineage B.1.351 (Beta) comprises one or more of the following mutations: L18F, D80A, D215G, Del242-244, R246I, N501Y, E484K, K417N, D614G, and A701V.

In some embodiments, the encoded mutant S protein based on VOC lineage P.1 (Gamma) comprises one or more of the following mutations: L18F, T20N, P26S, D138Y, R190S, K417T, E484K, N501Y, D614G, H655Y, T10271, and V1167F.

In some embodiments, the encoded mutant S protein based on VOC lineage B.1.617.2 (Delta) comprises one or more of the following mutations: T19R, G142D, Del156-157, R158G, L452R, T478K, D614G, P681R, and D950N.

In some embodiments, the encoded mutant S protein based on VOC lineage B.1.617.2 (Delta) comprises one or more of the following mutations: T19R, K77T, Del157-158, L452R, T478K, D614G, P681R, and D950N.

In some embodiments, the encoded mutant S protein based on VOC lineage B.1.617.2 (Delta) comprises one or more of the following mutations: T19R, E156G, Del157-158, S255F, L452R, T478K, D614G, P681R, and D950N.

In some embodiments, the encoded mutant S protein based on VOC lineage B.1.617.2 (Delta) comprises one or more of the following mutations: T19R, L452R, T478K, D614G, P681R, and D950N and additionally one or more mutations comprising G142D, R158G, A222V, S255F, E156G, T95I, K77T or deletions Del156-157 or Del157-158.

In some embodiments, the encoded mutant S protein based on VOC lineage B.1.1.529/BA.1 (Omicron) comprises one or more of the following mutations: A67V, Del69-70 (HV), T95I, G142D, Del143-145 (VYY), Del211 (N), L2121, Ins214 (EPE), G339D, S371 L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F.

In some embodiments, the encoded mutant S protein based on VOC lineage BQ.1 (Omicron) comprises one or more of the following mutations: T191, Del24-26 (LPS), A27S, Del69-70 (HV), G142D, V213G, G339D, S371F, S373P, S375F, T376A, D405N, R408S, K417N, N440K, K444T, L452R, N460K, S477N, T478K, E484A, F486V, Q493Q, Q498R, N501Y, Y505H, D614G, H655Y, N679K, P681H, N764K, D796Y, Q954H, and N969K.

In some embodiments, the encoded mutant S protein based on VOC lineage BQ.1.1 (Omicron) comprises one or more of the following mutations: T191, Del24-26 (LPS), A27S, Del69-70 (HV), G142D, V213G, G339D, R346T, S371F, S373P, S375F, T376A, D405N, R408S, K417N, N440K, K444T, L452R, N460K, S477N, T478K, E484A, F486V, Q493Q, Q498R, N501Y, Y505H, D614G, H655Y, N679K, P681H, N764K, D796Y, Q954H, and N969K.

In some embodiments, the encoded mutant S protein based on VOC lineage XBB (Omicron) comprises one or more of the following mutations: T191, Del24-26 (LPS), A27S, V83A, G142D, Del144 (Y), H146Q, Q183E, V213E, G339H, R346T, L368I, S371F, S373P, S375F, T376A, D405N, R408S, K417N, N440K, V445P, G446S, N460K, S477N, T478K, E484A, F486S, F490S, R493Q, Q498R, N501Y, Y505H, D614G, H655Y, N679K, P681H, N764K, D796Y, Q954H, and N969K.

In some embodiments, the encoded mutant N protein based on VOC lineage B.1.351 (Beta) comprises a T2051 mutation.

In some embodiments, the encoded mutant N protein based on VOC lineage P.1 (Gamma) comprises one or more of the following mutations: P80R, R203K, and G204R.

In some embodiments, the encoded mutant N protein based on VOC lineage P.1 (Gamma) comprises one or more of the following mutations: P80R, R203K, and G204K.

In some embodiments, the encoded mutant N protein based on VOC lineage B.1.617.2 (Delta) comprises one or more of the following mutations: D63G, R203M, G215C, and D377Y.

In some embodiments, the encoded mutant N protein based on VOC lineage B.1.617.2 (Delta) comprises one or more of the following mutations: D63G, R203M, and D377Y.

In some embodiments, the encoded mutant N protein based on VOC lineage B.1.617.2 (Delta) comprises one or more of the following mutations: D63G, R203M, D377Y, and R385K.

In some embodiments, the encoded mutant N protein based on VOC lineage B.1.1.529/BA.1 (Omicron) comprises one or more of the following mutations: P13L, Del31-33 (ERS), R203K, G204R.

In some embodiments, the encoded mutant N protein based on VOC lineage BQ.1 (Omicron) comprises one or more of the following mutations: P13L, Del31-33 (ERS), E136D, R203K, G204R, and S413R.

In some embodiments, the encoded mutant N protein based on VOC lineage BQ.1.1 (Omicron) comprises one or more of the following mutations: P13L, Del31-33 (ERS), E136D, R203K, G204R, and S413R.

In some embodiments, the encoded mutant N protein based on VOC lineage XBB (Omicron) comprises one or more of the following mutations: P13L, Del31-33 (ERS), R203K, G204R, and S413R.

In some embodiments, the recombinant sMVA vector used in the methods and compositions disclosed herein is used in a candidate vaccine composition referred to herein as sMVA-N/S (or COH04S1). COH04S1 is based on a recombinant sMVA vector capable of expressing S and N antigens of SARS-CoV-2. MVA vectors have a robust safety record and are known for inducing humoral and cellular immune responses that provide long-term protection against several infectious diseases, including smallpox and cytomegalovirus. In a mouse model, robust immunogenicity of COH04S1 was demonstrated, and pre-clinical data in hamsters and non-human primates demonstrating protection from upper and lower respiratory tract infections following SARS-CoV-2 challenge.

A fully synthetic modified vaccinia Ankara (sMVA)-based vaccine platform is used to develop COH04S1, a multi-antigenic poxvirus-vectored SARS-CoV-2 vaccine that co-expresses full-length spike (S) and nucleocapsid (N) antigens. SEQ ID NO: 33 shows the sequence of COH04S1. The DNA and protein sequences for the S antigen of COH04S1 are represented by SEQ ID NOs: 5 and 6 the Sequence Listing, and the DNA and protein sequences for the N antigen of COH04S1 are represented by SEQ ID NOs: 7 and 8 in the Sequence Listing. Additional constructs (e.g., sMVA-S/N, sMVA-S, sMVA-N) are also disclosed in International Application Publication No. WO 2021/236550, which is hereby incorporated by reference as if fully disclosed herein. In some embodiments, the sMVA-based vaccine comprises or consists of a nucleotide sequence set forth in SEQ ID NO: 33, or a nucleotide sequence that is at least about 80% identical (e.g., at least about 80%, at least about 85%, at least about 90%, at least about 95%, at least about 96%, at least about 97%, at least about 98%, at least about 99%, or about 100% identical) to the nucleotide sequence set forth in SEQ ID NO: 33.

As demonstrated in the working examples, COH04S1 was well-tolerated and induced S and N antigen-specific antibody and T-cell responses. No severe adverse events were reported following vaccination of healthy adults with COH04S1. Humoral and cellular responses against SARS-CoV-2 were measured after the first dose of the vaccine and seroconversion was achieved in 100% of the subjects after two doses. The pre-specified immunogenicity response (S or N IgG>4-fold increase within 56 days) was observed in 34/34 (100%) participants for S, and 32/34 (94.1%) for N protein (p<0.001 vs placebo (0/5)). Four-fold or more increase in SARS-CoV-2 neutralizing antibodies within 56 days was measured in 9/17 DL1, 8/8 DL2 and 8/9 DL3 (p<0.005 vs placebo (0/5)). Th1/Th2 (IFN-γ/IL-4) cellular responses increased with a median of 173% (IQR 53-307%) and median of 211% (IQR 80-358%) in combined DL1-3 for S and N respectively (p<0.001).

In this first-in-human Phase 1 trial with randomized expansion cohorts, vaccination with COH04S1 elicited robust and durable humoral and cellular immunity to both S and N vaccine antigens with no indication of objective safety concerns. An important immunological outcome was the predominance of Th1-biased T cell responses which indicate low risk of vaccine-associated enhanced respiratory disease (VAERD). Similarly, a Th1-biased immune response pre- and post-SARS-CoV-2 challenge in non-human primates vaccinated with COH04S18 was observed, demonstrating that COH04S1-induced immune-responses would not likely be the cause of inflammation even after viral challenge. Of note, COH04S1 functioned as expected without adverse events noted when administered before or after other mRNA- or adenovirus-based COVID-19 vaccines.

The historically unprecedented rapid deployment of SARS-CoV-2 vaccines has resulted in a drastic reduction of SARS-CoV-2 infections and SARS-CoV-2-related hospitalizations and deaths in the vaccinated population. Simultaneous with the worldwide spread of the pandemic virus, mutations have arisen that changed the transmissibility and immune control of the virus. Those that are particularly successful at evading immunity with great transmissibility are known as VOC Alpha, Beta, Gamma, and Delta with additional VOC likely to arise. The emergence of VOC was responsible for the lower-than-expected protective efficacy results observed in phase 3 clinical trials worldwide. This is the result of mutations of the S antigen in its receptor binding and N-terminal domains that confer resistance to NAb. Alternatively, it has been found that T cell epitopes remain intact despite variation in B cell epitopes which makes vaccines that elicit strong T cell immunity particularly valuable to sustain protection in the face of the decline of humoral immunity in the wake of mutations causing virus escape from neutralization. Additionally, T cell responses to SARS-CoV-2 can be present in convalescent individuals even in the absence of detectable antibody responses and contribute to survival in patients with COVID-19 and hematologic malignancies. Therefore, the inclusion of additional T cell immunodominant antigens beside S in next generation COVID-19 vaccines is seen as a strategy to widen the induction of pan-variant cellular responses that are less prone to viral escape selection given the intrinsic polymorphism of HLA molecules. N is a strong candidate for inclusion in a multi-antigenic COVID-19 vaccine given its abundant release during SARS-CoV-2 replication cycle and the presence of conserved T cell epitopes. In addition, recent reports have shown in rodents that nucleocapsid-based vaccines can mediate S-independent protective immunity.

As demonstrated herein, N was responsible for the induction of robust T cell responses of similar magnitude and phenotype as S-specific T cell responses thus supporting the inclusion of N in a vaccine formulation aimed at a broad induction of cellular responses. Additionally, considering that both S- and N-specific cellular responses reached maximum levels already after the first dose, COH04S1 can be used to generate N-specific cellular responses even in the context of a booster immunization to a prior S-only vaccine.

Differently from T cell responses but aligned with what has been shown with most SARS-CoV-2 vaccines based on platforms other than sMVA, maximal induction of NAb was achieved after two doses. Despite vaccine-induced bAb and cellular responses were comparable amongst DL, a dose effect was observed for NAb. However, in subjects immunized with DL1, a delay of one or two months in the administration of a second dose significantly increased peak NAb titers post-boost in comparison to subjects vaccinated following the 28-day interval schedule. Given that COH04S1 was equally well tolerated at all DL, COH04S1 can be safely used to induce durable SARS-CoV-2 specific humoral and cellular responses even when used at a DL that would allow easy scalability to mass production (DL1).

Disclosed herein is a method of vaccinating or protecting a subject against coronavirus disease 2019 (COVID-19) caused by a coronavirus infection comprising administering a first composition comprising a synthetic MVA vector or virus capable of expressing one or more DNA sequences encoding the spike (S) protein and the nucleocapsid (N) protein or variants or mutants thereof, wherein the method (i) prevents the coronavirus infection or (ii) prevents or reduces the severity of COVID-19 caused by the coronavirus infection.

In some embodiments of the method, the coronavirus infection is caused by a variant of concern including B.1.1.7 (Alpha), B.1.351 (Beta), P.1 (Gamma), B.1.617.2 (Delta), B.1.621 (Mu), C.37 (Lambda), C.1.2, BA.1 (Omicron), BA.2 (Omicron), BA.2.12.1 (Omicron), BA.4 (Omicron), BA.5 (Omicron), BA.2.75 (Omicron), BQ.1 (Omicron), BQ.1.1 (Omicron), and XBB (Omicron).

In some embodiments of the method, the composition is administered to the subject by intramuscular injection, intranasal instillation, intradermal injection, and/or scarification.

In some embodiments of the method, the composition is administered to the subject in a single dose, two doses, three doses, or more than three doses.

In some embodiments of the method, the first composition is a prime dose and further comprising administering a first booster dose subsequent to administration of the prime dose.

In some embodiments of the method, the interval between the prime dose and the first booster dose is about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, or more than 16 weeks.

In some embodiments of the method, the method further comprises administering a one or more additional booster doses subsequent to administration of the first booster dose.

In some embodiments of the method, the interval between each of the booster doses is about 8 weeks, about 9 weeks, about 10 weeks, about 11 weeks, about 12 weeks, about 13 weeks, about 14 weeks, about 15 weeks, about 16 weeks, about 6 months, or about 1 year.

In some embodiments of the method, the prime dose is between 1.0×10⁷ PFU/dose and 5.0×10⁸ PFU/dose, for example, about 1.0×10⁷ PFU/dose, about 1.5×10⁷ PFU/dose, about 2.0×10⁷ PFU/dose, about 2.5×10⁷ PFU/dose, about 3.0×10⁷ PFU/dose, about 3.5×10⁷ PFU/dose, about 4.0×10⁷ PFU/dose, about 4.5×10⁷ PFU/dose, about 5.0×10⁷ PFU/dose, about 5.5×10⁷ PFU/dose, about 6.0×10⁷ PFU/dose, about 6.5×10⁷ PFU/dose, about 7.0×10⁷ PFU/dose, about 7.5×10⁷ PFU/dose, about 8.0×10⁷ PFU/dose, about 8.5×10⁷ PFU/dose, about 9.0×10⁷ PFU/dose, about 9.5×10⁷ PFU/dose, about 1.0×10⁸ PFU/dose, about 1.5×10⁸ PFU/dose, about 2.0×10⁸ PFU/dose, about 2.5×10⁸ PFU/dose, about 3.0×10⁸ PFU/dose, about 3.5×10⁸ PFU/dose, about 4.0×10⁸ PFU/dose, about 4.5×10⁸ PFU/dose, or about 5.0×10⁸ PFU/dose.

In some embodiments of the method, the first or additional booster doses are between 1.0×10⁷ PFU/dose and 5.0×10⁸ PFU/dose, about 1.0×10⁷ PFU/dose, about 1.5×10⁷ PFU/dose, about 2.0×10⁷ PFU/dose, about 2.5×10⁷ PFU/dose, about 3.0×10⁷ PFU/dose, about 3.5×10⁷ PFU/dose, about 4.0×10⁷ PFU/dose, about 4.5×10⁷ PFU/dose, about 5.0×10⁷ PFU/dose, about 5.5×10⁷ PFU/dose, about 6.0×10⁷ PFU/dose, about 6.5×10⁷ PFU/dose, about 7.0×10⁷ PFU/dose, about 7.5×10⁷ PFU/dose, about 8.0×10⁷ PFU/dose, about 8.5×10⁷ PFU/dose, about 9.0×10⁷ PFU/dose, about 9.5×10⁷ PFU/dose, about 1.0×10⁸ PFU/dose, about 1.5×10⁸ PFU/dose, about 2.0×10⁸ PFU/dose, about 2.5×10⁸ PFU/dose, about 3.0×10⁸ PFU/dose, about 3.5×10⁸ PFU/dose, about 4.0×10⁸ PFU/dose, about 4.5×10⁸ PFU/dose, or about 5.0×10⁸ PFU/dose.

In some embodiments of the method, the first or second booster dose is at the same dosage as the prime dose or a lower dose than the prime dose.

In some embodiments of the method, the subject has previously received a different SARS-CoV-2 vaccine.

In some embodiments of the method, the previously received SARS-CoV-2 vaccine is an mRNA vaccine.

In some embodiments of the method, the previously received SARS-CoV-2 vaccine comprises an S antigen only.

In some embodiments of the method, a Th1-biased immune response is elicited in the subject.

Disclosed herein is a method of vaccinating or protecting a subject against coronavirus disease 2019 (COVID-19) caused by a coronavirus infection, or increasing an immune response in a subject who has previously received a SARS-CoV-2 vaccine, comprising administering to the subject a booster dose of a composition comprising a synthetic MVA vector comprising one or more DNA sequences encoding the Spike (S) protein and the Nucleocapsid (N) protein or variants or mutants of the S protein and N protein, wherein the method (i) prevents the coronavirus infection or (ii) prevents or reduces the severity of COVID-19 caused by the coronavirus infection.

In some embodiments of the method, the previously received SARS-CoV-2 vaccine is an mRNA-based SARS-CoV-2 vaccine composition.

In some embodiments of the method, the previously received SARS-CoV-2 vaccine is a SARS-CoV-2 vaccine composition targeting the S antigen only.

In some embodiments of the method, the subject received the previous SARS-CoV-2 vaccine at least one month, at least two months, at least 3 months, at least 4 months, at least 5 months, at least 6 months, at least 7 months, at least 8 months, at least 9 months, at least 10 months, at least 11 months, or at least 12 months before receiving the booster dose.

In some embodiments of the method, the coronavirus infection is caused by a variant of concern including B.1.1.7 (Alpha), B.1.351 (Beta), P.1 (Gamma), B.1.617.2 (Delta), B.1.621 (Mu), C.37 (Lambda), C.1.2, BA.1 (Omicron), BA.2 (Omicron), BA.2.12.1 (Omicron), BA.4 (Omicron), BA.5 (Omicron), BA.2.75 (Omicron), BQ.1 (Omicron), BQ.1.1 (Omicron), and XBB (Omicron).

In some embodiments of the method, the composition is administered to the subject by intramuscular injection, intranasal instillation, intradermal injection, and/or scarification.

In some embodiments of the method, the composition is administered to the subject in a single dose.

In some embodiments of the method, the single dose is between 1.0×10⁷ PFU/dose and 1.0×10⁸ PFU/dose, for example, about 1.0×10⁷ PFU/dose, about 1.5×10⁷ PFU/dose, about 2.0×10⁷ PFU/dose, about 2.5×10⁷ PFU/dose, about 3.0×10⁷ PFU/dose, about 3.5×10⁷ PFU/dose, about 4.0×10⁷ PFU/dose, about 4.5×10⁷ PFU/dose, about 5.0×10⁷ PFU/dose, about 5.5×10⁷ PFU/dose, about 6.0×10⁷ PFU/dose, about 6.5×10⁷ PFU/dose, about 7.0×10⁷ PFU/dose, about 7.5×10⁷ PFU/dose, about 8.0×10⁷ PFU/dose, about 8.5×10⁷ PFU/dose, about 9.0×10⁷ PFU/dose, about 9.5×10⁷ PFU/dose, or about 1.0×10⁸ PFU/dose.

The following examples are intended to illustrate various embodiments of the invention. As such, the specific embodiments discussed are not to be constructed as limitations on the scope of the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### EXAMPLES

### Example 1. Materials and Methods for Immunogenicity Studies in Healthy Adults

Materials and methods used in Examples 2-4 regarding the safety and immunogenicity resulting from COH04S1 vaccinations in healthy adults are described below.

Study Design and participants: A combined open-label (dose-level [DL]1-3) phase 1 study was conducted, followed by randomized expansion cohorts (NCT04639466) at the City of Hope Comprehensive Cancer Center (Duarte, CA, USA). The study met all ethics and regulatory requirements as determined by an external IRB review (Advarra IRB), and an independent external Data Monitoring Committee (DMC) which reviewed study plans and progress. The study is closed for accrual (separate amendments testing COH04S1 as a boost are pending). The primary safety data and immunogenicity analysis during the 56-day period post-vaccination are disclosed herein, along with additional follow-up to day 120 post-vaccination. Healthy participants 18 years and <55 years old were consented prior to screening for eligibility, which required absence of SARS-CoV-2 antibody (SCoV-2 Detect IgG ELISA, In-Bios) and negative nasopharyngeal wash by SARS-CoV-2 PCR (Simplexa for COVID-19, DiaSorin Molecular). In addition, all subjects had institutional normal CBC, clinical chemistry panel, normal electrocardiogram and troponin level, negative pregnancy test if female, body mass index s 38. Other exclusion criteria were based primarily on absence of COVID-19 risk factors as outlined by the Centers for Disease Control in a Jun. 25, 2020 guidance.

Randomization and masking: In this first-in-human trial, COH04S1 was first administered in an open label safety study to sentinel participants at DL1 (n=4), DL2 (n=7), and DL3 (n=6). Thereafter, a double-blind, randomized, placebo-controlled trial (RCT) evaluated DL1 given as one vaccine followed by placebo (DL1/placebo, n=14), two vaccine doses (DL1/DL1, n=14) or two placebo doses (placebo/placebo, n=5), with a separate randomization between DL2/DL3 (n=6). The study was intended to complete DL1 randomization of 15 DL1/placebo, 15 DL1/DL1, and 5 placebo/placebo, plus a separate randomization of 30 participants between DL2/DL2 vs DL3/DL3, but only the DL1 randomization could be completed once vaccines given emergency use authorization (EUA) become available to the general U.S. public. For the same reason, the blind was limited to 56 days post-vaccination at which point participants were informed of their vaccination status. Participants who received any placebo could pursue an EUA vaccine and only those who received one placebo could opt to receive a second COH04S1 dose. Vaccine and placebo were presented in identical unlabeled vials with serial numbers to ensure masking. RCT group allocation was masked from subjects and investigators.

Procedures: COH04S1 was generated using a synthetic clone of MVA with inserted S and N antigen sequences based on SARS-CoV-2 Wuhan strain as disclosed below. The vaccine was manufactured as a liquid formulation containing PBS with 7.5% lactose. Prior to each injection, COH04S1 was thawed and diluted with sterile diluent (PBS with 7.5% lactose) to the appropriate DL. Placebo consisted of PBS containing 7.5% lactose. Vaccine formulations and placebo in 1-0 mL volume were administered to the upper non-dominant arm by intramuscular injection on day 1 and day 28. An additional DL1 dose was administered at unblinding to DL1/placebo subjects who opted for a second dose of COH04S1.

Laboratory assessments included serum biochemistry tests, hematology, ECG and cardiac troponin test. For AE assessments, all adverse reactions (including those noted on scheduled appointments and calls, along with subject-reported AEs not per schedule) from the first dose to at least d120 for this report were collected. The investigators had two phone calls and face-to-face interviews within 7 days after each dose and at two weeks after each dose. Follow-up AE assessments were carried out on at least a monthly basis.

Blood samples were collected for immunological analyses at the time of vaccination, two weeks after each vaccination, and one, two, and three months after the second vaccination, with longer observation intervals subsequently. The same sample collection schedule applied to DL1/placebo individuals that were given another DL1 vaccination at day 56 resulting in additional samples and an extended timeline.

Serum S-, receptor binding domain (RBD)-, and N-specific IgG were measured using indirect ELISA and expressed as endpoint titers. Seroconversion was defined as a four-fold increase in S or N antibody endpoint titers relative to baseline. Serum neutralizing antibody (NAb) titers were measured using SARS-CoV-2 pseudovirus (PsV) based on the SARS-CoV-2 Wuhan S sequence with D614G substitution. Samples from subjects in the open label arm were also analyzed using PsV representing the Alpha, Beta, and Gamma variants of concern (VOC). The serum dilution that reduced PsV entry into susceptible cells by 50% was defined as 50% neutralization titer (NT50). Absolute numbers of S-, N-, and membrane (M)-specific cells secreting IFNγ and IL-4 were measured using ELISPOT and IFN-γ/IL-4 ratio used as a measure of Th1/Th2 polarization. Activated/cycling S- and N-specific T cells were longitudinally evaluated in open label subjects using CD137 multi-parameter flow cytometry assay.

sMVA vaccine stocks: COH04S1 is a double-plaque purified virus isolate derived from the sMVA-N/S vector (NCBI Accession #MW036243), with N and S antigen sequences inserted into the MVA deletion sites 2 and 3, respectively. It was generated using the three-plasmid system of the sMVA platform and fowlpox virus TROVAC as a helper virus for virus reconstitution. COH04S1 co-expresses full-length, unmodified S and N antigen sequences based on the Wuhan-Hu-1 reference strain (NCBI Accession #NC_045512). Sequence identity of COH04S1 seed stock virus was assessed by PacBio long-read sequencing. COH04S1 and sMVA vaccine stocks for animal studies were produced using chicken embryo fibroblasts (CEF) and prepared by 36% sucrose cushion ultracentrifugation and virus resuspension in 1 mM Tris-HCl (pH 9). Virus stocks were stored at −80° C. and titrated on CEF by plaque immunostaining. Viral stocks were validated for antigen insertion and expression by PCR, sequencing, and immunoblot.

COH04S351 is a double-plaque purified virus isolate with analogous vaccine construction compared to the original COH04S1 vector and co-expresses modified S and N antigen sequences based on the B.1.351 Beta variant. COH04S529 is a non-plaque purified virus isolate with analogous vaccine construction compared to the original COH04S1 sMVA-N/S vaccine vector and co-expresses modified S and N antigen sequences based on the Omicron BA.1 variant. COH04S1 and COH04S351 were generated using the sMVA platform. Virus stocks of the vaccine vectors and sMVA control vector were produced using chicken embryo fibroblasts (CEF) and prepared by 36% sucrose cushion ultracentrifugation and virus resuspension in 1 mM Tris-HCl (pH 9). Virus stocks were stored at −80° C. and titrated on CEF by plaque immunostaining as described. Viral stocks were validated for antigen insertion and expression by PCR, sequencing, and immunoblot.

COH04S1 generation: Three unique synthetic sub-genomic sMVA fragments were designed based on the MVA genome sequence published previously. The entire sMVA was cloned as three fragments in Escherichia coli as bacterial artificial chromosome (BAC) clones using highly efficient BAC recombination techniques. The full-length SARS-CoV-2 S and N antigen sequences were inserted into commonly used MVA insertion sites located at different positions within the three sMVA fragments. The sMVA SARS-CoV-2 virus was reconstituted with fowl pox virus (FPV) as a helper virus upon co-transfection of the DNA plasmids into BHK-21 cells, which are non-permissive for FPV. The virus stocks were propagated on chicken embryo fibroblast (CEF) cells, which are commonly used for MVA vaccine production. The infected CEF cells were grown further, and the infected cells were harvested, freeze-thawed and stored at −80° C., then titrated on CEF cells to grow expanded virus stocks. To transition vaccine candidates into clinical production, viruses were plaque purified and clones expanded. Clone COH04S1 was selected for clinical vaccine production and the clinical stock used in this trial was produced on CEF at the COH Center for Biomedicine and Genetics (CBG).

Enzyme-linked immunosorbent assay (ELISA) for IgG binding antibody detection: SARS-CoV-2-specific binding antibodies detected by indirect ELISA utilizing purified S, RBD, and N proteins (Sino Biological 40589-V08B1, 40592-V08H, 40588-V08B). Briefly, 96-well plates (Costar 3361) were coated with 100 ul/well of S, RBD, or N proteins at a concentration of 1 μg/ml in PBS and incubated overnight at 4° C. Plates were washed 5× with wash buffer (0.1% Tween-20/PBS), then blocked with 250 μl/well of assay buffer (0.5% casein/154 mM NaCl/10 mM Tris-HCl/0.1% Tween-20 [pH 7.6]/8% Normal goat serum) for 2 hours at room temperature. After washing, 3-fold diluted heat-inactivated serum in blocking buffer was added to the plates starting from a dilution of 1:150. Plates were wrapped in foil and incubated 2 hours at 37° C. Plates were washed and 1:3,000 dilution of anti-human IgG HRP secondary antibody (BioRad 204005) in assay buffer was added for 1 hour at room temperature. Plates were washed and developed with 1 Step TMB-Ultra (Thermo Fisher 34029). After 2-4 minutes the reaction was stopped with 1M H₂SO₄ and 450 nm absorbance was immediately quantified on FilterMax F3 (Molecular Devices). Positive and negative controls were included in each plate and consisted of serum pools of SARS-CoV-2 seropositive (S, RBD, and N endpoint titer 36450) and seronegative individuals (S, RBD, and N endpoint titer<150). Endpoint titers were calculated as the highest dilution to have an absorbance>0.100.

Pseudovirus production: SARS-CoV-2 pseudovirus was produced using a plasmid lentiviral system based on pALD-gag-pol, pALD-rev, and pALD-GFP (Aldevron). Plasmid pALD-GFP was modified to express Firefly luciferase (pALD-Fluc). Plasmid pCMV3-S(Sino Biological VG40589-UT) was utilized and modified to express SARS-CoV-2 Wuhan-Hu-1 S with D614G modification. Customized gene sequences cloned into pTwist-CMV-BetaGlobin (Twist Biosciences) were used to express SARS-CoV-2 VOC-specific S variants. All S antigens were expressed with C-terminal 19aa deletion. A transfection mixture was prepared 1 ml OptiMEM that contained 30 μl of TransIT-LT1 transfection reagent (Mirus MIR2300) and 6 μg pALD-Fluc, 6 μg pALD-gag-pol, 2.4 μg pALD-rev, and 6.6 μg S expression plasmid. The transfection mix was added to 5×10⁶ HEK293T/17 cells (ATCC CRL11268) seeded the day before in 10 cm dishes and the cells were incubated for 72 h at 37° C. Supernatant containing pseudovirus was harvested and frozen in aliquots at −80° C. Lentivirus was titrated using the Lenti-XTM p24 Rapid Titer Kit (Takara) according to the manufacturer's instructions.

Pseudovirus neutralization assay: SARS-CoV-2 pseudoviruses were titrated in vitro to calculate the virus stock amount that equals 100,000-200,000 relative luciferase units. Flat-bottom 96-well plates were coated with 100 μl poly-L-lysine (0.01%). Serial 2-fold serum dilutions starting from 1:20 were prepared in 50 μl media and added to the plates in triplicates, followed by 50 μl of pseudovirus. Plates were incubated overnight at 4° C. The following day, 10,000 HEK293T-ACE2 cells (32) were added to each well in the presence of 3 μg/ml polybrene and plates were incubated at 37° C. After 48 h of incubation, luciferase lysis buffer (Promega E1531) was added and luminescence was quantified using SpectraMax L (Molecular Devices) after adding One-Glo luciferin (Promega E6110, 100 μl/well). For each plate, positive (pseudovirus only) and negative (cells only) controls were added. The neutralization titer for each dilution was calculated as follows: NT=[1-(mean luminescence with immune sera/mean luminescence without immune sera)]×100. The titers that gave 50% neutralization (NT50) were calculated by determining the linear slope of the graph plotting NT versus serum dilution by using the next higher and lower NT using Microsoft Office Excel (v2019).

IFNγ/IL-4 T cells quantification by ELISPOT: Peripheral blood mononuclear cells (PBMCs) were isolated from fresh blood using Ficoll and counted using Luna-FL cell counter (Logos Biosystems). Frozen PBMCs were thawed and IFNγ/IL-4 secretion evaluated using Human IFNγ/IL-4 FluoroSpot FLEX kit (Mabtech, X-01A16B) following manufacturer instructions. Briefly, 150,000 cells/well in CTL-test serum free media (Immunospot CTLT-010) were added to duplicate wells and stimulated with peptide pools (15-mers, 11 aa overlap, >70% purity). Spike peptide library (GenScript) consisted of 316 peptides and was divided into 4 sub-pools spanning the S1 and S2 domains (1S1=1-86; 1S2=87-168; 2S1=169-242; 2S2=243-316; peptides 173 and 304-309 were not successfully synthesized therefore excluded from the pools). Nucleocapsid (GenScript) and Membrane (in house synthesized) libraries consisted of 102 and 53 peptides, respectively. Each peptide pool (2 μg/ml) and αCD28 (0.1 μg/ml, Mabtech) were added to the cells and plates were incubated for 48 h at 37° C. Control cells (50,000/well) were stimulated with PHA (10 μg/ml). After incubation, plates were washed with PBS and primary and secondary antibodies were added according to manufacturer's protocol. Fluorescent spots were acquired using CTL S6 Fluorocore (Immunospot). For each sample, spots in unstimulated DMSO-only control wells were subtracted from spots in stimulated wells. Zero spots were indicated as one. Total spike response was calculated as the sum of the response to each spike sub-pool. Fifty spots/10⁶ cells were chosen as the arbitrary threshold discriminating negative from positive samples for the calculation of the fold-increase.

CD137+ T cells quantification: SARS-CoV-2-specific T cells were longitudinally monitored by measuring concentrations of CD3+CD8+ and CD3+CD4+ T-cells expressing the 4-1 BB (CD137) activation marker following 24 hours stimulation with either S-15mer megapool (33) (overlapping 15-mers by 10aa) or N peptide library (Genscript), as previously detailed (34). PBMC for each time point were labeled and analyzed by fluorescence-activated cytometry (Gallios™, Beckman Coulter with Kaluza analysis software, Brea, CA) (35).

Outcomes: The primary objective of this trial was to evaluate the safety and tolerability of COH04S1 vaccine in healthy participants at three different DL: 1.0×10⁷ PFU/dose, 1.0×10⁸ PFU/dose, and 2.5×10⁸ PFU/dose. Dose escalation/expansion was based on the incidence of moderate toxicity (MOD) in sentinel participants, with subsequent safety constraints during the expansion cohorts. An MOD was defined as a grade 2 possibly, probably, or definitely attributable to the research treatment that persisted for seven days or more, or any grade 3 treatment-related AE that was an expected vaccine-associated side effects such as fever, chills, malaise, headache, and flu-like symptoms (myalgia and arthralgia) that resolved to grade 1 or less in <7 days. Treatment-related AEs of grade 3 or higher that did not qualify as MOD in any participant would halt all accrual on all dose levels. Toxicity was graded according to standard Division of AIDS (DAIDS) adult toxicity tables.

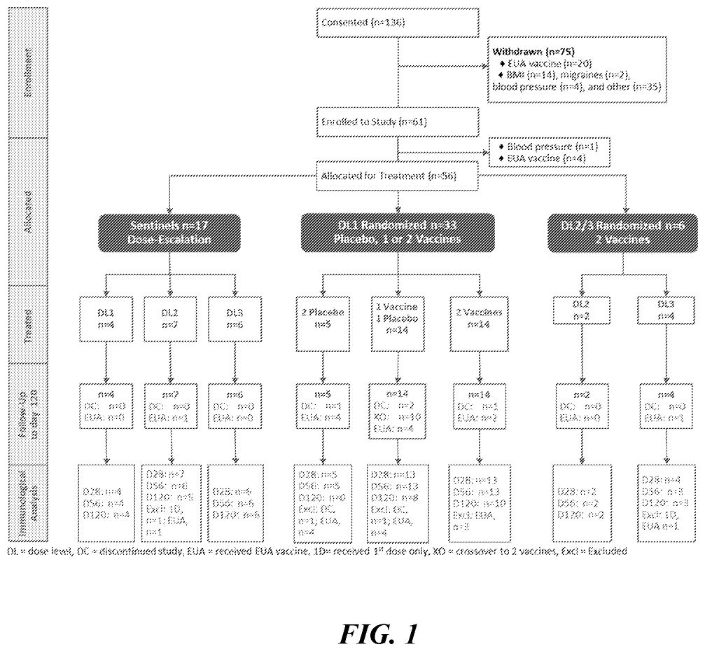

The protocol-defined primary immunologic endpoint was based on serum IgG against SARS-CoV-2. Specifically, a 4-fold rise from baseline value of IgG specific for S or N protein during the 56 d period post-vaccination was considered a positive immunogenicity response, provided the subject was not diagnosed with SARS-CoV-2. Secondary immunogenicity objectives included the longitudinal evaluation of SARS-CoV-2 S-, RBD- and N-specific IgG; NAb to ancestral Wuhan and variant SARS-CoV-2 strains; evaluation of SARS-CoV-2 S and N-specific T cell levels and Th1 vs Th2 polarization; activated/cycling phenotype markers on T cells, and durability of immune responses. Additionally, the role of two injections (prime on day 0 and boost on day 28 or day 56/90) was explored versus one injection at DL1 in which enrollment was double blinded and placebo controlled (FIG. 1). Disclosed herein are: (I) the primary safety endpoints up to day 120, (II) the primary immunogenicity endpoint evaluated up to one month post-second dose, and (111) secondary immunogenicity results up to day 120 (or three months post-second vaccination in DL1/placebo participants boosted at day 56/90).

Statistical analysis: Dose-escalation was explored based on a queue-based design (IQ 3+3) adapted to healthy subjects' study using a biologic and designed to rapidly complete the Phase I portion subject to specific traditional constraints on subject risk (9). This design uses a MOD as the event of concern and requires the first participant on each dose level to be observed for 7 days after injection before any additional participants were permitted on that dose level. The detailed decision grid is available in “VaccineDecisionGrid.xlsx” (oneq.netlify.app/). There were two key safety signals used to limit risk: MOD which were used to limit subject risk per the dose escalation design, and toxicities exceeding MOD which would halt all accrual on all dose levels. Additionally, if at any time a third or more of participants experienced an MOD on a dose level, that dose would hold accrual pending review by the DMC. By adapting to the subject queue, this design reduced the expected Phase I study duration when compared to a non-queue-based 3+3 design by approximately 21%.

The expansion cohorts were intended to provide additional safety data and to help guide dose selection based on biological correlatives. The design was expected to have between 19-23 subjects at a dose level receiving both prime and boost (including sentinel subjects). The conduct of the expansion cohorts was modified due to the EUA vaccines.

DL1 was designed to have an additional expansion cohort randomized between placebo/placebo (n=5), DL1/placebo (n=15), and DL1/DL1 (n=15). The placebo, recommended by the FDA, provided both a background level of adverse events, but also helped providing a control for immunogenicity. There was 82% power to detect a statistically significant difference (with a type I error of 10% using an Exact test) in the primary immunological response rate being the true response rate of DL1/DL1 82% and that of the placebo 20%.

Safety analysis was based on the full analysis set including all enrolled individuals who received at least one injection (56/56). For safety assessment, open label and RCT arms were analyzed individually. Immunogenicity analysis included all enrolled individuals that received at least one vaccination and provided samples for immunogenicity studies (54/56). For each DL data from open label subjects and RCT arm were pooled for immunological analysis given that subjects received the same vaccination regimens and immunogenicity was considered comparable.

Binding antibody (BAb) and NAb titers were disclosed based on seroconversion (4-fold increase or not) relative to baseline and on geometric mean titers (GMT), medians, range and interquartile range. 95% CIs for proportions were calculated using the Clopper-Pearson method. Prior approaches were followed for subjects with no baseline detectable antibody titers (10,11) where half the lower limit of quantification (LLOQ) was used as baseline (results were not sensitive to replacing half LLOQ with the LLOQ). Additional, post-hoc immunological analysis included analysis of post-prime and post-boost increase in S, RBD, N BAb and NAb compared to baseline, and the proportion of participants that seroconverted. Cellular responses measured by ELISPOT were disclosed based on median spot values. Samples with undetectable ELISPOT values were assigned a value of 1 for fold-increase calculation/plotting. To characterize Th cell polarization, for each time point the ratio of S- or N-specific IFNγ and IL-4 T cells was calculated, and an increase over baseline was considered a Th1-polarized response. Statistical comparisons used non-parametric tests (Wilcoxon, Fisher's Exact test, Kruskal-Wallis test), and Exact p-values for Pearson's test (StatXact version 12). Sentinel individuals with available baseline samples were longitudinally analyzed for amounts of S- and N-specific CD137+/CD4+ and CD137+/CD8+ T cells. Percent change was measured on the natural log scale (percent change=In(After/Before)). There was no attempt to address multiple comparisons issue with respect to these multiple exploratory endpoints in the context of this study. All calculations were performed in R version 4.02 or StatXact.

### Example 2. Safety Evaluation

The safety of COVID19 vaccinations as disclosed herein was evaluated in healthy adults. 56 participants initiated vaccination during a period of over 5 months (FIG. 1). There were 17 persons in the open label dose-escalation safety portion of the study assigned to the three DLs (4 DL1, 7 DL2, and 6 DL3). 16/17 sentinels received two vaccinations with one DL2 sentinel who only received one vaccination (see below). One DL2 sentinel subject decided to receive an EUA vaccine after day 56. The remaining 39 subjects were assigned to the RCT arms. In the DL1 RCT portion, 33 subjects were randomized between placebo/placebo (n=5), DL1/placebo (N=14), and DL1/DL1 (N=14). Two of those 33 randomized subjects discontinued the study (unrelated to adverse events) before the second vaccination and did not provide post-prime samples for the immunological analysis (one assigned to DL1/DL1 and one to DL1/placebo group). A separate RCT between DL2/DL2 and DL3/DL3 was planned for 30 subjects but closed at six subjects (two DL2/DL2, four DL3/DL3) due to accrual limitations when EUA vaccine availability became widespread. One subject on DL3 did not receive the second injection (see below).

Following unblinding at day 56, 10/13 subjects who were still enrolled in the DL1/placebo arm opted to receive a second DL1 vaccination at day 56 (9/10) or day 90 (1/10). The other three proceeded to receive an EUA vaccine. In the placebo arm 4/5 subjects received EUA vaccination at day 56 and one discontinued the study post-unblinding with no further follow-up. Subjects who received an EUA vaccine at any time during enrollment remained on the trial, but safety and immunological analysis is presented only up to the time of EUA vaccination. Detailed demographic characteristics of the participants are listed in Table 2.

TABLE 2
Demographic Characteristics

| | Overall, | | DL1/DL1, | | DL2/DL2, | | DL3/DL3, | | P/P, | | DL1/P, | |
|---|---|---|---|---|---|---|---|---|---|---|---|---|
| Characteristic | N = 56¹ | | N = 18¹ | | N = 9¹ | | N = 10¹ | | N = 5¹ | | N = 14¹ | |
| Age | 41 | (21, 55) | 46 | (26, 51) | 39 | (28, 55) | 32 | (26, 54) | 35 | (24, 46) | 40 | (21, 55) |
| Gender | | | | | | | | | | | | |
| Female | 31 | (55%) | 10 | (56%) | 6 | (67%) | 6 | (60%) | 2 | (40%) | 7 | (50%) |
| Male | 25 | (45%) | 8 | (44%) | 3 | (33%) | 4 | (40%) | 3 | (60%) | 7 | (50%) |
| BMI | 25.0 | (19.4, 30.0) | 25.3 | (19.7, 30.0) | 25.8 | (20.2, 30.0) | 22.4 | (19.4, 29.9) | 21.3 | (21.1, 28.5) | 25.0 | (22.2, 29.9) |
| Race | | | | | | | | | | | | |
| Asian | 10 | (18%) | 2 | (11%) | 1 | (11%) | 1 | (10%) | 2 | (40%) | 4 | (29%) |
| Non- | 2 | (3.6%) | 0 | (0%) | 1 | (11%) | 0 | (0%) | 0 | (0%) | 1 | (7.1%) |
| disclosed | | | | | | | | | | | | |
| Unknown | 8 | (14%) | 1 | (5.6%) | 3 | (33%) | 4 | (40%) | 0 | (0%) | 0 | (0%) |
| White | 36 | (64%) | 15 | (83%) | 4 | (44%) | 5 | (50%) | 3 | (60%) | 9 | (64%) |
| Ethnicity | | | | | | | | | | | | |
| Hispanic | 13 | (23%) | 4 | (22%) | 4 | (44%) | 3 | (30%) | 0 | (0%) | 2 | (14%) |
| Non- | 40 | (71%) | 14 | (78%) | 5 | (56%) | 5 | (50%) | 5 | (100%) | 11 | (79%) |
| Hispanic | | | | | | | | | | | | |
| Non- | 1 | (1.8%) | 0 | (0%) | 0 | (0%) | 0 | (0%) | 0 | (0%) | 1 | (7.1%) |
| disclosed | | | | | | | | | | | | |
| Unknown | 2 | (3.6%) | 0 | (0%) | 0 | (0%) | 2 | (20%) | 0 | (0%) | 0 | (0%) |

¹Median (Range); n (%). P = placebo

The AEs are shown in Tables 3-5. AEs were those expected following a vaccine injection, namely injection site reactions (42/51 non-placebo participants, 2/5 placebo participants) were most common followed by fatigue (34/51 non-placebo, 1/5 placebo) and headache (25/51 non-placebo, 2/5 placebo). Local injection sites reactions where all grade 1. No serious AEs and no unanticipated problems were reported.

TABLE 3
Local and systemic adverse reactions after one and two vaccinations
in the open-label dose-escalation portion of the trial

| | DL1/DL1, n = 4 | | DL2/DL2, n = 7 | | | DL3/DL3, n = 6 | |
|---|---|---|---|---|---|---|---|
| | 1^{st} shot, | 2^{nd} shot, | | | 2^{nd} shot, | 1^{st} shot, | 2^{nd} shot, |
| | n = 4 | n = 4 | 1^{st} shot, n = 7 | | n = 6 | n = 6 | n = 6 |
| AE | Grade 1 | Grade 1 | Grade 1 | Grade 2 | Grade 1 | Grade 1 | Grade 1 |
| Injection site | 3 (75%) | 3 (75%) | 6 (86%) | | 3 (50%) | 6 (100%) | 4 (67%) |
| reaction | | | | | | | |
| Fatigue | 1 (25%) | | 4 (57%) | 1 (14%) | 4 (67%) | 2 (33%) | 2 (33%) |
| Headache | 1 (25%) | 1 (25%) | 5 (71%) | | 3 (50%) | 3 (50%) | |
| Myalgia | | | 2 (29%) | | 2 (33%) | 1 (17%) | 1 (17%) |
| Chills | | | | | 1 (17%) | 2 (33%) | |
| Diarrhea | | | 1 (14%) | | | 1 (17%) | 1 (17%) |
| Fever | | | | | | 3 (50%) | |
| Nausea | | | 2 (29%) | | | | |
| Increased | | 1 (25%) | | | | | |
| alanine | | | | | | | |
| aminotransferase | | | | | | | |
| Generalized | | | 1 (14%) | | | | |
| muscle | | | | | | | |
| weakness | | | | | | | |
| Hypertension | | | 1 (14%) | | | | |
| Hypoglycemia | | | | | | | 1 (17%) |
| Hyponatremia | 1 (25%) | | | | | | |
| Insomnia | | | | | | | 1 (17%) |
| Nasal congestion | 1 (25%) | | | | | | |
| Pruritus | | | | | 1 (17%) | | |
| Rash | | | | | 1 (17%) | | |
| maculopapular | | | | | | | |
| Sinus | | 1 (25%) | | | | | |
| tachycardia | | | | | | | |
| Urticaria | | | | | 1 (17%) | | |
| Anxiety | | | | 1 (14%) | | | |

Grade 2 notes:
Anxiety and fatigue in same subject was classified a MOD toxicity. Both resolved within 2 weeks.

TABLE 4
Local and systemic adverse reactions after one, two, and
three vaccinations in the DL1 RCT portion of the trial

| | Placebo/Placebo | | DL1/Placebo | | | | DL1/DL1 | | |
|---|---|---|---|---|---|---|---|---|---|
| | n = 5 | | n = 14 | | | | n = 14 | | |
| | 1^{st} shot | 2^{nd} shot, | 1^{st} shot, | | 2^{nd} shot, | 3^{rd} shot, | 1^{st} shot, | | 2^{nd} shot, |
| | n = 5 | n = 5 | n = 14 | | n = 13 | n = 10 | n = 14 | | n = 13 |
| AE | Grade 1 | Grade 1 | Grade 1 | Grade 3 | Grade 1 | Grade 1 | Grade 1 | Grade 2 | Grade 1 |
| Injection site | 2 | 2 | 8 | | 3 | 9 | 5 | | 7 |
| reaction | (40%) | (40%) | (57%) | | (23%) | (90%) | (36%) | | (54%) |
| Fatigue | 1 | | 4 | | 4 | 6 | 7 | | 5 |
| | (20%) | | (29%) | | (31%) | (60%) | (50%) | | (38%) |
| Headache | 1 | 2 | 2 | | 2 | 5 | 5 | | 4 |
| | (20%) | (40%) | (14%) | | (15%) | (50%) | (36%) | | (31%) |
| Myalgia | | 1 | 2 | | | 2 | 3 | | 5 |
| | | (20%) | (14%) | | | (20%) | (21%) | | (38%) |
| Chills | | 1 | 3 | | 2 | 3 | 1 | | |
| | | (20%) | (21%) | | (15%) | (30%) | (7%) | | |
| Insomnia | | 1 | | | | 1 | 1 | | |
| | | (20%) | | | | (10%) | (7%) | | |
| Nausea | | 1 | | | | 1 | 1 | | |
| | | (20%) | | | | (10%) | (7%) | | |
| Sore throat | | | 1 | | | | 1 | | 1 |
| | | | (7%) | | | | (7%) | | (8%) |
| Cough | | | 1 | | | | | | 1 |
| | | | (7%) | | | | | | (8%) |
| Bilateral | | | | | | 1 | | | |
| hand | | | | | | (10%) | | | |
| swelling | | | | | | | | | |
| Blood | | | | | 1 | | | | |
| bilirubin | | | | | (8%) | | | | |
| increased | | | | | | | | | |
| Creatinine | | | | | | | 1 | | |
| increased | | | | | | | (7%) | | |
| Diarrhea | | | 1 | | | | | | |
| | | | (7%) | | | | | | |
| Fever | | | | 1 | | | | | |
| | | | | (7%) | | | | | |
| Hypocalcemia | 1 | | | | | | | | |
| | (20%) | | | | | | | | |
| Hypokalemia | | | 1 | | | | | | |
| | | | (7%) | | | | | | |
| Lymph node | | 1 | | | | | | | |
| adenopathy | | (20%) | | | | | | | |
| Malaise | | | | | | | 1 | | |
| | | | | | | | (7%) | | |
| Nasal | | | | | | | | | 1 |
| congestion | | | | | | | | | (8%) |
| Vomiting | | | | | | | 1 | | |
| | | | | | | | (7%) | | |
| Idiopathic | | | | | | | | 1 | |
| iritis | | | | | | | | (7%) | |
| Tender | | | | | | 1 | | | |
| breasts | | | | | | (10%) | | | |

TABLE 5
Local and systemic adverse reactions after one and two
vaccinations in the DL2/DL3 RCT portion of the trial

| | DL2/DL2, n = 2 | | DL3/DL3, n = 4 | | | |
|---|---|---|---|---|---|---|
| | 1st shot, n = 2 | 2nd shot, n = 2 | 1st shot, n = 4 | | 2nd shot, n = 3 | |
| AE | Grade 1 | Grade 1 | Grade 1 | Grade 2 | Grade 1 | Grade 2 |
| Injection site reaction | 2 (100%) | 2 (100%) | 3 (75%) | | 2 (67%) | |
| Fatigue | 2 (100%) | 1 (50%) | 2 (50%) | | 1 (33%) | 1 (33%) |
| Headache | 2 (100%) | 1 (50%) | | | 1 (33%) | |
| Chills | | 1 (50%) | 1 (25%) | | 1 (33%) | |
| Fever | | 1 (50%) | 1 (25%) | | | 1 (33%) |
| Myalgia | 1 (50%) | 1 (50%) | | | 1 (33%) | |
| Bronchospasm | | | | 1 (25%) | | |
| Cough | | 1 (50%) | | | | |
| Dizziness | | | 1 (25%) | | | |
| Dry eye | | | 1 (25%) | | | |
| Generalized muscle | 1 (50%) | | | | | |
| weakness | | | | | | |
| Insomnia | | | 1 (25%) | | | |
| Nausea | 1 (50%) | | | | | |
| Sore throat | | 1 (50%) | | | | |
| Vomiting | 1 (50%) | | | | | |
| Cornea tear | | | 1 (25%) | | | |

Table 3 provides the AEs for the 17 sentinel participants on DL1-3 where the one participant (DL2) experienced grade 2 anxiety and grade 2 fatigue on the first injection that lasted 2 weeks. This subject did not receive a second injection. Table 4 are the AEs for the DL1 randomized participants (placebo/placebo, DL1/placebo, DL1/DL1), where the grade 3 fever is noted that lasted less than 24 hours after the first DL1 injection (the participant received the 2nd injection but was DL1/placebo). More typical vaccine side-effects were observed in DL1/DL1 and DL1/placebo than placebo/placebo as expected.

In Table 5, describing AEs in the 6 subjects randomized to receive DL-2 vs DL-3, there was an episode of grade 2 bronchospasm that occurred on a DL-3 subject two weeks after the first injection during a seasonal asthma attack that was judged to be unrelated to the research injection of this known asthmatic person. This participant also had a cornea tear (grade 1) associated with a history of dry eye (grade 1), was put on steroids and therefore ineligible for a second injection.

Local and systemic AEs did not appear to be dose related, with empirically higher injection site reactions and fatigue in DL2 than DL3 or DL1, although the study was not powered for such comparisons.

### Example 3. Immunogenicity Response—Study 1

The immunogenicity resulting from COVID19 vaccinations in a first set of healthy adults was studied. Fifty-four subjects provided immunological samples following vaccination. Two of the fifty-four subjects did not get their planned day 28 injections (1 on DL2/DL2, and 1 on DL3/DL3) as noted above. Five received two placebo injections 28 days apart, 17 received two DL1 doses 28 days apart (DL1/DL1, 4 open label and 13 RCT), 8 received two DL2 doses 28 days apart (DL2/DL2, 6 open label, and 2 RCT); and 9 received two DL3 doses 28 days apart (DL3/DL3, 6 open label, 3 RCT). This resulted in 34 subjects with two vaccines on the planned schedule and 5 placebo/placebo subjects for the primary immunological comparisons.

The pre-specified primary immunogenicity response (IgG>4-fold increase within 56 days of either S or N) was observed in all 34 vaccinated participants (DL1-DL3) and none of the placebo participants. For S-specific IgG, 34/34 (100%) responded (0/5 for placebo, p<0·001), and for N-specific IgG 32/34 (94·1%) responded (15/17 DL1, 8/8 DL2, 9/9 DL3) with p<0·001 vs placebo (0/5) (Table 6).

TABLE 6
Summary humoral responses (subjects
with >4 fold increase in the parameters)

| | Placebo/ | | | | Total with |
|---|---|---|---|---|---|
| | Placebo | DL1/DL1 | DL2/DL2 | DL3/DL3 | Vaccine |
| | (N = 5) | (N = 17) | (N = 8) | (N = 9) | (N = 34) |
| S-IgG | 0/5 | 17/17 | 8/8 | 9/9 | 34/34 |
| (endpoint) | | | | | |
| N-IgG | 0/5 | 15/17 | 8/8 | 9/9 | 32/34 |
| (endpoint) | | | | | |
| RBD-IgG | 0/5 | 17/17 | 8/8 | 9/9 | 34/34 |
| (endpoint) | | | | | |
| NAb (NT50) | 0/5 | 9/17 | 8/8 | 8/9 | 25/34 |

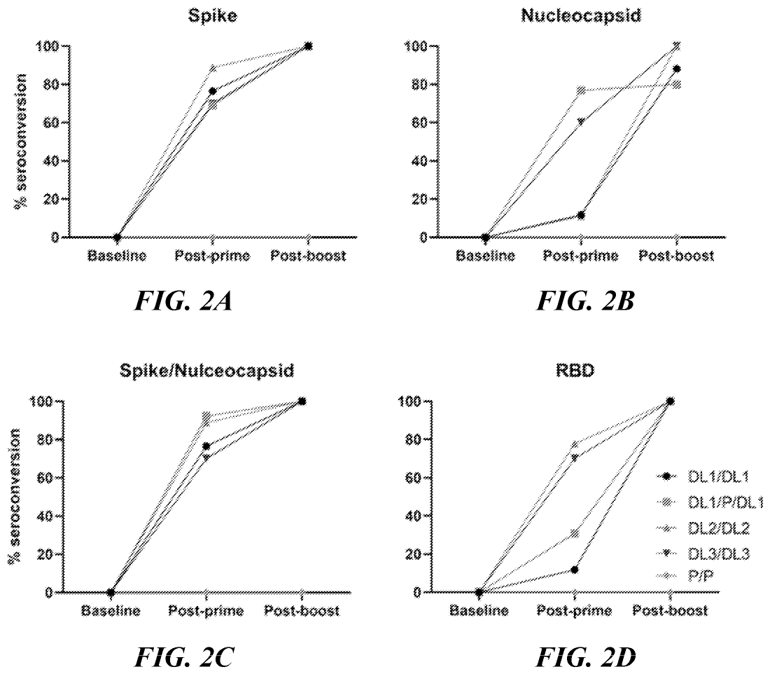

Seroconversion was achieved in 13/17 (76·5%, 95% CI 50·1-93·2) subjects after one DL1 dose, and 17/17 (100·0%, 80·5-100) subjects after two DL1 doses. In subjects vaccinated with DL2, seroconversion was achieved in 8/9 (88·9%, 51·8-99·7) subjects after one DL2 dose, and 8/8 (100·0%, 63·1-100) subjects after two DL2 doses. Seroconversion was achieved in 7/10 (70·0%, 34·8-93·3) subjects after one DL3 dose, and 9/9 (100·0%, 66·4-100) subjects after two DL3 doses. Additionally, in the subjects vaccinated with a DL1 dose followed by placebo and a late boost with DL1 (DL1/placebo/DL1) seroconversion was achieved in 12/13 (92·3%, 64-99·8) subjects after one DL1 dose, and 10/10 (100·0%, 69·2-100) subjects after a delayed DL1 booster vaccination. (FIGS. 2A-2D).

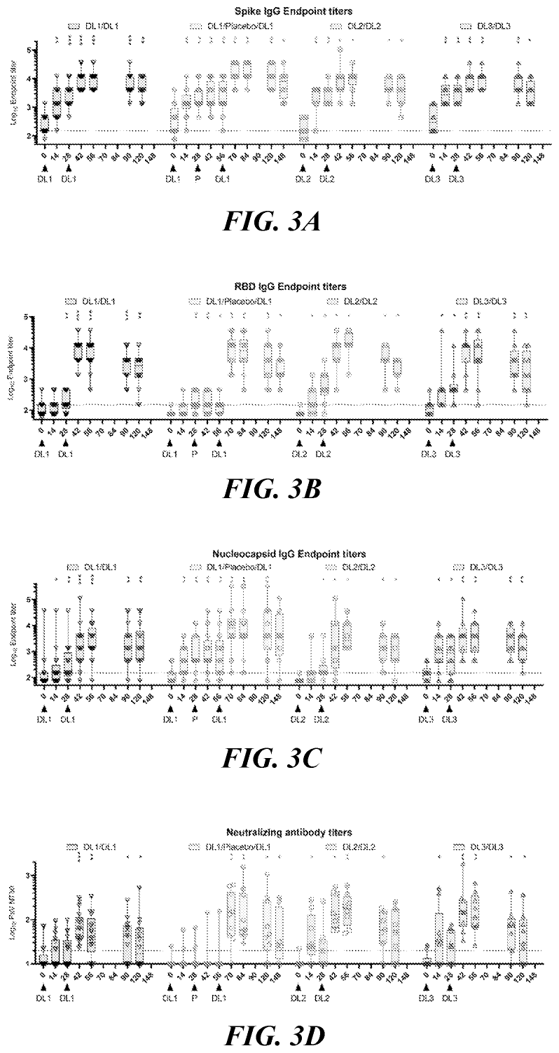

Geometric mean bAb endpoint titers for S at d0, d28 and d56 were DL1 242, 2748 and 9382, DL2 172, 2808 and 9232, DL3 361, 3251 and 9518, respectively. For RBD, the geometric means at d0, d28 and d56 were DL1 100, 176 and 6367, DL2 81, 532 and 12150, DL3 118, 561, and 6599, respectively. For Nucleocapsid, bAb geometric means at d0, d28 and d56 were DL1 151, 315, and 2319, DL2 81, 226, and 4050, DL3 152, 679, and 3585, respectively. All comparisons between day 0 vs. day 28, or day 28 vs. day 56 were statistically significant (Wilcoxon test p<0·05. FIGS. 3A-3C and Tables 7-9).

TABLE 7
Spike IgG Statistical Testing

| Group | p. value | comparison | n | significance |
|---|---|---|---|---|
| DL1/DL1 | 0.0011 | Day 0 vs. Day 14 | 17 | ** |
| | 0.0003 | Day 0 vs. Day 28 | 17 | *** |
| | 0.0003 | Day 0 vs. Day 42 | 17 | *** |
| | 0.0003 | Day 0 vs. Day 56 | 17 | *** |
| | 0.0007 | Day 0 vs. Day 90 | 15 | *** |
| | 0.0011 | Day 0 vs. Day 120 | 14 | ** |
| DL2/DL2 | 0.0090 | Day 0 vs. Day 14 | 9 | ** |
| | 0.0090 | Day 0 vs. Day 28 | 9 | ** |
| | 0.0141 | Day 0 vs. Day 42 | 8 | * |
| | 0.0140 | Day 0 vs. Day 56 | 8 | * |
| | 0.0223 | Day 0 vs. Day 90 | 7 | * |
| | 0.0223 | Day 0 vs. Day 120 | 7 | * |
| DL3/DL3 | 0.0090 | Day 0 vs. Day 14 | 10 | ** |
| | 0.0090 | Day 0 vs. Day 28 | 10 | ** |
| | 0.0088 | Day 0 vs. Day 42 | 9 | ** |
| | 0.0090 | Day 0 vs. Day 56 | 9 | ** |
| | 0.0091 | Day 0 vs. Day 90 | 9 | ** |
| | 0.0140 | Day 0 vs. Day 120 | 9 | * |
| DL1/placebo/ | 0.0090 | Day 0 vs. Day 14 | 13 | ** |
| DL1 | 0.0025 | Day 0 vs. Day 28 | 13 | ** |
| | 0.0025 | Day 0 vs. Day 42 | 13 | ** |
| | 0.0038 | Day 0 vs. Day 56 | 13 | ** |
| | 0.0059 | Day 0 vs. Day 70 | 10 | ** |
| | 0.0059 | Day 0 vs. Day 84 | 10 | ** |
| | 0.0059 | Day 0 vs. Day 118 | 10 | ** |
| | 0.0355 | Day 0 vs. Day 148 | 6 | * |

Serum samples were evaluated for the presence of S-specific IgG by ELISA and endpoint titers were statistically compared to baseline values using two-sided Wilcoxon paired signed-rank test. P-values less than 0.05 are italicized. *=p<0.05, **=p<0.01, ***=p<0.001, ****=p<0.0001.

TABLE 8
RBD IgG Statistical Testing

| Group | p. value | comparison | n | significance |
|---|---|---|---|---|
| DL1/DL1 | 0.0975 | Day 0 vs. Day 14 | 17 | |
| | 0.0084 | Day 0 vs. Day 28 | 17 | ** |
| | 0.0003 | Day 0 vs. Day 42 | 17 | *** |
| | 0.0003 | Day 0 vs. Day 56 | 17 | *** |
| | 0.0007 | Day 0 vs. Day 90 | 15 | *** |
| | 0.0016 | Day 0 vs. Day 120 | 14 | ** |
| DL2/DL2 | 0.0335 | Day 0 vs. Day 14 | 9 | * |
| | 0.0130 | Day 0 vs. Day 28 | 9 | * |
| | 0.0131 | Day 0 vs. Day 42 | 8 | * |
| | 0.0120 | Day 0 vs. Day 56 | 8 | * |
| | 0.0201 | Day 0 vs. Day 90 | 7 | * |
| | 0.0213 | Day 0 vs. Day 120 | 7 | * |
| DL3/DL3 | 0.0136 | Day 0 vs. Day 14 | 10 | * |
| | 0.0137 | Day 0 vs. Day 28 | 10 | * |
| | 0.0088 | Day 0 vs. Day 42 | 9 | ** |
| | 0.0089 | Day 0 vs. Day 56 | 9 | ** |
| | 0.0091 | Day 0 vs. Day 90 | 9 | ** |
| | 0.0090 | Day 0 vs. Day 120 | 9 | ** |
| DL1/placebo/ | 0.1736 | Day 0 vs. Day 14 | 13 | |
| DL1 | 0.0050 | Day 0 vs. Day 28 | 13 | ** |
| | 0.0077 | Day 0 vs. Day 42 | 13 | ** |
| | 0.0103 | Day 0 vs. Day 56 | 13 | * |
| | 0.0057 | Day 0 vs. Day 70 | 10 | ** |
| | 0.0057 | Day 0 vs. Day 84 | 10 | ** |
| | 0.0057 | Day 0 vs. Day 118 | 10 | ** |
| | 0.0350 | Day 0 vs. Day 148 | 6 | * |

Serum samples were evaluated for the presence of RBD-specific IgG by ELISA and endpoint titers were statistically compared to baseline values using two-sided Wilcoxon paired signed-rank test. P-values less than 0.05 are italicized. *=p<0.05, **=p<0.01, ***=p<0.001, ****=p<0.0001.

TABLE 9
Nucleocapsid IgG Statistical Testing

| Group | p. value | comparison | n | significance |
|---|---|---|---|---|
| DL1/DL1 | 0.0310 | Day 0 vs. Day 14 | 17 | * |
| | 0.0035 | Day 0 vs. Day 28 | 17 | ** |
| | 0.0005 | Day 0 vs. Day 42 | 17 | *** |
| | 0.0007 | Day 0 vs. Day 56 | 17 | *** |
| | 0.0016 | Day 0 vs. Day 90 | 15 | ** |
| | 0.0024 | Day 0 vs. Day 120 | 14 | ** |
| DL2/DL2 | 0.0890 | Day 0 vs. Day 14 | 9 | |
| | 0.0104 | Day 0 vs. Day 28 | 9 | * |
| | 0.0220 | Day 0 vs. Day 42 | 8 | * |
| | 0.0130 | Day 0 vs. Day 56 | 8 | * |
| | 0.0215 | Day 0 vs. Day 90 | 7 | * |
| | 0.0335 | Day 0 vs. Day 120 | 7 | * |
| DL3/DL3 | 0.0355 | Day 0 vs. Day 14 | 10 | * |
| | 0.0355 | Day 0 vs. Day 28 | 10 | * |
| | 0.0090 | Day 0 vs. Day 42 | 9 | ** |
| | 0.0090 | Day 0 vs. Day 56 | 9 | ** |
| | 0.0088 | Day 0 vs. Day 90 | 9 | ** |
| | 0.0091 | Day 0 vs. Day 120 | 9 | ** |
| DL1/placebo/ | 0.0057 | Day 0 vs. Day 14 | 13 | ** |
| DL1 | 0.0033 | Day 0 vs. Day 28 | 13 | ** |
| | 0.0035 | Day 0 vs. Day 42 | 13 | ** |
| | 0.0059 | Day 0 vs. Day 56 | 13 | ** |
| | 0.0058 | Day 0 vs. Day 70 | 10 | ** |
| | 0.0058 | Day 0 vs. Day 84 | 10 | ** |
| | 0.0091 | Day 0 vs. Day 118 | 10 | ** |
| | 0.0591 | Day 0 vs. Day 148 | 6 | |

Serum samples were evaluated for the presence of N-specific IgG by ELISA and endpoint titers were statistically compared to baseline values using two-sided Wilcoxon paired signed-rank test. P-values less than 0.05 are italicized. *=p<0.05, **=p<0.01, ***=p<0.001, ****=p<0.0001.

Subjects in DL1, 2, and 3 cohorts that presented with at least a 4-fold increase in NAb titers against D614G PsV within 56 days were 9/17, 8/8 and 8/9, respectively. This was statistically distinct from placebo (0/5) for each dose level (p<0·05, <0·001, <0·003, Fisher's Exact test), but indicated a dose effect (p<0·03, Exact Test).

The geometric means for the D614G PsV NAb titers at day 0, 28 and 56 were DL1 13·8, 17·8, and 43·4, DL2 10·9, 20·6, and 166·9, DL3 12·1, 23·7, and 136·6, respectively. Statistically significant increases in NAb titers compared to baseline were measured at day 14 for DL2, and DL3 (both p<0·05) but not DL1. At day 56 and 120, DL1, DL2, and DL3 all showed statistically significant increases compared to baseline (d56 all p<0·01; d120 p<0.01 DL1, and p<0.05 DL2 and DL3. FIG. 3D and Table 10).

TABLE 10
D614G Neutralizing Antibodies Statistical Testing

| Group | p. value | comparison | n | significance |
|---|---|---|---|---|
| DL1/DL1 | 0.0680 | Day 0 vs. Day 14 | 17 | |
| | 0.0684 | Day 0 vs. Day 28 | 17 | |
| | 0.0003 | Day 0 vs. Day 42 | 17 | *** |
| | 0.0027 | Day 0 vs. Day 56 | 17 | ** |
| | 0.0249 | Day 0 vs. Day 90 | 15 | * |
| | 0.0300 | Day 0 vs. Day 120 | 14 | * |
| DL2/DL2 | 0.0225 | Day 0 vs. Day 14 | 9 | * |
| | 0.1775 | Day 0 vs. Day 28 | 9 | |
| | 0.0141 | Day 0 vs. Day 42 | 8 | * |
| | 0.0078 | Day 0 vs. Day 56 | 8 | ** |
| | 0.0360 | Day 0 vs. Day 90 | 7 | * |
| | 0.0591 | Day 0 vs. Day 120 | 7 | |
| DL3/DL3 | 0.0223 | Day 0 vs. Day 14 | 10 | * |
| | 0.0754 | Day 0 vs. Day 28 | 10 | |
| | 0.0039 | Day 0 vs. Day 42 | 9 | ** |
| | 0.0039 | Day 0 vs. Day 56 | 9 | ** |
| | 0.0143 | Day 0 vs. Day 90 | 9 | * |
| | 0.0360 | Day 0 vs. Day 120 | 9 | * |
| DL1/placebo/ | 0.3711 | Day 0 vs. Day 14 | 13 | |
| DL1 | 0.3711 | Day 0 vs. Day 28 | 13 | |
| | 1.0000 | Day 0 vs. Day 42 | 13 | |
| | 1.0000 | Day 0 vs. Day 56 | 13 | |
| | 0.0092 | Day 0 vs. Day 70 | 10 | ** |
| | 0.0059 | Day 0 vs. Day 84 | 10 | ** |
| | 0.0143 | Day 0 vs. Day 118 | 10 | * |
| | 0.0360 | Day 0 vs. Day 148 | 8 | * |

Serum samples were evaluated for the presence of SARS-CoV-2-specific neutralizing antibodies using a S PsV based on the original SARS-CoV-2 Wuhan strain with D614G substitution. NT50s at different time points were statistically compared to baseline values using two-sided Wilcoxon paired signed-rank test. P-values less than 0.05 are italicized. *=p<0.05, **=p<0.01, ***=p<0.001, ****=p<0.0001.

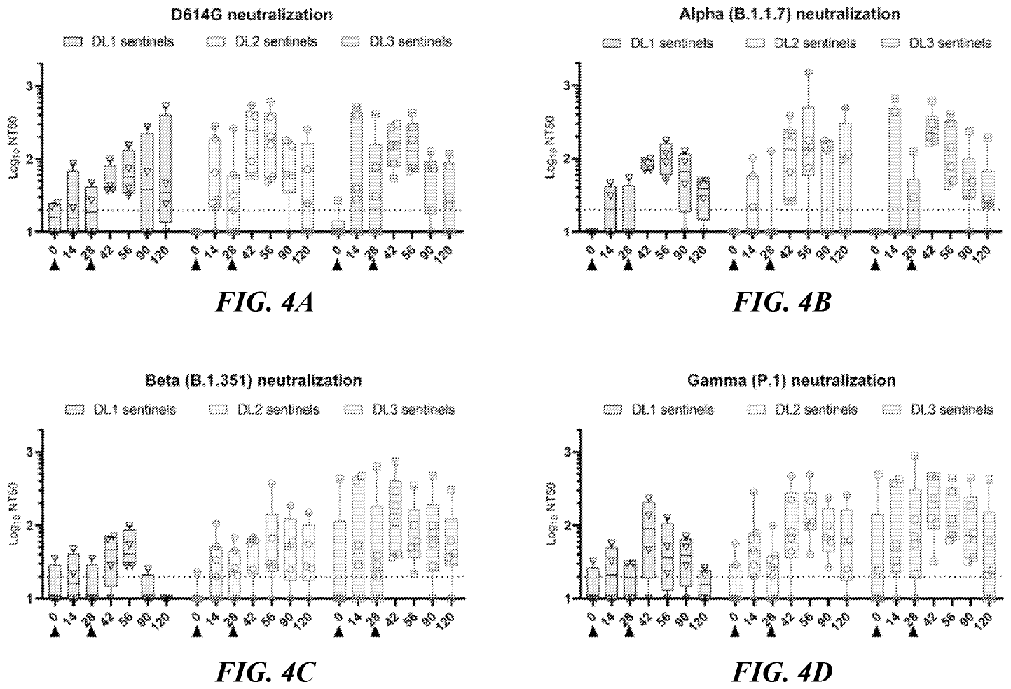

In the DL1/placebo/DL1 group, NAb titers geometric means at day 0, 28, 56 and 84 were 10·7, 12·4, 12·3, and 110·5. The increase of NAb titers from one month after the prime to one month after the boost injection was significantly higher in the DL1/Placebo/DL1 group than the DL1/DL1 group (Wilcoxon test p=0.03) and did not differ from DL2/DL2 and DL3/DL3 groups (p=0.71 Kruskal-Wallis). Samples from the open label portion of the study were longitudinally evaluated for the presence of NAb against VOC (FIGS. 4A-4D). NAb recognizing SARS-CoV-2 VOC tended to be lower in DL1 sentinels when compared to DL2 and DL3 sentinels. COH04S1 induced comparable NAb titers to the different VOC although antibodies to the beta VOC were uniformly lower.

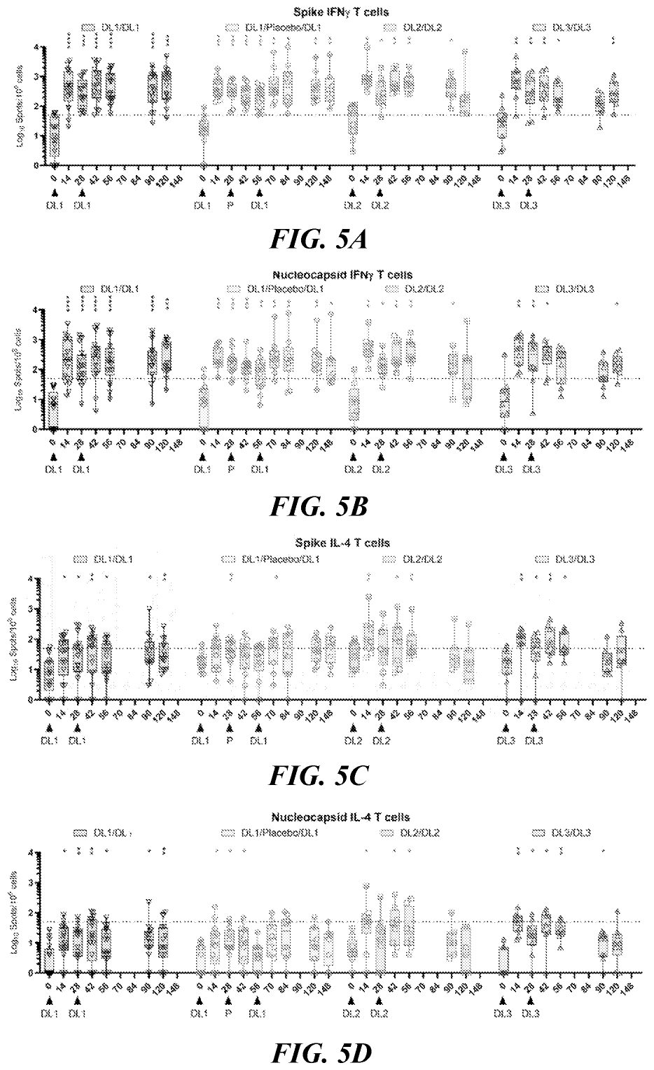

Median S-specific IFN-γ (Th) T-cells values in placebo/DL1/2/3 were 133, 2833, 5350, 1670, with median N of 333, 1735, 2465, 2400 respectively, at day 56 (p<0001 vs Placebo for each). In DL1/placebo/DL1 median S and N specific IFN-γ cells one month post-DL1 boost were 4320 (p<0001 vs Placebo) and 2050 (p<001 vs Placebo), respectively (FIGS. 5A-5B and Tables 11-12).

TABLE 11
Spike-Specific IFNγ T cells Statistical Testing

| Group | p. value | comparison | n | significance |
|---|---|---|---|---|
| DL1/DL1 | 0.0000 | Day 0 vs. Day 14 | 17 | **** |
| | 0.0003 | Day 0 vs. Day 28 | 17 | *** |
| | 0.0000 | Day 0 vs. Day 42 | 17 | **** |
| | 0.0000 | Day 0 vs. Day 56 | 17 | **** |
| | 0.0001 | Day 0 vs. Day 90 | 15 | **** |
| | 0.0001 | Day 0 vs. Day 120 | 14 | *** |
| DL2/DL2 | 0.0039 | Day 0 vs. Day 14 | 9 | ** |
| | 0.0117 | Day 0 vs. Day 28 | 9 | * |
| | 0.0078 | Day 0 vs. Day 42 | 8 | ** |
| | 0.0078 | Day 0 vs. Day 56 | 8 | ** |
| | 0.0313 | Day 0 vs. Day 90 | 7 | * |
| | 0.0781 | Day 0 vs. Day 120 | 7 | |
| DL3/DL3 | 0.0020 | Day 0 vs. Day 14 | 10 | ** |
| | 0.0020 | Day 0 vs. Day 28 | 10 | ** |
| | 0.0039 | Day 0 vs. Day 42 | 9 | ** |
| | 0.0273 | Day 0 vs. Day 56 | 9 | * |
| | 0.0742 | Day 0 vs. Day 90 | 9 | |
| | 0.0039 | Day 0 vs. Day 120 | 9 | ** |
| DL1/placebo/ | 0.0002 | Day 0 vs. Day 14 | 13 | *** |
| DL1 | 0.0002 | Day 0 vs. Day 28 | 13 | *** |
| | 0.0002 | Day 0 vs. Day 42 | 13 | *** |
| | 0.0002 | Day 0 vs. Day 56 | 13 | *** |
| | 0.0020 | Day 0 vs. Day 70 | 10 | ** |
| | 0.0020 | Day 0 vs. Day 84 | 10 | ** |
| | 0.0020 | Day 0 vs. Day 118 | 10 | ** |
| | 0.0078 | Day 0 vs. Day 148 | 8 | ** |

T cells were evaluated for the presence of S-specific T cells secreting IFNγ using ELISPOT. S-specific T cells/i10⁶ PBMCs at different time points were statistically compared to baseline values using two-sided Wilcoxon paired signed-rank test. P-values less than 0.05 are italicized. *=p<0.05, **=p<0.01, ***=p<0.001, ****=p<0.0001.

TABLE 12
Nucleocapsid-Specific IFNγ T cells Statistical Testing

| Group | p. value | comparison | n | significance |
|---|---|---|---|---|
| DL1/DL1 | 0.0000 | Day 0 vs. Day 14 | 17 | **** |
| | 0.0000 | Day 0 vs. Day 28 | 17 | **** |
| | 0.0000 | Day 0 vs. Day 42 | 17 | **** |
| | 0.0000 | Day 0 vs. Day 56 | 17 | **** |
| | 0.0001 | Day 0 vs. Day 90 | 15 | **** |
| | 0.0001 | Day 0 vs. Day 120 | 14 | *** |
| DL2/DL2 | 0.0039 | Day 0 vs. Day 14 | 9 | ** |
| | 0.0128 | Day 0 vs. Day 28 | 9 | * |
| | 0.0078 | Day 0 vs. Day 42 | 8 | ** |
| | 0.0078 | Day 0 vs. Day 56 | 8 | ** |
| | 0.0313 | Day 0 vs. Day 90 | 7 | * |
| | 0.0781 | Day 0 vs. Day 120 | 7 | |
| DL3/DL3 | 0.0020 | Day 0 vs. Day 14 | 10 | ** |
| | 0.0092 | Day 0 vs. Day 28 | 10 | ** |
| | 0.0195 | Day 0 vs. Day 42 | 9 | * |
| | 0.0547 | Day 0 vs. Day 56 | 9 | |
| | 0.1289 | Day 0 vs. Day 90 | 9 | |
| | 0.0391 | Day 0 vs. Day 120 | 9 | * |
| DL1/placebo/ | 0.0002 | Day 0 vs. Day 14 | 13 | *** |
| DL1 | 0.0002 | Day 0 vs. Day 28 | 13 | *** |
| | 0.0002 | Day 0 vs. Day 42 | 13 | *** |
| | 0.0033 | Day 0 vs. Day 56 | 13 | ** |
| | 0.0039 | Day 0 vs. Day 70 | 10 | ** |
| | 0.0020 | Day 0 vs. Day 84 | 10 | ** |
| | 0.0020 | Day 0 vs. Day 118 | 10 | ** |
| | 0.0156 | Day 0 vs. Day 148 | 8 | * |

T cells were evaluated for the presence of N-specific T cells secreting IFNγ using ELISPOT. N-specific T cells/10⁶ PBMCs at different time points were statistically compared to baseline values using two-sided Wilcoxon paired signed-rank test. P-values less than 0.05 are italicized. *=p<0.05, **=p<0.01, ***=p<0.001, ****=p<0.0001.

Prime vaccination resulted in a 4-fold increase in S or N-specific IFNγ T cells in all but one subject (in the DL1/DL1 group) for an overall 98% response rate post-prime (95% CI 89-100). The same response rate was observed within one month after the second immunization (98%, 89-100). At three months post-boost 31/38 (82%, 66-92) subjects had a sustained 4-fold increase in S- or N-specific IFNγ T cells. IL-4 secreting T cells were also induced at all COH04S1 DL, although to a much lower level than IFNγ-secreting T cells (FIGS. 5C-5D and Tables 13-14).

TABLE 13
Spike-Specific IL-4 T cells Statistical Testing

| Group | p. value | comparison | n | significance |
|---|---|---|---|---|
| DL1/DL1 | 0.0131 | Day 0 vs. Day 14 | 17 | * |
| | 0.0245 | Day 0 vs. Day 28 | 17 | * |
| | 0.0066 | Day 0 vs. Day 42 | 17 | ** |
| | 0.0280 | Day 0 vs. Day 56 | 17 | * |
| | 0.0144 | Day 0 vs. Day 90 | 15 | * |
| | 0.0084 | Day 0 vs. Day 120 | 14 | ** |
| DL2/DL2 | 0.0039 | Day 0 vs. Day 14 | 9 | ** |
| | 0.0578 | Day 0 vs. Day 28 | 9 | |
| | 0.0156 | Day 0 vs. Day 42 | 8 | * |
| | 0.0078 | Day 0 vs. Day 56 | 8 | ** |
| | 1.0000 | Day 0 vs. Day 90 | 7 | |
| | 0.9375 | Day 0 vs. Day 120 | 7 | |
| DL3/DL3 | 0.0092 | Day 0 vs. Day 14 | 10 | ** |
| | 0.0330 | Day 0 vs. Day 28 | 10 | * |
| | 0.0039 | Day 0 vs. Day 42 | 9 | ** |
| | 0.0117 | Day 0 vs. Day 56 | 9 | * |
| | 0.5933 | Day 0 vs. Day 90 | 9 | |
| | 0.6353 | Day 0 vs. Day 120 | 9 | |
| DL1/placebo/ | 0.0574 | Day 0 vs. Day 14 | 13 | |
| DL1 | 0.0042 | Day 0 vs. Day 28 | 13 | ** |
| | 0.0942 | Day 0 vs. Day 42 | 13 | |
| | 0.2163 | Day 0 vs. Day 56 | 13 | |
| | 0.0488 | Day 0 vs. Day 70 | 10 | * |
| | 0.1055 | Day 0 vs. Day 84 | 10 | |
| | 0.0645 | Day 0 vs. Day 118 | 10 | |
| | 0.0781 | Day 0 vs. Day 148 | 8 | |

T cells were evaluated for the presence of S-specific T cells secreting IL-4 using ELISPOT. S-specific T cells/106 PBMCs at different time points were statistically compared to baseline values using two-sided Wilcoxon paired signed-rank test. P-values less than 0.05 are italicized. *=p<0.05, **=p<0.01, ***=p<0.001, ****=p<0.0001.

TABLE 14
Nucleocapsid-Specific IL-4 T Cells Statistical Testing

| Group | p. value | comparison | n | significance |
|---|---|---|---|---|
| DL1/DL1 | 0.0158 | Day 0 vs. Day 14 | 17 | * |
| | 0.0071 | Day 0 vs. Day 28 | 17 | ** |
| | 0.0049 | Day 0 vs. Day 42 | 17 | ** |
| | 0.0427 | Day 0 vs. Day 56 | 17 | * |
| | 0.0280 | Day 0 vs. Day 90 | 15 | * |
| | 0.0086 | Day 0 vs. Day 120 | 14 | ** |
| DL2/DL2 | 0.0209 | Day 0 vs. Day 14 | 9 | * |
| | 0.0661 | Day 0 vs. Day 28 | 9 | |
| | 0.0225 | Day 0 vs. Day 42 | 8 | * |
| | 0.0141 | Day 0 vs. Day 56 | 8 | * |
| | 0.2188 | Day 0 vs. Day 90 | 7 | |
| | 1.0000 | Day 0 vs. Day 120 | 7 | |
| DL3/DL3 | 0.0020 | Day 0 vs. Day 14 | 10 | ** |
| | 0.0195 | Day 0 vs. Day 28 | 10 | * |
| | 0.0143 | Day 0 vs. Day 42 | 9 | * |
| | 0.0039 | Day 0 vs. Day 56 | 9 | ** |
| | 0.0391 | Day 0 vs. Day 90 | 9 | * |
| | 0.0753 | Day 0 vs. Day 120 | 9 | |
| DL1/placebo/ | 0.0144 | Day 0 vs. Day 14 | 13 | * |
| DL1 | 0.0118 | Day 0 vs. Day 28 | 13 | * |
| | 0.0178 | Day 0 vs. Day 42 | 13 | * |
| | 0.9188 | Day 0 vs. Day 56 | 13 | |
| | 0.0580 | Day 0 vs. Day 70 | 10 | |
| | 0.0592 | Day 0 vs. Day 84 | 10 | |
| | 0.0960 | Day 0 vs. Day 118 | 10 | |
| | 0.0759 | Day 0 vs. Day 148 | 8 | |

T cells were evaluated for the presence of N-specific T cells secreting IL-4 using ELISPOT. N-specific T cells/10⁶ PBMCs at different time points were statistically compared to baseline values using two-sided Wilcoxon paired signed-rank test. P-values less than 0.05 are italicized. *=p<0.05, **=p<0.01, ***=p<0.001, ****=p<0.0001.

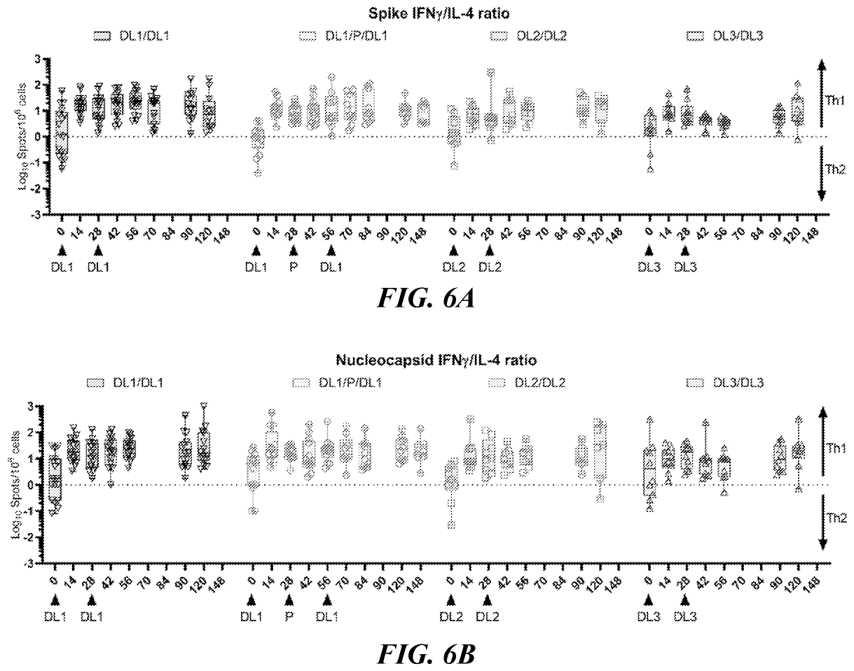

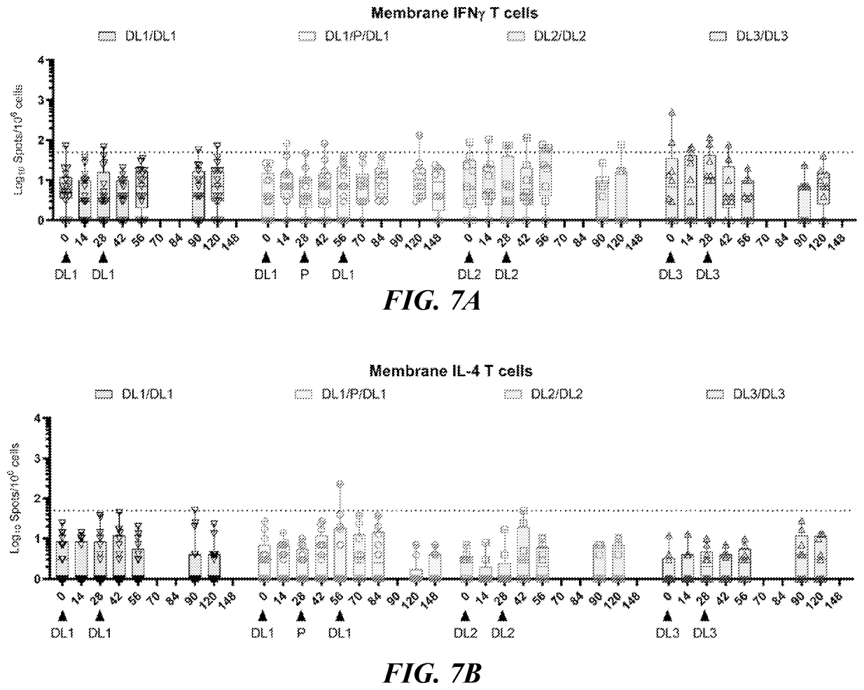

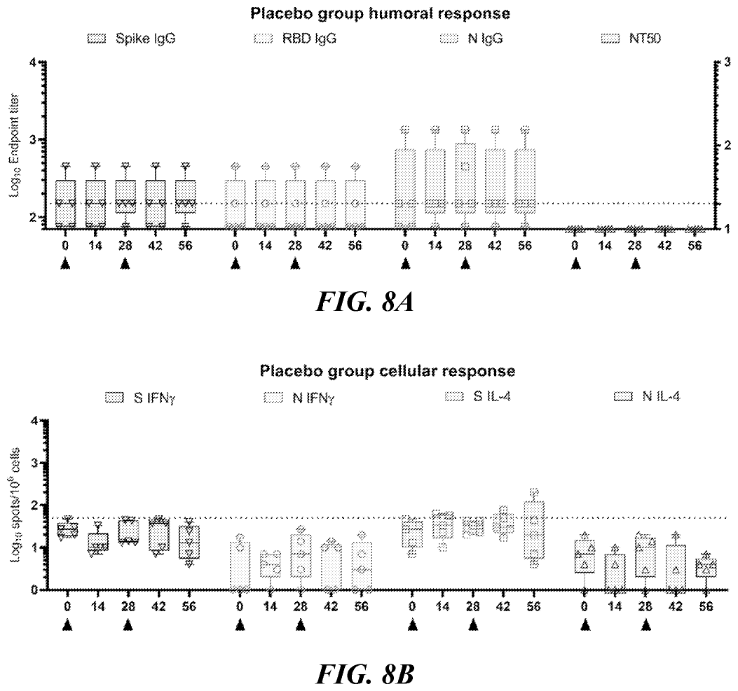

For S, Th1/Th2 (IFN-γ/IL-4) ratio increased with a median of 173% (IQR 53-307%) in combined DL1-3 (p<0·001), with a median 264% (IQR 92-386%), 159% (94-263%), and 70% (2-83%), respectively for DL1, DL2 and DL3 (p<0.05 Wilcoxon test against no-change except for DL3; FIGS. 6A-6B). For N, Th1/Th2 increased a median of 211% in combined DL1-3 (p<0·001), and median increases of 258% (IQR 121-400%), 211% (169-320%), 129% (104%-204%) respectively for DL1, DL2 and DL3 (p<0·05 Wilcoxon test against no-change except for DL3). There was no increase in M-specific T cell responses in any of the groups across time (FIGS. 7A-7B). Additionally, no significant changes in any immunological parameter were measured in placebo subjects (FIGS. 8A-8B).

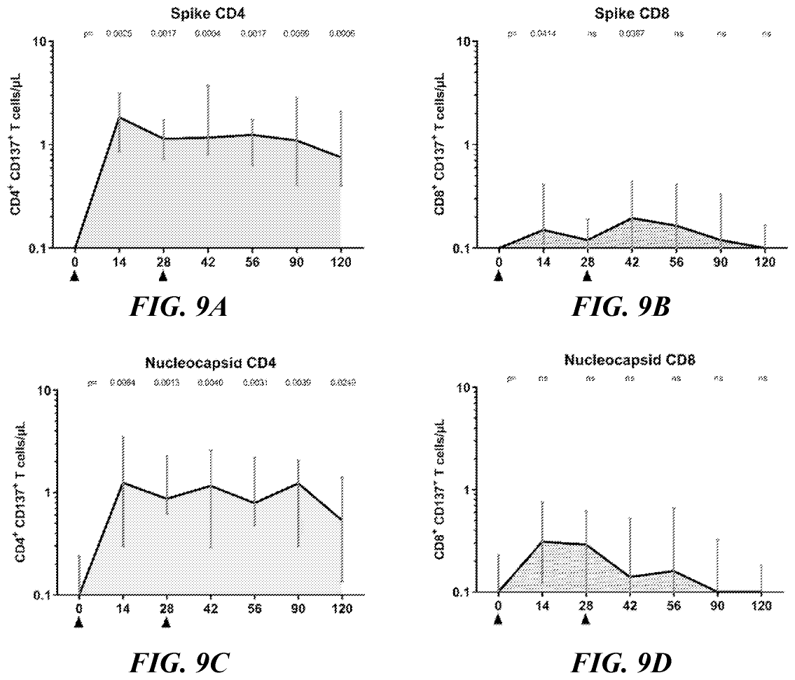

Expression of T cell activation marker CD137 following stimulation with S and N peptides was evaluated only on sentinel subjects (FIGS. 9A-9D). There was a statistically significant increase in both S-specific (257% increase, p<0·01) and N-specific (184% increase, p<0·01) CD137+/CD4+ T cells from day 0 to day 14, which did not have a significant further increase despite the boost on day 28 but remained significantly different from baseline through day 120. S-specific CD137+/CD8+ T cells had a transient increase of 75% over day 0-14, that was no longer significant at day 28, and N-specific CD137+/CD8+ T cells did not register a statistically significant response.

### Example 4. Immunogenicity Response—Study 2

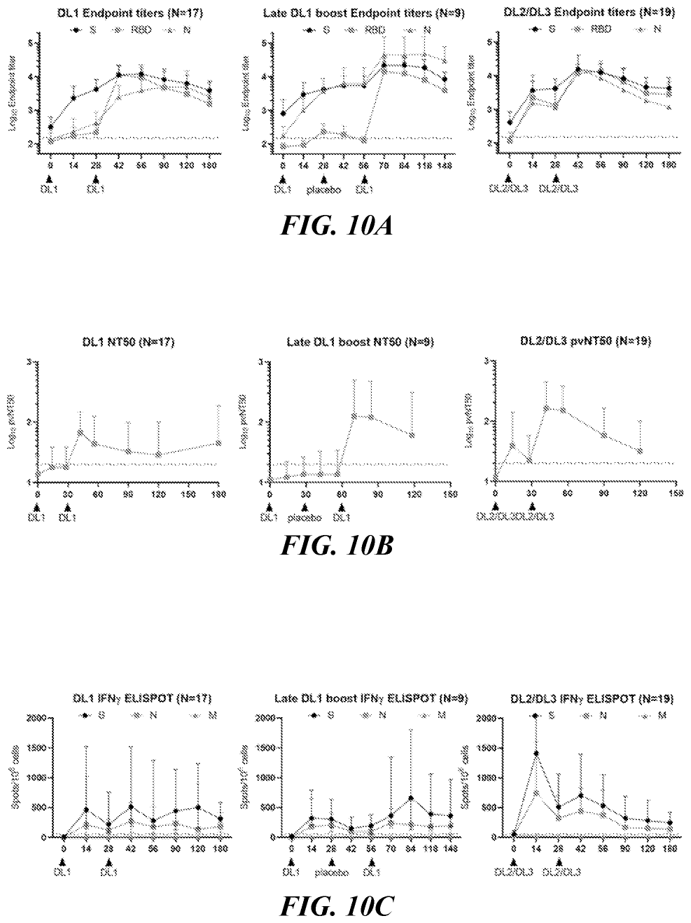

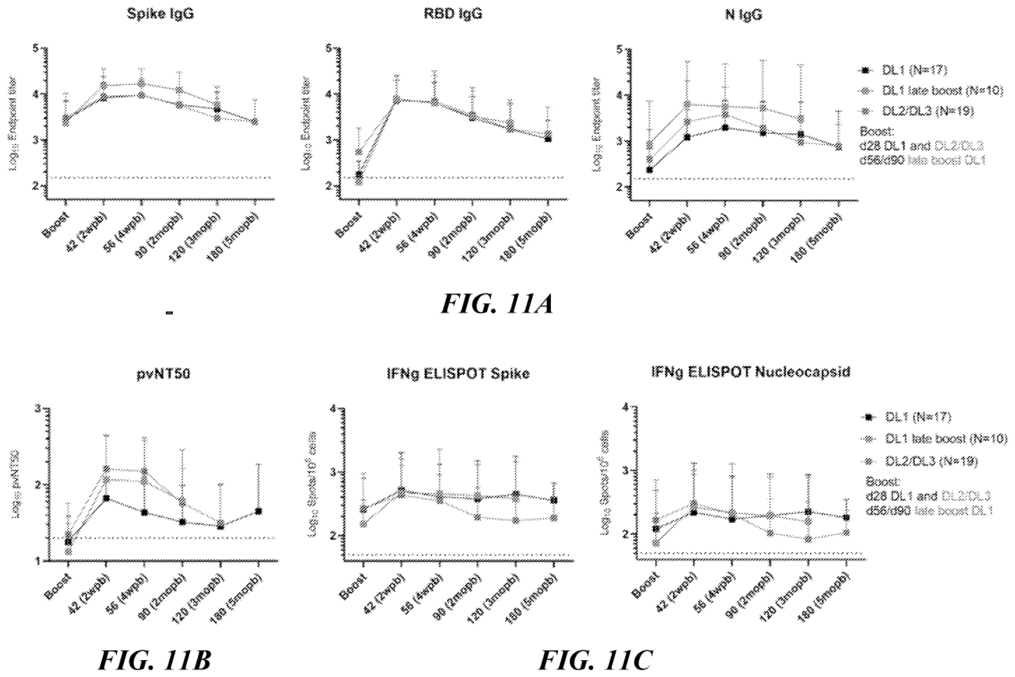

The immunogenicity resulting from COVID19 vaccinations in a second set of healthy adults was studied. Three groups received injections as follows: 2 vaccine (VV), 1 vaccine/1 placebo (VP) and 2 placebo (PP). Including four DL1 sentinels, seventeen participants received two vaccine injections separated by the standard 28-day interval. Additionally, research participants who had an intermediate placebo injection were unblinded at d56 and some received a second COH04S1 vaccination. As shown in FIGS. 10A-10C, individuals that were boosted at day 56 with DL1 showed an overall more robust humoral response to the booster than individuals who had the 28 d interval regime. This was particularly evident when comparing post-boost binding antibodies to Spike and nucleocapsid and neutralizing antibodies (FIGS. 11A-11B). On the contrary, comparable levels of T cell responses to Spike and Nucleocapsid were measured in DL1 volunteers independently of the vaccination schedule. Overall, the data is consistent that a delayed boost improves the response to vaccination in the cohort. This fact is evident when the delayed boost consisted of one of the EUA vaccines (Pfizer, J&J or Moderna). Because the protocol allowed unblinding at day 56, and research participants were encouraged to get a second dose of any EUA vaccine or COH04S1, a wide variety of schedules were permissible.

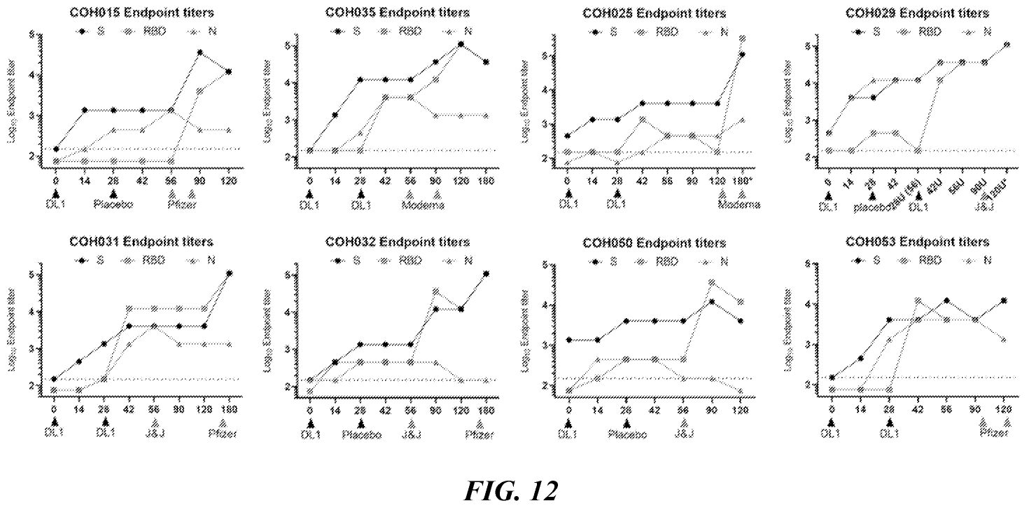

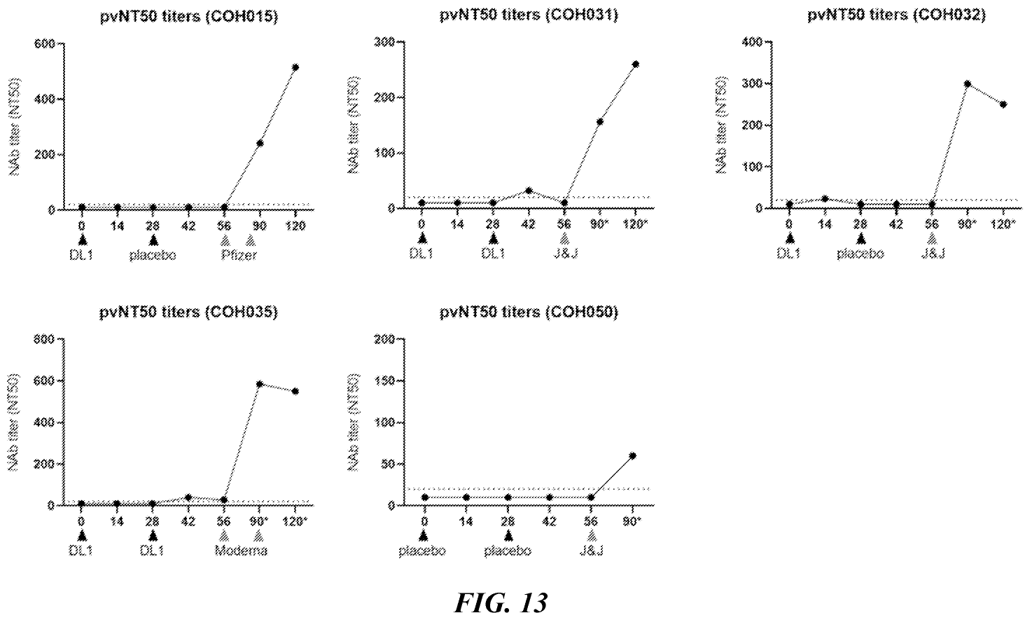

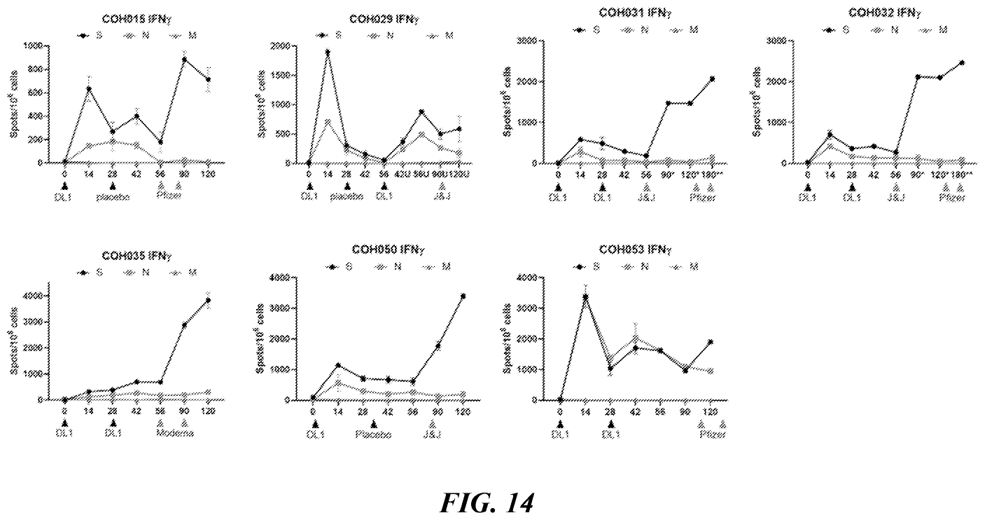

As shown in FIG. 12, with the exception of one volunteer receiving the J&J vaccine after two DL1 doses, all other volunteers immunized with one or two doses of COH04S1 showed a remarkable boost in bAb to S and RBD after immunization with a EUA vaccine. Similarly, neutralizing antibodies and cellular responses were boosted by an additional vaccination with a EUA vaccine (FIGS. 13 and 14). Since N is not included in any EUA vaccine a booster in N-specific humoral or cellular responses was not observed.

To determine if there was an improvement in responsiveness to greater amounts of vaccine, a randomization was started between DL2 and DL3 levels of vaccine. Unfortunately, since the EUA vaccines became widely available at this time, 6 randomized participants were added to 7 DL2 and 6 DL3 sentinels. As can be seen in FIGS. 10A-10C and 11A-11C, bAb titers in DL2/DL3 immunized volunteers were comparable or slightly lower than those measured in late-boosted DL1 volunteers. Additionally, in DL2/DL3 immunized volunteers neutralizing antibodies were overall comparable to late-boosted DL1 volunteers and higher than in DL1 individuals boosted 28 days post-prime. Cellular responses to spike and nucleocapsid tended to be lower in DL2/DL3-immunized volunteers than in DL1 immunized volunteers independently from the time of boost. The conclusion from these measurements is that greater amounts of vaccine provided to research participants in the DL2+DL3 cohorts did not increase the magnitude of immune responsiveness beyond what was measured in the late boost DL1 cohort which could provide a dose sparing of between 10-25-fold over the higher DLs.

### Example 5. Materials and Methods for Booster Vaccination Studies

Materials and methods used in Examples 6-7 regarding the safety and immunogenicity of COH04S1 booster vaccinations in healthy adults are described below.

SARS-CoV-2-specific IgA, IgG, and IgM measured in serum and saliva by ELISA: To evaluate humoral immunity with the COH04S1 vaccine, SARS-CoV-2 specific antibodies, including IgA, IgG, and IgM, in serum and saliva are measured by ELISA at various time points. The assay identifies SARS-CoV-2 antibodies specific for the S receptor-binding domain (RBD) that interacts with ACE2 on the surface of the cells, and the N protein that is one of the first B cell targets, during the initial phase of the SARS-CoV-2 infection [14]. The qualitative assays, based on previously established protocols [15], are developed to investigate Spike subunit 1 (S1)- and N-specific antibodies of the IgG, IgM and IgA subclasses in serum and saliva. Pools of SARS-CoV-2 convalescent serum or SARS-oCoV-2 negative serum will be used as a positive- and negative-controls (University of California at San Diego), respectively. End-point binding antibody titers are expressed as the reciprocal of the last sample dilution to give an OD value above the cut-off [15]. Antibody levels in recipients are graphed on a time plot and compared to baseline level in donors.

SARS-CoV-2-specific neutralizing antibodies: Evaluation of SARS-CoV-2 neutralizing antibody titers in serum samples of COH04S1 vaccinated volunteers are performed at various time points. SARS-CoV-2 lentiviral-pseudovirus is used for expressing the Spike antigen from the original Wuhan strain and infecting 293T cell lines engineered to express ACE2 [16]. Spike incorporation into the pseudovirus is verified and quantified by Western blot using Spike-specific antibodies and by ELISA [17]. Serum samples from Day 42 are also be tested for neutralization of live SARS-CoV-2, and this test is performed at the University of Louisville. As an exploratory endpoint, participants' serum samples are tested for their ability to neutralize new variants of concern (VOC) as they appear in the population. Examples include the UK variant (VOC 202012/01) and the South African variant (VOC 501Y.V2). Pseudoviruses carrying the VOC Spike sequences are used in a neutralization assay to measure neutralizing antibody titers to the VOC.

Th1 vs Th2 polarization: To evaluate the Th1 vs Th2 polarization of immune responses, perform dual fluorescence ELISPOT assay is performed to detect and quantify cells secreting IFN-gamma and IL-4. Briefly, isolated PBMC is stimulated with Spike and Nucleocapsid peptide libraries (15-mers with 11 aa overlap) using fluorospot plates coated with IFN-gamma and IL-4 capture antibodies. Following 48 h co-incubation, plates are washed, and IFN-gamma and IL-4 detection antibodies followed by fluorophore conjugates are added. Plates are read and analyzed with a fluorescent ELISPOT reader and number of spots after stimulation expressed following subtraction of background from unstimulated samples. As an exploratory endpoint, in selected samples, a cytokine-based cytofluorimetric analysis (ICS) is performed to analyzed multiple Th1 and Th2 cytokines. PBMC (1-2×10⁶) is stimulated for 16 hours with SARS-CoV-2-S or SARS-CoV-2-N overlapping peptide libraries (15-mers with 11 aa overlap). Lymphocytes are stained with viability dye and surface stained with antibodies to CD3, CD8 and CD4. After fixing and permeabilization, cells are stained intracellularly with antibodies against IFN-gamma, TNF-alpha, IL-2, IL-4, IL6, IL-13. After washing, cells are acquired using BD FACS Celesta Cell Analyzer and analyzed with FlowJo software.

SARS-CoV-2-specific T-cell responses and evolution of activated/cycling and memory phenotype markers on the surface of antigens-specific T cells: Cellular immunity to SARS-CoV-2-S and -N, major domains of antiviral T cell immunity are investigated in PBMC of COH04S1 vaccinated participants, using multiparameter flow cytometry as previously disclosed [13]. Frequencies of T lymphocyte precursors responsive to SARS-CoV-2-S or SARS-CoV-2-N overlapping peptide libraries are longitudinally monitored. In vaccine responders, SARS-CoV-2 specific T cells are further evaluated by measuring levels [13] of CD137 surface marker expressed on CD3+CD8+ and CD3+CD4+ T cells stimulated for 24 hours with either SARS-CoV-2-S or SARS-CoV-2-N overlapping peptide libraries. CD137 is expressed only on recently activated T cells, and its expression correlates with functional activation of T cells [18]. Measurements of CD137 levels is combined with immunophenotyping studies, by using antibodies to CD28 and CD45RA cell surface markers to assess and identify memory phenotype profiles percentage of effector memory (TEM and TEMRA), central memory (TCM) and naïve SARS-CoV-2-S or SARS-CoV-2-N specific T cells [12]. Additionally, the activated/cycling phenotype is assessed by using the CD38, HLA-DR, Ki67 and PD1 surface markers [19]. Approximately 300,000 events per sample are acquired on a Gallios flow cytometer and analyzed by Kaluza software.

SARS-CoV-2 specific T-cell responses and memory phenotype: Cellular immunity to SARS-CoV-2-S and -N antigens which are immunodominant markers of antiviral T cell immunity is investigated in PBMC of a maximum of 20 COH04S1 vaccinated participants at a minimum of 2 time points. The specimens are sent to La Jolla Institute for Immunology for testing using multiparameter flow cytometry as previously disclosed [20]. The extent of recognition of altered peptide sequences corresponding to four SARS-CoV-2 variants of concern (Alpha, Beta, Gamma, and Delta) and the ancestral Wuhan strain sequence is evaluated by flow cytometry. This can quantitate differences in recognition and binding properties of the altered sequences to CD4+ and CD8+ T cells compared to the ancestral Wuhan sequence composition.

SARS-CoV-2 IgG endpoint ELISA: The assay was developed in house for the detection and quantification of binding antibodies of the IgG type targeting Spike, RBD and Nucleocapsid. ELISA plates are coated with SARS-CoV-2 antigens, and serial dilutions of serum are added in duplicate wells. A secondary antibody is added next followed by a chemiluminescent substrate. The endpoint titer is the last serum dilution to result in an absorbance higher than 0.1 OD at 450 nm. Analysis of anti-Spike serum endpoint titers pre- and post-booster vaccination is considered a measure of vaccine boosting efficacy. RBD-specific binding antibodies are measured and correlated to neutralizing antibodies.

Nucleocapsid binding antibodies are measured pre- and post-booster vaccination to establish COH04S1 Nucleocapsid-specific IgG induction following a single shot in subjects naïve for N antigen.

Enzyme-linked immunosorbent assay (ELISA) for IgG binding antibody detection: SARS-CoV-2-specific binding antibodies detected by indirect ELISA utilizing purified S, RBD, and N proteins (Sino Biological 40589-V08B1, 40592-V08H, 40588-V08B). Briefly, 96-well plates (Costar 3361) were coated with 100 ul/well of S, RBD, or N proteins at a concentration of 1 μg/ml in PBS and incubated overnight at 4° C. Plates were washed 5× with wash buffer (0.1% Tween-20/PBS), then blocked with 250 μl/well of assay buffer (0.5% casein/154 mM NaCl/10 mM Tris-HCl/0.1% Tween-20 [pH 7.6]/8% Normal goat serum) for 2 hours at room temperature. After washing, 3-fold diluted heat-inactivated serum in blocking buffer was added to the plates starting from a dilution of 1:150. Plates were wrapped in foil and incubated 2 hours at 37° C. Plates were washed and 1:3,000 dilution of anti-human IgG HRP secondary antibody (BioRad 204005) in assay buffer was added for 1 hour at room temperature. Plates were washed and developed with 1 Step TMB-Ultra (Thermo Fisher 34029). After 2-4 minutes the reaction was stopped with 1M H2SO4 and 450 nm absorbance was immediately quantified on FilterMax F3 (Molecular Devices). Positive and negative controls were included in each plate and consisted of serum pools of SARS-CoV-2 seropositive (S, RBD, and N endpoint titer 36450) and seronegative individuals (S, RBD, and N endpoint titer<150). Endpoint titers were calculated as the highest dilution to have an absorbance>0.100.

Pseudovirus production: SARS-CoV-2 pseudovirus was produced using a plasmid lentiviral system based on pALD-gag-pol, pALD-rev, and pALD-GFP (Aldevron). Plasmid pALD-GFP was modified to express Firefly luciferase (pALD-Fluc). Plasmid pCMV3-S(Sino Biological VG40589-UT) was utilized and modified to express SARS-CoV-2 Wuhan-Hu-1 S with D614G modification. Customized gene sequences cloned into pTwist-CMV-BetaGlobin (Twist Biosciences) were used to express SARS-CoV-2 VOC-specific S variants. All S antigens were expressed with C-terminal 19aa deletion. A transfection mixture was prepared 1 ml OptiMEM that contained 30 μl of TransIT-LT1 transfection reagent (Mirus MIR2300) and 6 μg pALD-Fluc, 6 μg pALD-gag-pol, 2.4 μg pALD-rev, and 6.6 μg S expression plasmid. The transfection mix was added to 5×10⁶ HEK293T/17 cells (ATCC CRL11268) seeded the day before in 10 cm dishes and the cells were incubated for 72 h at 37° C. Supernatant containing pseudovirus was harvested and frozen in aliquots at −80° C. Lentivirus was titrated using the Lenti-XTM p24 Rapid Titer Kit (Takara) according to the manufacturer's instructions.

Pseudovirus neutralization assay: SARS-CoV-2 pseudoviruses were titrated in vitro to calculate the virus stock amount that equals 100,000-200,000 relative luciferase units. Flat-bottom 96-well plates were coated with 100 μl poly-L-lysine (0.01%). Serial 2-fold serum dilutions starting from 1:20 were prepared in 50 μl media and added to the plates in triplicates, followed by 50 μl of pseudovirus. Plates were incubated overnight at 4° C. The following day, 10,000 HEK293T-ACE2 cells (32) were added to each well in the presence of 3 μg/ml polybrene and plates were incubated at 37° C. After 48 h of incubation, luciferase lysis buffer (Promega E1531) was added and luminescence was quantified using SpectraMax L (Molecular Devices) after adding One-Glo luciferin (Promega E6110, 100 μl/well). For each plate, positive (pseudovirus only) and negative (cells only) controls were added. The neutralization titer for each dilution was calculated as follows: NT=[1-(mean luminescence with immune sera/mean luminescence without immune sera)]×100. The titers that gave 50% neutralization (NT50) were calculated by determining the linear slope of the graph plotting NT versus serum dilution by using the next higher and lower NT using Microsoft Office Excel (v2019).

IFNγ/IL-4 T cells quantification by ELISPOT: Peripheral blood mononuclear cells (PBMC) were isolated from fresh blood using Ficoll and counted using Luna-FL cell counter (Logos Biosystems). Frozen PBMCs were thawed and IFNγ/IL-4 secretion evaluated using Human IFNγ/IL-4 FluoroSpot FLEX kit (Mabtech, X-01A16B) following manufacturer instructions. Briefly, 150,000 cells/well in CTL-test serum free media (Immunospot CTLT-010) were added to duplicate wells and stimulated with peptide pools (15-mers, 11 aa overlap, >70% purity). Spike peptide library (GenScript) consisted of 316 peptides and was divided into 4 sub-pools spanning the S1 and S2 domains (1S1=1-86; 1S2=87-168; 2S1=169-242; 2S2=243-316; peptides 173 and 304-309 were not successfully synthesized therefore excluded from the pools). Nucleocapsid (GenScript) and Membrane (in house synthesized) libraries consisted of 102 and 53 peptides, respectively. Each peptide pool (2 μg/ml) and αCD28 (0.1 μg/ml, Mabtech) were added to the cells and plates were incubated for 48 h at 37° C. Control cells (50,000/well) were stimulated with PHA (10 μg/ml). After incubation, plates were washed with PBS and primary and secondary antibodies were added according to manufacturer's protocol. Fluorescent spots were acquired using CTL S6 Fluorocore (Immunospot). For each sample, spots in unstimulated DMSO-only control wells were subtracted from spots in stimulated wells. Zero spots were indicated as one. Total spike response was calculated as the sum of the response to each spike sub-pool. Fifty spots/10⁶ cells were chosen as the arbitrary threshold discriminating negative from positive samples for the calculation of the fold-increase.

CD137+ T cells quantification: SARS-CoV-2-specific T cells were longitudinally monitored by measuring concentrations of CD3+CD8+ and CD3+CD4+ T-cells expressing the 4-1 BB (CD137) activation marker following 24 hours stimulation with either S-15mer megapool (11) (overlapping 15-mers by 10aa) or N peptide library (Genscript), as previously detailed (12). PBMCs for each time point were labeled and analyzed by fluorescence-activated cytometry (Gallios™, Beckman Coulter with Kaluza analysis software, Brea, CA) (13).

### Example 6. Booster Trial in Healthy Adults

The booster study is designed as a single-center, double-blind, randomized, parallel trial to evaluate the safety profile of COH04S1 booster shot and immune response measured by the fold-increase in antibody against SARS-CoV-2 Spike protein at day 28 post-injection among healthy adult volunteers who have previously received a 2-shot mRNA vaccination. A >5-fold increase from baseline in anti-SARS-CoV-2 Spike IgG will be considered as a success in immune response.

Simon's 2 stage minimax design is used to assess which dose level of COH04S1 generates promising immune response after booster injection. 50% immune response rate is considered as lack of interest for further study, and 75% immune response rate as warranting further study. A total of 28 immune response-evaluable participants per arm is needed to have 85% power to detect the promising immune response with a 5% type I error rate.

The participants are stratified by age (18-<55 and 55+ years old) and randomized in a 1:1 ratio to receive COH04S1 at either DL1 or DL2. The interim analysis is performed in each arm independently when the first 11 participants have immune response available (by Ortho VITROS Anti-SARS-CoV-2 IgG Quantitative test at Day 28). If 6 out of 11 participants reach the target immune response, accrual will continue. Otherwise, if <5 out of 11 participants reach the targeted immune response, accrual could be suspended. Accrual will not be stopped when the results of interim analysis are pending due to ongoing evaluation of the immune response among the first 11 participants in an arm. The external DMC will review the interim analysis and decide regarding suspension of accrual. In the final analysis, if 19 out of 28 participants in an arm reach the targeted immune response, the immune response rate in this arm is promising. Otherwise, if s 18 of 28 participants in an arm reach the targeted immune response, the immune response rate is considered to be disappointing. If the true immune response rate is 50% in an arm, there is a 50% chance of suspending accrual early. Otherwise, if the true immune response rate is 75%, the chance of suspending accrual early is only 3.4%.

The same design and immune response boundaries are applied to each arm at the interim analysis and final analysis. If accrual to an arm is suspended at interim analysis or after safety review, or when 28 participants in an arm have immune response available, whichever comes first, randomization will stop. The differences in the immune response rate between 2 arms are not tested. The immune response rate, and other humoral and cellular immune response parameters are estimated in each arm separately. If both arms are found to be safe and worthy of further study based on primary immune response endpoint, the recommendation of a dose level to bring forward into larger trials will consider other parameters such as overall toxicity profile, secondary immune endpoints, and cost of manufacturing.

Data Analysis: The primary toxicity analysis is summarized in terms of type, severity, time of onset, duration, probable association with COH04S1 vaccine and reversibility or outcome. Participants who are unevaluable for MOD/DLT or immune response will be replaced.

The primary immune analysis evaluates fold increase in antibody to SARS CoV-2 Spike protein at day 28 post injection. The mean and 95% CI are calculated if the assumption of normal distribution is not violated. Otherwise, log or other form of transformation will be performed. Histogram charts are generated to show the fold increase. All participants with PIA results available will be included in the primary analysis.

Secondary analyses: Continuous immune response markers are summarized by means or geometric means and standard deviations if the assumption of normal distribution is not violated. Repeated immune response measurements at the multiple time points are analyzed using generalized estimating equations (GEE) or mixed regression models. Scatterplots of immune response markers across time points are generated to visualize the temporal patterns.

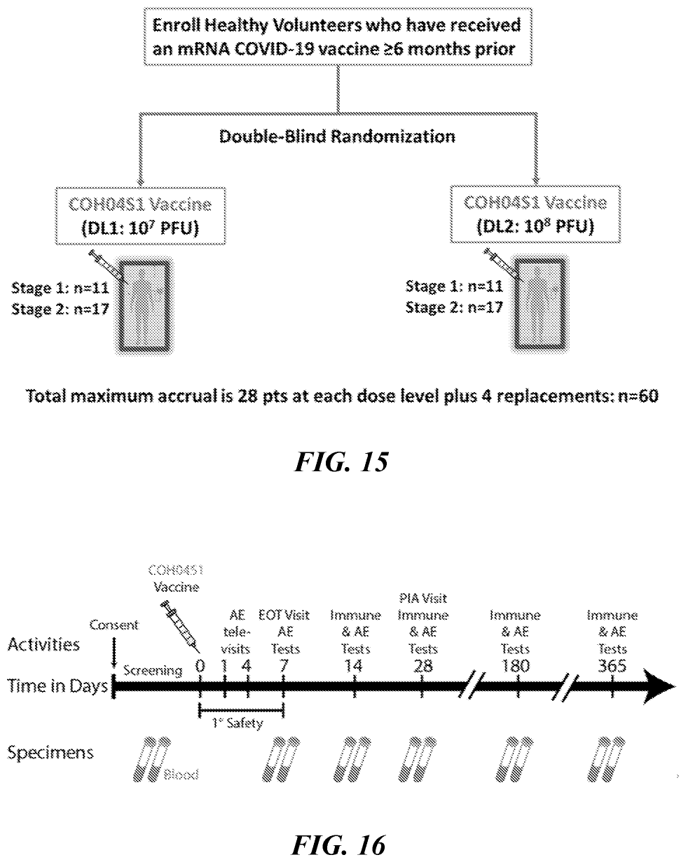

FIG. 15 is a study schema showing randomized parallel trial in healthy adults of 2 doses of COH04S1 as a booster following an initial mRNA SARS-CoV-2 vaccine. Volunteers who have received an mRNA COVID-19 vaccine at least 6 months prior are administered with a single IM booster COH04S1 vaccine injection (1.0×10⁷ PFU/dose or 1.0×10⁸ PFU/dose) of 0.9-1.1 mL volume in the upper arm on study day 0. Participants will be enrolled, treated and followed for a period of 365 days, as shown in FIG. 16.

The humoral immunity (IgA, IgG, and IgM) in serum and saliva are assessed by ELISA. Statistical power is based on positive serum IgG specific for the SARS-CoV-2 S protein after the vaccination. The neutralizing capability of the antibodies to prevent infection of a susceptible cell line are evaluated using a pseudo-type of the SARS-CoV-2 virus carrying the original Wuhan Spike sequence. To evaluate the Th1 vs Th2 polarization of immune responses, a SARS-CoV-2-specific ELISPOT is performed to measure IFN-gamma and IL-4 cytokine levels, by using overlapping peptide libraries specific for SARS-CoV-2. Additionally, functional activated/cycling and memory phenotype marker evolution on the surface of antigen specific T cells elicited as a result of the vaccination are evaluated.

All participants with intercurrent infections are tested for SARS-CoV-2 PCR assay, as a record any incidental COVID-19 infection during the study follow-up period, and the biological correlatives of infected participants are compared with those uninfected, along with recording the severity of disease to evaluate for the potential of vaccine-induced disease enhancement. Additionally, in depth analysis of Th1 and Th2 responses involving multiple cytokines are evaluated in selected samples using intracellular cytokine staining (ICS). Finally, as new variants of concern (VOC) begin circulating in the population, the neutralizing antibodies capable of neutralizing new VOC will be measured in trial participants using SARS-CoV-2 pseudoviruses carrying VOC Spike sequences.

### Example 7: Safety and Immunogenicity of COH04S1 as a Booster Vaccination in Healthy Adults

The objective was to test safety and immunogenicity of COH04S1 given as a single booster dose to healthy adults previously vaccinated with two doses of SARS-CoV-2 mRNA vaccines. Vaccine dose was blinded and randomized between 1×10⁷ pfu (DL1) and 1×10⁸ pfu (DL2). Primary objectives were safety evaluation of a single-dose COH04S1 boost at day 7 post-injection, and evaluation of the fold-increase in antibody against SARS-CoV-2 Spike (S) protein at day 28 post-injection.

COH04S1 was developed as a multiantigen synthetic modified vaccinia Ankara (sMVA) vector that co-expresses Wuhan-Hu-1-based S and nucleocapsid (N) antigens. The N antigen was included in COH04S1 primarily based on the rationale to broaden the stimulation of T cells, which are known to be less susceptible to antigen variation than NAb and therefore considered a critical second line of defense to provide long-term protective immunity against SARS-CoV-2. COH04S1 afforded protection against SARS-CoV-2 ancestral virus and Beta and Delta variants in Syrian hamsters and non-human primates and was safe and immunogenic in a Phase 1 clinical trial in healthy adults. Importantly, T cell responses to both the S and N antigens elicited in COH04S1-vaccinated individuals maintain potent cross-reactivity to SARS-CoV-2 Delta and Omicron BA.1 variants for up to six months post-vaccination, whereas NAb responses elicited by COH04S1, as shown for other COVID-19 vaccines, decrease and confer reduced neutralizing activity against Delta and Omicron BA.1 variants. Baseline characteristics of healthy volunteers are shown in Table 15. COH04S1 is currently being tested in multiple Phase 2 clinical trials in healthy volunteers and in cancer patients.

TABLE 15
Baseline Characteristics of Healthy Volunteers

| | Total |
|---|---|
| | (N = 18) |
| Age at consent, years | |
| N | 16 |
| Median | 50 |
| Interquartile range | 39, 59 |
| Range | (30-64) |
| Age at consent, years | |
| Missing | 2 (11.1%) |
| 18-54 | 10 (55.6%) |
| 55+ | 6 (33.3%) |
| Gender | |
| Female | 11 (61.1%) |
| Male | 7 (38.9%) |
| Ethnicity | |
| Hispanic or Latino | 3 (16.7%) |
| Non-Hispanic or Non-Latino | 8 (44.4%) |
| Non-disclosed | 1 (5.6%) |
| Unknown | 6 (33.3%) |
| Caucasian | 13 (72.2%) |
| Asian | 1 (5.6%) |
| Non-Disclosed | 1 (5.6%) |
| mRNA vaccinated previously | |
| Missing | 2 (11.1%) |
| Moderna | 4 (22.2%) |
| Pfizer | 12 (66.7%) |
| IgG mg/dL at Screening | |
| N | 15 |
| Median | 33 |
| Interquartile range | 11, 100 |
| Range | (2-100) |

Materials. Clinical-grade COH4S1 produced by COH GLP manufacturing facility using CEF cells was used for this Phase 2 clinical trial. Before each injection, COH014S1 was thawed and diluted with sterile diluent (phosphate-buffered saline with 7.5% lactose) to the appropriate dose (DL1=1×10⁷ pfu, DL2=1×10⁸ pfu). Critical materials and reagents are listed in Table 16.

TABLE 16
Critical Materials and Reagents

| Item | Manufacturer | Product ID/Lot # |
|---|---|---|
| Spike peptide library | Genscript | Custom order |
| Spike MegaPool (MP) | LJII | A gift from A. Grifoni/ |
| | | A. Sette (LJII) |
| Nucleocapsid peptide | Genscript | Custom made |
| library | | |
| Membrane peptide library | COH peptide core | Custom made |
| S1 + S2 | SinoBiological | 40589-V08B1 |
| N | SinoBiological | 40588-V08B |
| RBD | SinoBiological | 40592-V08H |
| Goat anti-human IgG-HRP | BioRad | 204005 |
| Natural Goat Serum NGS | Vector Labs | S-1000-500-1 |
| WHO reference panel | NIBSC | 20/268 |
| Hammerstein Casein | Thermo Scientific | J12840-22 |
| TMB-ELISA | Thermo Scientific | 34029 |
| VITROS ® Anti- | OrthoVitros | N/A |
| SARS-CoV-2 IgG | | |
| Quantitative Test | | |
| Spike-D614G expression | Twist Bioscience | Custom order |
| vector | | |
| Spike-Beta expression | Twist Bioscience | Custom order |
| vector | | |
| Spike-Delta expression | Twist Bioscience | Custom order |
| vector | | |
| Spike-Omicron BA.1 | Twist Bioscience | Custom order |
| expression vector | | |
| pALD-Lenti system | Aldevron | pALD-LentiEGFP-K |
| | | (modified to |
| | | express Luciferase) |
| | | pALD-GagPol-K |
| | | pALD-Rev-K |
| HEK293T/17 | ATCC | CRL11268 |
| TransIT-Lenti | Mirus | MIR6600 |
| transfection reagent | | |
| Lenti-XTM p24 Rapid | Takara | |
| Titer Kit | | |
| HEK293T-ACE2 cells | | A gift from J. Bloom |
| | | (Fred Hutchinson |
| | | Cancer Research Center) |
| Viability Dye | Invitrogen | L34975 |
| L/D Near IR | | |
| Anti-CD3 | Biolegend | 300440 |
| Anti-CD4 | BD | 566703 |
| Anti-CD8 | BD | 301040 |
| Anti-CD137 | Biolegend | 309810 |
| Anti-OX40 | Biolegend | 350012 |
| Anti-CD69 | BD | 555531 |
| Anti-CD14/CD19 | Biolegend | 561391/561121 |
| Anti-CD45RA | Biolegend | 304156 |
| Anti-CCR7 | Biolegend | 353236 |
| Human Serum | BioIVT | HP1022HI |
| PHA | Roche | 11082132001 |
| Human IFN-g/IL-4 | Mabtech | X-01A16B-10 |
| FluoroSpot Flex Kit | | |
| Benzonase Nuclease | Santa Cruz | SC-202391A |
| | Biotech | |

FIG. 16 shows the study schema used. COH04S1 was injected as a single booster dose in healthy volunteers. The use of DL1 (1×10⁷ pfu) or DL2 (1×10⁸ pfu) was randomized and blinded to the study participants, the data investigator(s) or data collector(s) and the data analyzer(s). Blood collection for serum and PBMC evaluation was carried out at days 14, 28, 180, and 365 post-vaccination.

SARS-CoV-2 binding antibodies. Binding antibody titers were evaluated by ELISA. ELISA plates were coated overnight with 1 μg/ml of S (S1+S2, 40589-V08B1, SinoBiological), RBD (40592-V08H, SinoBiological), or N (40588-V08B, SinoBiological) in coating buffer (1×PBS pH 7.4). Plates were washed 5 times with 250 μl/well PBST (PBS pH 7.4+0.1% Tween-20). Plates were blocked with 250 μl/well of assay diluent (154 mM NaCl/0.5% Casein/10 mM Tris-HCl/0.1% Tween-20 pH 7.6/8% NGS) for 2 hours at 37° C. Sample dilutions and WHO standards were prepared in assay diluent. Serum samples were diluted 1:150, 1:900, 1:4500, and 1:13500). WHO standards (High, Medium, Low S, Low) were initially diluted 1:9 and then further diluted 1:6. Sample dilutions and WHO standards were added to the plate (100 μl/well) after washing 5× and incubated wrapped in foil at 37° C. and 5% CO₂. After washing 5×, 1:3,000 dilution of HRP-conjugated anti-human IgG secondary antibody was added and incubated for one additional hour at room temperature. After 7× washing, plates were developed using 1-Step Ultra TMB-ELISA for 3 (S), 4 (N), and 5 (RBD) minutes after which the reaction was stopped with 1M H2SO4. Plates were read at 450 nm wavelengths using FilterMax F3 microplate reader. Sample concentration expressed in BAU/ml was extrapolated from the standard curve obtained with the assigned WHO standard values transformed based on the assay dilutions (Table 17).

TABLE 17
WHO standards assigned values and values used
to create standard curve in FilterMax F3

| Antigen | Standard | Cat# | WHO BAU/ul | FilterMax BAU/ml |
|---|---|---|---|---|
| S | High | 20/150 | 832 | 616.296 |
| | Mid | 20/148 | 241 | 178 |
| | Low-S/High-N | 20/144 | 86 | 63.704 |
| | Low | 20/140 | 53 | 39.259 |
| RBD | High | 20/150 | 817 | 605.185 |
| | Mid | 20/148 | 205 | 151.852 |
| | Low-S/High-N | 20/144 | 66 | 48.889 |
| | Low | 20/140 | 45 | 33.33 |
| N | High | 20/150 | 713 | 528.148 |
| | Mid | 20/148 | 295 | 218.519 |
| | Low-S/High-N | 20/144 | 146 | 108.148 |
| | Low | 20/140 | 12 | 8.889 |

SARS-CoV-2 pseudovirus neutralization assay. Serum samples were heat inactivated, diluted using 2-fold serial dilutions in complete DMEM. Diluted serum samples were co-incubated overnight at 4° C. with pseudotyped luciferase lentiviral vector expressing SARS-CoV-2 Spike glycoprotein on the envelope in a poly-L-lysine coated 96-well plate. The amount of pseudovirus was pre-determined based on the target relative luciferase units (RLU) of each variant and ranged between 5×10⁵ and 2×10⁶. Next day, the 96 well plates were allowed to equilibrate to room temperature. HEK293T cells overexpressing ACE-2 receptor were then seeded at a density of 1×10⁵ cells/ml in complete DMEM containing 10 μg/ml of polybrene. The cells were incubated for 48 hours at 37° C. and 5% CO₂ atmosphere. Following incubation, media was aspirated, and the cells lysed in a shaker at room temperature using 40 μl/well of Luciferase Cell Culture Lysis Reagent. Cell lysates were transferred to white 96-well plates and Luciferase activity were measured by sequential injection of 100 μl/well of Luciferase Assay Reagent substrate. RLU were quantified using a microplate reader with injector at a 570 nm wavelength.

IFNγ/IL-4 ELISpot. FluoroSpot plates were prepared by adding 15 μl/well of 35% EtOH for less than a minute. Plates were washed 5× with 200 μl/well of sterile H2O. IFNγ and IL-4 capture antibodies were diluted to 15 μg/ml in sterile PBS and 100 μl/well of antibody were added in each well and incubated overnight at 4° C. PBMCs were thawed, and 1 ml RPMI with benzonase (50 U/ml) was added to the tube. Cells were transferred to a 15 ml conical pre-filled with 12 ml RPMI with benzonase (50 U/ml). Conicals were centrifuged at 300×G for 10 minutes. Media was aspirated and cells resuspended in 12 ml of fresh, warm media without benzonase. Conicals were centrifuged at 300×G for 10 minutes. Cells were resuspended in 2 ml RPMI medium and rested for 2 hours at 37° C./5% CO₂. Coated plates were washed 5× with sterile PBS and 200 μl/well of CTL test medium added to each well and the plate incubated at 37° C./5% CO₂ for at least 30 minutes. Conicals were centrifuged at 300×G for 10 minutes and resuspended in 1 ml CTL test medium. Cells were counted and resuspended to 3×10⁶ cells/ml in CTL test medium. Genscript Spike peptide library consisting of 316 peptides was divided into four sub-libraries: 1S1 (peptides 1-86), 2S1 (87-168), 1S2 (169-242, excluding peptide 173), 2S2 (243-316, excluding peptides 304-309). Peptide dilutions were prepared in CTL test media added with anti-CD28 0.2 μg/ml as shown in Table 18. 50 μl/well of peptide mix were added to the corresponding rows in the FluoroSpot plate. 50 μl/well of cell suspension (1.5×10⁵ cells) were added to the corresponding columns in the FluoroSpot plate. 5×10⁴ cells/wells were added to the PHA controls. Plates were wrapped in foil and incubated 37° C./5% CO₂. After 40-42 hours, plates were washed 5× with PBS. IFNγ and IL-4 detection antibodies were diluted 200× with 0.1% BSA/PBS and sterile filtered (0.22 μm). 100 μl/well of detection antibody in CTL test medium were added to each well and incubated 2 hours at room temperature. Plates were washed 5× with PBS. Fluorophore-conjugated antibodies were diluted 200× in 0.1% BSA/PBS and sterile filtered (0.22 μm). 100 μl/well of detection antibody in CTL test medium were added to each well and incubated 1 hour at room temperature. Plates were washed 5× with PBS. 50 μl/well of fluorescence enhancer were added to each well and incubated 10 minutes at room temperature away from light. Fluorescence enhancer was removed by flicking the plate and plates were dried away from light under the airflow of a biological cabinet. Plates were scanned (490 nm and 550 nm wavelength) and analyzed using ImmunoSpot plate reader.

AIM and T cell memory markers. PBMCs were thawed and counted, and concentration adjusted to 10×10⁶ cells/ml using HR-5 media. 1 million cells (100 μl) were plated in 96 well plates and stimuli were added at a concentration of 2 μg/ml (2×) in 100 ul HR-5 media. Plates were incubated for 24 hours at 37° C./5% CO₂. After the stimulation, plates were spun at 2000 rpm at 4° C. for 5 min. In each well, 50 μl of antibody mix was added to each well (Table 19) and incubated 15 minutes at room temperature in the dark. After incubation, 150 μl PBS were added in each well and plates were spun at 2000 rpm at 4° C. for 3 min. Plates were further washed with 150 μl PBS and spun at 2000 rpm at 4° C. for 3 min. Cells were resuspended in 250 μL of PBS and maintained at 4° C. until acquisition.

TABLE 18
Stimuli dilution

| | | | | Assay | |
|---|---|---|---|---|---|
| | Stock | Dilution | Dilution | Conc. | Volume/ |
| Stimulation | (mg/ml) | (ug/ml) | Factor | (ug/ml) | well (ul) |
| DMSO | | | 2 | | 50 |
| 1S1 | 1 | 2 | 2 | 1 | 50 |
| 2S1 | 1 | 2 | 2 | 1 | 50 |
| 1S2 | 1 | 2 | 2 | 1 | 50 |
| 2S2 | 1 | 2 | 2 | 1 | 50 |
| N | 1 | 2 | 2 | 1 | 50 |
| M | 1 | 2 | 2 | 1 | 50 |
| PHA | 5 | 10 | 2 | 5 | 50 |

TABLE 19
Antibody mix composition

| # | Ab | Fluorochrome | Dilution |
|---|---|---|---|
| 1 | CD3 | FITC | 1:40 |
| 2 | CD45RA | PerCp | 1:20 |
| 3 | CD137 | APC | 1:40 |
| 4 | LIVE/DEAD | NearIR | 1:1000 |
| 5 | CD4 | BV421 | 1:50 |
| Dump channel | CD14 | V500 | 1:100 |
| Dump channel | CD19 | V500 | 1:100 |
| 7 | CD8 | BV605 | 1:100 |
| 8 | CD69 | PE | 1:10 |
| 9 | CCR7 | PE/Dazzle | 1:20 |
| 10 | OX40 | PE-Cy7 | 1:20 |

Statistics. Statistical evaluation was pursued using GraphPad Prism (v8.3.0). Wilcoxon matched-pairs signed rank test was used to compare baseline values to post-vaccination values. As of Nov. 3, 2022, 24 volunteers received a booster dose of COH04S1. Of these, 8 reached day 180 time point. Analysis is provided for a subgroup of samples without stratification by DL given that the trial was blinded.

Immunogenicity results. As of Nov. 3, 2022, 24 volunteers have received a booster dose of COH04S1. Of these, 8 had reached day 180 timepoint. Analysis is provided for a subgroup of samples without stratification by DL given that the trial is still blinded.

SARS-CoV-2 binding antibodies. Most volunteers showed a robust increase in S- and RBD-specific IgG titers after COH04S1 booster dose (FIG. 22). However, in volunteers with a baseline titer approaching and above 103 BAU/ml the increase in IgG titer was more modest indicating a more relevant boosting effect in the presence of lower pre-existing SARS-CoV-2-specific IgG titers. N-specific IgG developed in 8/12 volunteers with 4/12 volunteers not showing an increase or having a titer<1 BAU/ml before and after COH04S1 booster vaccination.

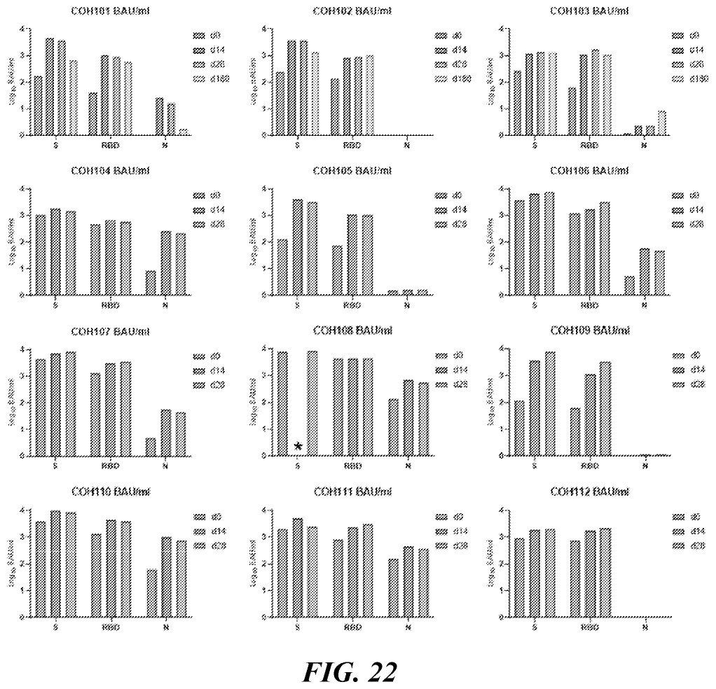

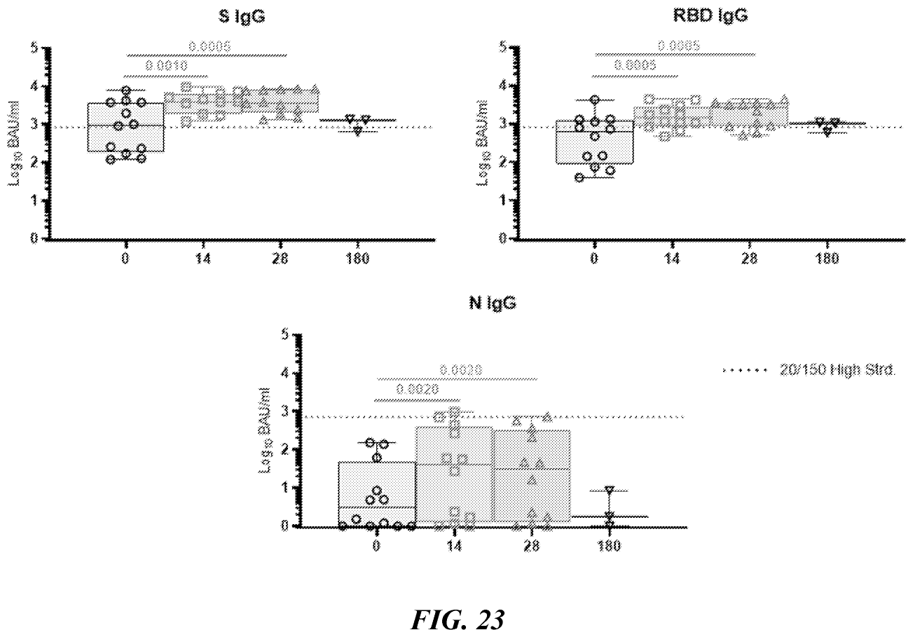

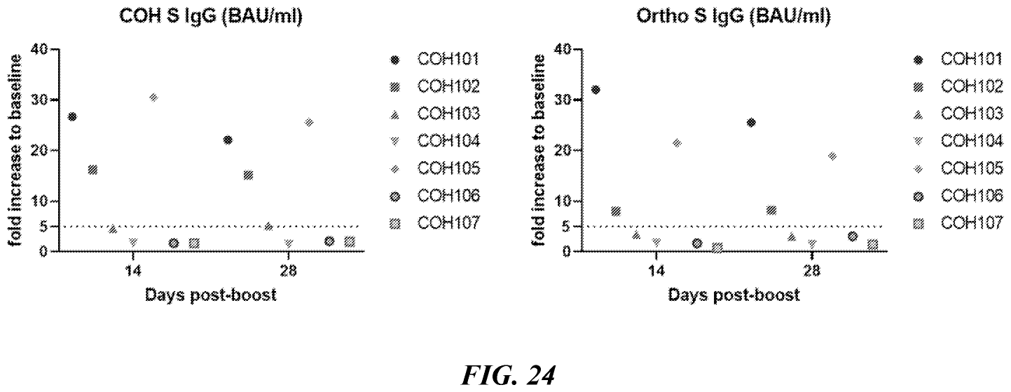

Statistical evaluation revealed a significant increase in S-specific, RBD-specific, and N-specific IgG titers at day 14 and 28 after COH04S1 booster vaccination (FIG. 23). With the primary immunogenicity endpoint being a >5-fold increase in S IgG titers at day 28 compared to baseline as evaluated by OrthoVitros IgG quantitative ELISA, we evaluated the response rates for the available samples using both COH quantitative ELISA and OrthoVitros quantitative ELISA (FIG. 24). Despite minor differences in the fold-increase rate, the two assays were overall concordant. COH ELISA showed that 4/7 volunteers had >5-fold increase in S-specific IgG titers one-month post-booster dose, with COH103 having a borderline 5-fold increase in titer. OrthoVitros quantitative ELISA showed that 3/7 volunteers had a 5-fold increase in S-specific IgG titers one-month post-booster dose. With the exception of COH103, the other “non-responders” had a S-specific IgG baseline titer>103 BAU/ml.

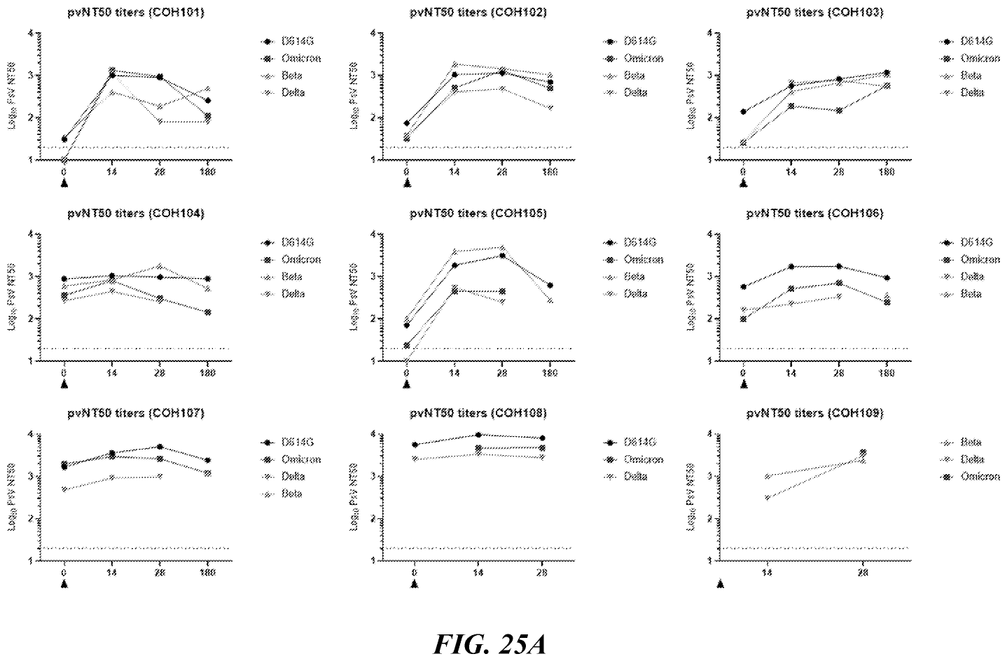

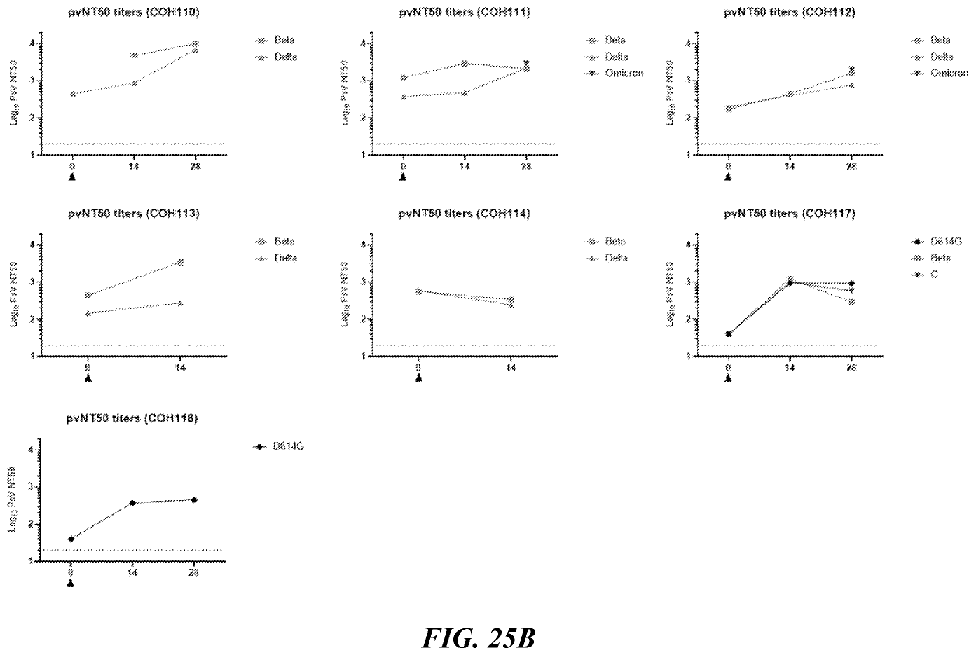

SARS-CoV-2 neutralizing antibodies. Titers of neutralizing antibodies against ancestral SARS-CoV-2 and SARS-CoV-2 Beta, Delta, and Omicron BA.1 VOC were measured in serum of volunteers at baseline and at 14, 28 and 180 days after COH04S1 booster vaccination. In most volunteers, COH04S1 booster vaccination resulted in an increase in NAb titers against SARS-CoV-2 and its VOC compared to baseline, although the increase was less pronounced in volunteers with baseline NT50 around or above 103 (FIG. 25).

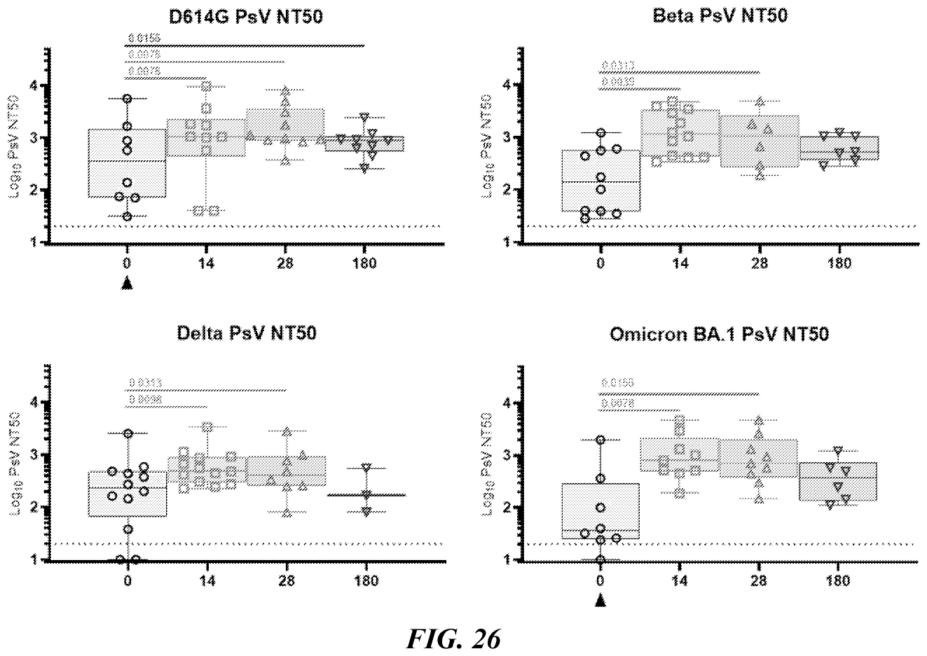

Statistical evaluation revealed a significant increase in SARS-CoV-2 specific NAb titers at 14 and 28 days post-booster vaccination for all the strains evaluated (FIG. 26). Remarkably, median NT50 titers approaching 103 were measured in boosted volunteers against Omicron BA.1 which is known to escape NAb resulting in lower titers. As a confirmation of that, baseline NAb titers against Omicron BA.1 were the lowest across strains with median NT50 around 30 compared to median baseline titers above 100 for ancestral, beta and delta strains. At day 180 post-booster only NAb titers against D614G ancestral strain were significantly higher than at baseline. However, fewer samples were evaluated using the other strains, resulting in a non-significant difference compared to baseline.

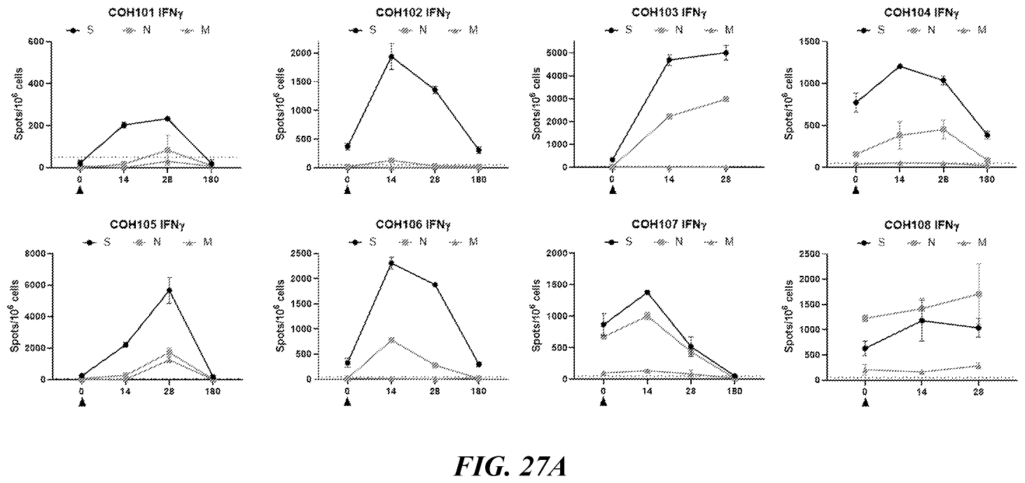

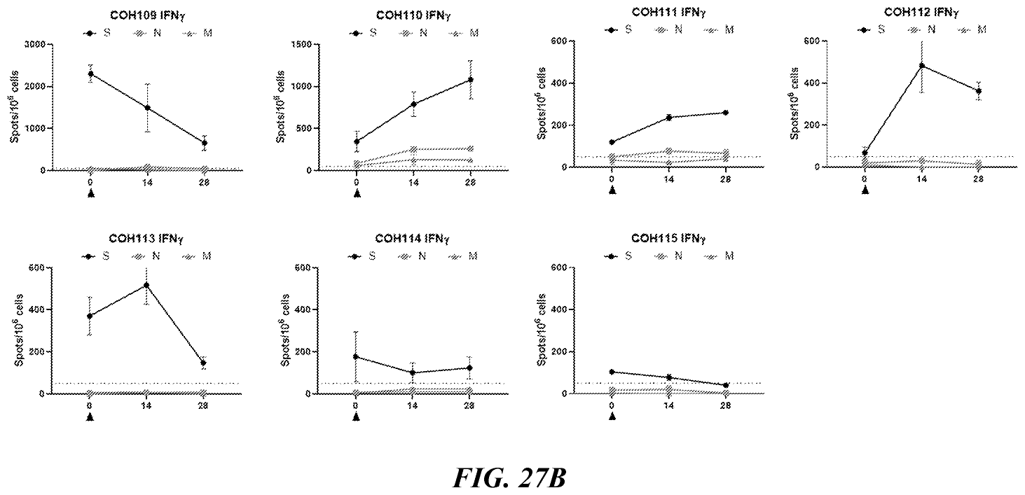

IFNγ/IL-4 T cell responses. T cells secreting IFNγ and/or IL-4 cytokines upon stimulation with SARS-CoV-2 S-, N-, and M-specific peptide libraries were measured in PBMCs from volunteers at 14, 28, and 180 days post-booster vaccination with COH04S1 using FluoroSpot assay. An increase in S- and/or N-specific T cells secreting IFNγ was observed in most volunteers after COH04S1 booster dose (FIG. 27). Most volunteers had a more robust response to S than N, possibly due to pre-existing S-specific T cells following vaccination with a S-based vaccine.

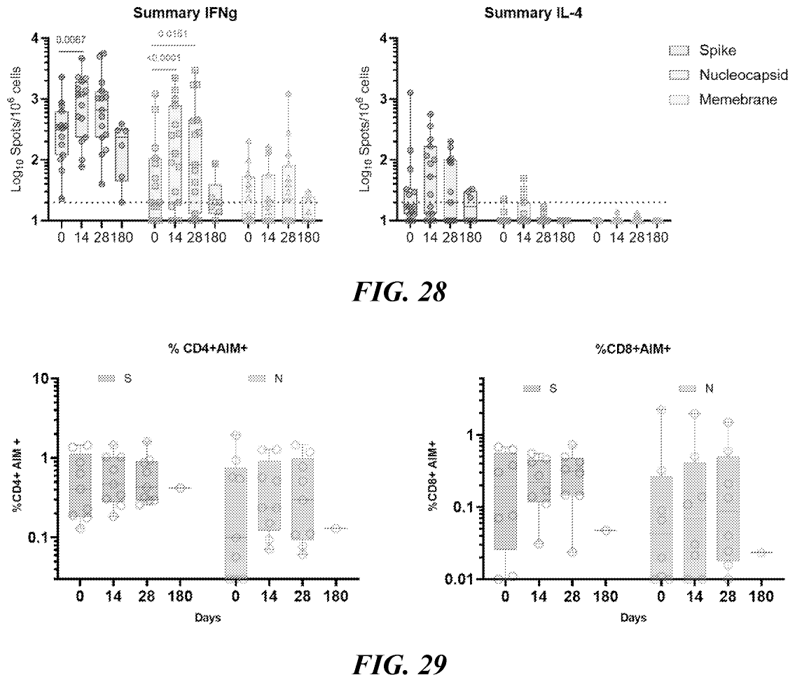

Statistical evaluation indicated that both S- and N-specific T cell responses were significantly elevated after one dose of COH04S1 compared to baseline (FIG. 28). While S-specific T cell responses were significantly elevated at day 14 post-booster dose, N-specific T cell responses were significantly elevated at both 14 and 28 days post-booster dose. IL-4 responses against all antigens were low throughout the study. Both S- and N-specific T cells secreting IL-4 did not significantly increase following vaccination with COH04S1 (FIG. 28).

Activation-induced marker positive T cells. T cells expressing activation induced markers (AIM+) upon stimulation with SARS-CoV-2 S and N peptides were evaluated in samples of COH04S1 boosted volunteers at baseline and at days 14, 28, and 180 post-vaccination. As shown in FIG. 29, no significant increase in S- and N-specific AIM+CD4+ and CD8+ T cells was measured at any of the time points compared to baseline.

The study showed that a single dose booster vaccination with COH04S1 at 1×10⁷ (DL1) or 1×10⁸ (DL2) resulted in a significant increase in S- and N-specific IgG and IFNγ T cells, and SARS-CoV-2 specific NAb.

### REFERENCES

The references, patents and published patent applications listed below, and all references cited in the specification above are hereby incorporated by reference in their entirety, as if fully set forth herein.
1. Krause P R, Fleming T R, Longini I M, et al. SARS-CoV-2 Variants and Vaccines. N Engl J Med 2021; 385(2): 179-86.
2. Edara V V, Pinsky B A, Suthar M S, et al. Infection and Vaccine-Induced Neutralizing-Antibody Responses to the SARS-CoV-2 B.1.617 Variants. N Engl J Med 2021; 385(7): 664-6.
3. Liu Y, Liu J, Xia H, et al. BNT162b2-Elicited Neutralization against New SARS-CoV-2 Spike Variants. N Engl J Med 2021; 385(5): 472-4.
4. Lopez Bernal J, Andrews N, Gower C, et al. Effectiveness of Covid-19 Vaccines against the B.1.617.2 (Delta) Variant. N Engl J Med 2021; 385(7): 585-94.
5. Chiuppesi F, Salazar M D, Contreras H, et al. Development of a multi-antigenic SARS-CoV-2 vaccine candidate using a synthetic poxvirus platform. Nat Commun 2020; 11(1): 6121.
6. Volz A, Sutter G. Modified Vaccinia Virus Ankara: History, Value in Basic Research, and Current Perspectives for Vaccine Development. Adv Virus Res 2017; 97: 187-243.
7. Aldoss I, La Rosa C, Baden L R, et al. Poxvirus Vectored Cytomegalovirus Vaccine to Prevent Cytomegalovirus Viremia in Transplant Recipients: A Phase 2, Randomized Clinical Trial. Ann Intern Med 2020; 172(5): 306-16.
8. Chiuppesi F, Nguyen V H, Park Y, et al. Synthetic Multiantigen MVA Vaccine COH04S1 Protects Against SARS-CoV-2 in Syrian Hamsters and Non-Human Primates. bioRxiv 2021.
9. Frankel P H, Chung V, Tuscano J, et al. Model of a Queuing Approach for Patient Accrual in Phase 1 Oncology Studies. JAMA Netw Open 2020; 3(5): e204787.
10. Goepfert P A, Fu B, Chabanon A L, et al. Safety and immunogenicity of SARS-CoV-2 recombinant protein vaccine formulations in healthy adults: interim results of a randomised, placebo-controlled, phase 1-2, dose-ranging study. Lancet Infect Dis 2021; 21(9): 1257-70.
11. Falsey A R, Frenck R W, Jr., Walsh E E, et al. SARS-CoV-2 Neutralization with BNT162b2 Vaccine Dose 3. N Engl J Med 2021.
12. Haas E J, McLaughlin J M, Khan F, et al. Infections, hospitalisations, and deaths averted via a nationwide vaccination campaign using the Pfizer-BioNTech BNT162b2 mRNA COVID-19 vaccine in Israel: a retrospective surveillance study. Lancet Infect Dis 2021.
13. Wang P, Nair M S, Liu L, et al. Antibody Resistance of SARS-CoV-2 Variants B.1.351 and B.1.1.7. Nature 2021.
14. Planas D, Veyer D, Baidaliuk A, et al. Reduced sensitivity of SARS-CoV-2 variant Delta to antibody neutralization. Nature 2021; 596(7871): 276-80.
15. Fernandez J, Bruneau N, Fasce R, et al. Neutralization of alpha, gamma, and D614G SARS-CoV-2 variants by CoronaVac vaccine-induced antibodies. J Med Virol 2021.
16. Shinde V, Bhikha S, Hoosain Z, et al. Efficacy of NVX-CoV2373 Covid-19 Vaccine against the B.1.351 Variant. N Engl J Med 2021; 384(20): 1899-909.
17. Madhi S A, Baillie V, Cutland C L, et al. Efficacy of the ChAdOx1 nCoV-19 Covid-19 Vaccine against the B.1.351 Variant. N Engl J Med 2021; 384(20): 1885-98.
18. Kustin T, Harel N, Finkel U, et al. Evidence for increased breakthrough rates of SARS-CoV-2 variants of concern in BNT162b2-mRNA-vaccinated individuals. Nat Med 2021; 27(8): 1379-84.
19. Rydyznski Moderbacher C, Ramirez S I, Dan J M, et al. Antigen-Specific Adaptive Immunity to SARS-CoV-2 in Acute COVID-19 and Associations with Age and Disease Severity. Cell 2020; 183(4): 996-1012 e19.
20. Tarke A, Sidney J, Methot N, et al. Impact of SARS-CoV-2 variants on the total CD4(+) and CD8(+) T cell reactivity in infected or vaccinated individuals. Cell Rep Med 2021; 2(7): 100355.
21. Redd A D, Nardin A, Kared H, et al. CD8+ T-Cell Responses in COVID-19 Convalescent Individuals Target Conserved Epitopes From Multiple Prominent SARS-CoV-2 Circulating Variants. Open Forum Infect Dis 2021; 8(7): ofab143.
22. Sekine T, Perez-Potti A, Rivera-Ballesteros O, et al. Robust T Cell Immunity in Convalescent Individuals with Asymptomatic or Mild COVID-19. Cell 2020; 183(1): 158-68 e14.
23. Bange E M, Han N A, Wileyto P, et al. CD8(+) T cells contribute to survival in patients with COVID-19 and hematologic cancer. Nat Med 2021; 27(7): 1280-9.
24. Dutta N K, Mazumdar K, Gordy J T. The Nucleocapsid Protein of SARS-CoV-2: a Target for Vaccine Development. J Virol 2020; 94(13).
25. Ferretti A P, Kula T, Wang Y, et al. Unbiased Screens Show CD8(+) T Cells of COVID-19 Patients Recognize Shared Epitopes in SARS-CoV-2 that Largely Reside outside the Spike Protein. Immunity 2020; 53(5): 1095-107 e3.
26. Dangi T, Class J, Palacio N, Richner J M, Penaloza MacMaster P. Combining spike- and nucleocapsid-based vaccines improves distal control of SARS-CoV-2. Cell Rep 2021; 36(10): 109664.
27. Matchett W E, Joag V, Stolley J M, et al. Cutting Edge: Nucleocapsid Vaccine Elicits Spike-Independent SARS-CoV-2 Protective Immunity. J Immunol 2021; 207(2): 376-9.
28. Flaxman A, Marchevsky N G, Jenkin D, et al. Reactogenicity and immunogenicity after a late second dose or a third dose of ChAdOx1 nCoV-19 in the U K: a substudy of two randomised controlled trials (COV001 and COV002). Lancet 2021; 398(10304): 981-90.
29. Kristiansen P A, Page M, Bernasconi V, et al. WHO International Standard for anti-SARS-CoV-2 immunoglobulin. Lancet 2021; 397(10282): 1347-8.
30. Antoine G, Scheiflinger F, Dorner F, Falkner F G. The Complete Genomic Sequence of the Modified Vaccinia Ankara Strain: Comparison With Other Orthopoxviruses. Virology. 10 May 1998; 244(2):365-96.
31. Scheiflinger F, Dorner F, Falkner F G. Construction of Chimeric Vaccinia Viruses by Molecular Cloning and Packaging. Proceedings of the National Academy of Sciences of the United States of America. 1 Nov. 1992; 89(21):9977-81.
32. Crawford K H D, Eguia R, Dingens A S, Loes A N, Malone K D, Wolf C R, et al. Protocol and Reagents for Pseudotyping Lentiviral Particles with SARS-CoV-2 Spike Protein for Neutralization Assays. Viruses. 2020; 12(5).
33. Tarke A, Sidney J, Kidd C K, Dan J M, Ramirez S I, Yu E D, et al. Comprehensive analysis of T cell immunodominance and immunoprevalence of SARS-CoV-2 epitopes in COVID-19 cases. Cell Rep Med. 2021; 2(2):100204.
34. Aldoss I, La Rosa C, Baden L R, Longmate J, Ariza-Heredia E J, Rida W N, et al. Poxvirus Vectored Cytomegalovirus Vaccine to Prevent Cytomegalovirus Viremia in Transplant Recipients: A Phase 2, Randomized Clinical Trial. Ann Intern Med. 2020; 172(5):306-16.
35. La Rosa C, Longmate J, Martinez J, Zhou Q, Kaltcheva T I, Tsai W, et al. MVA vaccine encoding CMV antigens safely induces durable expansion of CMV-specific T cells in healthy adults. Blood. 2017; 129(1):114-25.
36. Tay, M. Z., et al., The trinity of COVID-19: immunity, inflammation and intervention. Nat Rev Immunol, 2020. 20(6): p. 363-374.
37. Bagarazzi, M. L., et al., Immunotherapy Against HPV16/18 Generates Potent TH1 and Cytotoxic Cellular Immune Responses. Science translational medicine, 2012. 4(155).
38. Ou, X., et al., Characterization of Spike Glycoprotein of SARS-CoV-2 on Virus Entry and Its Immune Cross-Reactivity With SARS-CoV. Nature communications, 27 Mar. 2020. 11(1): p. 1620.
39. Wolfl, M., et al., T-cells in stem cell transplants: dissecting the good, the bad and the ugly. J Stem Cells Regen Med, 2007. 2(1): p. 26.
40. Grifoni, A., et al., Targets of T Cell Responses to SARS-CoV-2 Coronavirus in Humans with COVID-19 Disease and Unexposed Individuals. Cell, 2020. 181(7): p. 1489-1501 e15.

1. A method of vaccinating or protecting a subject against coronavirus disease 2019 (COVID-19) or preventing or reducing the severity of COVID-19 caused by a coronavirus infection or boosting an immune response to coronavirus infection in a subject, comprising administering a composition comprising a synthetic modified vaccinia Ankara (sMVA) vector or virus capable of expressing one or more DNA sequences encoding a spike (S) protein and a nucleocapsid (N) protein or variants or mutants thereof, wherein the method (i) prevents the coronavirus infection or (ii) prevents or reduces the severity of COVID-19 caused by the coronavirus infection.

2-4. (canceled)

5. A method of treating COVID-19 in a subject, comprising administering a composition comprising a synthetic modified vaccinia Ankara (sMVA) vector or virus capable of expressing one or more DNA sequences encoding a spike (S) protein and a nucleocapsid (N) protein or variants or mutants thereof.

6. (canceled)

7. The method of claim 1, wherein the coronavirus infection is caused by the Wuhan-Hu-1 reference strain or a variant of concern (VOC) selected from the group consisting of B.1.1.7 (Alpha), B.1.351 (Beta), P.1 (Gamma), B.1.617.2 (Delta), B.1.621 (Mu), C.37 (Lambda), C.1.2, BA.1 (Omicron), BA.2 (Omicron), BA.2.12.1 (Omicron), BA.4 (Omicron), BA.5 (Omicron), BA.2.75 (Omicron), BQ.1 (Omicron), BQ.1.1 (Omicron), and XBB (Omicron).

8. The method of claim 1, wherein the composition is administered to the subject by intramuscular injection, intranasal instillation, intradermal injection, and/or scarification.

9. The method of claim 1, wherein the composition is administered to the subject in a single dose, two doses, three doses, four doses, or more than four doses.

10. The method of claim 9, wherein the composition is administered to the subject in a single dose.

11. The method of claim 9, wherein the composition is administered to the subject (1) in two doses, wherein one of the doses is a booster dose; (2) in three doses, wherein at least one of the doses is a booster dose; (3) in four doses, wherein at least one of the doses is a booster dose (4) four doses, wherein at least one of the doses is a booster dose.

12-14. (canceled)

15. The method of claim 1, wherein the composition is administered in a prime dose and a first booster dose subsequent to the prime dose.

16. The method of claim 15, wherein the interval between the prime dose and the first booster dose is about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, or more than 16 weeks.

17. The method of claim 1, wherein the composition is administered in a prime dose, a first booster dose subsequent to the prime dose, and two or more additional booster doses subsequent to the first booster dose.

18. The method of claim 17, wherein the interval between each of the booster doses is about 8 weeks, about 9 weeks, about 10 weeks, about 11 weeks, about 12 weeks, about 13 weeks, about 14 weeks, about 15 weeks, about 16 weeks, about 6 months, about 1 year, or more than about 1 year.

19. The method of claim 15, wherein the prime dose or the first and/or additional booster doses are between about 1.0×10⁷ PFU/dose and 5.0×10⁸ PFU/dose, for example, about 1.0×10⁷ PFU/dose, about 1.5×10⁷ PFU/dose, about 2.0×10⁷ PFU/dose, about 2.5×10⁷ PFU/dose, about 3.0×10⁷ PFU/dose, about 3.5×10⁷ PFU/dose, about 4.0×10⁷ PFU/dose, about 4.5×10⁷ PFU/dose, about 5.0×10⁷ PFU/dose, about 5.5×10⁷ PFU/dose, about 6.0×10⁷ PFU/dose, about 6.5×10⁷ PFU/dose, about 7.0×10⁷ PFU/dose, about 7.5×10⁷ PFU/dose, about 8.0×10⁷ PFU/dose, about 8.5×10⁷ PFU/dose, about 9.0×10⁷ PFU/dose, about 9.5×10⁷ PFU/dose, about 1.0×10⁸ PFU/dose, about 1.5×10⁸ PFU/dose, about 2.0×10⁸ PFU/dose, about 2.5×10⁸ PFU/dose, about 3.0×10⁸ PFU/dose, about 3.5×10⁸ PFU/dose, about 4.0×10⁸ PFU/dose, about 4.5×10⁸ PFU/dose, or about 5.0×10⁸ PFU/dose.

20. (canceled)

21. The method of claim 15, wherein the first and/or additional booster doses are the same dosage as the prime dose or at a lower dosage than the prime dose.

22. The method of claim 1, wherein the subject has previously received a different SARS-CoV-2 vaccine.

23. The method of claim 22, wherein the previously received SARS-CoV-2 vaccine is an mRNA-, adenovirus-, or protein-based vaccine.

24. The method of claim 22, wherein the previously received SARS-CoV-2 vaccine is Pfizer-BioNTech COVID-19 vaccine (BNT162b2), Moderna COVID-19 vaccine (mRNA-1273), Janssen COVID-19 vaccine (Ad26.COV2.S), Novavax COVID-19 vaccine adjuvanted, or Oxford-AstraZeneca ChAdOx1 nCoV-19 vaccine (AZD1222).

25. The method of claim 22, wherein the previously received SARS-CoV-2 vaccine comprises an S antigen or a coding sequence for an S antigen only.

26. The method of claim 22, wherein a Th1-biased immune response is elicited in the subject.

27-98. (canceled)

99. The method of claim 17, wherein the prime dose or the first and/or additional booster doses are between about 1.0×10⁷ PFU/dose and 5.0×10⁸ PFU/dose, for example, about 1.0×10⁷ PFU/dose, about 1.5×10⁷ PFU/dose, about 2.0×10⁷ PFU/dose, about 2.5×10⁷ PFU/dose, about 3.0×10⁷ PFU/dose, about 3.5×10⁷ PFU/dose, about 4.0×10⁷ PFU/dose, about 4.5×10⁷ PFU/dose, about 5.0×10⁷ PFU/dose, about 5.5×10⁷ PFU/dose, about 6.0×10⁷ PFU/dose, about 6.5×10⁷ PFU/dose, about 7.0×10⁷ PFU/dose, about 7.5×10⁷ PFU/dose, about 8.0×10⁷ PFU/dose, about 8.5×10⁷ PFU/dose, about 9.0×10⁷ PFU/dose, about 9.5×10⁷ PFU/dose, about 1.0×10⁸ PFU/dose, about 1.5×10⁸ PFU/dose, about 2.0×10⁸ PFU/dose, about 2.5×10⁸ PFU/dose, about 3.0×10⁸ PFU/dose, about 3.5×10⁸ PFU/dose, about 4.0×10⁸ PFU/dose, about 4.5×10⁸ PFU/dose, or about 5.0×10⁸ PFU/dose.

100. The method of claim 15, wherein the first and/or additional booster doses are the same dosage as the prime dose or at a lower dosage than the prime dose.
